(19) United States
(12) Patent Application Publication
(10) Pub. No.: US 2018/0228557 A1
(43) Pub. Date: Aug. 16, 2018

(54) ARTICULATING ROBOTIC PROBES, SYSTEMS AND METHODS INCORPORATING THE SAME, AND METHODS FOR PERFORMING SURGICAL PROCEDURES

(71) Applicants: Ian J. Darisse, Southborough, MA (US); Gabriel A. Johnston, Raynham, MA (US); Richard Walker, Jr., W. Bridgewater, MA (US); Leland Witherspoon, Elk Grove, CA (US); David Warnock, Medfield, MA (US); Thomas Calef, Bridgewater, MA (US); Eric Daley, Franklin, MA (US); Stephen Tully, Quincy, MA (US); Amoz Cruz, Wrentham, MA (US); Jonathan O'Keefe, North Attleboro, MA (US); JEffrey Cerier, Franklin, MA (US); Emily Triggs, Sawston, Cambridge (GB); Simon Grover, Barrington, Cambridge (GB); Andras Pungor, Bridgewater, MA (US); J. Christopher Flaherty, Auburndale, FL (US); R. Maxwell Flaherty, Auburndale, FL (US)

(72) Inventors: Ian J. Darisse, Southborough, MA (US); Gabriel A. Johnston, Raynham, MA (US); Richard Walker, Jr., W. Bridgewater, MA (US); Leland Witherspoon, Elk Grove, CA (US); David Warnock, Medfield, MA (US); Thomas Calef, Bridgewater, MA (US); Eric Daley, Franklin, MA (US); Stephen Tully, Quincy, MA (US); Amoz Cruz, Wrentham, MA (US); Jonathan O'Keefe, North Attleboro, MA (US); JEffrey Cerier, Franklin, MA (US); Emily Triggs, Sawston, Cambridge (GB); Simon Grover, Barrington, Cambridge (GB); Andras Pungor, Bridgewater, MA (US); J. Christopher Flaherty, Auburndale, FL (US); R. Maxwell Flaherty, Auburndale, FL (US)

(73) Assignee: Medrobotics Corporation, Raynham, MA (US)

(21) Appl. No.: 15/567,109

(22) PCT Filed: Apr. 20, 2016
(86) PCT No.: PCT/US16/28374

§ 371 Date: Oct. 17, 2017

Related U.S. Application Data
(60) Provisional application No. 62/150,223, filed on Apr. 20, 2015.
(60) Provisional application No. 62/299,249, filed on Feb. 24, 2016.

(51) Int. Cl. A61B 34/30; A61B 1/05; A61B 1/12; A61B 50/13; A61B 1/00; A61B 1/005; A61B 1/008

(52) U.S. Cl. CPC A61B 34/30; A61B 1/05; A61B 1/126; A61B 50/13; A61B 1/0016; A61B 1/0057; A61B 17/3201; A61B 1/008; A61B 1/00055; A61B 1/00117; A61B 1/00114; A61B 1/00009; A61B 1/00158; A61B 1/00149

(57) ABSTRACT

A system for performing a medical procedure comprises a first assembly and a second assembly. The first assembly comprises an articulating probe assembly and a first housing. The articulating probe assembly comprises an outer probe and an inner probe. The outer probe comprises: multiple articulating outer links and a first connector. The inner probe comprises multiple articulating inner links and a second connector. The first housing comprises: a proximal portion; a distal portion; and an opening positioned in the first housing distal portion. The articulating probe is constructed and arranged to pass through the first housing opening. The second assembly comprises: a first carriage constructed and arranged to operably engage the first connector of the outer probe; a second carriage constructed and arranged to operably engage the second connector of the inner probe; a dual linear drive assembly configured to independently translate the first carriage and the second carriage; and a second housing comprising a proximal portion and a distal portion. The first assembly is constructed and arranged to operably attach to the second assembly. Methods of performing a medical procedure are also described.

### RELATED APPLICATIONS

This application claims the benefit of U.S. Provisional Application No. 62/150,223, filed Apr. 20, 2015, the content of which is incorporated herein by reference in its entirety.

This application claims the benefit of U.S. Provisional Application No. 62/299,249, filed Feb. 24, 2016, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, the content of which is incorporated herein by reference in its entirety.

This application is related to U.S. patent application Ser. No. 14/892,750, filed Nov. 20, 2015, the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, the content of which is incorporated herein by reference in its entirety.

This application is related to U.S. patent application Ser. No. 13/880,525, filed Apr. 19, 2013, now U.S. Pat. No. 8,992,421, the content of which is incorporated herein by reference in its entirety.

This application is related to U.S. patent application Ser. No. 14/587,166, filed Dec. 31, 2014, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US12/40414, filed Jun. 1, 2012, the content of which is incorporated herein by reference in its entirety.

This application is related to U.S. patent application Ser. No. 14/119,316, filed Nov. 21, 2013, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, the content of which is incorporated herein by reference in its entirety.

This application is related to U.S. patent application Ser. No. 13/884,407, filed May 9, 2013, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US12/32279, filed Apr. 5, 2012, the content of which is incorporated herein by reference in its entirety.

This application is related to U.S. patent application Ser. No. 14/008,775, filed Sep. 30, 2013, the content of which is incorporated herein by reference in its entirety.

This application is related to U.S. patent application Ser. No. 14/944,665, filed Nov. 18, 2015, the content of which is incorporated herein by reference in its entirety.

This application is related to U.S. patent application Ser. No. 14/945,685, filed Nov. 19, 2015, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US12/54802, filed Sep. 12, 2012, the content of which is incorporated herein by reference in its entirety.

This application is related to U.S. patent application Ser. No. 14/343,915, filed Mar. 10, 2014, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, the content of which is incorporated herein by reference in its entirety.

This application is related to U.S. patent application Ser. No. 13/812,324, filed Jan. 25, 2013, the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US12/70924, filed Dec. 20, 2012, the content of which is incorporated herein by reference in its entirety.

This application is related to U.S. patent application Ser. No. 14/364,195, filed Jun. 10, 2014, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US13/54326, filed Aug. 9, 2013, the content of which is incorporated herein by reference in its entirety.

This application is related to U.S. patent application Ser. No. 14/418,993, filed Feb. 2, 2015, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US14/10808, filed Jan. 9, 2014, the content of which is incorporated herein by reference in its entirety.

This application is related to U.S. patent application Ser. No. 14/759,020, filed Jan. 9, 2014, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US13/43858, filed Jun. 3, 2013, the content of which is incorporated herein by reference in its entirety.

This application is related to U.S. patent application Ser. No. 14/402,224, filed Nov. 19, 2014, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US13/38701, filed May 20, 2014, the content of which is incorporated herein by reference in its entirety.

This application is related to U.S. patent application Ser. No. 14/888,541, filed Nov. 2, 2015, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US14/36571, filed May 2, 2014, the content of which is incorporated herein by reference in its entirety.

This application is related to U.S. patent application Ser. No. 14/888,189, filed Oct. 30, 2015, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US14/67091, filed Nov. 24, 2014, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US15/34424, filed Jun. 5, 2015, the content of which is incorporated herein by reference in its entirety.

This application is related to U.S. patent application Ser. No. 11/630,279, filed Dec. 20, 2006, published as U.S. Patent Application Publication No. 2009/0171151, the content of which is incorporated herein by reference in its entirety.

### BACKGROUND

As less invasive medical techniques and procedures become more widespread, medical professionals such as surgeons may require articulating surgical tools, such as endoscopes, to perform such less invasive medical techniques and procedures that access interior regions of the body via a body orifice such as the mouth.

### SUMMARY

According to one aspect of the inventive concepts, a system for performing a medical procedure comprises a first assembly and a second assembly. The first assembly comprises an articulating probe assembly and a first housing. The articulating probe assembly comprises an outer probe and an inner probe. The outer probe comprises: multiple articulating outer links; and a first connector. The inner probe comprises multiple articulating inner links; and a second connector; The first housing comprises: a proximal portion; a distal portion; and an opening in the first housing distal portion. The articulating probe is constructed and arranged to pass through the first housing opening. The second assembly comprises: a first carriage constructed and arranged to operably engage the first connector of the outer probe; a second carriage constructed and arranged to operably engage the second connector of the inner probe; a dual linear drive assembly configured to independently translate the first carriage and the second carriage; and a second housing comprising a proximal portion and a distal portion. The first assembly is constructed and arranged to operably attach to the second assembly.

In some embodiments, the first assembly is constructed and arranged to be used in fewer clinical procedures than the second assembly. The first assembly can be constructed and arranged to be used in a single clinical procedure.

In some embodiments, the first assembly comprises a stabilizing element configured to resist deformation of the first housing.

In some embodiments, the second assembly comprises an adaptor portion and a base portion. The base portion can include at least one motor configured to drive at least a portion of the dual linear drive assembly. The base portion can include at least one motor configured to adjust tension or rigidity in the outer probe and/or the inner probe. The adaptor portion can include the dual linear drive assembly. The system can comprise a second adaptor portion which can be attachable to the base portion.

In some embodiments, the second assembly comprises a stabilizing element configured to resist deformation of the first housing.

In some embodiments the stabilizing element comprises one of pins or holes positioned on the second assembly for mating with the other of pins or holes positioned on the first assembly.

In some embodiments, at least one of the first connector or the second connector extends beyond the first housing in a direction of the second housing. The first connector and/or the second connector can extend into the second housing when the first assembly is attached to the second assembly.

In some embodiments, the first connector and the second connector are offset from each other. The first connector and the second connector can be horizontally offset from each other. The first connector and the second connector can be vertically offset from each other.

In some embodiments, the first carriage comprises a connecting portion constructed and arranged to removably engage the first connector. The connecting portion can be configured to rotate. The first carriage can further comprise a retractable projection constructed and arranged to limit rotation of the connecting portion when the projection is in an advanced position. The connecting portion can comprise a keyed geometry constructed and arranged to engage the first connector. The connecting portion keyed geometry can be constructed and arranged to not engage the second connector. The connecting portion and/or the first connector can comprise a ramp element. The connecting portion and/or the first connector can comprise a spring-loaded element. The connecting portion and/or the first connector can comprise an advanceable pin. The connecting portion can comprise a magnetic element configured to cause a magnetic attraction force between the connecting portion and the first connector. The connecting portion and/or the first connector comprises an electromagnet. The connecting portion can be constructed and arranged to further provide a connection selected from the group consisting of: electrical; optical; fluid; and combinations thereof.

In some embodiments, the first carriage is constructed and arranged to vertically receive and engage the first connector. The first assembly can be constructed and arranged to attach to the second assembly when the first carriage and the first connector are in a home position.

In some embodiments, the first carriage is constructed and arranged to horizontally receive and engage the first connector. The first assembly can be constructed and arranged to attach to the second assembly when the first carriage and the first connector are in various relative positions.

In some embodiments, the first carriage is configured to at least advance the outer probe. The first carriage can be further configured to retract the outer probe relative to the opening in the first housing distal portion. The first carriage can be configured to advance and retract the outer probe via the first connector.

In some embodiments, at least one outer cable can be configured to at least one of change a rigidity of the inner probe or to steer the inner probe. The second assembly can comprise a drive assembly constructed and arranged to control the at least one outer cable, and the drive assembly can be configured to retract the outer probe by retracting the at least one outer cable. The first carriage can be configured to advance the first connector.

In some embodiments, the second carriage is constructed and arranged to advance the inner probe. The second carriage can be further configured to retract the inner probe. The second carriage can be configured to advance and retract the inner probe via the second connector.

In some embodiments, at least one inner cable can be configured to at least one of change a rigidity of the inner probe or to steer the inner probe. The second assembly can comprise a drive assembly constructed and arranged to control the at least one inner cable, and the drive assembly can be configured to retract the inner probe by retracting the at least one inner cable. The second carriage can be configured to advance the second connector.

In some embodiments, the system further comprises a proximal latching assembly constructed and arranged to removably attach the first housing proximal portion and the second housing proximal portion. The proximal latching assembly can comprise a magnet. The system can further comprise a distal latching assembly constructed and arranged to removably attach the first housing distal portion and the second housing distal portion.

In some embodiments, the probe assembly further comprises a distal link. The system can further comprise a camera system with a distal portion, and the distal link can be configured to receive the distal portion of the camera system. The distal link can be configured to laterally receive the camera system distal portion. In some embodiments, the distal link includes a camera seat and wherein the distal portion of the camera system is configured to snap-fit into the camera seat.

In some embodiments, the outer probe comprises at least one clip constructed and arranged to operably attach to a cable. The system can further comprise a camera system with a camera system cable, and the outer probe clip can be constructed and arranged to attach to the camera system cable.

In some embodiments, the dual linear drive assembly comprises a component selected from the group consisting of: lead screw; ball screw; hydraulic piston; pneumatic piston; magnetic drive; inch-worm drive; belt drive; and combinations thereof.

In some embodiments, the dual linear drive assembly comprises a first linear drive and a second linear drive, and the first linear drive can be positioned spaced apart in a horizontal direction relative to the second linear drive.

In some embodiments, the system further comprises an introducer comprising a pathway aligned with the first housing opening. The introducer can be constructed and arranged to be attached to the first assembly and/or the second assembly. The introducer can comprise a first portion and a second portion configured to laterally approach and surround the probe assembly. The introducer can be constructed and arranged to be used in multiple clinical procedures. The introducer can be constructed and arranged to be used in more clinical procedures than the first assembly. The introducer can comprise an opening configured to receive a cable that attaches to the outer probe. The introducer can comprise an opening configured to receive a projection from the outer probe. The introducer can comprise at least one tool support. The introducer can comprise two tool supports and a connector positioned between the tool supports. The introducer can comprise a first introducer, and the system can further comprise a second introducer different than the first introducer.

In some embodiments, the system can further comprise at least one tool support. In some embodiments, the system can further comprise an attachment mechanism that removably couples the at least one tool support to the introducer. In some embodiments, the at least one tool support includes two tool supports and the system further comprises a connector positioned between the tool supports.

In some embodiments, the introducer can comprise a first portion and a second portion that couple to each other to redirect the articulating probe assembly relative to the opening of the first housing.

In some embodiments, the system further comprises an electronics module. The electronics module can be positioned in the first assembly and/or the second assembly. The electronics module can comprise a memory storage element. The memory storage element can comprise an EEPROM. The electronics module can be configured to record information selected from the group consisting of: model number; manufacture date; configuration information; probe length information; set-up information; status information; activation information; use information; probe position information; functionality information; and combinations of one or more of these.

In some embodiments, the system further comprises a user interface. The user interface can be configured to send commands to the second assembly to steer, advance and/or retract the inner probe and/or the outer probe. The user interface can comprise a component selected from the group consisting of: joystick; keyboard; mouse; switch; touchscreen; touch pad; trackball; display; touchscreen; audio element; speaker; buzzer; light; LED; and combinations of one or more of these.

In some embodiments, the system further comprises a sterile barrier. The sterile barrier can be configured to surround at least a portion of the first housing and/or the second housing. The system can further comprise a camera cable, and the sterile barrier can be constructed and arranged to not surround the camera cable. The sterile barrier can comprise an opening constructed and arranged to allow the probe assembly to advance through the opening.

In some embodiments, the system further comprises a camera system. The camera system can comprise a distal portion attachable to the outer probe. The outer probe can comprise a distal link with a recess, and the camera system distal portion can be insertable into the distal link recess. The distal link can comprise a camera irrigation channel. The camera system can comprise a camera cable constructed and arranged to be attached to the outer probe and/or the first housing.

In some embodiments, the system further comprises a video processor. The system can further comprise a light, and the video processor can be configured to perform closed loop control of brightness produced by the light. The video processor can be configured to perform a function selected from the group consisting of: adjust tone mapping and/or gamma correction such as to enhance dark regions of an image; provide an unsharp masking filter; enhance edge features; enhance visualization of blood and/or blood vessels; manipulate color balance; manipulate contrast, RGB gamma correction and/or individual RGB gain; automatically rotate image based on probe orientation; digitize and packetize video information; detect a delay by processing a sync signal; enter an alert state; and combinations of one or more of these. The video processor can comprise a PID loop configured to provide an illumination feature. The system can further comprise a camera sensor, and the video processor can be configured to monitor brightness levels by averaging the light collected by the camera sensor.

In an aspect, an articulating probe system comprises: an articulating probe constructed and arranged to articulate in at least one degree of motion and to transition between a flexible state and a rigid state; a feeder assembly in communication with the articulating probe to apply forces on the articulating probe to cause the probe to articulate and to transition between the flexible state and rigid state; a feeder cart on a plurality of wheels that allow manual movement of the cart in a horizontal direction; and a feeder support arm that couples the feeder assembly to the feeder cart.

In some embodiments, at least one of the plurality of wheels comprises a locking wheel.

In some embodiments, the articulating arm includes first and second segments that pivot relative to one another at a pivot joint and wherein one or more pistons are mounted between the first and second segments to support a weight of an upper one of the first and second segments.

In some embodiments, the plurality of wheels comprises caster wheels.

In an aspect, an articulating probe system comprises: an articulating probe constructed and arranged to articulate in at least one degree of motion and to transition between a flexible state and a rigid state, the articulating probe comprising a plurality of links between a proximal link and a distal link; the distal link of the articulating probe including a side port constructed and arranged to receive a distal end of an elongate tool; and a tool support in communication with the articulating probe for supporting the elongate tool, the tool support including a tool tube that extends from the tool support at an intermediate portion to the side port of the distal link at a distal portion, the tool tube having a first flexibility in the intermediate portion and having a second flexibility in the distal portion; the second flexibility being greater in flexibility than the first flexibility.

In some embodiments, the tool tube is circular in cross-section and surrounds a side surface of an inserted tool.

In some embodiments, an intermediate link of the articulating probe between the proximal and distal links includes a side port and wherein the tool tube extends through the side port of the intermediate link between the tool support and the side port of the distal link.

In some embodiments, the distal portion of the tool tube includes rib features of reduced outer diameter.

In some embodiments, the distal portion of the tool tube comprises a material that is different than a material of the intermediate portion.

In some embodiments, the distal portion of the tool tube has a wall thickness that is less than a wall thickness of the intermediate portion.

In an aspect, an articulating probe system comprises: an articulating probe constructed and arranged to articulate in at least one degree of motion and to transition between a flexible state and a rigid state, the articulating probe comprising a plurality of links between a proximal link and a distal link; the distal link of the articulating probe including a side port constructed and arranged to receive a distal end of an elongate tool; and a tool support in communication with the articulating probe for supporting the elongate tool, the tool support including a tool tube that extends from the tool support at an intermediate portion to the side port of the distal link at a distal portion, wherein an intermediate link of the articulating probe between the proximal and distal links includes a side port and wherein the tool tube extends through the side port of the intermediate link between the tool support and the side port of the distal link.

In some embodiments, the tool tube comprises a first flexibility in the intermediate portion and comprises a second flexibility in the distal portion; and the second flexibility being greater in flexibility than the first flexibility.

In some embodiments, the tool tube is circular in cross-section and surrounds a side surface of an inserted tool.

In some embodiments, the tool tube is fixedly attached to the side port of the intermediate link.

In some embodiments, the tool tube slides freely through the side port of the intermediate link.

In an aspect, an articulating probe system comprises: an articulating probe constructed and arranged to articulate in at least one degree of motion and to transition between a flexible state and a rigid state, the articulating probe comprising a plurality of links between a proximal link and a distal link; the distal link of the articulating probe including a side port constructed and arranged to receive a distal end of an elongate tool; and a probe introducer including a neck and a base, a probe channel through the base and neck through which the articulating probe freely passes, the probe channel having an outlet from the base, a tool support coupled to the base of the probe introducer, in communication with the articulating probe for supporting the elongate tool, the tool support having an outlet from the base, wherein an outlet of the probe channel extends a greater distance in a distal direction than the outlet of the tool support.

In some embodiments, the articulating probe system further comprises a flange about the outlet of the probe channel.

In some embodiments, the flange is integral with the base of the probe introducer.

In some embodiments, the flange is coupled to the base of the probe introducer.

In some embodiments, the tool support includes a tool tube that extends from the tool support at an intermediate portion to the side port of the distal link at a distal portion, wherein an intermediate link of the articulating probe between the proximal and distal links includes a side port and wherein the tool tube extends through the side port of the intermediate link between the tool support and the side port of the distal link.

In an aspect, an articulating probe system comprises: an articulating probe constructed and arranged to articulate in at least one degree of motion and to transition between a flexible state and a rigid state, the articulating probe comprising a plurality of links between a proximal link and a distal link; the plurality of links including a channel constructed and arranged to receive a distal end of an elongate tool, a portion of the elongate tool positioned in the channel, a distal end of the elongate tool being fixed to a distal link of the plurality of links; a feeder assembly in communication with the articulating probe to apply forces on the articulating probe to cause the probe to articulate and to transition between the flexible state and rigid state; a portion of the elongate tool being fixedly attached at an attachment position to the feeder assembly, a service loop in the elongate tool provided between attachment position and the channel, wherein a length in the service loop of the elongate tool is greater than a length of the articulating probe when positioned in its greatest extent of curvature.

In some embodiments, the tool comprises a camera and wherein the service loop of the elongate tool comprises an electrical wire.

In some embodiments, the tool comprises a camera and wherein the service loop of the elongate tool comprises a fiber optic.

In some embodiments, the feeder assembly comprises a carriage for driving the articulating probe in a distal direction and wherein a length of the service loop is greater than a combined length of: the length of the articulating probe when positioned in its greatest extent of curvature; and a distance of the carriage when in a greatest extent in the distal direction.

In an aspect, an articulating probe system comprises: an articulating probe constructed and arranged to articulate in at least one degree of motion and to transition between a flexible state and a rigid state, the articulating probe comprising a plurality of links between a proximal link and a distal link, the plurality of links comprising a plurality of inner links and a plurality of outer links; the plurality of links including a channel constructed and arranged to receive a distal end of an elongate tool, a portion of the elongate tool positioned in the channel, a distal end of the elongate tool being fixed to a distal link of the plurality of links; a feeder assembly in communication with the articulating probe to apply forces on the articulating probe to cause the probe to articulate and to transition between the flexible state and rigid state, and to cause one of the plurality of inner links and plurality of outer links to perform a steering and locking operation and the other of the plurality of inner links and plurality of outer links to perform a locking operation; a portion of the elongate tool being fixedly attached at an attachment position to the feeder assembly, a service loop in the elongate tool provided between attachment position and the channel, wherein a length in the service loop of the elongate tool is greater than a length of the one of the plurality of inner links and plurality of outer links during its greatest extent when in the steering operation.

In some embodiments, the tool comprises a camera and wherein the service loop of the elongate tool comprises an electrical wire.

In some embodiments, the tool comprises a camera and wherein the service loop of the elongate tool comprises a fiber optic.

In some embodiments, the feeder assembly comprises a carriage for driving the articulating probe in a distal direction and wherein a length of the service loop is greater than a combined length of: the one of the plurality of inner links and plurality of outer links during its greatest extent when in the steering operation; and a distance of the carriage when in a greatest extent in the distal direction.

In an aspect, an articulating probe system comprises: an articulating probe constructed and arranged to articulate in at least one degree of motion and to transition between a flexible state and a rigid state, the articulating probe comprising a plurality of links between a proximal link and a distal link; a feeder assembly in communication with the articulating probe to apply forces on the articulating probe to cause the probe to articulate and to transition between the flexible state and rigid state; a plurality of cables in communication with the plurality of links; the feeder assembly further comprising: cable bobbins at which proximal ends of the plurality of cables are wound; motor assemblies, each corresponding to one of the cable bobbins, for driving the cable bobbins, the motor assemblies including motion resistance mechanisms that substantially prevent rotation of the bobbins as a result of forces transferred through the cables, as encountered by the articulating probe.

In some embodiments, the motor assemblies comprise: a motor; a gear assembly; and a capstan in communication with the cable bobbin.

In some embodiments, the gear assembly comprises a worm gear assembly.

In some embodiments, the gear assembly comprises at least one of a ratchet and pawl mechanism or a magnetic position holding assembly.

In some embodiments, the motor comprises one of a stepper motor, a closed-loop servomotor, and a DC motor having a shorted drive inductor.

In an aspect, an articulating probe system comprises: an articulating probe constructed and arranged to articulate in at least one degree of motion and to transition between a flexible state and a rigid state, the articulating probe comprising a plurality of links between a proximal link and a distal link, the plurality of links comprising a plurality of inner links and a plurality of outer links; a feeder assembly in communication with the articulating probe to apply forces on the articulating probe to cause the probe to articulate and to transition between the flexible state and rigid state, and to cause one of the plurality of inner links and plurality of outer links to perform a steering and locking operation and the other of the plurality of inner links and plurality of outer links to perform a locking operation; a plurality of steering cables in communication with the one of the plurality of inner links and plurality of outer links; a locking cable in communication with the other of the plurality of inner links and plurality of outer links; the feeder assembly further comprising: cable bobbins at which proximal ends of the plurality of steering cables and a proximal end of the locking cable are wound; motor assemblies, each corresponding to one of the cable bobbins, for driving the cable bobbins, the motor assemblies including motion resistance mechanisms that substantially prevent rotation of the bobbins as a result of forces transferred through the steering cables and locking cables, as encountered by the articulating probe.

In some embodiments, the motor assemblies comprise: a motor; a gear assembly; and a capstan in communication with the cable bobbin.

In some embodiments, the gear assembly comprises a worm gear assembly.

In some embodiments, the gear assembly comprises at least one of a ratchet and pawl mechanism or a magnetic position holding assembly.

In some embodiments, the motor comprises one of a stepper motor, a closed-loop servomotor, and a DC motor having a shorted drive inductor.

In an aspect, an articulating probe system comprises: an articulating probe constructed and arranged to articulate in at least one degree of motion and to transition between a flexible state and a rigid state, the articulating probe comprising a plurality of links between a proximal link and a distal link, a feeder assembly in communication with the articulating probe to apply forces on the articulating probe to cause the probe to articulate and to transition between the flexible state and rigid state; a plurality of cables in communication with the plurality of links; the feeder assembly further comprising: cable bobbins at which proximal ends of the plurality of cables are wound; motor assemblies, each corresponding to one of the cable bobbins, for driving the cable bobbins; motor mounts to which the motor assemblies are mounted, the motor mounts being movably coupled to a chassis of the feeder assembly; load cells in contact with motor mounts for measuring a force applied to the motor mounts.

In some embodiments, the motor assemblies comprise: a motor; a gear assembly; and a capstan in communication with the cable bobbin.

In some embodiments, the load cell measures a force applied to the motor mounts by the cables.

In some embodiments, the feeder assembly further comprises a low-resistance bearing for movably coupling the motor mounts to the chassis of the feeder assembly.

In some embodiments, the feeder assembly further comprises a biasing spring that pre-loads the load cell by applying a biasing force on the motor mounts.

In some embodiments, the feeder assembly further comprises an adjustment screw that ensures contact by the motor mounts against the load cells.

In some embodiments, the feeder assembly further comprises a load cell electronics module for receiving signals generated by the load cell.

In an aspect, an articulating probe system comprises: an articulating probe constructed and arranged to articulate in at least one degree of motion and to transition between a flexible state and a rigid state, the articulating probe comprising a plurality of links between a proximal link and a distal link, the plurality of links comprising a plurality of inner links and a plurality of outer links; a feeder assembly in communication with the articulating probe to apply forces on the articulating probe to cause the probe to articulate and to transition between the flexible state and rigid state, and to cause one of the plurality of inner links and plurality of outer links to perform a steering and locking operation and the other of the plurality of inner links and plurality of outer links to perform a locking operation; a plurality of steering cables in communication with the one of the plurality of inner links and plurality of outer links; a locking cable in communication with the other of the plurality of inner links and plurality of outer links; the feeder assembly further comprising: cable bobbins at which proximal ends of the plurality of steering cables and a proximal end of the locking cable are wound; motor assemblies, each corresponding to one of the cable bobbins, for driving the cable bobbins; motor mounts to which the motor assemblies are mounted, the motor mounts being movably coupled to a chassis of the feeder assembly; load cells in contact with motor mounts for measuring a force applied to the motor mounts.

In some embodiments, the motor assemblies comprise: a motor; a gear assembly; and a capstan in communication with the cable bobbin.

In some embodiments, the load cell measures a force applied to the motor mounts by the cables.

In some embodiments, the feeder assembly further comprises a low-resistance bearing for movably coupling the motor mounts to the chassis of the feeder assembly.

In some embodiments, the feeder assembly further comprises a biasing spring that pre-loads the load cell by applying a biasing force on the motor mounts.

In some embodiments, the feeder assembly further comprises an adjustment screw that ensures contact by the motor mounts against the load cells.

In some embodiments, the feeder assembly further comprises a load cell electronics module for receiving signals generated by the load cell.

In an aspect, an articulating probe system comprises: an articulating probe constructed and arranged to articulate in at least one degree of motion and to transition between a flexible state and a rigid state, the articulating probe comprising a plurality of links between a proximal link and a distal link; a feeder assembly in communication with the articulating probe to apply forces on the articulating probe to cause the probe to articulate and to transition between the flexible state and rigid state; and a position sensor at the feeder assembly to determine whether a change in position of the feeder assembly has occurred.

In some embodiments, the position sensor determines whether a change in at least one of a vertical or horizontal position of the feeder assembly has occurred.

In some embodiments, the position sensor determines whether a change in an orientation of the feeder assembly has occurred.

In some embodiments, the position sensor comprises at least one of an accelerometer, a gyroscope or a position switch.

In some embodiments, the articulating probe system further comprises a control system that, in response to a detection of change in position of the feeder assembly by the position sensor, initiates a calibration procedure of the articulating probe system.

In an aspect, an articulating probe system comprises: an articulating probe constructed and arranged to articulate in at least one degree of motion and to transition between a flexible state and a rigid state, the articulating probe comprising a plurality of links between a proximal link and a distal link, a feeder assembly in communication with the articulating probe to apply forces on the articulating probe to cause the probe to articulate and to transition between the flexible state and rigid state, the feeder assembly comprising: a base assembly including motor assemblies for driving first coupling mechanisms; and a top assembly including second coupling mechanisms in communication with the first coupling mechanisms and for applying the forces on the articulating probe in response to the first coupling mechanisms, the top assembly including the articulating probe, the top assembly removably attachable to the base assembly; and a pivot position between the top assembly and the base assembly about which the top assembly rotates relative to the base assembly during attachment of the top assembly to the base assembly and during removal of the top assembly from the base assembly, the probe at a first position of the feeder assembly and the pivot position at a second position of the feeder assembly, the second position located such that, during removal of the top assembly from the base assembly, the probe of the top assembly moves in an upward direction relative to a patient location.

In some embodiments, during removal of the top assembly from the base assembly, the probe of the top assembly moves in an upward direction relative to a patient location and in a direction away from the patient location.

In some embodiments, during removal of the top assembly from the base assembly, the first coupling mechanisms release from the second coupling mechanisms, thereby releasing forces applied to the articulating probe.

In an aspect, an articulating probe system comprises: an articulating probe constructed and arranged to articulate in at least one degree of motion and to transition between a flexible state and a rigid state, the articulating probe comprising a plurality of links between a proximal link and a distal link, a feeder assembly in communication with the articulating probe to apply forces on the articulating probe to cause the probe to articulate and to transition between the flexible state and rigid state, the feeder assembly comprising: a base assembly including motor assemblies for driving first coupling mechanisms; and a top assembly including second coupling mechanisms in communication with the first coupling mechanisms and for applying the forces on the articulating probe in response to the first coupling mechanisms, the top assembly removably attachable to the base assembly at a seated position; and a sensor constructed and arranged to determine whether the top assembly is in the seated position on the base assembly.

In some embodiments, a portion of the sensor is attached to a handle that secures the top assembly to the base assembly.

In some embodiments, the handle includes a cam that secures the top assembly to a latch on the base assembly.

In some embodiments, the articulating probe further comprises a bumper that provides tactile feedback of proper handle engagement.

In some embodiments, the bumper is coupled to the handle.

In some embodiments, the bumper is coupled to the base.

In some embodiments, the bumper is adjustable in height.

In some embodiments, the sensor comprises a magnet and magnetic field sensor assembly.

In some embodiments, the handle is at the top assembly, wherein the magnet is coupled to the handle and wherein the magnetic field sensor assembly is at the base assembly.

In some embodiments, the magnetic field sensor assembly comprises a filter plate that limits the magnetic field emitted by the magnet to a selective region to further increase precision of the magnetic field sensor assembly.

In an aspect, an articulating probe system comprises: an articulating probe constructed and arranged to articulate in at least one degree of motion and to transition between a flexible state and a rigid state, the articulating probe comprising a plurality of links between a proximal link and a distal link, a feeder assembly in communication with the articulating probe to apply forces on the articulating probe to cause the probe to articulate and to transition between the flexible state and rigid state, the feeder assembly comprising: a base assembly including motor assemblies for driving first coupling mechanisms; and a top assembly including second coupling mechanisms in communication with the first coupling mechanisms and for applying the forces on the articulating probe in response to the first coupling mechanisms, the top assembly including the articulating probe, the top assembly removably attachable to the base assembly, wherein one of the top assembly and base assembly includes a heel and the other of the top assembly and base assembly includes a registration plate at which the top assembly and base assembly interface relative to each other during seating of the top assembly to the base assembly, the heel including a ridge that interfaces with the plate so that the top assembly can rotate slightly about the ridge as it is seated on the base assembly to provide angular play in the seating process.

In some embodiments, the top assembly includes the heel and the base assembly includes the registration plate.

In some embodiments, the articulating probe system further comprises plungers that urge the heel against the plate in a horizontal direction.

In some embodiments, the plungers comprise ball plungers.

In an aspect, an articulating probe system comprises: an articulating probe constructed and arranged to articulate in at least one degree of motion and to transition between a flexible state and a rigid state, the articulating probe comprising a plurality of links between a proximal link and a distal link, a feeder assembly in communication with the articulating probe to apply forces on the articulating probe to cause the probe to articulate and to transition between the flexible state and rigid state, the feeder assembly comprising: a base assembly including motor assemblies for driving first coupling mechanisms; and a top assembly including second coupling mechanisms in communication with the first coupling mechanisms and for applying the forces on the articulating probe in response to the first coupling mechanisms, the top assembly including the articulating probe, the top assembly removably attachable to the base assembly, the top assembly including: a plurality of cables in communication with the plurality of links; and cable bobbin assemblies at which proximal ends of the plurality of cables are wound, the cable bobbin assemblies corresponding to the second coupling mechanisms and comprising: a bobbin shaft coupled to a bobbin plate; a bobbin including a bore, the bobbin constructed and arranged to rotate about the bobbin shaft; a spring between a bottom of the bobbin and the bobbin plate, the spring biased to urge the bobbin in a direction away from the bobbin plate; and an o-ring about the bobbin shaft, the o-ring constructed and arranged to resist rotation of the bobbin about the bobbin shaft when the bobbin is in a first position whereby the o-ring is seated between the bore and the bobbin shaft.

In some embodiments, the o-ring is constructed and arranged to rest above a top of the bobbin to thereby allow free rotation of the bobbin, when the bobbin is in a second position, in engagement with a corresponding first coupling mechanism of the base.

In some embodiments, the o-ring is constructed and arranged to interface with a top of the bobbin to moderately resist rotation of the bobbin, when the bobbin is in a third position, under an upward force of the spring and no longer in engagement with a corresponding first coupling mechanism of the base.

In some embodiments, wherein the first position corresponds with a shipment or installation position of the bobbin, wherein the second position corresponds with an operative position of the bobbin and wherein the third position corresponds with a post-operative position of the bobbin.

In some embodiments, the articulating probe system further comprises grooves on an outer surface of the bobbin for locating the proximal end of the cable.

In some embodiments, the articulating probe system further comprises a cable clip over the bobbin that limits cable movement.

In some embodiments, the articulating probe system further comprises a counter bore on the bobbin shaft in which the o-ring is seated.

In some embodiments, the articulating probe system further comprises a counter bore on the bobbin bore.

In some embodiments, the articulating probe system further comprises a washer between the spring and the bottom of the bobbin

In an aspect, an articulating probe system comprises: an articulating probe constructed and arranged to articulate in at least one degree of motion and to transition between a flexible state and a rigid state, the articulating probe comprising a plurality of links between a proximal link and a distal link, a feeder assembly in communication with the articulating probe to apply forces on the articulating probe to cause the probe to articulate and to transition between the flexible state and rigid state, the feeder assembly comprising: a base assembly including motor assemblies for driving first coupling mechanisms; and a top assembly including second coupling mechanisms in communication with the first coupling mechanisms and for applying the forces on the articulating probe in response to the first coupling mechanisms, the top assembly including the articulating probe, the top assembly removably attachable to the base assembly; a sterile drape constructed and arranged for installation between the base assembly and top assembly, the sterile drape including: a sheet of material; an alignment plate on the sheet of material in alignment with the first and second coupling mechanisms and including pre-formed apertures to operate as pass-throughs for the first and second coupling mechanisms; a removable shield on at least one of the sheet of material in the region of the alignment plate or on the alignment plate or both.

In some embodiments, the removable shield is positioned at a sterile surface of the sheet of material.

In an aspect, a sterile drape constructed and arranged for installation between a base assembly and top assembly of an articulating probe system, the system including an articulating probe, and a feeder assembly including a non-sterile base having first coupling mechanisms and a sterile top assembly including second coupling mechanisms, the sterile top assembly including the probe, the sterile drape including: a sheet of material; an alignment plate on the sheet of material in alignment with the first and second coupling mechanisms and including pre-formed apertures to operate as pass-throughs for the first and second coupling mechanisms; a removable shield on at least one of the sheet of material in the region of the alignment plate or on the alignment plate or both.

In some embodiments, the removable shield is positioned at a sterile surface of the sheet of material.

In an aspect, an articulating probe comprises: a plurality of outer links, each outer link comprising a first longitudinal axis, a concave first surface and a convex second surface opposite the first surface; and an inner link channel along the longitudinal axis in a center region thereof; a plurality of inner links, each inner link comprising a first longitudinal axis, a concave first surface and a convex second surface opposite the first surface; and an opening along the longitudinal axis in a center region thereof; the plurality of inner links being positioned in the inner link channels of the plurality of outer links, and slideable relative to the plurality of outer links.

In some embodiments, the plurality of inner links comprises between 10 and 300 inner links, such as between 50 and 150 inner links, such as between 75 and 95 inner links, such as approximately 84 or 85 inner links.

In some embodiments, the inner links comprise a length between 0.05″ and 1.0″, such as between 0.1″ and 0.5″, such as approximately 0.2″.

In some embodiments, the inner links comprise an effective outer diameter of between 0.1″ and 1.0″, such as an effective outer diameter of between 0.2″ and 0.8″, such as an effective outer diameter of approximately 0.35″.

In some embodiments, the inner links comprise a cable lumen in a central region thereof.

In some embodiments, the inner link cable lumen is of a diameter between 0.01″ and 0.9″, such as a diameter between 0.02″ and 0.3″, such as a diameter of approximately 0.07″.

In some embodiments, the inner link cable lumen comprises an hour-glass profile.

In some embodiments, the concave first surface of the inner links comprises a radius of curvature of between 0.1″ to 1.0″, such as a radius of between 0.3″ and 0.7″, such as a radius of approximately 0.55″.

In some embodiments, the convex second surface of the inner links comprises a radius of curvature of between 0.1″ to 1.0″, such as a radius of between 0.3″ and 0.7″, such as a radius of approximately 0.55″.

In some embodiments, a distal-most inner link of the plurality of inner links comprises a tapered convex surface.

In some embodiments, the tapered convex surface of the distal-most inner link of the plurality of inner links lies at an angle relative to the longitudinal axis that is less than an angle of a taper of the convex surface of other inner links of the plurality of inner links.

In some embodiments, the articulating probe comprises more inner links than outer links, such as at least 10% more inner links than outer links, such as at least 50% more inner links than outer links, such as at least 100% more inner links than outer links, such as at least 200% more inner links than outer links, such as at least 300% more inner links than outer links, such as at least 500% more inner links than outer links.

In some embodiments, the plurality of outer links comprises between 5 and 150 outer links, such as between 10 and 100 outer links, such as between 20 and 80 outer links, such as approximately 46 or 56 outer links.

In some embodiments, the outer links comprise a length between 0.1″ and 2.0″, such as between 0.2″ and 1.0″, such as approximately 0.4″.

In some embodiments, the outer links comprise an effective outer diameter of between 0.2″ and 2.0″, such as an effective outer diameter of between 0.4″ and 1.6″, such as an effective outer diameter of approximately 0.68″.

In some embodiments, the outer links include at least one cable lumen, the cable lumen comprising a diameter between 0.06″ and 0.4″, such as a diameter between 0.01″ and 0.2″, such as a lumen with a minimum diameter of approximately 0.047″.

In some embodiments, the outer link cable lumens comprise an hour-glass profile.

In some embodiments, a plurality of distal-most outer links comprise material of lubricity that is greater than other outer links of the plurality of links

In some embodiments, a plurality of distal-most outer links of greater lubricity comprise between 2 and 10 outer links, such as between 2 and 7 outer links.

In some embodiments, one or more outer links comprise an opaque material.

In some embodiments, the distal-most outer link comprises an opaque material.

In some embodiments, the outer links are configured to articulate in a cascading order, in a direction from distal to proximal, during a steering operation.

In some embodiments, the concave first surface of the outer links comprises a radius of curvature of between 0.1″ to 1.0″, such as a radius of between 0.3″ and 0.8″, such as approximately 0.57″.

In some embodiments, the convex second surface of the outer links comprises a cone with a taper between 5° to 70°, such as a taper of between 10° and 65°, such as a taper of approximately 23°.

In some embodiments, working channels are formed between corresponding recesses at the outer surfaces of the inner links and inner surfaces of the outer links

In some embodiments, working channel recesses of the inner links and/or outer links comprise hour-glass profiles or tapered profiles.

In some embodiments, the hour-glass profile minimizes the maximum diameter of the channel or recess, such as would be necessary if the channel or recess had a single, straight taper.

In some embodiments, the outer links are constructed and arranged such that, during a steering operation whereby the outer links undergo articulation, a distal outer link begins to articulate prior to a next-distal-most outer link, in a cascading articulation arrangement.

In some embodiments, a taper angle of the first concave surface of the outer links is varied from link to link in the distal-most outer links to provide the cascading articulation arrangement.

In some embodiments, variation of the taper angle of the first concave surface of the outer links modifies a mating force between adjacent links to provide the cascading articulation arrangement.

In some embodiments, the taper angle varies from link to link between 10° and 65°, such as increasing from 10° in 1° increments or increasing from 10° in 5° increments.

In some embodiments, a characteristic of the outer links is varied from link to link in the distal-most outer links to provide the cascading articulation arrangement, such as a characteristic selected from the group consisting of: other geometric changes such as a geometric change affecting interface force; material change such as a sequential set of lubricity values that decreases; changes in contacting surface area that cause the desired cascade; and combinations of these.

In an aspect, a method of compensating for extraneous movement in an articulating probe system controlled at a human interface device (HID), comprises: monitoring steering commands as motion presented to the HID by an operator at a sampling rate; integrating the steering commands to produce an integrated command output; and controlling the articulating probe system in response to the integrated steering command.

In some embodiments, the method further comprises applying a scale factor to modify the sampling rate of the monitoring of the steering commands.

In an aspect, a method of calibrating a control system of an articulating probe system having load cells measuring cable tension in cables controlling steering and locking of first and second link systems of the probe system, comprises: rotate a cable motor assembly to slacken a corresponding cable; measure load cell data under “zero-tension” with cable slackened; and initiate operation of the probe including steering and locking of the probe based on the measured load cell data under “zero-tension”.

In some embodiments, the method further comprises determining an orientation of a feeder of the probe system and initiating operation of the probe further in response to the determined orientation.

In some embodiments, the method further comprises performing the calibration operation on a plurality of the cable motor assembly and initiating operation of the probe further in response to multiple measured load cell data under “zero-tension”.

In an aspect, a method of calibrating a control system of an articulating probe system having load cells measuring cable tension in cables controlling steering and locking of first and second link systems of the probe system, comprises: monitor a position of a feeder of the probe system; first determine whether a change in position of the feeder system exceeds a first threshold; in the event the change in position exceeds the first threshold, determine whether a change in position of the feeder system is less than a second threshold; in the event the change in position is less than the second threshold, perform an adjustment of compensation values of the system; and in the event the change in position is greater than the second threshold, initiate a re-calibration of the probe system.

In some embodiments, in the event the change in position is greater than the first threshold and the second threshold, further initiate an alarm signal.

In an aspect, a method of preventing application of excessive force in an articulating probe system having load cells measuring cable tension in cables controlling steering and locking of first and second link systems of the probe system, comprising: measure cable tension during operation using a load cell; in event cable tension is greater than a first threshold amount, initiate an alarm; determine whether a steering mode is currently performed; and in event steering mode is currently performed, determine whether cable tension is greater than a second threshold amount; in event cable tension is greater than a second threshold amount, determine a direction of steering and whether the direction of steering matches a determined curvature of the probe; in event of match, the steering operation is halted; in event of no match tension is released in the cable; following match determination and compensation, cable tension is measured and compared to a third threshold; and in event cable tension is greater than the third threshold amount, initiate an alarm.

In an aspect, a method of preventing unintended motion in an articulating probe system having load cells measuring cable tension in cables controlling steering and locking of first and second link systems of the probe system, comprises: receive a steering command from an operator; assess the steering command for “aggressive” movement based on at least one of velocity or acceleration of movement; and adjust tension of cables in response to assessment.

In one aspect, provided is a robotic introducer system, comprising a first assembly, a second assembly, and a third assembly. The first assembly comprises a cable control assembly. The first assembly is constructed and arranged for use in a plurality of medical procedures. The second assembly comprises a distal link extension assembly, the second assembly constructed and arranged for fewer uses than the first assembly. The third assembly is coupled between the first and second assemblies. The third assembly comprises an articulating probe assembly to which the distal link extension assembly is removably coupled, and which is controlled by the cable control assembly. The third assembly is constructed and arranged for fewer uses than the second assembly.

In an embodiment, the first assembly further comprises a console system.

In an embodiment, the console system comprises a monitor for displaying images related to a medical procedure of the plurality of medical procedures.

In an embodiment, wherein the console system comprises a human interface device (HID).

In an embodiment, the first assembly comprises a base unit to which the third assembly is coupled.

In an embodiment, the cable control assembly is constructed and arranged to control a movement of the articulating probe assembly.

In an embodiment, the first assembly comprises a brace that attaches the first assembly to at least one of a floor, table or other supporting object.

In an embodiment, the first assembly comprises a handle that permits an operator to move the first assembly relative to the at least one of the floor, table or other supporting object.

In an embodiment, the first assembly is not sterilized for use in the plurality of medical procedures.

In an embodiment, the first assembly is coupled to at least two different second assemblies.

In an embodiment, the second assembly comprises at least one tool guide tube.

In an embodiment, the system further comprises at least one tool constructed and arranged to be slidingly received by the at least one tool guide tube.

In an embodiment, the at least one tool comprises a tool selected from the group consisting of: suction device; ventilator; light; camera; grasper; laser; cautery; clip applier scissors; needle; needle driver; scalpel; RF energy delivery device; cryogenic energy delivery device; and combinations thereof.

In an embodiment, the at least one tool is positioned at a patient to perform a medical procedure on the patient.

In an embodiment, the medical procedure comprises a transoral surgery procedure.

In an embodiment, the transoral surgery procedure comprises a resection at or near at least one of a base of a tongue, tonsils, a base of a skull, a hypopharynx, a larynx, a trachea, an esophagus, a stomach, or a small intestine.

In an embodiment, the medical procedure comprises at least one of a single or multiport transaxilla, thoracoscopic, pericardial, laparoscopic, transgastric, transenteric, transanal, or transvaginal procedure.

In an embodiment, the single or multiport transaxilla procedure comprises a laryngectomy.

In an embodiment, the single or multiport thoracoscopic procedure comprises a mediastinal nodal dissection.

In an embodiment, the single or multiport pericardial procedure comprises measuring and treating arrhythmias.

In an embodiment, the single or multiport single or multiport laparoscopic procedure comprises a revision of bariatric lap-band procedures.

In an embodiment, the single or multiport transgastric or transenteric procedure comprises at least one of a cholecystectomy or a splenectomy.

In an embodiment, the single or multiport transanal or transvaginal procedure comprises at least one of a hysterectomy, oophorectomy, cystectomy or colectomy.

In an embodiment, the at least one tool guide tube comprises an outer guide tube and an inner guide tube that is slidingly received by the outer guide tube.

In an embodiment, the at least one tool guide tube is coupled to the distal link extension assembly.

In an embodiment, the distal link extension assembly comprises at least one side port, and in an embodiment, each tool guide tube of the at least one tool guide tube is coupled to a side port of the at least one side port.

In an embodiment, the distal link extension assembly further comprises a first side port coupled to a first tool guide tube and a second side port coupled to a second tool guide tube.

In an embodiment, the at least one side port comprises a working channel.

In an embodiment, the system further comprises a tool extending through the working channel.

In an embodiment, the system further comprises a lighting fiber extending through the working channel that transmits light from a light source.

In an embodiment, the lighting fiber is for a single use.

In an embodiment, the lighting fiber is reusable.

In an embodiment, the distal link extension assembly comprises a camera assembly.

In an embodiment, the distal link extension assembly comprises a distal link body having a central opening that is configured to receive the camera assembly.

In an embodiment, the distal link body comprises a first side port and a second side port extending therefrom.

In an embodiment, each of the first and second side ports comprises a working channel for receiving a tool.

In an embodiment, the camera assembly comprises a lens assembly that generates images of objects related to at least one of the medical procedures.

In an embodiment, the camera assembly comprises a calibration adjustment nut in communication with the lens assembly for providing focus adjustments to a lens of the camera assembly.

In an embodiment, the camera assembly comprises a camera sensor that processes the images.

In an embodiment, the lens assembly comprises a lens barrel comprising an interior region that houses and provides for a precise alignment of one or more optics.

In an embodiment, the lens assembly comprises one or more spacers positioned between two or more of the one or more optics for providing axial and/or radial alignment of the two or more optics.

In an embodiment, the one or more optics include one or more lenses.

In an embodiment, the one or more optics include a polarizing or filtering lens that controls glare, reflected lights from instruments, or other undesirable effects.

In an embodiment, the one or more optics filter infrared (IR) or visible wavelengths.

In an embodiment, the filtering lens is constructed and arranged to allow wavelengths to pass ranging from 400 to 700 nm.

In an embodiment, the filtering lens is constructed and arranged to block infrared wavelengths.

In an embodiment, the filtering lens is constructed and arranged to block ultraviolet wavelengths.

In an embodiment, the filtering lens is constructed and arranged to block LISA laser wavelengths.

In an embodiment, the lens assembly is constructed and arranged for more uses than the second assembly.

In an embodiment, the camera assembly comprises a working channel that extends through the camera assembly.

In an embodiment, the camera assembly is constructed and arranged for more uses than the second assembly.

In an embodiment, the distal link extension assembly further comprises a lighting assembly that outputs electromagnetic radiation.

In an embodiment, the electromagnetic radiation comprises light.

In an embodiment, the lighting assembly comprises a diffusing lens for providing a uniform field of view.

In an embodiment, the lighting assembly comprises a printed circuit board comprising a light source.

In an embodiment, the light source comprises an electron stimulated light source.

In an embodiment, the electron stimulated light source comprises at least one of an electron stimulated luminescence light source, an incandescent light source, an electroluminescent light source, or a gas discharge light source.

In an embodiment, the incandescent light source comprises an incandescent light bulb.

In an embodiment, the gas discharge light source comprises a fluorescent lamp.

In an embodiment, the electroluminescent light source comprises a light-emitting diode (LED).

In an embodiment, the LED is constructed and arranged to produce 1-100 lumens.

In an embodiment, the LED is constructed and arranged to provide a color temperature range between 2700K and 7000K.

In an embodiment, the LED is a multicolor LED.

In an embodiment, the light source comprises a laser light source.

In an embodiment, the laser light source comprises a vertical cavity surface emitting laser (VCSEL).

In an embodiment, the light source comprises at least one optical fiber, which is constructed and arranged to transmit light to and from the lighting assembly.

In an embodiment, the lighting assembly comprises a light source coupled to an optical fiber. In an embodiment, the optical fiber is coupled to a distal lens. In an embodiment, the electromagnetic radiation is output from the light source through the optical fiber to the distal lens.

In an embodiment, the working channel of the distal link extension assembly is constructed and arranged to receive at least one tool.

In an embodiment, the at least one tool comprises a tool selected from the group consisting of: suction device; ventilator; light; camera; grasper; laser; cautery; clip applier; scissors; needle; needle driver; scalpel; RF energy delivery device; cryogenic energy delivery device; and combinations thereof.

In an embodiment, the second assembly further comprises an introduction device that is constructed and arranged to slidingly receive the articulating probe assembly.

In an embodiment, the articulating probe assembly is slidingly positioned in the introduction device.

In an embodiment, the second assembly comprises at least one tool guide tube constructed and arranged to slidingly receive a tool.

In an embodiment, the at least one tool guide tube is directly anchored to the introduction device.

In an embodiment, the second assembly further comprises a base coupled to the introduction device.

In an embodiment, the second assembly further comprises at least one inner guide tube slidingly received by the at least one tool guide tube and anchored to the distal link extension assembly.

In an embodiment, the second assembly further comprises a guide tube support.

In an embodiment, the second assembly further comprises at least one outer guide tube coupled between the guide tube support and the base.

In an embodiment, the guide tube support comprises a dogbone connector.

In an embodiment, the guide tube support comprises a tool entrance opening in communication with the tool guide tube.

In an embodiment, the system further comprises an uninterrupted tool path from the tool entrance opening, the tool guide tube, and a tool exit port of the distal link extension assembly.

In an embodiment, the base comprises a collar that surrounds at least a portion of the introduction device.

In an embodiment, the collar extends in a lateral direction relative to a direction of extension of the introduction device.

In an embodiment, the collar has first and second openings and in an embodiment, first and second outer guide tubes of the tool guide tube are coupled to one side of the first and second openings, and first and second inner guide tubes extend from the first and second outer guide tubes, respectively, at a second side of the first and second openings.

In an embodiment, the second assembly is cleaned, disinfected and/or resterilized between uses.

In an embodiment, the second assembly is coupled to at least two third assemblies over the lifetime of the second assembly.

In an embodiment, the second assembly is coupled to each of the at least two third assemblies in different procedures.

In an embodiment, the articulating probe assembly comprises a plurality of links that are constructed and arranged to facilitate a manipulation of the articulating probe assembly.

In an embodiment, the distal link extension assembly of the second assembly is coupled to a distal connecting link at a distal end of the plurality of links of the articulating probe assembly.

In an embodiment, the third assembly is constructed and arranged for a single use.

In an embodiment, the articulating probe assembly comprises at least one multi-link inner probe and a multi-link outer probe. In an embodiment, the inner and outer probes are steerable by the cable control assembly.

In an embodiment, the third assembly comprises a probe feeder that is coupled to the first assembly for controlling a movement of the articulating probe assembly.

In another aspect, provided is a robotic introducer system, comprising: an articulating probe assembly; a distal link extension assembly coupled to a distal end of the probe assembly; at least one side port extending from the distal link extension assembly, the at least one side port constructed and arranged to receive a tool; and an optical assembly at the distal link extension assembly. The optical assembly comprises a lens providing a first field of view for a user; and an optical redirector that provides a second field of view for the user, the second field of view including a view of the tool received at the at least one side port.

In an embodiment, the second field of view comprises the at least one side port.

In an embodiment, the optical assembly is removably coupled to the probe assembly.

In an embodiment, the optical redirector comprises at least one of a mirror or a prism.

In an embodiment, the at least one side port comprises a first side port constructed and arranged to receive a first tool and a second side port constructed and arranged to receive a second tool.

In an embodiment, the system further comprises a second optical redirector that provides a third field of view for the user.

In another aspect, provided is a robotic introducer system, comprising: an articulating probe assembly; and a distal link extension assembly coupled to a distal end of the articulating probe assembly, the distal link extension assembly including a base; a body movably positioned in the base; an optical lens coupled to the body; and a plurality of body articulating cables extending along the probe assembly and the base that moves the body to change a field of view of the lens when a force is applied to at least one of the cables.

In an embodiment, the articulating probe assembly and the body are independently controllable.

In an embodiment, the articulating probe assembly comprises a plurality of probe links, and in an embodiment, the distal link extension assembly is adjacent a distal link of the plurality of probe links.

In an embodiment, the articulating probe assembly comprises at least one steering cable that terminates at the distal link of the plurality of probe links.

In an embodiment, the at least one steering cable and the plurality of body articulating cables are independently controllable.

In an embodiment, a lower region of the body is convex.

In an embodiment, the base comprises a concave region into which the convex lower region of the body is positioned.

In an embodiment, the convex lower region of the body is a semi-spherical body portion.

In an embodiment, the convex lower region of the body is a semi-ellipsoidal body portion.

In an embodiment, the concave region is a semi-ellipsoidal cavity portion.

In an embodiment, a lower region of the body is concave, and the base comprises a convex region onto which the concave lower region of the body is positioned.

In an embodiment, the body is ball-shaped.

In an embodiment, the system further comprises a plurality of guide holes, each of the plurality of body articulating cables extending through a guide hole of the plurality of guide holes.

In an embodiment, the articulating probe assembly includes a plurality of probe links.

In an embodiment, each of the plurality of probe links comprises a guide hole, and in an embodiment, each of the plurality of guide holes are aligned with each other to receive an articulating body cable.

In an embodiment, the system further comprises a plurality of tubes extending through the plurality of guide holes along the articulating probe assembly that advance and retract with respect to the articulating probe assembly for articulating the probe assembly, a distal end of each of the plurality of tubes coupled to the base.

In an embodiment, the plurality of body articulating cables extend through the plurality of tubes, and move independently of the plurality of tubes.

In an embodiment, the plurality of body articulating cables and the plurality of tubes operate to pan, tilt, or zoom the body.

In an embodiment, the plurality of tubes are spaced equidistantly about the articulating probe assembly.

In an embodiment, the system further comprises a camera assembly positioned in the body, the camera assembly comprising the optical lens.

In another aspect, provided is a method of deploying a robotic introducer system, comprising: providing a first assembly comprising a cable control assembly for use in a plurality of medical procedures; providing a second assembly comprising a distal link extension assembly for fewer uses than the first assembly; coupling a third assembly between the first and second assemblies, the third assembly comprising an articulating probe assembly to which the distal link extension assembly is removably coupled, the third assembly constructed and arranged for fewer uses than the second assembly; and controlling, by the cable control assembly, the articulating probe assembly.

In an embodiment, the method comprises the robotic introducer system including additional features as claimed.

In another aspect, provided is a robotic introducer system as described in reference to the figures.

In another aspect, provided is a method of using a robotics introducer system as described in reference to the figures.

In another aspect, provided is a method of performing a medical procedure as described in reference to the figures.

In one aspect, a tool positioning system comprises an introduction device constructed and arranged to slidingly receive an articulating probe; a first tool support comprising at least one guide element constructed and arranged to slidingly receive a first tool, wherein the first tool support is oriented toward a first operator location; and a second tool support comprising at least one guide element constructed and arranged to slidingly receive a second tool, wherein the second tool support is oriented toward a second operator location.

In some embodiments, at least one of the first tool or the second tool is positioned at a patient to perform a medical procedure on the patient.

In some embodiments, the medical procedure comprises a transoral surgery procedure. In some embodiments, the transoral surgery procedure includes a resection at or near at least one of a base of a tongue, tonsils, a base of a skull, a hypopharynx, a larynx, a trachea, an esophagus, a stomach, or a small intestine.

In some embodiments, the medical procedure includes at least one of a single or multiport transaxilla, thoracoscopic, pericardial, laparoscopic, transgastric, transenteric, transanal, or transvaginal procedure.

In some embodiments, the single or multiport transaxilla procedure includes a laryngectomy.

In some embodiments, the single or multiport thoracoscopic procedure includes a mediastinal nodal dissection.

In some embodiments, the single or multiport pericardial procedure includes measuring and treating arrhythmias.

In some embodiments, the single or multiport single or multiport laparoscopic procedure includes a revision of bariatric lap-band procedures.

In some embodiments, the single or multiport transgastric or transenteric procedure includes at least one of a cholecystectomy or a splenectomy.

In some embodiments, the single or multiport transanal or transvaginal procedure includes at least one of a hysterectomy, oophorectomy, cystectomy or colectomy.

In some embodiments, the first tool support is coupled to the second tool support.

In some embodiments, the first tool support and the second tool support are coupled to each other at a common element.

In some embodiments, a connection at the common element maintains a fixed distance between the first tool support and the second tool support.

In some embodiments, a connection at the common element maintains a fixed orientation between the first tool support and the second tool support.

In some embodiments, the at least one of the first and second tool supports moves linearly relative to the common element.

In some embodiments, the first tool support and second tool support are fixed in position relative to each other.

In some embodiments, positions of the first and second tool supports are maintained during an operation of the tool positioning system.

In some embodiments, the first tool support and second tool support are fixed in orientation relative to each other.

In some embodiments, orientations of the first and second tool supports are maintained during an operation of the tool positioning system.

In some embodiments, at least one of the first tool support or second tool support is rotatable relative to the other.

In some embodiments, at least one of the first tool support or the second tool support is rotatable relative to the other at a common element to which each of the first and second tool supports is coupled.

In some embodiments, at least one of the first and second tool supports is locked in a fixed position relative to the common element.

In some embodiments, the system further comprises a locking mechanism that locks the at least one of the first and second tool supports in the fixed position.

In some embodiments, at least one of the first tool support and the second tool support is directly anchored to the introduction device.

In some embodiments, at least one of the first tool support and second tool support is bonded to the introduction device.

In some embodiments, at least one of the first tool support and second tool support is welded to the introduction device.

In some embodiments, the system further comprises a base, wherein the first tool support and the second tool support are coupled to the base.

In some embodiments, the introduction device is coupled to the base.

In some embodiments, the base comprises a collar that surrounds at least a portion of the introduction device.

In some embodiments, the collar extends in a lateral direction relative to a direction of extension of the introduction device.

In some embodiments, the collar has first and second openings aligned with the first and second tool supports.

In some embodiments, the collar has first and second openings, wherein the first and second tool supports extend through the first and second openings.

In some embodiments, at least one of the first tool support or the second tool support comprises at least one guide element that rotatably engages the base.

In some embodiments, the at least one of the first tool support and the second tool support comprises a gimbal which rotatably engages the at least one guide element at the base.

In some embodiments, the at least one guide element of the first tool support comprises a mid-portion that rotatably engages the base.

In some embodiments, the first tool support rotatably engages the base and the second tool support rotatably engages the base.

In some embodiments, the at least one guide element of the first tool support is fixedly attached to the base.

In some embodiments, the at least one guide element of the first tool support comprises a mid-portion that rotatably engages the base.

In some embodiments, the at least one of the first or second tool supports moves linearly relative to the base.

In some embodiments, the system is constructed and arranged to slidingly receive two tools.

In some embodiments, the system is constructed and arranged to slidingly receive three tools.

In some embodiments, the system is constructed and arranged to slidingly receive four tools.

In some embodiments, the system is constructed and arranged to slidingly receive five or more tools.

In some embodiments, the at least one guide element of the first tool support is constructed and arranged to receive a shaft of the first tool, and wherein the at least one guide element of the second tool support is constructed and arranged to receive a shaft of the second tool.

In some embodiments, the first tool is positioned at a first side of a distal end of the articulating probe and the second tool is positioned at a second side of the distal end of the articulating probe relatively opposite the first side.

In some embodiments, the first tool is controlled by an operator at the first operator location at the first side of the distal end of the articulating probe, and the second tool is controlled by an operator at the second operator location at the second side of the distal end of the articulating probe.

In some embodiments, the first tool and a third tool are positioned at a first side of a distal end of the articulating probe and the second tool and a fourth tool are positioned at a second side of the distal end of the articulating probe relatively opposite the first side.

In some embodiments, the first and third tools are controlled by an operator at the first operator location at the first side of the distal end of the articulating probe, and the second and fourth tools are controlled by an operator at the second operator location at the second side of the distal end of the articulating probe.

In some embodiments, at least one of the first tool support or the second tool support comprises a funnel shaped proximal end.

In some embodiments, at least one guide element of at least one of the first tool support or the second tool support comprises an inner guide element and an outer guide element.

In some embodiments, the outer guide element comprises a first tube and the inner guide element comprises a second tube slidingly positioned in the first tube.

In some embodiments, the inner guide element movably extends from the outer guide element.

In some embodiments, at least a portion of the inner guide element is flexible.

In some embodiments, the system further comprises a third tool support, the third tool support comprising at least one guide element constructed and arranged to slidingly receive a third tool.

In some embodiments, the third tool support is oriented toward the first operator location.

In some embodiments, the system further comprises a connector coupled to the first tool support and the third tool support, wherein the connector is constructed and arranged to maintain a relative position between the first tool support and the third tool support.

In some embodiments, the system further comprises a fourth tool support, the fourth tool support comprising at least one guide element constructed and arranged to slidingly receive a fourth tool.

In some embodiments, the fourth tool support is oriented toward the second operator location.

In some embodiments, the system further comprises a connector coupled to the second tool support and the fourth tool support, wherein the connector is constructed and arranged to maintain a relative position between the second tool support and the fourth tool support.

In some embodiments, the system further comprises a connector coupled to a proximal end of each of the first and third tool supports, and a connector attached to a proximal end of each of the second and fourth tool supports.

In some embodiments, the system further comprises a connector coupled to the first tool support and the second tool support, wherein the connector is constructed and arranged to maintain a relative position between the first tool support and second tool support.

In some embodiments, the connector is rotatably coupled to the first tool support.

In some embodiments, the connector is rotatably coupled to the first tool support and the second tool support.

In some embodiments, the connector is attached to a proximal end of the first and second tool supports.

In some embodiments, the connector extends in a direction that is transverse the directions of extension of proximal ends of the first and second tool supports.

In some embodiments, the system further comprises a fixation point on the connector constructed and arranged to attach to a stabilizing brace.

In some embodiments, the system further comprises a third tool support and a connector coupled to the first, second and third tool supports, wherein the connector is constructed and arranged to maintain a relative position between the first, second, and third tool supports.

In some embodiments, the at least one guide element of the first tool support or the second tool support comprises a hollow elongate member.

In some embodiments, the hollow elongate member comprises a structure selected from the group consisting of: a hollow tube, a coil such as a helical coil, a plastic tube such as a braided plastic tube, and combinations thereof.

In some embodiments, at least a portion of the hollow elongate member is rigid.

In some embodiments, at least a portion of the hollow elongate member is flexible.

In some embodiments, the first operator location and the second operator location comprise side-by-side locations.

In some embodiments, the first tool support is constructed and arranged to provide tool access to a patient's head.

In some embodiments, the first tool support is constructed and arranged to provide tool access to a patient's esophagus.

In some embodiments, the first operator location and the second operator location comprise face-to-face locations.

In some embodiments, the first tool support is constructed and arranged to provide tool access to at least one of a patient chest or a patient abdomen.

In some embodiments, the system further comprises a fixation point constructed and arranged to attach to a stabilizing brace.

In some embodiments, the first tool support comprises the fixation point.

In some embodiments, the system further comprises a connector coupled to the first tool support and the second tool support.

In some embodiments, the connector is constructed and arranged to maintain a relative position between the first tool support and second tool support, wherein the connector comprises the fixation point.

In some embodiments, the introduction device comprises the fixation point.

In some embodiments, the system further comprises a base coupling the first tool support and the second tool support, wherein the base comprises the fixation point.

In some embodiments, the system further comprises a brace attachable to the fixation point.

In some embodiments, the brace is further attachable to a location selected from the group consisting of: a floor, a patient operating table, an articulating probe feeder, and combinations thereof.

In some embodiments, the system further comprises a second fixation point constructed and arranged to attach to a stabilizing brace.

In some embodiments, the system further comprises a first brace for attachment to the first fixation point and a second brace for attachment to the second fixation point.

In some embodiments, the system further comprises the articulating probe.

In some embodiments, the articulating probe comprises a distal link.

In some embodiments, the distal link comprises at least a first sideport coupled to the first tool support and a second sideport coupled to the second tool support.

In some embodiments, the system further comprises a third tool support, wherein the distal link comprises at least a first sideport coupled to the first tool support, a second sideport coupled to the second tool support and a third sideport coupled to the third tool support.

In some embodiments, the first, second and third sideports are symmetrically spaced about a periphery of the distal link.

In some embodiments, the first, second and third sideports are asymmetrically spaced about a periphery of the distal link.

In some embodiments, the first and second sideports are positioned 30° to 180° apart about a periphery of the distal link.

In some embodiments, the system further comprises a fourth tool support wherein the distal link further comprises a fourth sideport coupled to the fourth tool support.

In some embodiments, the system further comprises a fifth tool support wherein the distal link further comprise a fifth sideport coupled to the fifth tool support.

In some embodiments, the system further comprises a controller constructed and arranged to manipulate the articulating probe.

In some embodiments, the system further comprises a first human interface device oriented toward the first operator location, the first human interface generating a first control signal received by the controller for manipulating the articulating probe.

In some embodiments, the system further comprises a tool wherein the tool comprises the first human interface device.

In some embodiments, the system further comprises a second human interface device oriented toward the second operator location and constructed and arranged to generate a second control signal received by the controller for manipulating the articulating probe.

In some embodiments, the system further comprises a tool wherein the tool comprises the second human interface device.

In some embodiments, the system further comprises a connector coupled to the first tool support and the second tool support, wherein the connector is constructed and arranged to maintain a relative position between the first tool support and second tool support, wherein the first human interface device is positioned on the connector.

In some embodiments, the human interface device on the connector communicates with the controller via a wireless connection.

In some embodiments, the system further comprises at least one tool constructed and arranged to be slidingly received by at least one of the first tool support or the second tool support.

In some embodiments, the at least one tool comprises at least two tools, wherein each tool comprises a shaft constructed and arranged to be slidingly received by at least one of the first tool support or the second tool support.

In some embodiments, the at least one tool comprises a tool selected from the group consisting of: a suction device, a ventilator, a light, a camera, a grasper, a laser, a cautery, a clip applier, a scissors, a needle, a needle driver, a scalpel, an RF energy delivery device, a cryogenic energy delivery device, and combinations thereof.

In another aspect, a tool positioning system comprises a first tool support comprising at least one guide element constructed and arranged to slidingly receive a first tool, wherein the first tool support is oriented toward a first operator location; a second tool support comprising at least one guide element constructed and arranged to slidingly receive a second tool, wherein the second tool support is oriented toward a second operator location; and a base that couples the first tool support and the second tool support.

In some embodiments, the system further comprises an introduction device coupled to the base.

In some embodiments, the base comprises a collar that surrounds at least a portion of the introduction device.

In some embodiments, the collar extends in a lateral direction relative to a direction of extension of the introduction device.

In some embodiments, the collar has first and second openings aligned with the first and second tool supports.

In some embodiments, the collar has first and second openings, wherein the first and second tool supports extend through the first and second openings.

In some embodiments, at least one of the first tool support or the second tool support comprises at least one guide element that rotatably engages the base.

In some embodiments, the at least one of the first tool support and the second tool support comprises a gimbal which rotatably engages the at least one guide element at the base.

In some embodiments, the least one guide element of the first tool support comprises a mid-portion that rotatably engages the base.

In some embodiments, the first tool support rotatably engages the base and the second tool support rotatably engages the base.

In some embodiments, the at least one guide element of the first tool support is fixedly attached to the base.

In some embodiments, the at least one guide element of the first tool support comprises a mid-portion that rotatably engages the base.

In another aspect, a tool positioning system comprises a first tool support comprising at least one first guide element constructed and arranged to slidingly receive a first tool; a second tool support comprising at least one second guide element constructed and arranged to slidingly receive a second tool; and a first connector attached to the first tool support and the second tool support, wherein the connector is constructed and arranged to maintain a distance between the first tool support and second tool support.

In some embodiments, the first connector is fixedly attached to at least the first tool support or the second tool support.

In some embodiments, the first connector is rotatably attached to at least the first tool support or the second tool support.

In some embodiments, the system further comprises a gimbal which rotatably engages the at least one first or second guide element at the base.

In some embodiments, the first connector comprises a first opening and a second opening each constructed and arranged to operably engage a tool support of the first and second tool supports.

In some embodiments, the first opening and the second opening are constructed and arranged to position the first tool support and the second tool support in a non-parallel configuration.

In some embodiments, at least one of the first opening or the second opening comprises a funnel-shaped opening.

In some embodiments, the first connector further comprises a third opening constructed and arranged to operably engage a third tool support.

In some embodiments, a single operator operates a tool extending from each of the first, second, and third tool supports from an operator location.

In some embodiments, the first connector comprises a rigid structure.

In some embodiments, the first connector comprises at least a portion that is flexible.

In some embodiments, the first connector comprises an operator shapeable structure.

In some embodiments, the first connector comprises a malleable structure.

In some embodiments, the first connector comprises a hinged portion.

In some embodiments, the first connector is constructed and arranged to be shaped after at least one of the application of heat or the removal of heat.

In some embodiments, the first connector is constructed and arranged to be attachable to at least one of the first tool support or the second tool support.

In some embodiments, the first connector is constructed and arranged to be detachable to at least one of the first tool support or the second tool support.

In some embodiments, the system further comprises a second connector attachable to the first tool support and the second tool support, wherein the second connector is constructed and arranged to maintain a relative position between the first tool support and the second tool support.

In some embodiments, the first connector is constructed and arranged to position the first tool support and the second tool support in a first geometry, and the second connector is constructed and arranged to position the first tool support and the second tool support in a second geometry different than the first geometry.

In some embodiments, the first connector differs from the second connector by at least one of length, shape or curvature.

In some embodiments, the system further comprises a third tool support comprising at least one guide element constructed and arranged to slidingly receive a shaft of a tool.

In some embodiments, the first connector further maintains a position of the third tool support relative to the first tool support and the second tool support.

In some embodiments, the system further comprises a fourth tool support comprising at least one guide element constructed and arranged to slidingly receive a shaft of a tool.

In some embodiments, the system further comprises a second connector constructed and arranged to maintain a relative position between the second tool support and the fourth tool support, wherein the first connector is constructed and arranged to maintain a relative position between the first tool support and the third tool support.

In some embodiments, a single operator operates a tool extending from each of the first, second, and third tool supports from an operator location,

In some embodiments, a first operator operates tools extending from two of the first, second, and third tool supports, and a second operator operates a tool extending from the other of the first, second, and third tool supports.

In some embodiments, the first connector can be removably coupled to the first and second tool supports.

In some embodiments, the first connector is replaced with a third connector having different dimensions than the first connector.

In some embodiments, the inventive concepts comprise an articulating probe as described in reference to the figures.

In some embodiments, the inventive concepts comprise a surgical tool as described in reference to the figures.

In some embodiments, the inventive concepts comprise a controller as described in reference to the figures.

In some embodiments, the inventive concepts comprise a method of controlling a robotic system as described in reference to the figures.

In some embodiments, the inventive concepts comprise a human interface device as described in reference to the figures.

In some embodiments, the inventive concepts comprise a method of performing a medical procedure as described in reference to the figures.

An introduction assembly for an articulated probe, comprising: a feeding mechanism having at least one actuator for controlling the articulated probe; and an introduction device having a proximal end fixed in a positional relationship to the feeding mechanism, wherein the introduction device is configured to receive the articulated probe and provide a supporting force to the articulated probe.

In some embodiments, the introduction device is further configured to guide the articulated probe into a region of interest.

In some embodiments, the region of interest is selected from the group consisting of: the esophagus; the gastrointestinal tract; the pericardial space; the peritoneal space; and combinations thereof.

In some embodiments, the introduction device is connected to the feeding mechanism.

In some embodiments, the introduction device is configured to be disconnected from the feeding mechanism.

In some embodiments, the introduction device further comprises: a support member configured to support the articulated probe; an entrance positioned at the proximal end of the support member configured to guide the articulated probe into proximity with the support member; and an exit positioned at a distal end of the support member configured to guide the articulated probe from the support member into a region of interest.

In some embodiments, the introduction assembly further comprises a tool shaft guide.

In some embodiments, the tool shaft guide is configured to performing one or more of the following functions: slidingly receive a shaft of a tool; guide the shaft of a tool; provide a supporting force for a tool; and combinations thereof.

In some embodiments, the introduction assembly further comprises a collar attaching the tool shaft guide to the introduction device.

In some embodiments, the tool shaft guide is rotatably attached to the introduction device.

In some embodiments, the tool shaft guide is rotatably attached to the introduction device with one degree of freedom.

In some embodiments, the tool shaft guide is rotatably attached to the introduction device with multiple degrees of freedom.

In some embodiments, the introduction assembly further comprises a second tool shaft guide.

In some embodiments, the first tool shaft guide comprises a first geometry and the second tool shaft guide comprises a second geometry different than the first geometry.

In some embodiments, the tool shaft guide comprises multiple coaxial tubes.

In some embodiments, the tool shaft guide comprises a first tube comprising a first rigidity and a second tube comprising a second rigidity different than the first rigidity.

In some embodiments, the first tube slidingly receives the second tube.

In some embodiments, the first tube rigidity is greater than the second tube rigidity.

In some embodiments, the tool shaft guide comprises a proximal end and a tapered opening positioned on the proximal end.

In some embodiments, the tool shaft guide comprises a first portion and a second portion.

In some embodiments, the tool shaft guide further comprises a joint connecting the first portion and the second portion.

In some embodiments, the joint is selected from the group consisting of: a spherical joint; a hinged joint; and combinations thereof.

In some embodiments, the tool shaft guide comprises a bend portion constructed and arranged to allow an operator to modify the geometry of the tool shaft guide.

In some embodiments, the bend portion comprises a plastically deformable material.

In some embodiments, the articulated probe comprises a plurality of proximal links and a plurality of distal links.

In some embodiments, the plurality of proximal links and plurality of distal links are outer links.

In some embodiments, at least one of the plurality of proximal links comprises a first diameter, and at least one of the plurality of distal links comprises a second diameter, wherein the first diameter is less than the second diameter.

In some embodiments, the plurality of distal links are constructed and arranged to remain external to the introduction device.

In some embodiments, the introduction device comprises a distal end, and wherein one or more of the plurality of proximal links are constructed and arranged to pass through the introduction device distal end.

In another aspect, an introduction device for an articulated probe comprises: a support member configured to support an articulated probe; an entrance positioned at a proximal end of the support member configured to guide the articulated probe into proximity with the support member; and an exit positioned at a distal end of the support member configured to guide the articulated probe from the support member into a surrounding environment.

In some embodiments, the surrounding environment is selected from the group consisting of: the esophagus; the gastrointestinal tract; the pericardial space; the peritoneal space; and combinations thereof.

In some embodiments, the proximal end is configured to be attached to a feeding mechanism, and the entrance is configured to guide the articulated probe from the feeding mechanism into proximity with the support member.

In some embodiments, the proximal end is configured to be integral with the distal end of the feeding mechanism.

In some embodiments, the proximal end is configured to be removably attached to the distal end of the feeding mechanism.

In some embodiments, the distal end is configured to be inserted into a lumen.

In some embodiments, the lumen comprises a lumen of a patient's body.

In some embodiments, the support member comprises a rigid material.

In some embodiments, the support member comprises a flexible material.

In some embodiments, the support member comprises an axially curved member.

In some embodiments, the support member comprises a cylindrical tube.

In some embodiments, an inner diameter of the support member is larger than the outer diameter of the articulated probe.

In some embodiments, the support member comprises a first surface and a second surface.

In some embodiments, the first surface faces the second surface.

In some embodiments, a cross section perpendicular to the first surface and the second surface is substantially a circle.

In some embodiments, the support member surrounds a lumen.

In some embodiments, the clamp is selected from the group consisting of: a lever, a cam, an expandable member such as a balloon; a piston such as a hydraulic or pneumatic piston; an electromagnetically activated actuator such as a solenoid; and combinations thereof.

In some embodiments, the clamp is configured to prevent the articulated probe from moving in one or more of the following ways: movement in a radial direction; movement in an axial direction; rotation; and combinations thereof.

In some embodiments, the support member surrounds a lumen.

In some embodiments, the inner diameter of the support member is larger than the outer diameter of the articulated probe.

In some embodiments, the clamp comprises a balloon configured to controllably expand and apply pressure on an outer surface of the articulated probe, such that the articulated probe can be stabilized in an axial direction; stabilized in a radial direction; and/or stabilized to prevent rotation relative to the introduction device.

In some embodiments, the clamp is configured to transmit a force between the support member and the articulated probe, said force applied to a surface area of the articulated probe of at least one square millimeter.

In some embodiments, the clamp is configured to transmit a force between the support member and the articulated probe, said force applied to a surface area of the articulated probe of at least ten square millimeters.

In some embodiments, the clamp is configured to transmit a force between the support member and the articulated probe, said force applied to a surface area of the articulated probe of at least one hundred square millimeters.

In some embodiments, the introduction device further comprises at least one channel extending at least partially along a longitudinal axis of the support member.

In some embodiments, the at least one channel comprises two or more channels.

In some embodiments, the two or more channels are positioned equidistantly apart on the introduction device.

In some embodiments, the at least one channel is constructed and arranged to slidingly receive the shaft of one or more tools.

In some embodiments, the at least one channel comprises a curvilinear channel.

In some embodiments, the introduction device further comprises a tool shaft guide.

In some embodiments, the tool shaft guide is configured to perform one or more of the following functions: slidingly receive a shaft of a tool; guide the shaft of a tool; provide a supporting force for a tool; and combinations thereof.

In some embodiments, the introduction device further comprises a collar attaching the tool shaft guide to the introduction device.

In some embodiments, the tool shaft guide is rotatably attached to the introduction device.

In some embodiments, the tool shaft guide is rotatably attached to the introduction device with one degree of freedom.

In some embodiments, the tool shaft guide is rotatably attached to the introduction device with multiple degrees of freedom.

In some embodiments, the introduction device further comprises a second tool shaft guide.

In some embodiments, the first tool shaft guide comprises a first geometry and the second tool shaft guide comprises a second geometry different than the first tool shaft guide geometry.

In some embodiments, the tool shaft guide comprises multiple coaxial tubes.

In some embodiments, the tools shaft guide comprises a first tube comprising a first rigidity and a second tube comprising a second rigidity different than the first rigidity.

In some embodiments, the first tube slidingly receives the second tube.

In some embodiments, the first tube rigidity is greater than the second tube rigidity.

In some embodiments, the tool shaft guide comprises a proximal end and a tapered opening positioned on the proximal end.

In some embodiments, the tool shaft guide comprises a first portion and a second portion.

In some embodiments, the tool shaft guide further comprises a joint connecting the first portion and the second portion.

In some embodiments, the joint is selected from the group consisting of: a spherical joint; a hinged joint; and combinations thereof.

In some embodiments, the tool shaft guide comprises a bend portion constructed and arranged to allow an operator to modify the geometry of the tool shaft guide.

In some embodiments, the bend portion comprises a plastically deformable material.

In some embodiments, the articulated probe comprises a plurality of proximal links and a plurality of distal links.

In some embodiments, the plurality of proximal links and plurality of distal links are outer links.

In some embodiments, at least one of the plurality of proximal links comprises a first diameter, and at least one of the plurality of distal links comprises a second diameter, wherein the first diameter is less than the second diameter.

In some embodiments, the plurality of distal links are constructed and arranged to remain external to the introduction device.

In some embodiments, the introduction device comprises a distal end, and wherein one or more of the plurality of proximal links are constructed and arranged to pass through the introduction device distal end.

In some embodiments, the introduction device further comprises at least one tool channel on an outer surface of the introduction device and extending along a longitudinal axis of the introduction device, configured to guide a filament into a probe side port located on an outer surface of an articulated probe.

In some embodiments, the tool channel comprises a shaft connected to a tool port positioned on an outer surface of the introduction device. In some embodiments, the at least one tool channel comprises a closed ring configured to slidingly receive the filament.

In some embodiments, the at least one tool channel comprises a ring and a slot in said ring, wherein the slot is configured to receive the filament.

In some embodiments, the slot is further configured to release the filament.

In another aspect, a method of introducing an articulated probe to a region of interest comprises: providing a support member configured to support an articulated probe and having a proximal end with an entrance and a distal end with an exit; inserting the support member into the region of interest; inserting the articulated probe into the entrance; and extending the articulated probe out of the exit such that a distal end of the articulated probe leaves the support member and enters the region of interest.

In some embodiments, inserting the articulated probe into the entrance is performed prior to inserting the support member into the region of interest.

In some embodiments, the method further comprises advancing a distal end of the articulated probe to a location proximate the exit prior to inserting the support member into the region of interest.

In some embodiments, the distal end of the articulated probe is advanced while the probe is in a flexible state.

In some embodiments, the distal end of the articulated probe is advanced manually.

In some embodiments, the distal end of the articulated probe is advanced by transitioning an outer sleeve of the articulated probe between a rigid state and a flexible state. In some embodiments, the method further comprises: providing a feeding mechanism, wherein the proximal end is configured to be fixed in a positional relationship to the feeding mechanism, and wherein the articulated probe is guided from the feeding mechanism into the entrance.

In some embodiments, the region of interest comprises a lumen.

In some embodiments, the region of interest is selected from the group consisting of: the esophagus; the gastrointestinal tract; the pericardial space; the peritoneal space; and combinations thereof.

In some embodiments, the support member comprises an axially curved member.

In some embodiments, the support member comprises a cylindrical tube.

In some embodiments, an inner diameter of the support member is larger than the outer diameter of the articulated probe.

In some embodiments, the method further comprises controllably clamping the articulated probe within the support member so as to stabilize the articulated probe.

In some embodiments, the clamp comprises a balloon configured to controllably expand and apply pressure on an outer surface of the articulated probe, such that the articulated probe can be stabilized in an axial and/or radial direction within the support member.

In some embodiments, the method further comprises: providing at least one channel extending at least partially along a longitudinal axis of the support member; and extending a filament through the channel.

In some embodiments, the method further comprises: providing at least one tool channel on an outer surface of the support member and extending along a longitudinal axis of the support member, configured to guide a filament into a probe side port located on an outer surface of the articulated probe; and extending a filament through the tool channel.

In some embodiments, the tool channel comprises a shaft connected to a tool port positioned on an outer surface of the support member.

In some embodiments, the system is configured to perform an esophageal procedure.

In some embodiments, the system is configured to perform an esophageal procedure selected from the group consisting of: an esophageal diagnostic procedure; an esophageal therapeutic procedure; a tissue biopsy procedure; a brachytherapy procedure; a drug delivery procedure; a procedure in which energy is delivered to esophageal tissue; a laryngectomy; a mediastinal nodal dissection; a vocal cord procedure; a supraglottic laryngectomy; a vocal chord biopsy; a cordotomy; a resection of the epiglottis; a hemi-epiglottidectomy; a synechia resection of the vocal cords; and combinations thereof.

In some embodiments, the system is configured to perform a colorectal procedure.

In some embodiments, the system is configured to perform a colorectal procedure selected from the group consisting of: a colorectal diagnostic procedure; a colorectal therapeutic procedure; a tissue biopsy procedure; a brachytherapy procedure; a drug delivery procedure; a procedure in which energy is delivered to colorectal tissue; a colectomy; a polypectomy; a minimally invasive transanal full thickness resection of an early rectal tumor; a transanal total mesorectal excision; a natural orifice transluminal endoscopic surgery; and combinations thereof.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIG. 1 is a perspective illustrative view of an articulating probe system, in accordance with embodiments of the present inventive concepts.

FIGS. 2A-2C are graphic demonstrations of an articulated probe device, in accordance with embodiments of the present inventive concepts.

FIG. 3 is a perspective view of a portion of a tool positioning system, in accordance with embodiments of the present inventive concepts.

FIG. 4A is a perspective view of a tool support inner tube, in accordance with embodiments of the present inventive concepts.

FIG. 4B is a side view of the interface of the distal end of an introducer, a tool support and an articulating probe, in accordance with embodiments of the present inventive concepts.

FIG. 4C is a perspective view of the interface of the distal end of an introducer, a tool support and an articulating probe, in accordance with embodiments of the present inventive concepts.

FIG. 5A is an exploded design schematic of a detachable feeder top assembly 300 for an articulating probe, in accordance with embodiments of the present inventive concepts.

FIG. 5B is an illustrative internal view of a feeder system, in accordance with embodiments of the present inventive concepts.

FIG. 6A is an illustrative perspective view of a force-transfer driving subassembly of a top assembly, consistent with embodiments of the present inventive concepts.

FIG. 6B is a perspective view of a force-transfer driving subassembly of a top assembly, in accordance with embodiments of the present inventive concepts.

FIG. 6C is an illustrative side-perspective view of a ninety-degree gear transfer subassembly of the force-transfer driving assembly of FIGS. 6A-6B, in accordance with embodiments of the present inventive concepts.

FIG. 6D is another illustrative perspective view of a force-transfer driving subassembly of FIGS. 6A-6C, in accordance with embodiments of the present inventive concepts.

FIG. 6E is an illustrative perspective view of a bearing mounting block for a lead screw of the force-transfer driving assembly of FIGS. 6A-6D, in accordance with embodiments of the present inventive concepts.

FIG. 6F is an illustrative perspective view of a bearing mounting block for a lead screw of the force-transfer driving assembly of FIGS. 6A-6E, in accordance with embodiments of the present inventive concepts.

FIG. 7A is a perspective view of internal components of a top assembly of a feeder assembly, in accordance with embodiments of the present inventive concepts.

FIG. 7B is a perspective view of the distal end of a feeder assembly with an energy chain removed for illustrative clarity, in accordance with embodiments of the present inventive concepts.

FIG. 8 is a schematic illustration of a capstan drive assembly, in accordance with embodiments of the present inventive concepts.

FIG. 8A is a cutaway perspective front view of a feeder assembly, in accordance with embodiments of the present inventive concepts.

FIG. 8B is a close-up cutaway perspective front view of a gear box of a feeder assembly, in accordance with embodiments of the present inventive concepts.

FIG. 9 is a partial cutaway perspective front view of a feeder assembly, in accordance with embodiments of the present inventive concepts.

FIG. 10 is a schematic view of a safety system, in accordance with embodiments of the present inventive concepts.

FIG. 11 is a perspective illustrative view of an articulating probe system, in accordance with embodiments of the present inventive concepts.

FIG. 12 is a perspective top view of a base assembly, in accordance with embodiments of the present inventive concepts.

FIG. 13 is a bottom view of a top assembly, in accordance with embodiments of the present inventive concepts.

FIG. 14 is a perspective cutaway view of a handle of a top assembly of a feeder assembly of an articulating probe system, in accordance with embodiments of the present inventive concepts.

FIG. 15 is a perspective cutaway view of a base assembly of a feeder assembly of an articulating probe system, in accordance with embodiments of the present inventive concepts.

FIGS. 15A-15C are perspective views of proximity sensor componentry, in accordance with embodiments of the present inventive concepts.

FIG. 16 is a perspective partial cutaway view of a base assembly of a feeder assembly of an articulating probe system, in accordance with embodiments of the present inventive concepts.

FIG. 16A is a section view of a base assembly and of the interaction of a heel and base cutout, in accordance with embodiments of the present inventive concepts.

FIG. 16B is a close-up perspective view of a cam engagement assembly of a base assembly, in accordance with embodiments of the present inventive concepts.

FIG. 17A is a side view of a cable bobbin of a top assembly, positioned in a shipping condition, in accordance with embodiments of the present inventive concepts.

FIG. 17B is a side view of a cable bobbin of a top assembly, positioned in an operating condition, in accordance with embodiments of the present inventive concepts.

FIG. 17C is a side view of a cable bobbin of a top assembly, in a release condition, in accordance with embodiments of the present inventive concepts.

FIG. 18 is a top view of a sterile drape assembly, in accordance with embodiments of the present inventive concepts.

FIG. 18A is a magnified view of a portion of the drape assembly of FIG. 18, in accordance with embodiments of the present inventive concepts.

FIGS. 19A-19F are various views of an inner link, in accordance with embodiments of the present inventive concepts.

FIGS. 20A-20F are various views of an outer link, in accordance with embodiments of the present inventive concepts.

FIG. 21 is a side sectional view of a portion of an articulating probe, in accordance with embodiments of the present inventive concepts.

FIG. 22 is a side sectional view of the distal portion of an outer link mechanism, in accordance with embodiments of the present inventive concepts.

FIGS. 22A and 22B are two magnified views of the conical to spherical interface of two outer links of FIG. 22, in accordance with embodiments of the present inventive concepts.

FIGS. 23 and 24 are a schematic view of a steering module, and a flow chart of a steering method, respectively, in accordance with embodiments of the present inventive concepts.

FIG. 25 is a flow chart of a safety method for performing a calibration, in accordance with embodiments of the present inventive concepts.

FIG. 26 is a flow chart of a method for preventing and/or detecting excessive force, in accordance with embodiments of the present inventive concepts.

FIG. 27 is a flow chart of a method for detecting and/or reducing unintended motion of an articulating probe, in accordance with embodiments of the present inventive concepts.

FIG. 28 is a flow chart of a calibration procedure, in accordance with embodiments of the present inventive concepts.

FIG. 29 is a perspective view of a robotic introducer system, in accordance with embodiments of the present inventive concepts.

FIG. 30 is a perspective view of the second assembly of FIG. 29, in accordance with embodiments of the present inventive concepts.

FIG. 31A is a perspective view of the distal link extension assembly of FIGS. 29 and 30, in accordance with embodiments of the present inventive concepts.;

FIG. 31B is an exploded view of the distal link extension assembly of FIG. 31A, in accordance with embodiments of the present inventive concepts.

FIG. 31C is an exploded view of the lighting assembly of FIG. 31B, in accordance with embodiments of the present inventive concepts.

FIG. 32A is a perspective view of the camera assembly of FIGS. 31A and 31B, in accordance with embodiments of the present inventive concepts.

FIG. 32B is an exploded view of the camera assembly of FIGS. 31A, 31B, and 32A, in accordance with embodiments of the present inventive concepts.

FIG. 33A is a perspective view of the lens assembly of FIGS. 32A and 32B, in accordance with embodiments of the present inventive concepts.

FIG. 33B is a cross-sectional view of the lens assembly of FIGS. 32A, 32B, and 33A, in accordance with embodiments of the present inventive concepts.

FIG. 33C is an exploded view of the lens assembly of FIGS. 32A, 32B, 33A and 33B, in accordance with embodiments of the present inventive concepts.

FIG. 34 is a flowchart illustrating a method for assembling a robotic introducer system to perform an operation, in accordance with embodiments of the present inventive concepts.

FIG. 35 is a flowchart illustrating a method for assembling a robotic introducer system to perform an operation, in accordance with embodiments of the present inventive concepts.

FIG. 36 is a cross-sectional view of an optical assembly, in accordance with embodiments of the present inventive concepts.

FIG. 37 is a view of a display at a console, the display including a displayed image generated from the optical assembly of FIG. 36, in accordance with embodiments of the present inventive concepts.

FIG. 38 is a cross-sectional view of a robotic introducer system comprising a distal camera assembly, in accordance with embodiments of the present inventive concepts.

FIG. 39A is a perspective view of the distal end of an articulating probe including a set of attaching elements, in accordance with embodiments of the present inventive concepts.

FIG. 39B is a perspective view of the proximal end of a distal link extension assembly including a set of attaching elements that can mate with the attaching elements of the articulating probe of FIG. 39A, in accordance with embodiments of the present inventive concepts.

FIG. 40 is a top view of a tool positioning system for performing a medical procedure, in accordance with embodiments of the present inventive concepts.

FIG. 41 is a top view of a tool positioning system for performing a medical procedure, in accordance with other embodiments of the present inventive concepts.

FIG. 42 is a perspective view of a tool positioning system, in accordance with an embodiment of the present inventive concepts.

FIG. 43 is a cross-sectional front view of a tool positioning system, in accordance with embodiments of the present inventive concepts.

FIG. 44 is a perspective view of a tool positioning system having multiple connectors, in accordance with an embodiment of the present inventive concepts.

FIG. 45 is a perspective view of a tool positioning system having three tools in communication with a connector, in accordance with an embodiment of the present inventive concepts.

FIG. 46 is a perspective view of a distal end of a tool positioning system, in accordance with embodiments of the present inventive concepts.

FIGS. 47A-47D are perspective views of distal links having multiple side ports, in accordance with embodiments of the present inventive concepts.

FIG. 48 illustrates a top view of an embodiment of the introduction device attached to a feeding mechanism, in accordance with embodiments of the present inventive concepts.

FIG. 49 illustrates a side perspective view of the embodiment of the introduction device illustrated in FIG. 48, in accordance with embodiments of the present inventive concepts.

FIG. 50 illustrates a view of the embodiment of the introduction device, in accordance with embodiments of the present inventive concepts.

FIG. 51 illustrates a side cross-sectional view of an embodiment of an introduction device having a tool port and attached to a feeding mechanism, in accordance with embodiments of the present inventive concepts.

FIG. 52 illustrates a side perspective view of an introduction device having a pair of tool ports and attached to a feeding mechanism, in accordance with embodiments of the present inventive concepts.

FIG. 53 illustrates a perspective view of an introduction device, in accordance with embodiments of the present inventive concepts.

FIG. 54 illustrates a side perspective view of an introduction device, in accordance with embodiments of the present inventive concepts.

FIG. 55 illustrates a side perspective view of an introduction device, in accordance with embodiments of the present inventive concepts.

FIG. 56 illustrates a side perspective view of an introduction device, in accordance with embodiments of the present inventive concepts.

FIG. 57 illustrates a flow chart of a method of introducing an articulated probe to a body lumen, in accordance with embodiments of the present inventive concepts.

FIGS. 58A and 58B are schematic diagrams of embodiments of a robotic introducer system including a first assembly and a second assembly in accordance with the present inventive concepts.

FIG. 59A is an exploded perspective view of an embodiment of the robotic introducer system of FIGS. 58A and 58B, in accordance with embodiments of the present inventive concepts.

FIGS. 59B and 59C are a top view and side perspective view, respectively, of the second assembly of the robotic introducer system of FIG. 59A, in accordance with embodiments of the present inventive concepts.

FIG. 59D is a bottom perspective view of the first assembly of the robotic introducer system of FIG. 59A, in accordance with embodiments of the present inventive concepts.

FIGS. 59E, 59F, 59G, and 59H are side perspective views of the interaction of the first assembly and second assembly of the robotic introducer system of FIG. 59A, in accordance with embodiments of the present inventive concepts.

FIGS. 59I, 59J, and 59K are exploded perspective, bottom, and side views, respectively, of the first assembly of the robotic introducer system of FIG. 59A, in accordance with embodiments of the present inventive concepts.

FIG. 59L is a perspective view of a latching mechanism for securing the first assembly of the robotic introducer system of to the second assembly of the robotic introducer system of FIG. 59A, in accordance with embodiments of the present inventive concepts.

FIGS. 60A1-60A4 are schematic views depicting an alternative embodiment for the interface of inner and outer carts with inner and outer probes, in accordance with embodiments of the present inventive concepts.

FIGS. 60B1-60B2 and 60C1-60C2 are schematic views detailing features of the FIG. 60A, in accordance with embodiments of the present inventive concepts.

FIGS. 60D1-60D2 are schematic views depicting another embodiment for the interface of the inner and outer carts with the inner and outer probes, in accordance with embodiments of the present inventive concepts.

FIGS. 60E1-60E2 are schematic views depicting another embodiment for the interface of the inner and outer carts with the inner and outer probes, in accordance with embodiments of the present inventive concepts.

FIGS. 60F1-60F5 are schematic views depicting another embodiment for the interface of the inner and outer carts with the inner and outer probes, in accordance with embodiments of the present inventive concepts.

FIG. 60G is a schematic view depicting another embodiment for the interface of the inner and outer carts with the inner and outer probes, in accordance with embodiments of the present inventive concepts.

FIG. 61A is a perspective view of a distal outer link of the outer probe in accordance with embodiments of the present inventive concepts.

FIG. 61B is a perspective view of a camera system in accordance with embodiments of the present inventive concepts.

FIG. 61C is a perspective view of a first assembly including the distal outer link of FIG. 61A and suitable for receiving a camera system in accordance with embodiments of the present inventive concepts.

FIGS. 61D and 61E are close-up perspective views of the first assembly in accordance with embodiments of the present inventive concepts.

FIG. 61F-1 and FIG. 61F-2 are perspective and top views respectively, of an outer link including a camera cable clip in accordance with embodiments of the present inventive concepts.

FIG. 61G-1 and FIG. 61G-2 are perspective and top views respectively, of an outer link including a camera cable recess in accordance with embodiments of the present inventive concepts.

FIGS. 62A, 62B, 63A and 63B are side views of a sterile drape assembly according to embodiments of the present inventive concepts.

FIGS. 64A and 64B are flow diagrams of a method for applying a sterile drape at a robotic introducer system, in accordance with embodiments of the present inventive concepts.

FIGS. 65 and 65A, are schematic views of a system, in accordance with embodiments of the present inventive concepts.

FIG. 66A is a perspective view of a removable introducer having a clam-shell configuration, in accordance with other embodiments of the present inventive concepts.

FIGS. 66B-66D are perspective views of the removable introducer of FIG. 66A in various stages of assembly, in accordance with embodiments of the present inventive concepts.

FIGS. 67A-67E are perspective views of a removable introducer having a clam-shell configuration, in accordance with embodiments of the present inventive concepts.

FIG. 68 is a flowchart illustrating a method for assembling a robotic system to performing one or more operations, in accordance with an embodiment of the present inventive concepts.

FIGS. 69A, 69B, 69C, 69D and 69E, are rear perspective, rear cutaway perspective, bottom perspective, bottom cutaway perspective, and front perspective views respectively, of an embodiment of the distal link of the outer probe, in accordance with the present inventive concepts.

FIG. 70A is a cutaway perspective view of a disposable portion of a feeder assembly, in accordance with some embodiments of the present inventive concepts.

FIG. 70B is a view illustrating a bobbin having a plurality of castellation features for mating with a plurality of castellation features of a bobbin plate, in accordance with some embodiments of the present inventive concepts.

FIG. 71A is a cutaway perspective view of a magnetic latch assembly at a proximal end of a feeder disposable portion, in accordance with some embodiments of the present inventive concepts.

FIG. 71B is a view of an underside of the feeder disposable portion of FIG. 71A, in accordance with some embodiments of the present inventive concepts.

FIG. 72A is a perspective view of a connector assembly, in accordance with an embodiment of the present inventive concepts.

FIG. 72B is a perspective view of the connector assembly of FIG. 72A coupled to a disposable portion of a feeder assembly, in accordance with an embodiment of the present inventive concepts.

FIGS. 72C-72F are perspective views of a connector assembly, in accordance with embodiments of the present inventive concepts.

### DETAILED DESCRIPTION OF EMBODIMENTS

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on” or “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

It will be further understood that when a first element is referred to as being “in”, “on”, “at” and/or “within” a second element, the first element can be positioned: within an internal space of the second element, within a portion of the second element (e.g. within a wall of the second element); positioned on an external and/or internal surface of the second element; and combinations of one or more of these, but is not limited thereto.

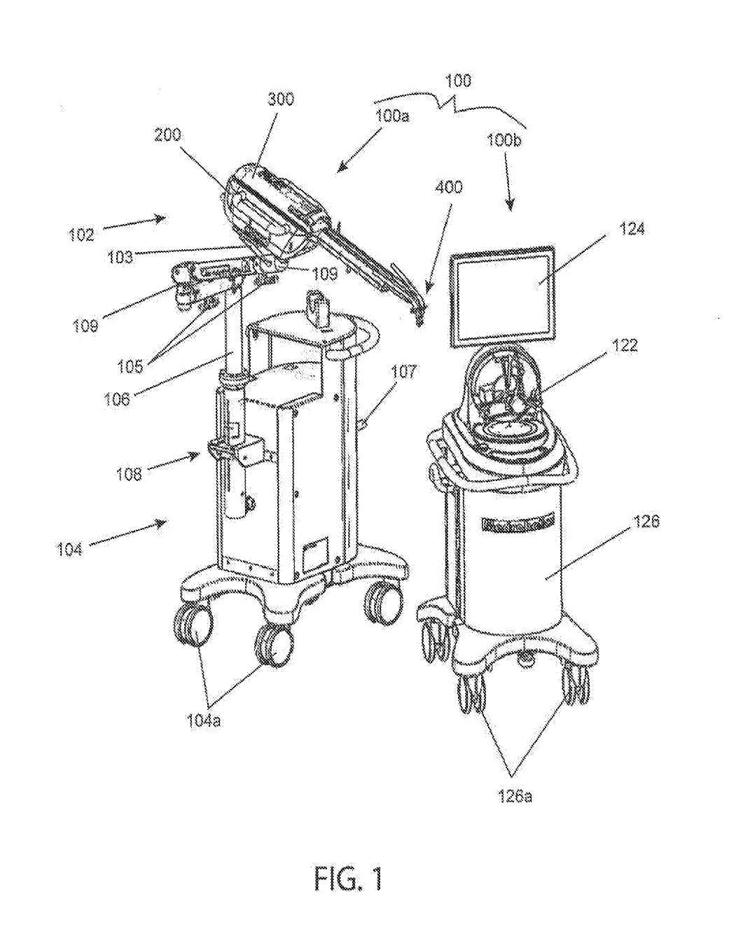

FIG. 1 is a perspective illustrative view of an articulating probe system 100 according to an embodiment of inventive concepts. In some embodiments, the articulating probe system 100 comprises a feeder unit 100a and an interface unit 100b (also referred to as console 100b). The feeder unit 100a, also referred to as a feeding mechanism, may comprise a feeder assembly 102 mounted to a feeder cart 104 at a feeder support arm 106. Feeder support arm 106 is adjustable in height, such as via rotation of crank handle 107 which is operably connected to vertical height adjuster 108 which slidingly connects feeder support 106 to feeder cart 104. Feeder support arm 106 can include one or more sub-arms or segment that pivot relative to each other at one or more mechanical joints 109 that can be locked and/or unlocked clamps 105 by one or more or related coupling devices. This configuration permits a range of angles, orientations positions, degrees of motion, and so on for positioning the feeder assembly 102 relative to a patient location. In some embodiments, one or more feeder supports 103 are attached between feeder support arm 106 and feeder assembly 102, such as to partially support the weight of feeder assembly 102 to ease positioning feeder assembly 102 relative to feeder support arm 106 (for example, when one or more joints 109 of feeder support arm 106 are in an unlocked position permitting manipulation of the feeder assembly 102). Feeder support 103 can comprise a hydraulic or pneumatic support piston, similar to the gas springs used to support tail gates of automobiles or trucks. In some embodiments, two segments of feeder support arm 106 are connected with a support piston (not shown) for example a support piston positioned at one of the segments, such as to support the weight of feeder assembly 102, or simply base assembly 200 alone. The feeder assembly 102 may include a base assembly 200 and a feeder top assembly 300 that is removably attachable to the base assembly 200. In some embodiments, a first feeder top assembly 300 can be replaced with another or second top assembly 300, after one or more uses (e.g. in a disposable manner). A use may include an operator or procedure performed or a human patient or multiple procedures or operators performed on the same patient. In some embodiments, base assembly 200 and top assembly 300 are fixedly attached to each other.

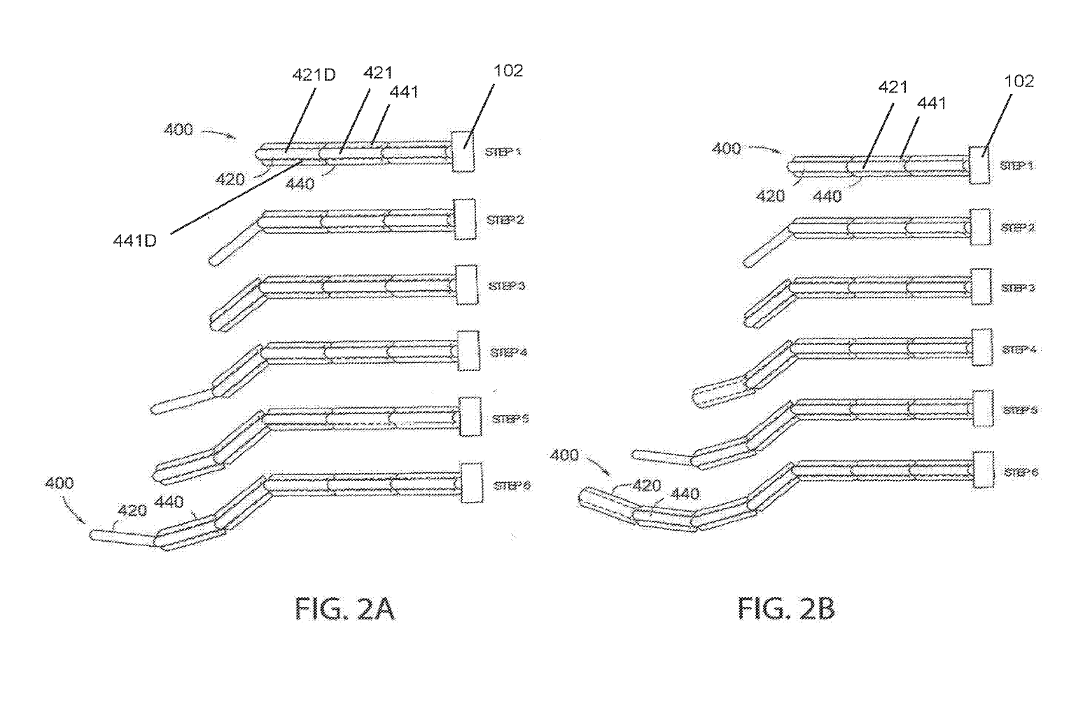

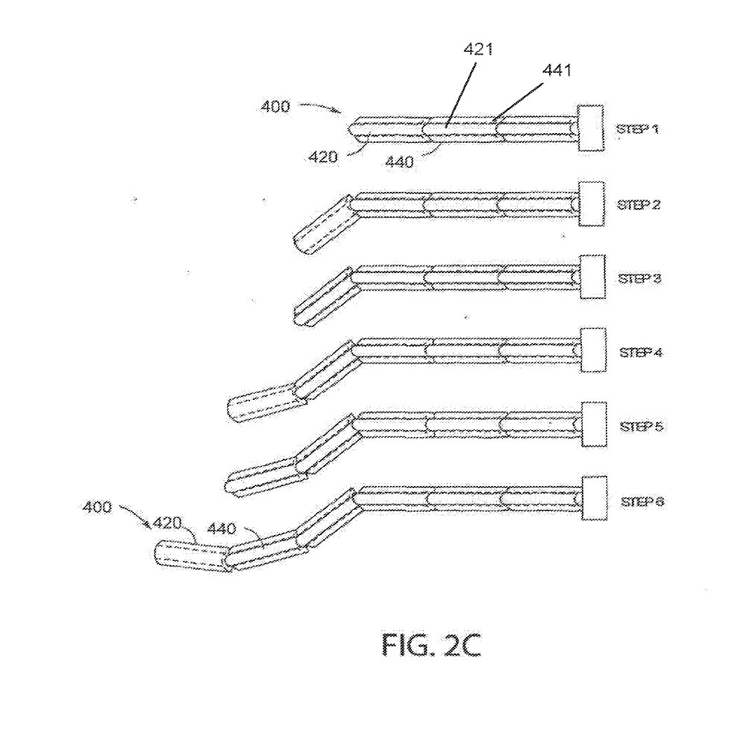

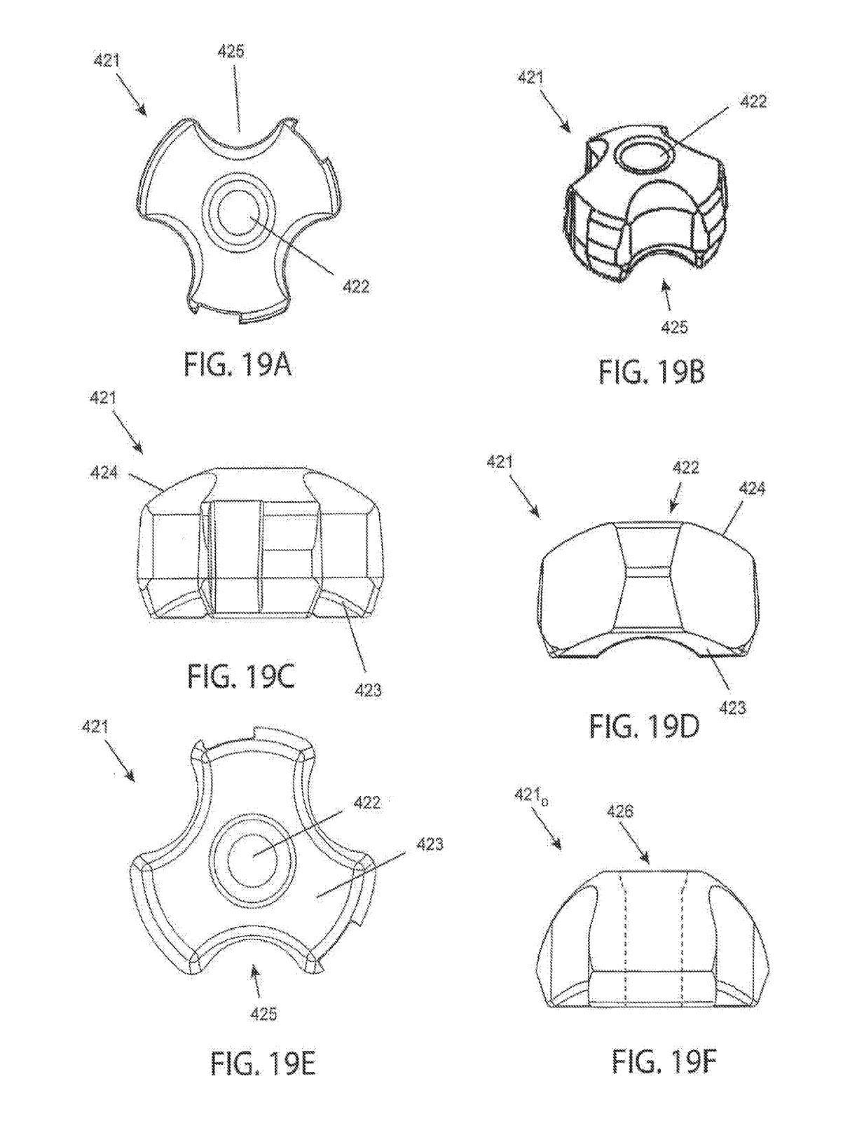

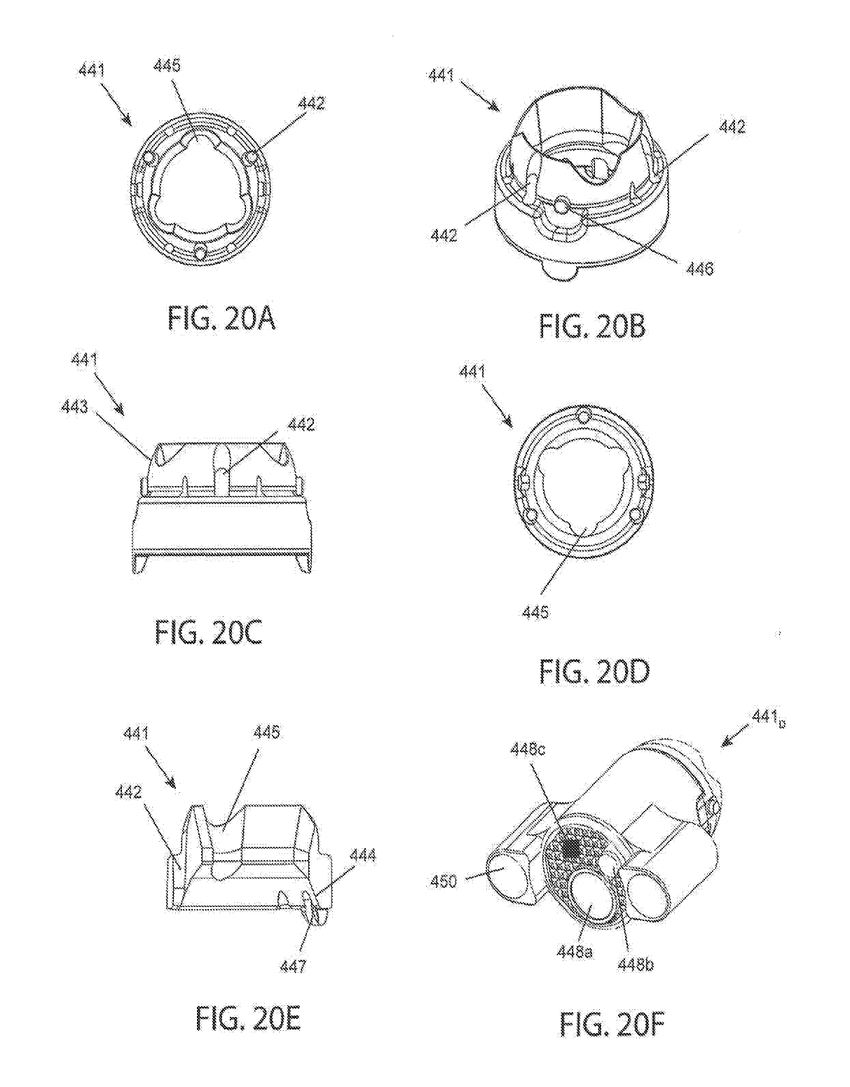

The top assembly 300 includes an articulating probe 400 for example comprising a link assembly including an inner link mechanism comprising a plurality of inner links, and an outer link mechanism comprising a plurality of outer links, as described in connection with various embodiments herein, to for example FIGS. 2A-2C and/or FIGS. 19A-19F and FIGS. 20A-20F. In some embodiments, articulating probe 400 comprises an inner mechanism of articulating links and an outer mechanism of articulating links, such as those described in applicant's co-pending International PCT Application Serial No. PCT/US2012/70924, filed Dec. 20, 2012, or U.S. patent application Ser. No. 14/364,195, filed Jun. 10, 2014, the content of which is incorporated herein by reference in its entirety. The position, configuration and/or orientation of the probe 400 are manipulated by a plurality of driving motors and cables positioned in the base assembly 200. The feeder cart 104 can be mounted on wheels 104a to allow for manual manipulation of its position. Feeder cartwheels 104a can include one or more locking features used to lock cart 104 in position after a manipulation or movement of probe 400, base assembly 200, and/or other elements of feeder assembly 102. In some embodiments, mounting of the feeder assembly 102 to a moveable feeder cart 104 is advantageous, such as to provide a range of positioning options for an operator, versus mounting of feeder assembly 102 to the operating table or other fixed structure.

In some embodiments, the base assembly 200 is operably connected to the interface unit 100b, such connection typically including electrical wires, optical fibers, or wireless communications, for transmission of power and/or data, or mechanical transmission conduits such as mechanical linkages or pneumatic/hydraulic delivery tubes (wired connections not shown). The interface unit 100b includes a human interface device HID 122 for receiving tactile commands from a surgeon, technician and/or other operator of system 100, and a display 124 for providing visual and/or auditory feedback. The interface unit 100b can likewise be positioned on an interface cart 126, which is mounted on wheels 126a (e.g. lockable wheels) to allow for manual manipulation of its position.

FIGS. 2A-2C are graphic demonstrations of a highly articulated probe device, according to embodiments of the present inventive concepts. A highly articulating robotic probe 400, according to the embodiment shown in FIGS. 2A-2C, comprises essentially two concentric mechanisms, an outer mechanism and an inner mechanism, each of which can be viewed as a steerable mechanism. FIGS. 2A-2C show the concept of how different embodiments of the probe 400 operate. Referring to FIG. 2A, the inner mechanism can be referred to as a first mechanism or inner link mechanism 420. The outer mechanism can be referred to as a second mechanism or outer link mechanism 440. Each mechanism can alternate between being rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume the outer link mechanism 440 is rigid and the inner link mechanism 420 is limp, as seen in step 1 in FIG. 2A. Now, the inner link mechanism 420 is both pushed forward by feeder assembly 102 (see e.g. FIG. 1), described herein, and its “head” or distal end is steered, as seen in step 2 in FIG. 2A. Now, the inner link mechanism 420 is made rigid and the outer link mechanism 440 is made limp. The outer link mechanism 440 is then pushed forward until it catches up or is coextensive with the inner link mechanism 420, as seen in step 3 in FIG. 2A. Now, the outer link mechanism 440 is made rigid, the inner link mechanism 420 limp, and the procedure then repeats. One variation of this approach is to have the outer link mechanism 440 be steerable as well. The operation of such a device is illustrated in FIG. 2B. In FIG. 2B it is seen that each mechanism is capable of catching up to the other and then advancing one link beyond. According to one embodiment, the outer link mechanism 440 is steerable and the inner link mechanism 420 is not. The operation of such a device is shown in FIG. 2C.

In medical applications, operation, procedures, and so on once the probe 400 may arrive at a desired location, the operator, such as a surgeon, can slide one or more tools through one or more working channels of outer link mechanism 440, inner link mechanism 420, or one or more working channels formed between outer link mechanism 440 and inner link mechanism 420, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of probe 400, such as working channels comprising one or more radial projections extending from outer link mechanism 440, these projections including one or more holes sized to slidingly receive one or more tools. As described with reference to other embodiment, working channels may be of outer location of the probe 400.

In addition to clinical procedures such as surgery, probe 400 can be used in numerous applications including but not limited to: engine inspection, repair or retrofitting; tank inspection and repair; surveillance applications; bomb disarming; inspection or repair in tightly confined spaces such as submarine compartments or nuclear weapons; structural inspections such as building inspections; hazardous waste remediation; biological sample and toxin recovery; and combination of these. Clearly, the device of the present disclosure has a wide variety of applications and should not be taken as being limited to any particular application.

Inner link mechanism 420 and/or outer link mechanism 440 are steerable and inner link mechanism 420 and outer link mechanism 440 can each be made both rigid and limp, allowing probe 400 to drive anywhere in three-dimensions while being self-supporting. Probe 400 can “remember” each of its previous configurations and for this reason, probe 400 can retract from and/or retrace to anywhere in a three dimensional volume such as the intracavity spaces in the body of a patient such as a human patient.

The inner link mechanism 420 and outer link mechanism 440 each include a series of links, i.e. inner links 421 and outer links 441 respectively, that articulate relative to each other. In some embodiments, the outer links are used to steer and lock the probe, while the inner links are used to lock the probe. In “follow the leader” fashion, while the inner links 421 are locked, the outer links 441 are advanced beyond a distal-most inner link 421D. The outer links 441 are steered into position by the system steering cables, and then locked by locking the steering cables. The cable of the inner links 421 is then released and the inner links 421 are advanced to follow the outer links. The procedure progresses in this manner until a desired position and orientation are achieved. The combined inner 421 and outer links 441 may include working channels for temporary or permanent insertion of tools at the surgery site. In some embodiments, the tools can advance with the links during positioning of the probe. In some embodiments, the tools can be inserted through the links following positioning of the probe.

One or more outer links 441 can be advanced beyond the distal-most inner link prior to the initiation of an operator controlled steering maneuver, such that the quantity extending beyond the distal-most inner link will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of probe 400. In some embodiments, an operator can select the number of links used for steering (e.g. to select between 1 and 10 links to be advanced prior to each steering maneuver).

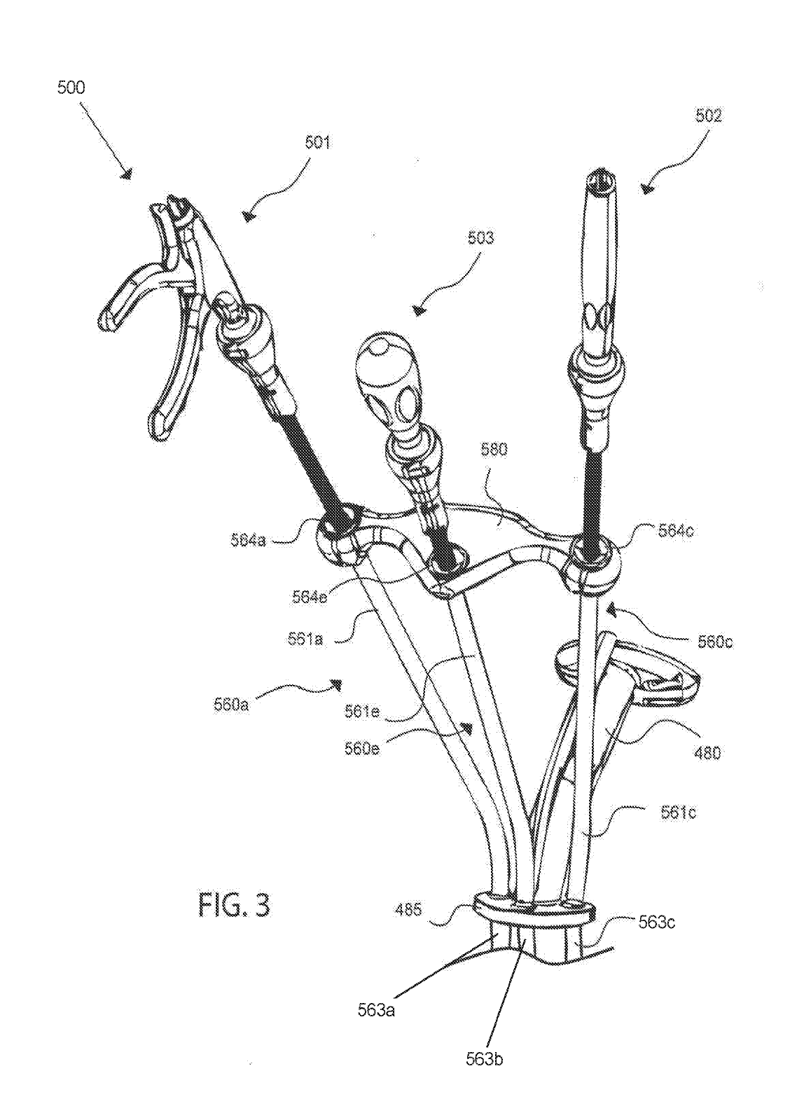

FIG. 3 is a perspective view of a portion of a tool positioning system 500 in accordance with the inventive concepts. The tool positioning system 500 comprises an introduction device, introducer 480, one or more tools supports 560, such as a first tool support 560a and a second tool support 560c. In some embodiments, system 500 includes at least three tool supports 560, such as when system 100 further comprises a third tool support 560e. Tool supports 560,a, c, e (generally, 560) are each constructed and arranged to slidingly receive a tool, for example, a shaft of a tool, described herein.

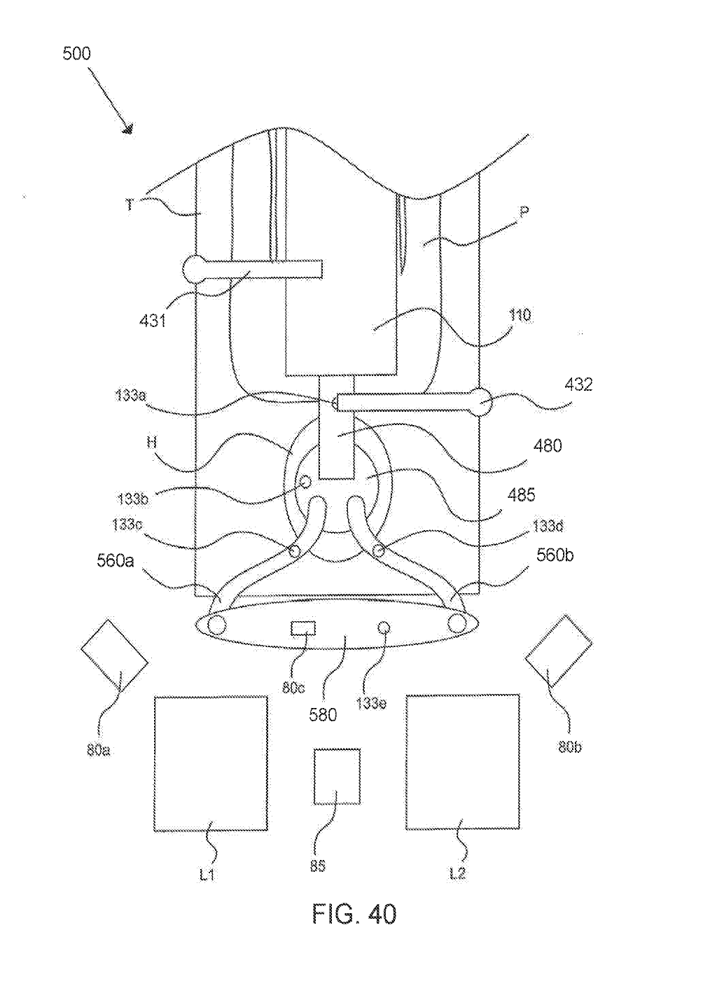

The introduction decan be constructed and arranged to slidingly receive an articulating probe such as the articulating probe 400 (see FIG. 1), and support, stabilize, and/or guide the articulating probe to a region of interest. As shown by way of example in FIGS. 40 and 51, the region of interest may be a lumen of a body of a patient (P), such as a cavity at the patient's head (H), e.g., a nose or mouth, or an opening formed by an incision. In clinical applications, typical regions of interest can include but not be limited to the esophagus or other locations within the gastrointestinal tract, the pericardial space, the peritoneal space, and combinations thereof. The region of interest may alternatively be a mechanical device, a building, or another open or closed environment or application in which the articulation probe system 100 of FIG. 1 can be used.

In some embodiments, system 100 is configured to perform one or more esophageal procedures, such as an esophageal procedure selected from the group consisting of: an esophageal diagnostic procedure; an esophageal therapeutic procedure; a tissue biopsy procedure; a brachytherapy procedure; a drug delivery procedure; a procedure in which energy is delivered to esophageal tissue; a laryngectomy; a mediastinal nodal dissection; a vocal cord procedure; a supraglottic laryngectomy; a vocal chord biopsy; a cordotomy; a resection of the epiglottis; a hemi-epiglottidectomy; a synechia resection of the vocal cords; and combinations of one or more of these. In some embodiments system 100 is configured to perform one or more colorectal procedures, such as a colorectal procedure selected from the group consisting of: a colorectal diagnostic procedure; a colorectal therapeutic procedure; a tissue biopsy procedure; a brachytherapy procedure; a drug delivery procedure; a procedure in which energy is delivered to colorectal tissue; a colectomy; a polypectomy; a minimally invasive transanal full thickness resection of an early rectal tumor; a transanal total mesorectal excision; a natural orifice transluminal endoscopic surgery; and combinations of one or more of these. In the embodiment of FIG. 3, three tools 501, 502, 503 are inserted into tool supports 560a, 560c and 560e, respectively. A single operator can operate tool positioning system 500, including any or all three tools 501, 502, 503. Alternatively, two or more operators can operate tool positioning system 500, including any or all three tools 501, 502, 503.

Three tool supports 560a, 560c, 560e extend between a base 485 and a connector 580. Connector 580 can connect and/or otherwise provide a stabilizing force between two or more tool ports 560 as shown. Each of tool supports 560a, 560c and 560e can include a funnel-shaped opening, 564a, 564c and 564e respectively, on their proximal end, such as to create a smooth entry for tool insertion. The base 485 may include a collar having first, second, and third openings aligned with the first, second, and third tool supports 560a, 560c, 560e, respectively. First, second, and third tool supports 560a, 560c, 560e, may include guide elements 561a, 561c, 561e (generally, 561) here which, can extend through the first, second, and third openings of the base 485 so that mid-portions of the guide elements 561 are positioned in the openings during operation. The base 485 can include a fourth opening for receiving introducer 480. In some embodiments, introducer 480 comprises base 485 for example, the base 485 is integrated with the body of the introducer 480.

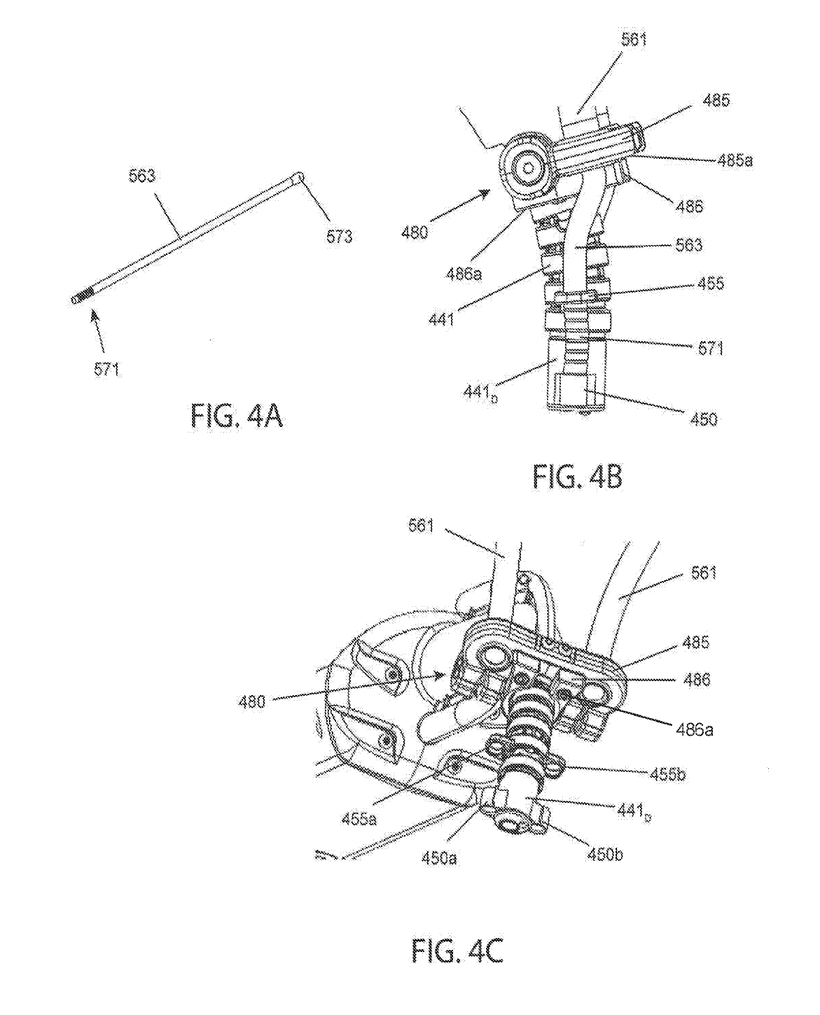

Tool suppers 560a, c, e may also include inner tubes 563a, c, e, (see FIG. 4A) that align with or mate with guide elements 561a, c, e, respectively. In some embodiments, inner tubes extend through guide elements at the base 485.

At least one tool 501, 502, 503 can have a shaft, shown inserted into tool supports 560a, 560c and 560e, respectively, constructed and arranged to be slidingly received by one or more tool supports 560. One or more of tools 501, 502, 503 can be selected from the group consisting of: suction device; ventilator; light; camera; grasper; laser; cautery; clip applier; scissors; needle; needle driver; scalpel; RF energy delivery device; cryogenic energy delivery device; and combinations thereof. A tool 501, 502, 503 can include a rigid and/or a flexible tool shaft.

The connector 580 is attached to first, second, and/or third tool supports 560a, 560c, 560e and can be constructed and arranged to maintain a relative distance between the tool supports 560a, 560c and/or 560e. The connector 580 can be fixedly attached to one or more of the tool supports 560. Alternatively, the connector 580 can be rotatably attached to one or more of the tool supports 560. The connector 580 can be constructed and arranged to be attachable to and/or detachable from the tool supports 560, such as when multiple connectors 580 (e.g. with different separation distances and/or other differences) are provided in system 100 such that different arrangements of tool supports 560 can be accomplished.

The base 485 can be fixedly attached to one or more of the tool supports 560. Alternatively, the base 485 can be rotatably attached to one or more of the tool supports 560. A gimbal (not shown) can be positioned at the base 485 and rotatably engage one or more guide elements 561 at the base 485.

A single operator can operate one or more of: the tool 501 extending from the first tool support 560a, the tool 502 extending from the second tool support 560c, and/or the tool 503 extending from the third tool support 560e, for example, from a single operator location. In particular, tool 501 can extend through 561a and 563a, tool 502 extends through 561c, 563c, and tool 503 can extend through 561e, 563e Alternatively, one operator can operate two tools of the tools 501, 502, 503, and another operator can operate the remaining tool of the tools 501, 502, 503.

FIG. 4A is a perspective view of a tool support inner tube 563, in accordance with embodiments of the present inventive concepts. FIG. 4B is a side view of the interface of the distal end of an introducer 480, a tool support and an articulating probe, in accordance with embodiments of the present inventive concepts. FIG. 4C is a perspective view of the interface of the distal end of an introducer 480, a tool support and an articulating probe in accordance with embodiments of the present inventive concepts.

Referring to FIGS. 4A, 4B and 4C, and with reference to the tool positioning system of FIG. 3, a distal end of an introducer 480 and its base 485 are shown. A distal link 441_{D} of articulating probe 400 includes first and second distal side ports 450a, 450b, at which tools can be slidingly supported. A tool support outer tube 561 extends from a top portion of the base 485. A tool support inner tube 563 is slidably positioned within the tool support outer tube 561 (note that tool support inner tubes 563 have been removed from FIG. 4C for illustrative clarity). In some embodiments, the tool support inner tube 563 is anchored (e.g. fixedly, rotatably or otherwise attached), at its distal end, to the respective one of the first and second distal side ports 450a, 450b. In this manner, as the distal outer link 441_{D} of the probe is advanced (e.g. in a longitudinal direction), the tool support outer tube 561 remains fixed in position, while the tool support inner tube increases in length of extension from the base 485.

In some embodiments, one or more intermediate outer links 441 can include one or more side ports, such as the two intermediate side ports 455a, 455b shown (generally, intermediate side ports 455), through which the tool support inner tube 563 can slidingly pass. The intermediate side ports 455 operate as a locator and/or support for the tool support inner tube to prevent inadvertent buckling or bending of the tool support inner tube 563, and/or to otherwise provide a smooth translation of one or more tool shafts or other filaments passing through a tool support 560.

In some embodiments, as shown in FIG. 4A the tool support inner tube 563 can include a flexibility enhancement feature at its distal portion 571. In the present embodiment, the tool support inner tube 563 includes rib features on distal portion 571, the indents of the ribs being of reduced outer diameter. Such ribbing provides for enhanced flexibility in the distal region of the tool support inner tube 563. Full steering capability of the distal outer link 441_{D} and proximate outer links 441 of the articulating probe 400 is highly desired for proper probe operation. By enhancing the relative flexibility of the tool support inner tube 563, any interference with steering capability by the tube 563 is mitigated or prevented. As shown at least at FIG. 4A, a proximal end of the tool support inner tube 563 can include a funnel-shaped feature 573 to aid in tool insertion.

In various embodiments, a flexibility enhancement feature of distal portion 571 can comprise a ribbed portion, a portion that has a different material composition than the main body portion (e.g. a more flexible material or other more flexible material composition), a portion that has walls that are relatively thinner than the main body portion and/or other applicable mechanisms for enhancing flexibility.

In some embodiments, the base 485 of the introducer 480 includes a flange 486 that projects from the undersurface 485a of the base 485. The flange 486 is positioned to communicate with (e.g. extend) the channel of the introducer 480, through which the articulating probe 400 passes. In this manner, the flange 486 provides additional support for probe 400 proximate the point at which it leaves introducer 480. With reference to FIG. 4B, it can be seen that the surface 486a of flange 486 at which probe 400 exits is more distal (e.g. lower on the page) than the surface of base 485 at which tool support inner tube 563 exits. In this manner, probe 400 is further supported, reducing its moment arm relative to the point at which it exits the introducer 480. At the same time, the exit location of tool support inner tube 563 is maintained by not passing through flange 486, such as to allow for angulation of a tool passing through inner tube 563 at a pivot location proximal to the exit location of probe 400 from flange 486. Flange 486 can comprise an attachable component (e.g. attachable to the remainder of introducer 480), or it can be fixedly attached (e.g. a single piece construction of introducer 480). In some embodiments, multiple attachable flanges 486 are provided to provide different configurations for the support of probe 400.

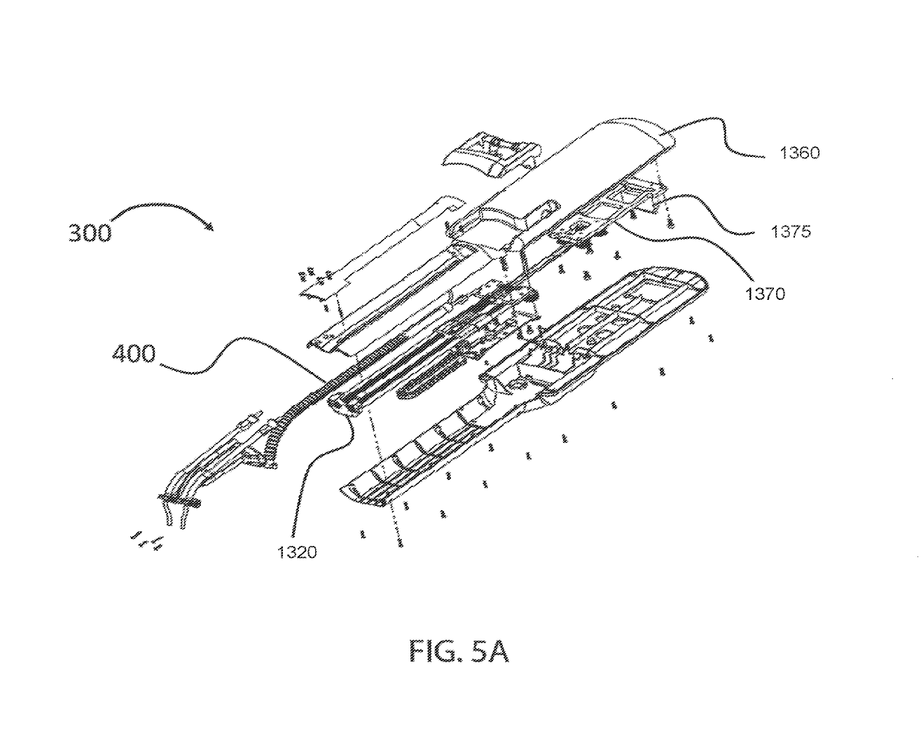

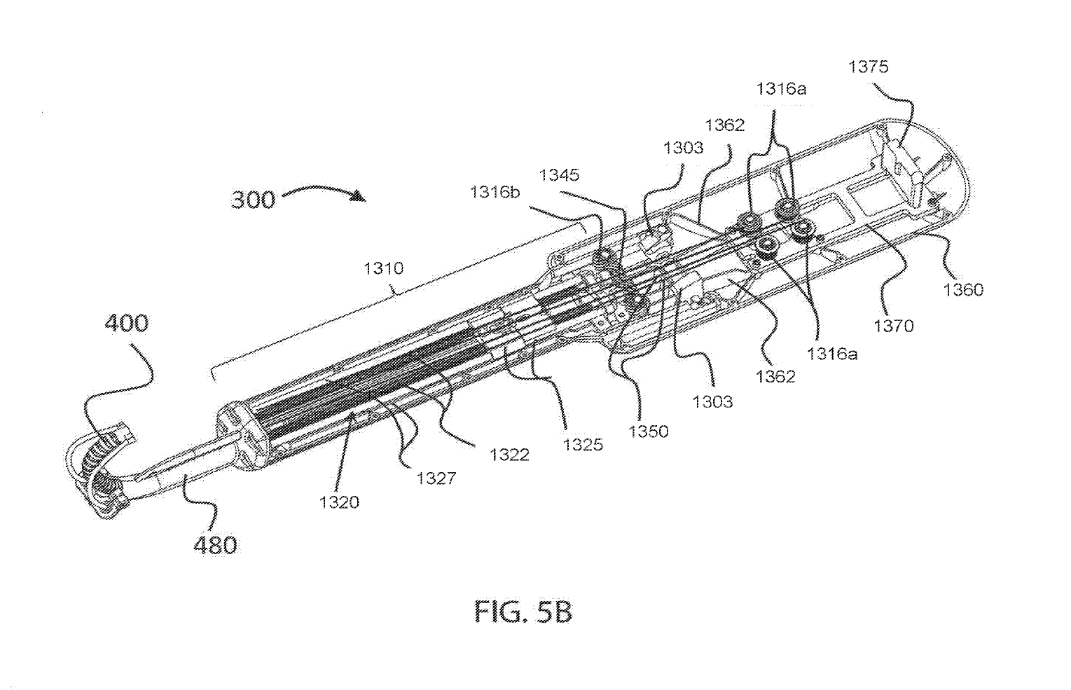

FIG. 5A is an exploded design schematic of a detachable feeder top assembly 300 for an articulating probe, such as articulating probe 400 described herein, according to an embodiment of inventive concepts. FIG. 5B is an illustrative internal view of a feeder system according to an embodiment of inventive concepts. In an embodiment, the feeder top assembly 300 includes a housing 1360 having a stabilization plate 1370, at which plurality of cable bobbins 1316a are positioned. Housing 1360 is typically an injection molded plastic housing, such as a reinforced plastic housing. In an embodiment, the stabilization plate 1370 is mounted to housing 1360 proximate reinforced housing ribs 1362. In an embodiment, cables 1350 extend through an articulating probe 400 comprising both inner and outer links (e.g., the links of inner link mechanism 420 and outer link mechanism 440 of FIGS. 2A-2C). Each cable 1350 may have an end that is wrapped around bobbin 1316a. A rotation of a bobbin 1316a along the length or shorter the amount of cable 1350, extending through the probe 400. In an embodiment, the cables 1350 can be used to steer and/or reversibly tighten to “lock”/stiffen either or both of the inner link mechanism 420 or outer link mechanism 440 such as is described herein. In an embodiment, one or more cables 1350 can be used to lock the links and two or more cables 1350 can be used to steer the links. For example, three cables 1350 can be designated for steering the links of outer link mechanism 440 of FIGS. 2A-2C in three dimensions. These three cables 1350 can also be used for locking the outer link mechanism 440. The remaining cable(s) 1350 can be used for locking the links 421 of inner link mechanism 420. In an embodiment, when using cables 1350 for locking, the forces applied can be distributed over cables 1350. For example, if a 36 lb force is applied for locking the outer link mechanism 440 connected to three cables, then a force of 12 lbs can be applied to each of the connected cables 1350. In an embodiment, three of the bobbins 1316a are configured to control the outer links 440, such as to steer, feed cable for articulating probe 400 advancement, retract cable for probe 400 retraction, transition probe 400 from a limp to a rigid state (e.g. to lock), and to transition probe 400 from a rigid to a limp state (e.g. to become flexible). In this embodiment, one bobbin 1316a is typically used to control the inner links 420, such as to feed a cable 1350 for probe 400 advancement, retract a cable 1350 for probe 400 retraction, transition probe 400 from a limp to a rigid state (e.g. to lock), and to transition probe 400 from a rigid to a limp state (e.g. to become flexible). In some embodiments, the forces exerted by the bobbins 1316a on cables 1350 can exceed 1, 10, 30 and/or 50 pounds, such as to sufficiently lock the attached inner or outer links 441 of probe 400. In configurations in which four cables 1350 are used to steer and lock links of the probe 400, collective forces exerted by the bobbins 1316a can exceed 95 pounds, such as when 50 pounds is applied to lock the inner links 421 (e.g. with a single cable) and 15 pounds per cable is used to lock the outer links 441 (e.g. with three cables). In various embodiments, the amount of force applied is related to the size (including diameter and length) of the links of the inner link mechanism 420 and outer link mechanism 440 and also to the smoothness of the steering of the links. Greater force may be necessary to lock and stabilize a set of larger and/or longer links, including when the links are extended or retracted with respect to each other.

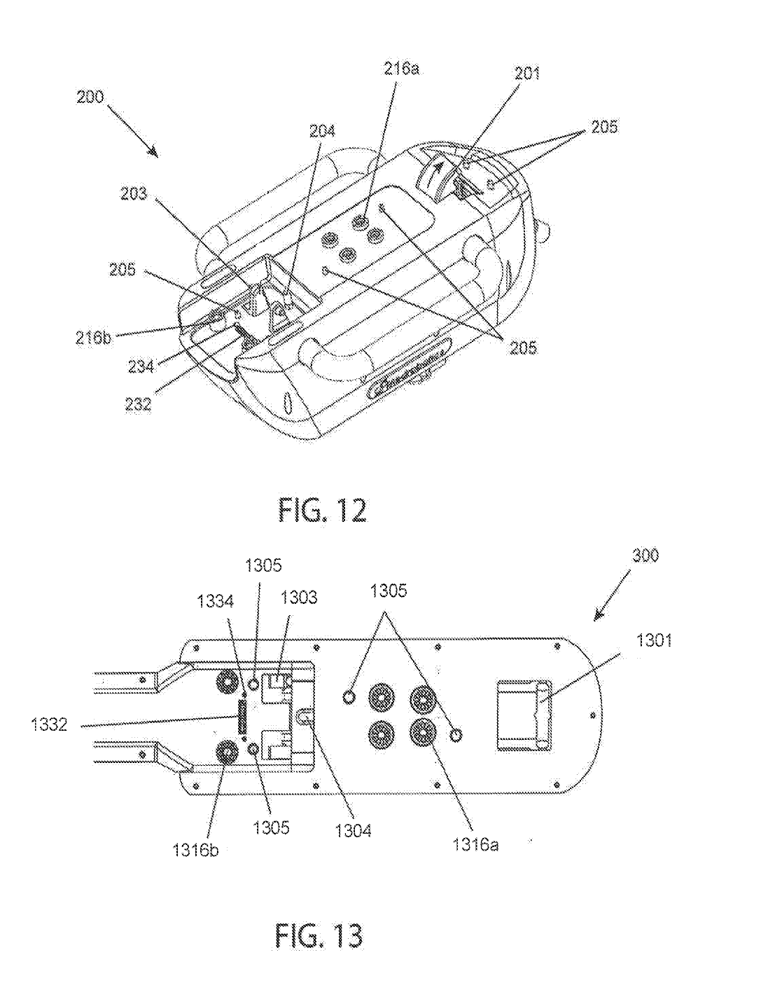

A heel plate 1375 (also referred to as heel herein) is fixedly attached to the stabilization plate 1370 and can lockably engage with base assembly 200 as described herein. Cams 1303 are also attached to the housing 1360 which are arranged to lockably engage with base assembly 200. In an embodiment, cams 1303 can articulate and are spring loaded, so as to rotate downward upon engaging latch prongs (such as engagement assembly 203 of FIG. 12). In an embodiment, the spring loaded cams 1303 provide up to about 20 pounds of tension. The heel plate 1375 and cams 1303 interlock with base assembly 200 and thereby stabilize and aid in the resistance of undesired motion, including lateral motion, of the feeder system and base assembly 200 during the transfer of power (e.g. cable applied force) to the probe 400 such as via bobbins 1316a. In an embodiment, the top assembly 300 is configured to be detachable from base assembly 200, such as to be cleaned or replaced with another top assembly 300 (e.g. a new, sterile top assembly 300, such as when probe 400 is exposed to biological or toxic materials.

A carriage drive segment 1310 is attached distally to a reinforced introducer 480, through which probe 400 extends. Introducer 480 can be used for guiding the probe 400's initial path through or toward a target area such as, for example, when introducer 480 comprises an outer surface similar to a body cavity shape found in a majority of patients. Probe 400 can be configured to rapidly advance through introducer 480, prior to fine motion control used after probe 400 exits introducer 480.

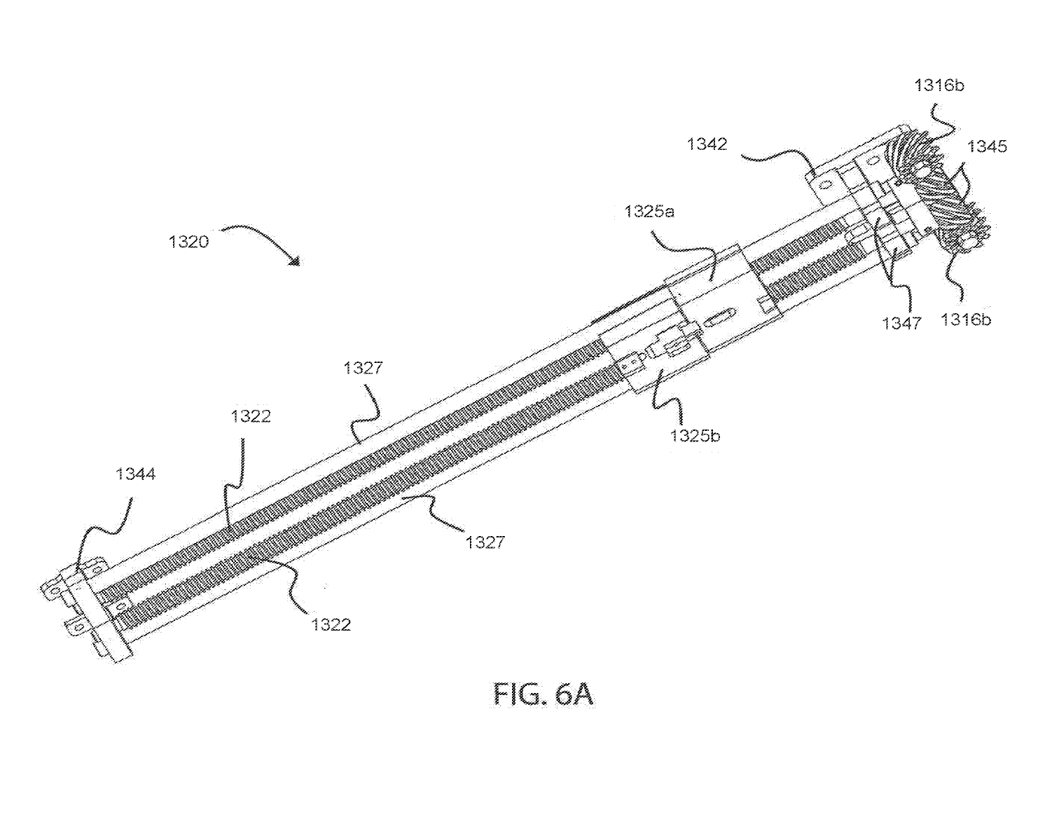

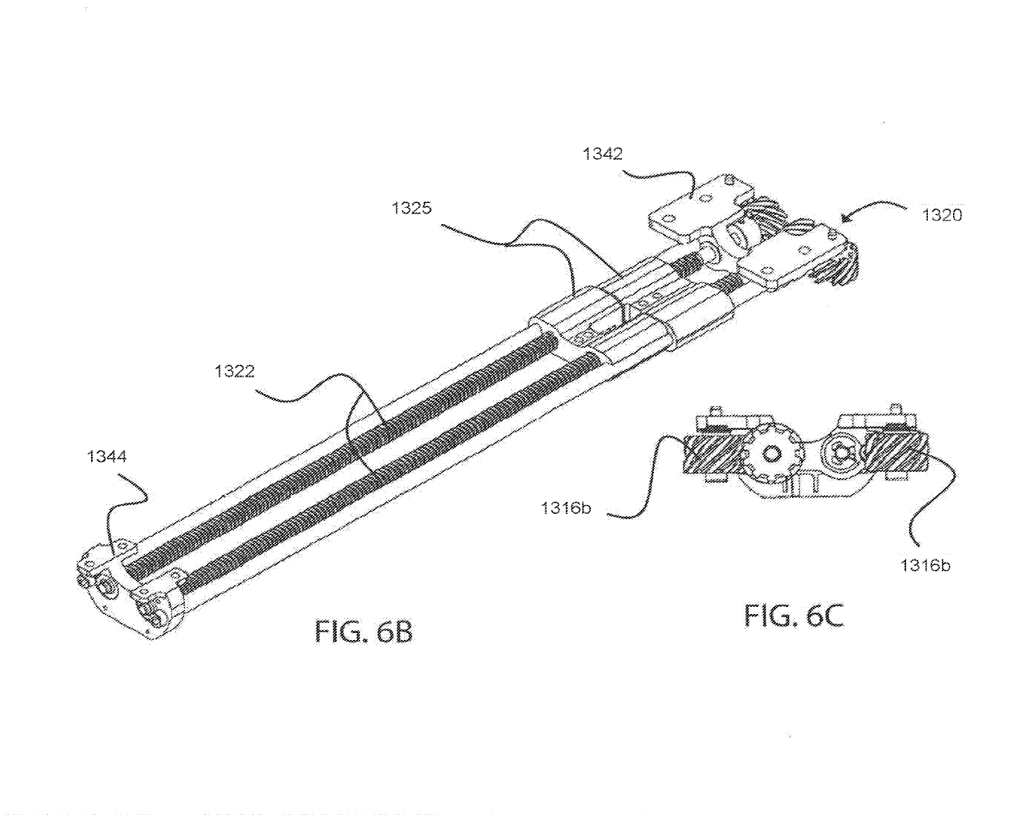

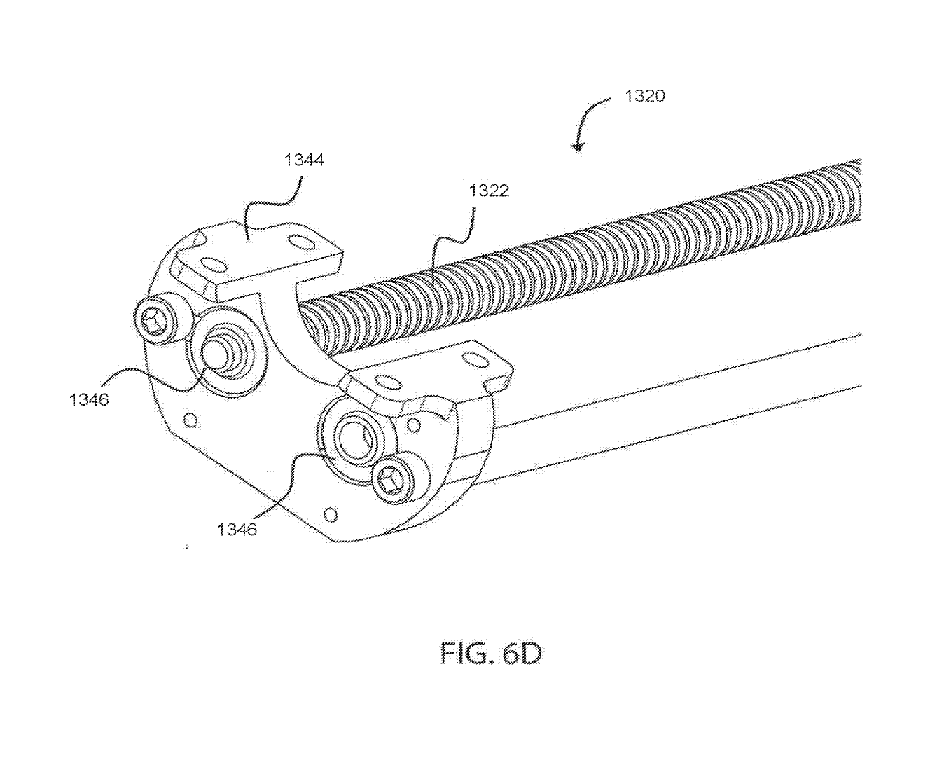

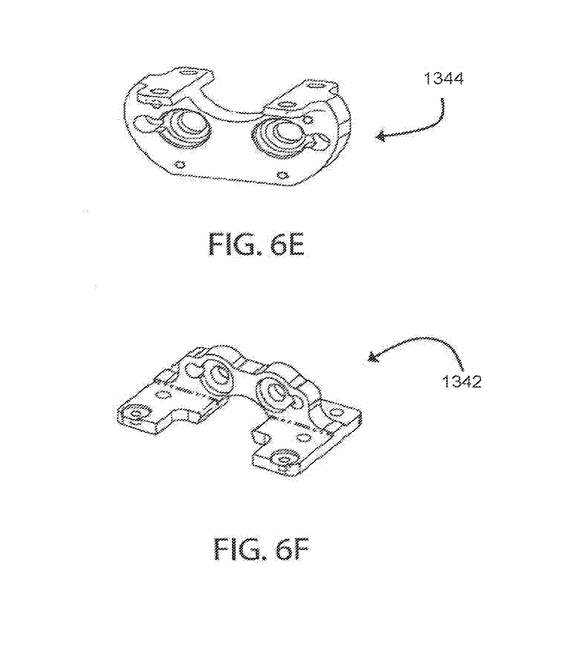

Referring to FIGS. 5A, 5B and 6A, an illustrative perspective view of a force-transfer driving subassembly 1320 of the top assembly 300 is shown. Top assembly 300 includes a carriage drive segment 1310 which is configured to independently drive two carriage assemblies, carriages 1325a, b (generally, 1325), along two lead screws 1322. Lead screws 1322 can comprise a pitch configured to cause lead screws 1322 to be non-back drivable. In an embodiment, one carriage 1325b drives an outer link mechanism 440 and one carriage 1325a drives an inner link mechanism 420 as described, for example, with respect to FIGS. 2A-2C. The lead screws 1322 are driven by a ninety-degree gear assembly including gears 1316b and gears 1345. In an embodiment, gears 1316b and 1345 include helical threads so as to increase overall contact between them and further stabilize force transfer between base assembly 200 and probe 400. Lead screws 1322 are secured within bearing mounting blocks 1342 and 1344 that are mounted to housing 1360. In an embodiment, bearing mounting block 1342 includes thrust bearings 1347 for further stabilizing a force transfer between gears 1345 and lead screws 1322. In an embodiment, carriages 1325 include grooves to slidably ride upon guide rails 1327, which aid in ensuring linear movement of carriages 1325 and providing additional stabilization of the subassembly 1320, top assembly 300, and probe 400, or a combination thereof so as to resist undesired movement during force transfer, such as undesired torque or compression of top assembly 300. Guide rails 1327 can further prevent undesired relative movement between the carriages 1325, particularly when unequal forces are applied to them. In an embodiment, guide rails 1327 are slidingly received and fixed within bearing blocks 1344 and 1342 in order to maintain substantially parallel configuration to maintain stability of the top assembly 300. Bearing blocks 1344, 1342 may have through holes or the like for insertion of screen or related coupling devices to secures the subassembly 1320 to articulation probe 400. In an embodiment, guide rails 1327 are configured to have square, rectangular, round, slotted, or other various cross sectional shapes configured to slidingly engage a receiving portion of carriages 1325. In one embodiment, guide rails 1327 have a rectangular cross section configured to prevent undesired twisting along one or more axes of top assembly 300 (e.g. the major axis of top assembly 300). The dual screw and rail configuration helps, in particular, to resist twisting and bending of the feeder system. In an embodiment, force-transfer driving subassembly 1320 is a separate subassembly that is secured into the housing 1360 to minimize the deflection of the housing during force transfer, such as when housing 1360 comprises a plastic, injection-molded housing. In an embodiment, the carriages 1325 include reinforced bushings to engage with the lead screws and/or rails. In an embodiment, the bushings are coated and/or filled with Teflon or a similarly lubricious material.

FIG. 6B is a perspective view of a force-transfer driving subassembly 1320 of the top assembly 300 according to an embodiment of inventive concepts. FIG. 6C is an illustrative side-perspective view of a ninety-degree gear transfer subassembly of the force-transfer driving assembly of FIG. 6B. FIG. 6D is another illustrative perspective view of a force-transfer driving subassembly 1320 of FIG. 6B, with one lead screw 1322 and other components removed for illustrative clarity. In an embodiment, the mounting block 1344 includes spherical bearings 1346 to help ensure proper alignment between the lead screw 1322 and the bearing mounting block 1344. FIG. 6E is an illustrative perspective view of a bearing mounting block 1344 for a lead screw of the force-transfer driving assembly of FIGS. 6A-6B according to an embodiment of inventive concepts.

FIG. 6F is an illustrative perspective view of a bearing mounting block 1342 for a lead screw 1322 of the force-transfer driving assembly 1320 of FIGS. 6A-6B. As discussed above, in an embodiment, bearing mounting block 1342 includes thrust bearings 1347 for further stabilizing the force transfer between gears 1345 and lead screws 1322.

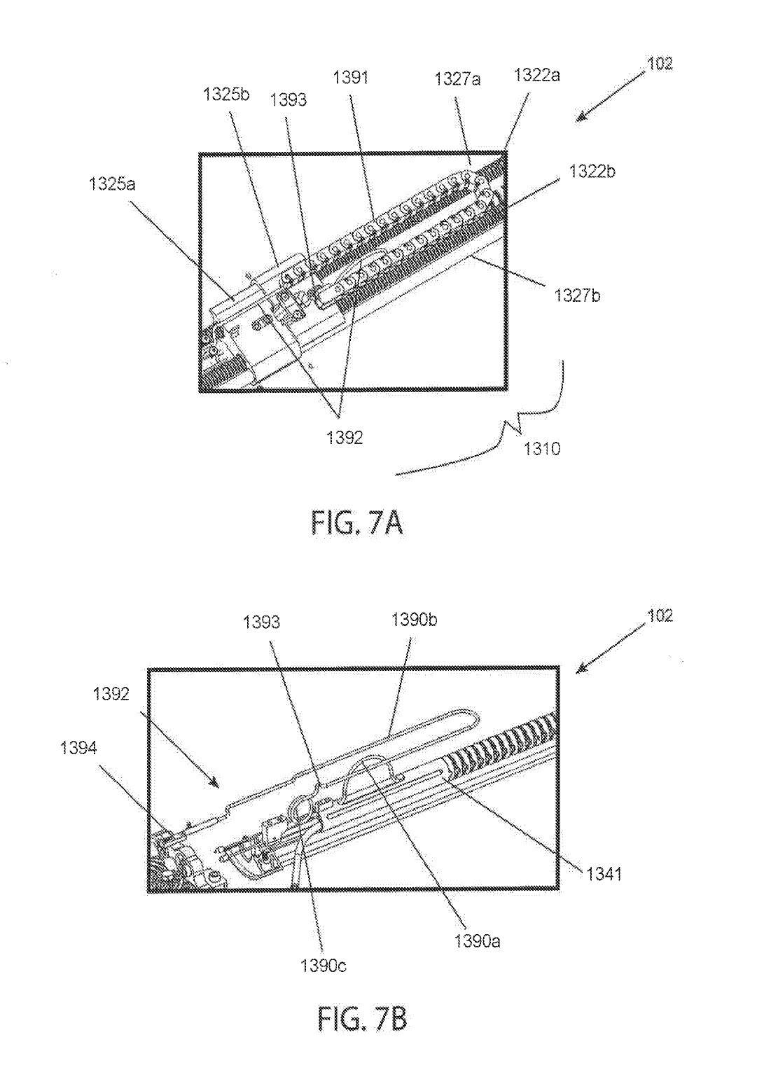

FIG. 7A is a perspective view of internal components of a top assembly 300 of a feeder assembly 102 in accordance with inventive concepts. Feeder assembly 102 includes a carriage drive segment 1310 including first and second carriages 1325a, 1325b which glide along first and second guide rails 1327a, 1327b. First carriage 1325a communicates with a first lead screw 1322a, and a second carriage 1325b communicates with a second lead screw 1322b. In this manner, rotation of the lead screw 1322a, 1322b is translated to linear movement of the corresponding carriage 1325a, 1325b for driving the carriage 1325a, 1325b in a linear path along the guide rails 1327a, 1327b. In some embodiments, the first carriage 1325b comprises an inner carriage in communication with inner link mechanism 420 of probe 400, while the second carriage 1325b comprises an outer carriage in communication with outer link mechanism 440 of probe 400. The carriages 1325a, 1325b are each coupled to a proximal-most link of the inner and outer link mechanisms 420, 440 so that the mechanisms can be independently advanced and retracted in a longitudinal direction. An energy chain 1391 is coupled at a first end to a fixed (non-moving) portion of top assembly 300, and at a second end to the second carriage 1325b. Segments of the energy chain 1391 extend and retract as carriage 1325b moves relative to non-moving portions of top assembly 300. The energy chain 1391 can be employed as a protective mechanism for wires and flexible filaments that extend through the links of probe 400 from the feeder mechanism. The energy chain 1391 can comprise a chain-like construction having a central aperture for receiving flexile filaments such as conduit 1392. In some embodiments, energy chain 1391 provides a bias such that it changes curvature while remaining substantially in a single plane.

In some embodiments, the conduit 1392 comprises a camera cable over which electrical and optical signals, for example, data signals, power signals, and the like, are transferred between a camera optic mounted to a distal link of the inner and outer link mechanisms and the base assembly 200. As the probe 400 extends in a distal direction during a procedure, additional cable is allowed to freely pass in the distal direction, so as not to interfere with steering of the probe. As the probe 400 is steered in a particular orientation that is off-axis, relative to the axis of extension, additional conduit, e.g., cable is required to be fed into the probe 400. In addition, in some embodiments, the number of outer links used for a steering maneuver can vary, as described herein. In such a case, the conduit 1392 is freely allowed to pass through the links to the feeder, and the length of the conduit 1392 passing through the probe varies in response to the number of links used in the steering maneuver. Accordingly, as shown in FIG. 7B the conduit 1392 can include one or more service loops 1390a, 1390b, 1390c. The service loops 1390a, 1390b, 1390c provide for additional slack conduit that can be fed into and removed from the probe 400, depending on the position of the distal end of the probe 400 relative to the feeder base ###.

In some embodiments, the first service loop 1390a in the conduit 1392 provides for maximum steering of the current quantity of distal-most outer links used in a steering maneuver (e.g. as selected by an operator). The first service loop 1390a may include a bend that permits for free movement of the conduit 1392 into and out of the probe 400 during the steering maneuvers. In some embodiments, conduit 1392 comprises a camera cable and the first service loop 1390a is coupled at a first end at a camera optic positioned in the distal-most outer link 441_{D} of probe 400 and is coupled at a second end 1393 to the second carriage 1325b. The length of the first service loop 1390a may be selected to support all possible configurations of articulating probe 400 that could possibly be encountered during a cumulative set of steering maneuvers (e.g. to support steering of the scope in its minimum bend radius at furthest advancement of outer link mechanism 440). In this manner, steering operations can occur in probe 400 without interference from tension in the conduit 1392 due to insufficient conduit length. In the present example embodiment, the first service loop 1390a passes through an aperture in a most-proximal outer link 441_{D} of probe 400. In some embodiments, the first service loop 1390a comprises third service loop 1390c as shown (e.g. comprising multiple physical loops of conduit 1392 collectively configured to support all potential steering maneuvers of probe 400).

In some embodiments, a second service loop 1390b in conduit 1392 provides for advancement and retraction of probe 400. The second service loop 1390B includes a loop portion that permits for free movement of second carriage 1325b e.g. while driving the outer link mechanism 440. In some embodiments, conduit 1392 comprises a camera cable and the second service loop 1390b is coupled at a first end at a camera connector 1394 to a camera circuit board and is coupled at a second end 1393 to the second carriage 1325b. The length of the second service loop 1390b is chosen to be longer than the maximum distance of linear translation of the second carriage 1325b, such as to accommodate all ranges of translation of second carriage 1325b. As shown in FIG. 7A, the second service loop 1390b can be protected and seated by the energy chain 1391.

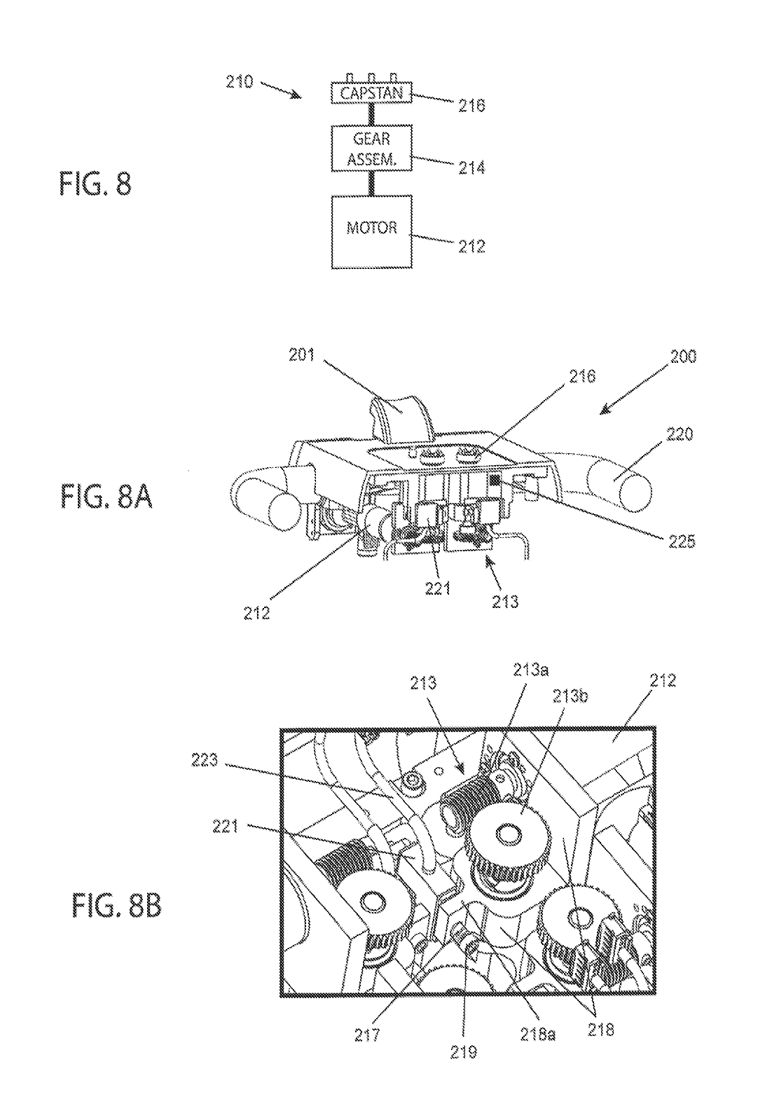

FIG. 8 is a schematic illustration of a capstan drive assembly, in accordance with embodiments the present inventive concepts. FIG. 8A is a cutaway perspective front view of a feeder assembly, in accordance with embodiments the present inventive concepts. FIG. 8B is a close-up cutaway perspective front view of a gear box of a feeder assembly, in accordance with the present inventive concepts.

Referring to FIG. 8, in some embodiments, a plurality of drive assemblies 210 are provided in the base assembly 200 of the feeder assembly 102. Each drive assembly 210 includes, in some embodiments, a motor 212, a gear assembly 214 and a capstan 216. The capstan 216 is constructed and arranged to mate with a corresponding bobbin on the top assembly 300. In alternative embodiments, the drive assembly 210 can include a bobbin, rather than a capstan, in which case, top assembly 300 includes a corresponding capstan.

The drive assemblies 210 and corresponding capstans 216 drive bobbins on top assembly 300, the bobbins in turn driving cables on top assembly 300, the cables used to control the operation of probe 400. In various embodiments, motor 212 can comprise any of a number of suitable motor types, including, but not limited to, a brushless DC motor, a stepper motor, a closed-loop servo motor. In various embodiments, a motor linkage encoder or position sensor may be included (e.g. in motor 212 and/or gear assembly 214) for providing closed-loop operation. The gear assembly 214 may comprise a mechanical assembly, for example, providing up to a 20:1 gear ratio, which can be connected to motor 212 to correspondingly reduce the rotational displacement provided by motor 212 (e.g. and correspondingly increase the torque provided). Additionally or alternatively, motor 212 itself may optionally include the gear assembly, for example providing a gear reduction of up to 16:1.

In accordance with the present inventive concepts, motor 212 and gear assembly 214 can be configured to resist cable motion at the bobbins. In this manner, the bobbins rotate only when driven by the motor, and resist other inherent motion that may otherwise be transferred through the cable from probe 400. In this manner, the motors 212 are substantially resistant to back-driving by forces applied by the steering cables. With enhanced motion resistance capability, the motors 212 can be powered down when not in use, for example, between motion cycles (e.g. steering and/or translation maneuvers), conserving energy, reducing heat output and extending lifespan of drive assembly 210. Also, when an external force is applied to probe 400, for example, when probe 400 is in contact with tissue, there is no need to power the motors of the probe to resist undesired probe motion.

Such enhanced motion resistance can be achieved in any of a number of approaches. In some embodiments, a worm gear gearing mechanism can be employed for drive assembly 210. Such worm-gear gearing mechanisms are inherently non-backdrivable. In other embodiments, a stepper motor having a suitable retention force can be applied. In another embodiment, a DC motor with a short-circuited drive inductor can be employed, since any rotation relative to the motor magnets is resisted in this configuration. In other embodiments, mechanical gears with anti-rotation elements, for example pawls or ratchets, can be employed. In other embodiments, magnetic-based position-holding assemblies can be employed to provide a motor retention force.

Referring to FIGS. 8A, 8B, base assembly 200 includes a base handle 220 for positioning the base, a motor 212, a gear assembly 213 and a capstan 216. Gear assembly 213 comprises a worm 213a and a mating gear 213b. In the close-up view of FIG. 8B, it can be seen that motor 212 drives worm gear assembly 213. The threads of the worm 213a mesh with gear 213b for driving the capstan (not shown) and corresponding bobbin. Any counter-rotational force of the gear 213b applied by the cable attached to the corresponding bobbin is resisted by the interface of gear 213b and worm 213a. In this manner, the cable is locked in place due to the inherent locking (i.e. anti-backdrivable nature) of the mechanical relationship between the worm 213a and gear 213b.

In some embodiments, a motor 212 is attached to the chassis of the base assembly 200 at a motor mount 218. In some embodiments, a plurality motor mounts 218 are each rotatably mounted to the chassis of the base assembly 200 and rotate about the axle of gear 213b. In some embodiments, the motor mount 218 is constructed and arranged to rotate with minimal resistance. In some embodiments, the motor mount 218 rotates on a low resistance bearing. In some embodiments, a portion 218a of the motor mount 218 rotates to interface with a load cell 221 mounted to the chassis of the base assembly 200. A load cell includes a cable 223 for providing load information to feeder unit 100a and/or interface unit 100b.

In this manner, motor mount 218 engages with load cell 221 to provide a measured force that can be correlated to cable tension in the cable applied to the bobbin corresponding with the given motor 212. The cable tension applies a torsional force on the bobbin and the associated engaged capstan. This in turn applies a torque to the gear 213b (e.g. of gear assembly 213) and thus motor 212 and motor mount 218. The motor mount 218 tends to rotate as cable tension is applied. Such rotation applies force to the load cell 221. In this manner, the force measured at the load cell can be correlated to cable tension.

In some embodiments, the interface of the motor mount 218 and load cell 221 can include an adjustment screw 219 for ensuring and/or adjusting contact therebetween. A biasing spring 217 can be further included for ensuring a minimum load is always present on the load cell 221. This configuration avoids load cell measurements near zero force, which can be a desired avoidance in such applications.

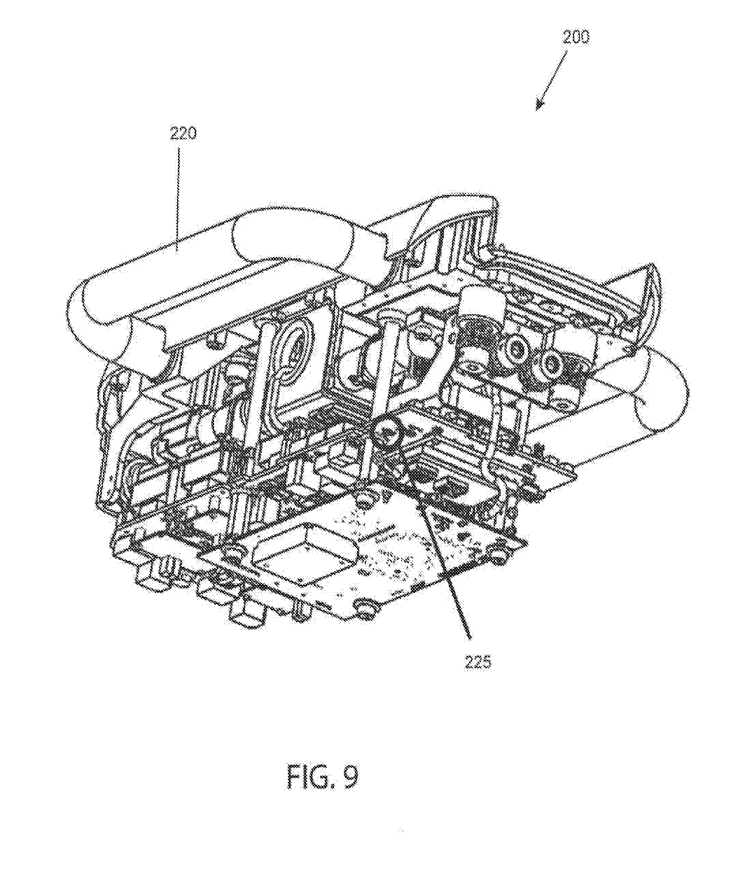

FIG. 9 is a partial cutaway perspective front view of a feeder assembly, in accordance with embodiments the present inventive concepts.

In some embodiments, the base assembly 200 of the feeder assembly 102 can include a position sensor, such as position sensor 225 shown in FIG. 8A mounted to a circuit board of base assembly 200. In some embodiments, the position sensor 225 can measure a relative position (e.g. orientation and/or location in 3D space) of the feeder assembly 102, at one or more time intervals during use, such as to determine whether feeder assembly 102 has been moved and/or to determine a geometric orientation of feeder assembly 102. Position sensor 225 can comprise a motion sensor, a displacement sensor and/or an accelerometer, or the like. In some embodiments, a multidimensional level switch, for example a bank of mercury switches, a gyroscope, or other sensor that provides angular orientation with respect to gravity may be employed for sensor 225. For purposes of the present description, the term “position sensor” is meant to include all types of sensors capable of measuring the position or displacement of an object in one or more degrees of freedom.

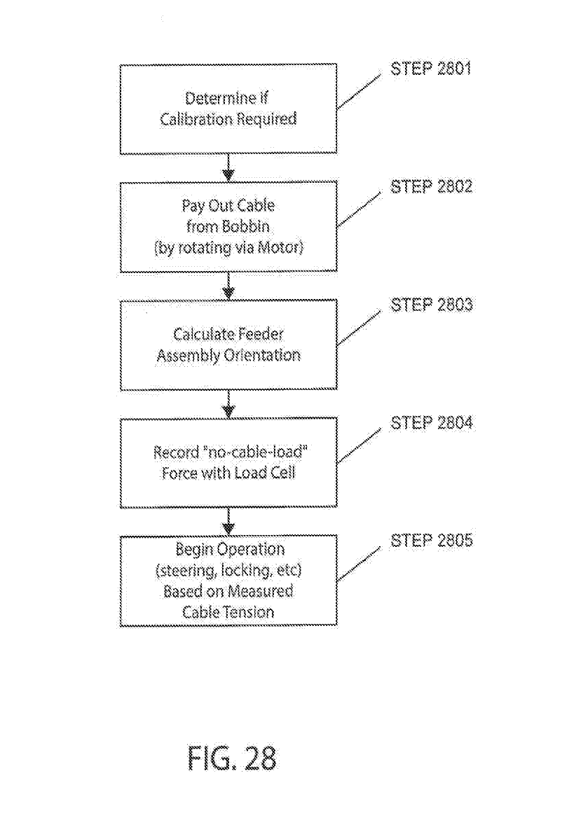

As described herein, the forces operating on the cables of probe 400 and/or the forces applied to one or more load cells 221, can change depending on the position and angular orientation of probe 400. This is also true of the forces that operate on the cables and/or the forces applied to one or more load cells 221 as a function of the position and angular orientation of other portions of feeder assembly 102. Accordingly, during a procedure, one or more calibration procedures can be performed based on the current position and angular orientation of feeder assembly 102, such as the calibration procedure described herein in reference to FIG. 28. Upon detection of a certain amount of feeder assembly 102 motion, as detected by the position sensor 225, the system may re-calibrate to account for variation in forces applied to the cables and/or load cell 221, as a result of the change in position of feeder assembly 102.

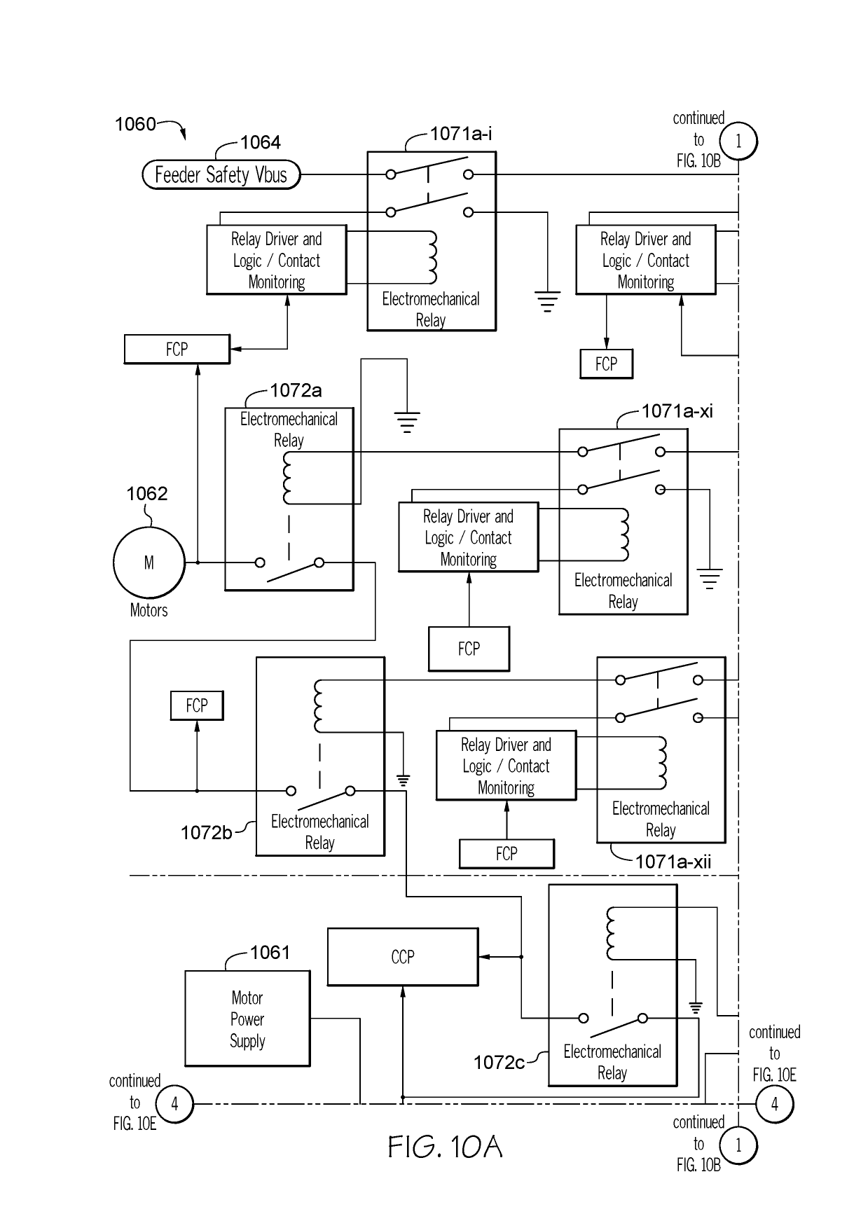

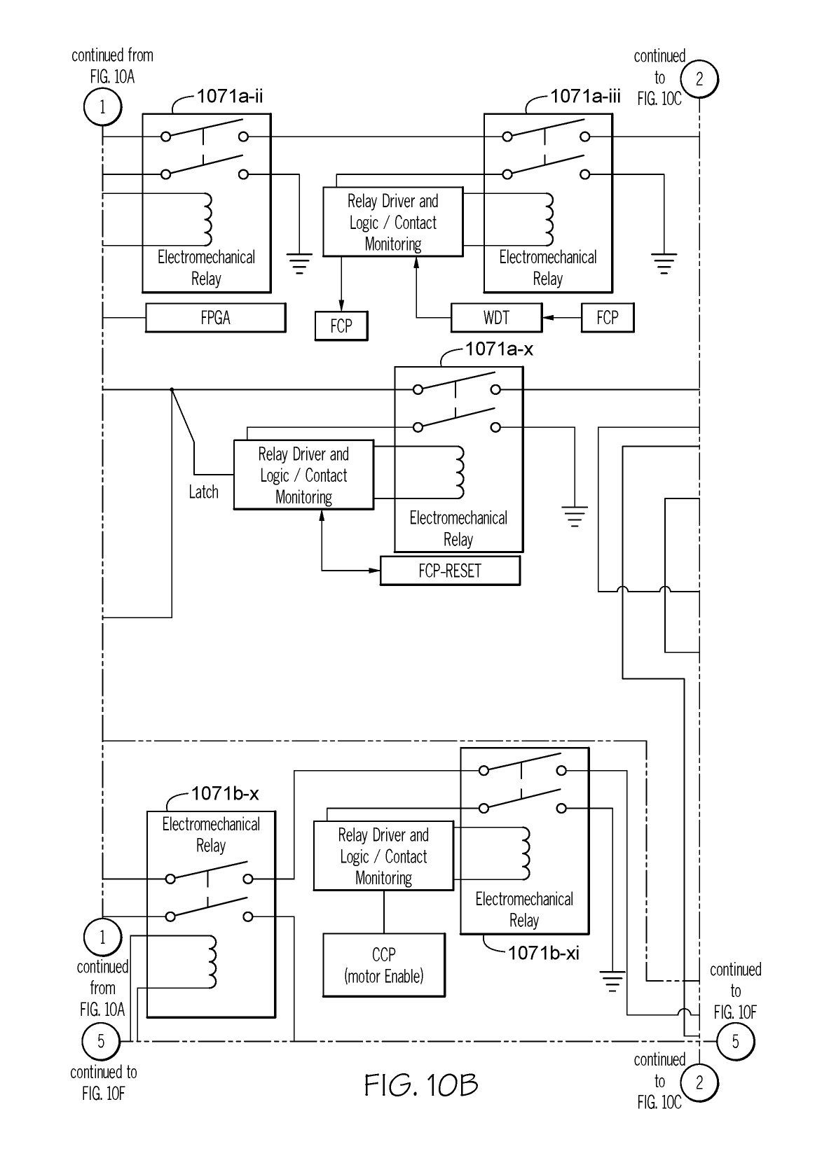

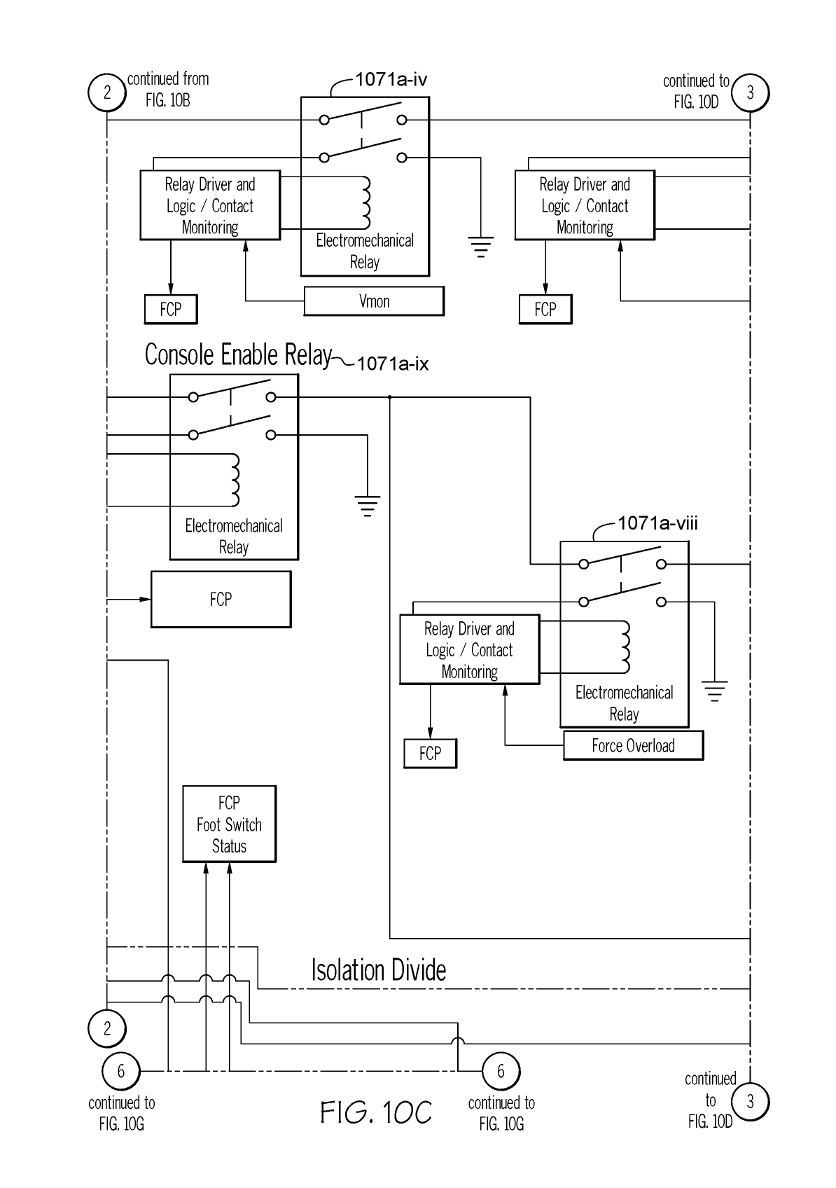

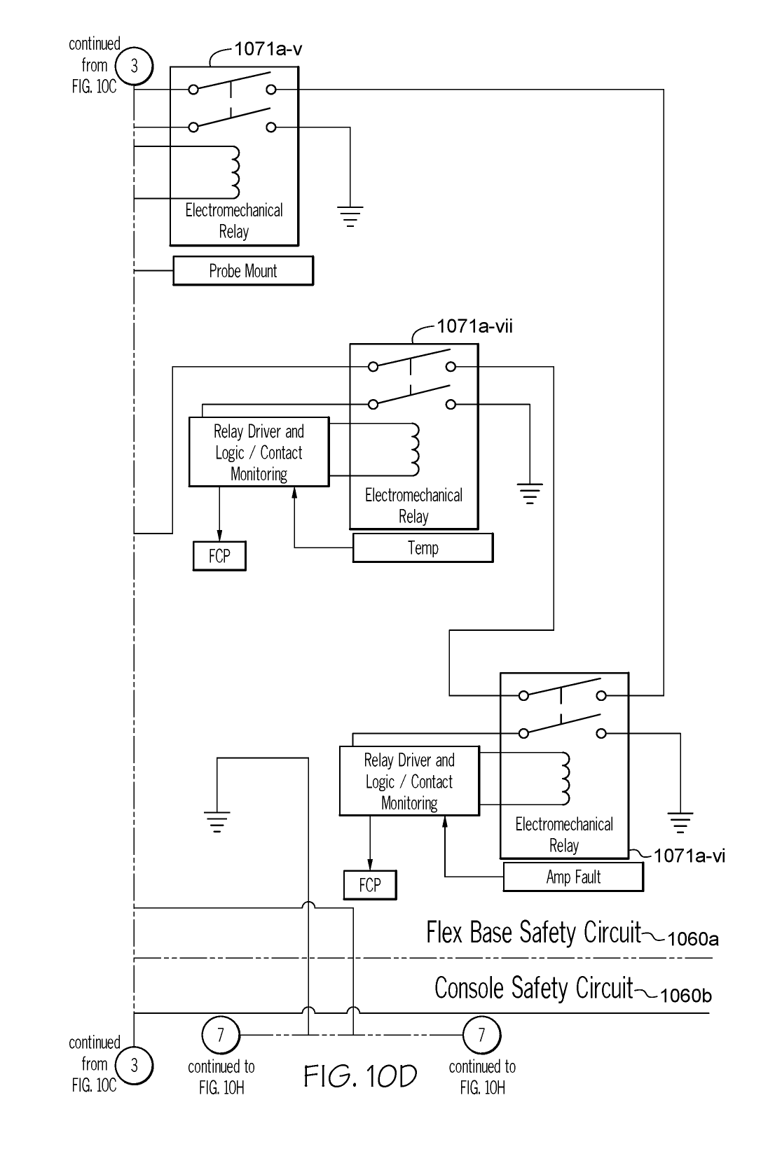

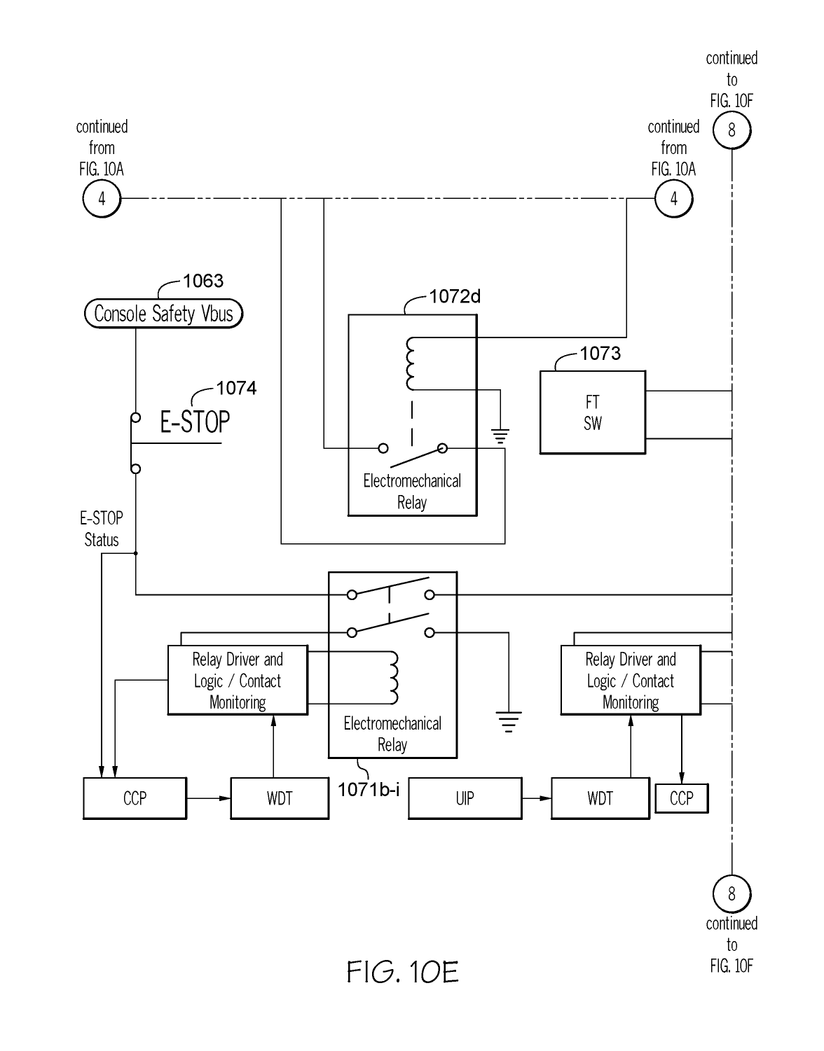

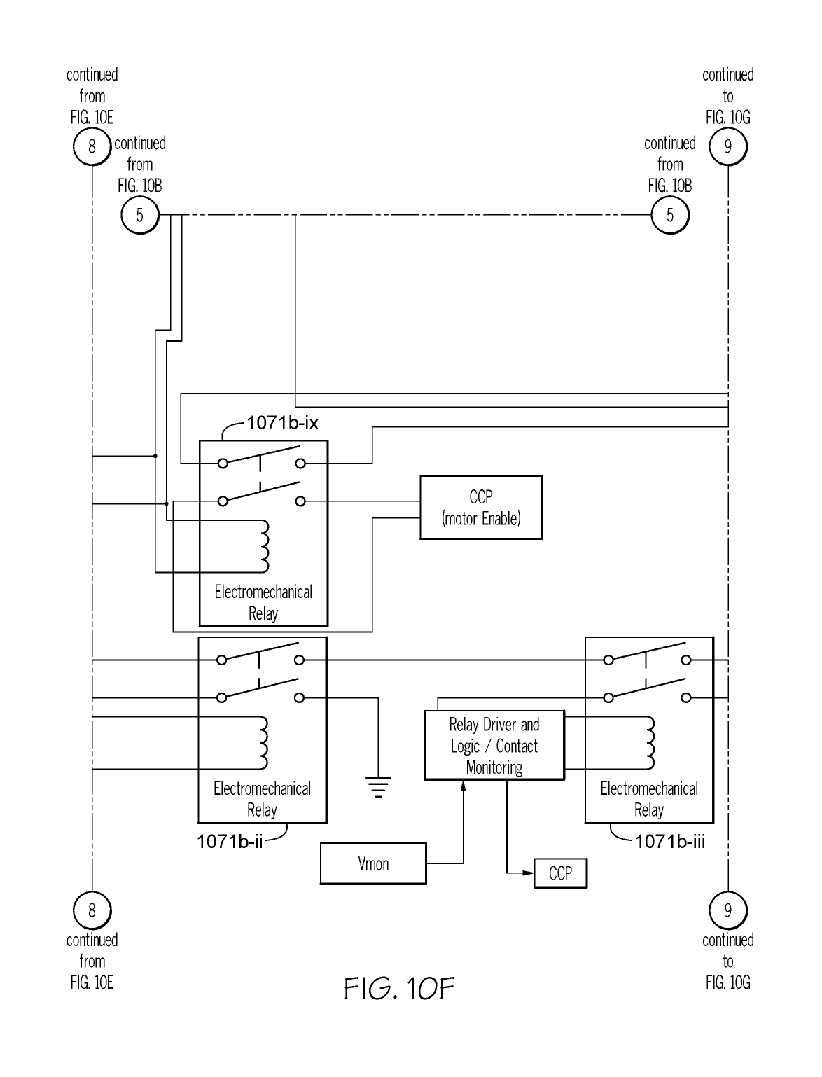

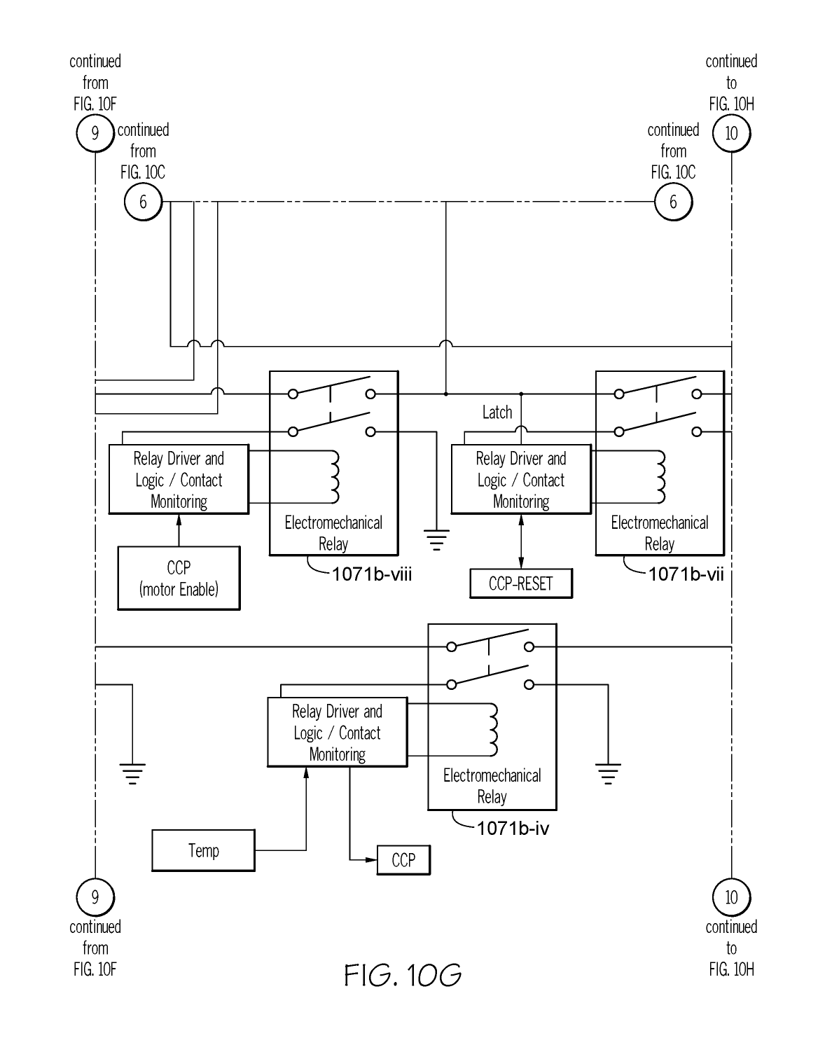

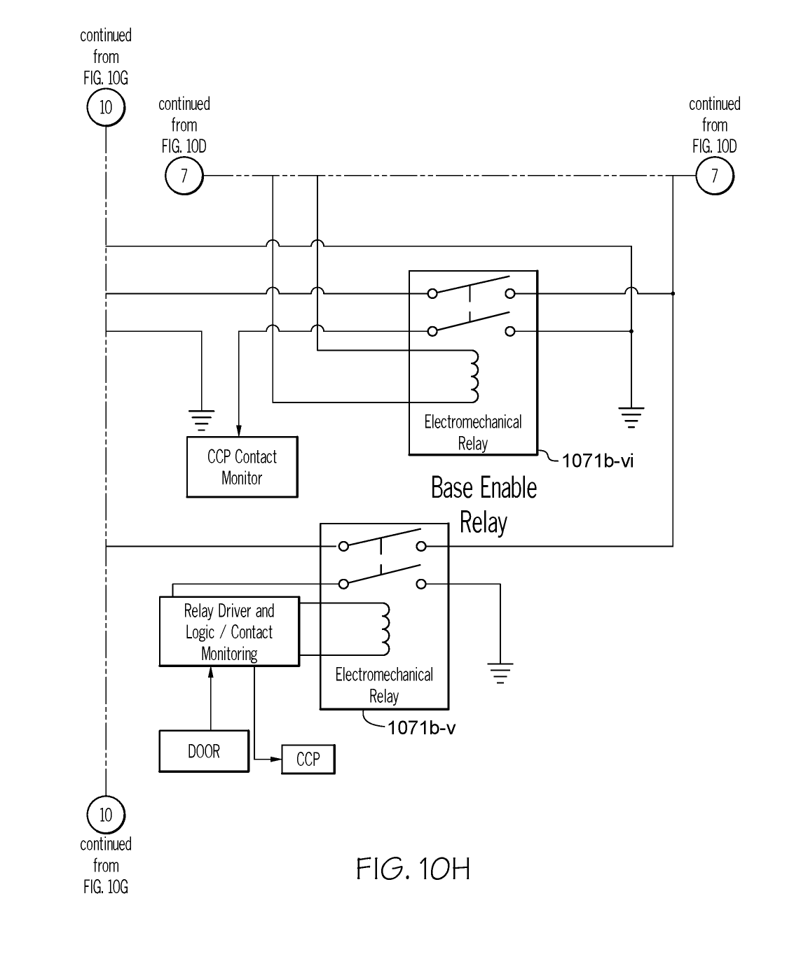

Referring now to FIG. 10, a schematic of a safety system 1060 is illustrated, consistent with the present inventive concepts. Safety system 1060 may be part of or otherwise communicate with base assembly 200, console 100b or a combination thereof. Safety system 1060 comprises a series of switches, including safety relays 1071, power relays 1072, and at least one user activated switch, such as foot switch 1073 and/or emergency switch 1074 (singly or collectively switch). System 100 of the present inventive concepts, further comprises a power supply, motor power supply 1061, and one or more motors, motor 1062 (e.g. a cable drive motor or carriage drive motor such as motors 212 described herein). Safety system 1060 can comprise a series of mechanical, electro-mechanical or electronic relays or switches, configured to control power to one or more power relays 1072 or other electrical components of the present inventive concepts. Power relays 1072 can comprise a series of electro-mechanical or electronic relays, configured to connect and/or disconnect (herein after “control”) power (e.g. power supplied from motor power supply 1061) to one or more motors (e.g. motors 1062) or other electrical components of the present inventive concepts, such as one or more motors configured to control the tension on a cable used to steer and/or lock all or a portion of articulating probe 400 and/or a motor configured to translate a carriage assembly of the present inventive concepts. In some embodiments, multiple switches are connected in series, such that if any single switch is in an “open position” (such as an open switch, or an unpowered relay, such as to create an open circuit), any or all motors of the system are disconnected from the motor power supply.

Safety system 1060 further comprises a safety bus in interface unit 100b (also referred to as console 100b), console safety bus, bus 1063. Safety system 1060 further comprises a safety bus in feeder unit 100, feeder safety bus, bus 1064. In some embodiments, multiple safety relays 1071 are connected in series, such that with all safety relays 1071 in a closed position, bus 1063 and/or bus 1064 are electrically connected to one or more power relays 1072, such as one or more power relays connected in series, such that the one or more power relays 1072 are in a closed position, and motors 1062 are electrically connected to motor power supply 1061, as is described in detail herein.

Safety system 1060 can include one or more electronic modules, such as one or more electronic modules positioned in one or more of: top assembly 300, base assembly 200 and interface unit 100b. In some embodiments, a first safety subsystem, 1060a is positioned in the base assembly 200 and a second safety subsystem 1060b is positioned in interface unit 100b. Safety subsystems 1060a and 1060b can be interconnected such that an open switch in either subsystem, will open one or more power relays 1072, disconnecting power from any or all motors 1062. This particular configuration can provide an advantage when system 100 includes patient electrical isolation circuitry, such as isolation circuitry positioned between interface unit 100b and feeder unit 100a.

Switches can be configured to monitor system parameters (e.g. via the control inputs to each relay 1071), such that system “fault” results in the opening of the relay 1071 configured to detect the fault which has occurred. Relays 1071, as well as switches 1073 and 1074, form a state machine that determines whether or not the motor power relays 1072 under their control can be closed based on the state of a number of inputs (e.g. all inputs, relays and switches must be closed in order for power relays 1072 to close).

Safety system 1060, including each sub-system 1060a and 1060b can detect momentary drop-outs of any monitored parameter and render system 100 in a “safe state”, where any or all motors 1062 are disconnected from motor power supply 1061, by opening the respective safety relay 1071 which in turn interrupts the control current to the power relays 1072.

Each safety relay 1071 is serially connected (e.g. arranged in a “chain” connection scheme, such as the serial connection of relays shown), and all must be closed in order for the power relays 1072 to close.

All safety relay 1071a contact statuses in the base assembly 200 are monitored by a processor in feeder unit 100a, the feeder control processor (FCP), which can be positioned in base assembly 200.

All safety relays 1071b contact statuses in the interface unit 100b are monitored by a processor within interface unit 100b, the console control processor (CCP).

### Base Assembly Relays

As described above, base assembly 200 can include one or more safety relays 1071a, or other switches, as shown. The relays and/or switches can interrupt feeder safety bus 1064 when in an open position. Each relay or switch must be closed (e.g. not to interrupt bus 1064) in order to power (e.g. close) one or more power relays 1072a within base assembly 200.

Feeder Control Processor (FCP) controls a first safety relay 1071a-i. In some embodiments, this relay is closed when all software checks have been passed. If a software parameter monitored by FCP is outside of an acceptable range, the resulting signal will open the associated safety relay 1071a-i.

An FPGA can be included and control a safety relay 1071a-ii as shown. The FPGA closes the safety relay 1071a-ii in the absence of motor encoder position or communication errors. The detection of any errors will result in the opening of the associated safety relay 1071a-ii.

A FCP Watch Dog Timer (WDT) can be included and control a safety relay 1071a-iii as shown. The FCP WDT monitors the proper performance of the FCP and must be asserted continuously (e.g. no less often than every 135 ms), failure to do so (e.g. due to a software crash, FCP hardware failure or similar adverse event) will result in the WDT opening the associated safety relay 1071a-iii.

A Voltage Monitor (VMON) can be included and control a safety relay 1071a-iv as shown. The VMON circuitry monitors supply voltages on the base assembly 200, and the 15V and 28 V supplies that power electronics in base assembly 200. The critical supply voltage powering the FCP is redundantly monitored. Voltages monitored must remain at all times within a predetermined (e.g. ±10%) window of the nominal voltage otherwise a VMON error results, opening the associated safety relay 1071a-iv.

Probe Mount detection circuitry can be included and control a safety relay 1071a-v as shown. This circuitry detects the presence of the top assembly 300. If top assembly 300 is not detected, then the associated safety relay 1071a-v will be open.

Amplifier Fault (Amp Fault) detection circuitry can be included and control a safety relay 1071a-vi as shown. This circuitry detects proper function of an amplifier circuit. If a fault is detected, the associated safety relay 1071a-vi will open.

A Temperature Sensor (Temp) can be included and control a safety relay 1071a-vii as shown. The temperature sensor measures ambient temperature with the base assembly 200 and should it rise above a maximum allowable value (e.g. 60° C.), the associated safety relay 1071a-vii will open.

Force Overload circuitry can be included and control a safety relay 1071a-viii as shown. This circuitry monitors the tension on any or all steering cables (e.g. steering cables used to steer and/or lock probe 112 of system 100). If the monitored tension rises above a preset maximum value, the associated safety relay 1071a-viii will open.

A Console Enable Relay 1071a-ix can be included as shown. For this relay to close, all safety relays 1071b in the console 100b, except the Base Enable Relay ### and CCP Reset controlled relay ###, and foot switch enabled relay ###, must be closed.

A FCP Reset Signal can be included and control a safety relay 1071a-x as shown. All preceding relays 1071a must be closed and the reset circuit must be strobed by a rising edge pulse from the FCP for this relay 1071a-x to close. The control circuitry (e.g. the circuitry which monitors the FCP Reset signal and controls the state of the associated safety relay 1071a-x) is configured as a latch and the input controlled by the FCP is designed to respond only to the rising edge of the strobe signal. AC coupling is employed so that if the associated FCP port is stuck in the high state, the circuitry will not allow this relay 1071a-x to close. However, once closed the FCP can no longer open relay 1071a-x. (Relay 1071a-x is a latching relay with two inputs, one is the status of the safety circuit which must be good in order to close, and the other is a strobe pulse from the FCP. Once strobed, the relay closes and remains closed until a fault is detected elsewhere in the safety circuit.) An interruption of any of the preceding relays for a time period (typically well<10 ms) will result in this relay 1061a-x opening.

Two safety relays 1071a-xi and 1072a-xii can be configured as separate enable relays which are independently controlled and monitored by the FCP, as shown. Both relays 1071a-xi and 1072a-xii must be closed in order to close the two motor power control relays 1072a and 1072b located on a Relay Daughter Board PCA located in console 100b.

### Console Relays

As described above, console 100b can include one or more safety relays 1071b illustrated FIG. 10, or other switches, as shown. The relays and/or switches can interrupt console safety bus 1063 when in an open position. Each relay or switch must be closed (e.g. not to interrupt bus 1063) in order to power (e.g. close) one or more power relays 1072b within console 100b.

An operator accessible emergency stop switch, E-STOP 1074, can be included as shown. The CCP monitors the status of the E-STOP switch to provide a signal correlating to an operator invoked emergency stop (e.g. a signal which can correlate to a message displayed on display 124 of FIG. 1).

A CCP Watch Dog Timer (WDT) can be included and control a safety relay 1071b-i as shown. The CCP WDT monitors the proper performance of the CCP and must be asserted continuously (e.g. no less often than every 135 ms), failure to do so (e.g. due to a software crash, CCP hardware failure or similar adverse event) will result in the WDT opening the associated safety relay 1071b-i.

A User Interface Processor (UIP) WDT can be included and control a safety relay 1071b-ii as shown. The UIP WDT can monitor the proper performance of the UIP and must be asserted continuously (e.g. no less often than every 135 ms), failure to do so (e.g. due to a software crash, UIP hardware failure or similar adverse event) will result in the WDT opening the associated safety relay 1071b-ii.

A Voltage Monitor (VMON) can be included and control a safety relay 1071b-iii as shown. The VMON circuitry monitors supply voltages on the Safety PCA, and the main power supply that powers electronics in the interface unit 100b. Voltages monitored must remain at all times within a predetermined (e.g. ±10%) window of the nominal voltage otherwise a VMON error results, opening the associated safety relay 1071b-iii.

A Temperature Sensor (Temp) can be included and control a safety relay 1071b-iv as shown. The temperature sensor measures ambient temperature with the interface unit 100b enclosure and should it rise above a maximum allowable value (e.g. 60° C.), the associated safety relay 1071b-iv will open.

A Door Sensor can be included and control a safety relay 1071b-v as shown. The Door Sensor is operated by a switch based safety interlock, which, if the interface unit 100b doors and/or circuit board holder are not properly in place, will result in the opening of the associated safety relay 1071b-v.

A Base (Feeder) Enable Relay 1071b-vi can be included as shown. For this relay to close, all safety relays 1071a in the base assembly 200, except the Console Enable Relay and FCP Reset controlled relay, must be closed.

A CCP Reset Signal can be included and control a safety relay 1071b-vii as shown. All preceding relays 1071b must be closed and the reset circuit must be strobed by a rising edge pulse from the CCP for this relay 1071b-vii to close. The control circuitry (e.g. the circuitry which monitors the CCP Reset signal and controls the state of the associated safety relay 1071b-vii) is configured as a latch and the input controlled by the CCP is designed to respond only to the rising edge of the strobe signal. AC coupling is employed so that if the associated CCP port is stuck in the high state, the circuitry will not allow this relay 1071b-vii to close. However, once closed the CCP can no longer open relay 1071b-vii. (Relay 1071b-vii is a latching relay with two inputs, one is the status of the safety circuit which must be good in order to close, and the other is a strobe pulse from the CCP. Once strobed, the relay closes and remains closed until a fault is detected elsewhere in the safety circuit.) An interruption of any of the preceding relays for a time period (typically well<10 ms) will result in this relay 1061b-vii opening.

A Footswitch (FTSW) 1073 can be included and control a safety relay 1071b-ix as shown. Footswitch 1073 is controlled by an external footswitch. Footswitch FTSW is configured such that if the associated footswitch is not activated (e.g. depressed) by an operator, it will result in the opening of the associated safety relay 1071b-ix.

Two safety relays 1071b-x and 1071b-xi can be configured as separate enable relays which are independently controlled and monitored by the CCP, as shown. Both relays 1071b-x and 1071-xi must be closed before the FTSW 1073 can close the two console motor power control relays 1072c and 1072d located on a Relay Daughter Board PCA located in console 100b.

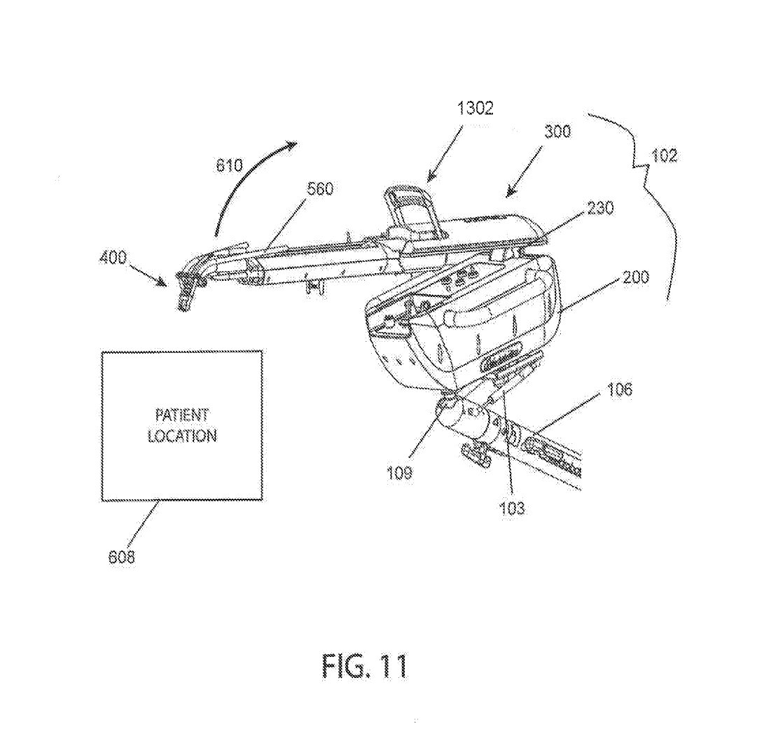

FIG. 11 is a perspective illustrative view of an articulating probe system feeder assembly 102 according to an embodiment of inventive concepts. As described, feeder assembly 102 may include base assembly 200 and top assembly 300 FIG. 12 is a perspective top view of base assembly 200 in accordance with embodiments of the inventive concepts. FIG. 13 is a bottom view of top assembly 300 in accordance with embodiments of the inventive concepts.

As described herein, in some embodiments, feeder assembly 102 can be mounted to a feeder cart 104 see e.g. FIG. 1 at a feeder support arm 106. Feeder support arm 106 can be adjustable in height and can include a plurality of sub-arms that pivot relative to each other. This adjustable configuration permits a range of orientations for positioning feeder assembly 102 relative to a patient location 608. Feeder assembly 102 includes base assembly 200 and top assembly 300 that can be constructed and arranged to be removably attachable to base assembly 200. Top assembly 300 includes an articulating probe 400 for example comprising a link assembly including an inner link mechanism 420 comprising a plurality of inner links 421, and an outer link mechanism 440 comprising a plurality of outer links 441, as described in connection with various embodiments herein (see e.g. FIG. 2). The position, configuration (e.g. flexibility) and/or orientation of probe 400 is manipulated by a plurality of driving motors and associated cables positioned in base assembly 200 and/or top assembly 300.

In an embodiment, feeder assembly 102 can be positioned relative to feeder support arm 106 over one or more degrees of freedom at a universal joint 109. One or more feeder supports 103 may be mounted between the base assembly 200 of the feeder assembly 102 and the feeder support arm 106, for supporting the weight of the base assembly 200 and/or feeder assembly 102 (i.e. the weight of both base assembly 200 and top assembly 300) in the region of the universal joint 109.

In some embodiments, top assembly 300 is removably attachable to the base assembly 200. In some embodiments, a hook 201 can be provided on base assembly 200 and a mating heel 1301 (see FIG. 13) can be provided on top assembly 300, to serve as a locator joint for initially seating top assembly 300 relative to base assembly 200. Once initially seated, hook 201 and heel 1301 can operate as a pivot for further seating top assembly 300 and base assembly 200. Top assembly 300 can be pivoted in a direction opposite arrow indicator 610 until completely seated. At this time, handle 1302 can be manually manipulated to lock top assembly 300 in position. A heel engagement assembly 230 can be spring loaded, for example in a direction indicated by arrow, to support mechanical play during the seating process and subsequently apply a retaining force between top assembly 300 and base assembly 200.

In some embodiments, electrical connectors 232, 1332 can include mating grounding connections (e.g. mating elements holes 234 and pins 1334 shown in FIGS. 12 and 13) that ensure proper grounding of top assembly 300. Mating surfaces of the connectors 232, 1332 can also be configured to accommodate the pivotal relationship of top assembly 300 relative to base assembly 200. In some embodiments, connectors 232, 1332 are constructed and arranged to provide non-electrical connections, such as fluid connections (e.g. transfer of fluids such as liquids or gases and/or transfer of fluid driven force such as hydraulic or pneumatic force) or mechanical connections (e.g. connections of one or more mechanical linkages). In some embodiments, connectors 232, 1332 are collectively constructed and arranged to provide a wiping force between one or more male pins prior to or during insertion into a female receptacle, such as to remove contamination from the male pins). In some embodiments, connector 232 and/or hole 234 are contained within a floating assembly, not shown but such as a floating circuit board which is biased in a neutral position by one or more springs that allow position adjustment in one or more degrees of freedom during connection of top assembly 300 to base assembly 200, such as to assist in alignment (e.g. alignment of multiple conductor electrical connections).

With reference to FIGS. 12 and 13, at the time top assembly 300 becomes completely seated on base assembly 200, capstans 216a, 216b on base assembly 200 become engaged with corresponding bobbins 1316a and gears 1316b on top assembly 300. In some embodiments, the mating capstans 216a and bobbins 1316a can comprise cable drive capstan/bobbin pairs for driving the steering and locking cables of the inner mechanism 420 and/or outer mechanism 440 of probe 400. In some embodiments, the mating capstans 216b and gears 1316b can comprise carriage drive capstan/gear pairs for driving the inner link and outer link carriages 1325a, 1325b, respectively, of probe 400 (see, e.g. FIGS. 5A. 5B. 6A).

In some embodiments, mating electrical connectors 232, 1332 on the base assembly 200 and top assembly 300 engage at the time of seating. The mating electrical connectors 232, 1332 serve as a pathway for electrical signals and/or other transmissions that are transferred between the base assembly 200 and top 300 assemblies.

Once seated, feeder assembly 102 can be positioned relative to a patient location 608 for a procedure. During a procedure, any of a number of emergencies can happen, which may require immediate removal of the probe 400 from the patient. In accordance with embodiments of the present inventive concepts, top assembly 300 can be manipulated by an operator to manually release the handle 1302, and top assembly 300 can be pivoted in a direction up and away from the patient location 608, for example, in a direction indicated by arrow 610, using the interface of the hook 201 and heel 1301 (also referred to as heel plate 1375 herein) as a pivot point. This arrangement provides an element of safety, as removal of the probe in this direction is highly desirable. At the same time, as top assembly 300 is released from base assembly 200, the capstans 216a, 216b and corresponding bobbins 1316a and gears 1316b become released from each other, immediately releasing the tension from all cables of probe 400. Such immediate release of cable tension is highly desirable for emergency situations, causing probe 400 to be in a limp or otherwise malleable state, allowing quick removal of the probe 400 from the patient regardless of the geometric configuration of probe 400 prior to the release. The emergency release can be performed in various system 100 failure or non-system related emergencies, such as when power is not being supplied to system 100.

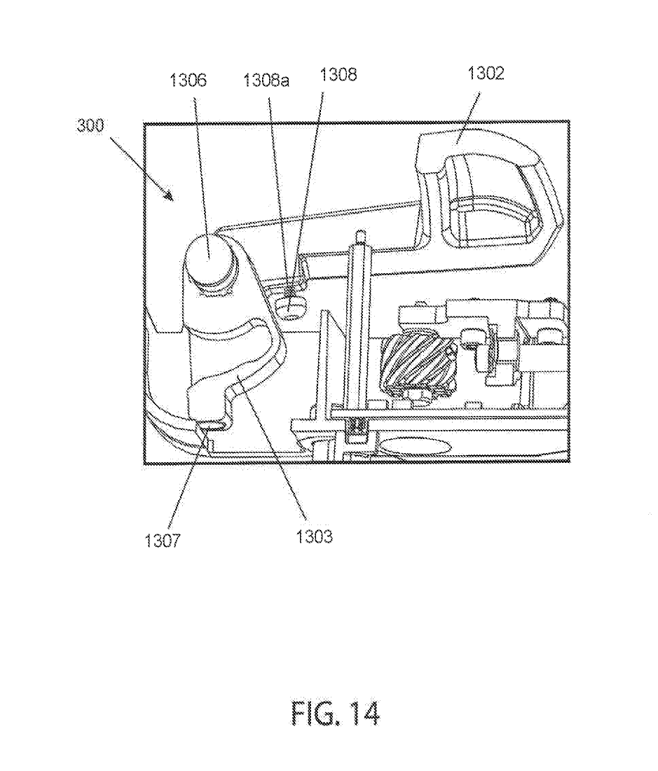

Referring to FIGS. 11-14, in some embodiments, top assembly 300 can include a cam 1303 that is actuated by handle 1302 (see FIG. 14). During seating, the cam 1303 can engage a corresponding cam engagement assembly 203 on base assembly 200, for locking top assembly 300 in a fixed, aligned position relative to base assembly 200. As top assembly 300 becomes fully seated, an alignment pin 204 on the base assembly engages a locator hole 1304 on top assembly 300, ensuring proper alignment. In some embodiments, alignment pin 204 or locator hole 1304, or both, can include tapered upper surfaces to accommodate mechanical play to assist in the alignment process. It should be appreciated that one or more alignment pins in base assembly 200 can be replaced with receiving holes, where the one or more mating holes of top assembly 300 are each accordingly replaced with an alignment pin configured to mate with the receiving hole of base assembly 200.

In some embodiments, a set of alignment pins, pins 205 and corresponding location holes 1305 can further be included for positioning a sterile drape between top assembly 300 and base assembly 200. In some embodiments, top assembly 300, including the probe 400 is a sterile apparatus that comes in contact with the patient, while the base assembly 200 and feeder arm support 106 and feeder cart 104 are not sterile. For this reason, a sterile drape can be applied between top assembly 300 and base assembly 200. The alignment pins 205 and location holes 1305 communicate with similarly positioned apertures on the drape for ensuring proper positioning of the drape during a procedure.

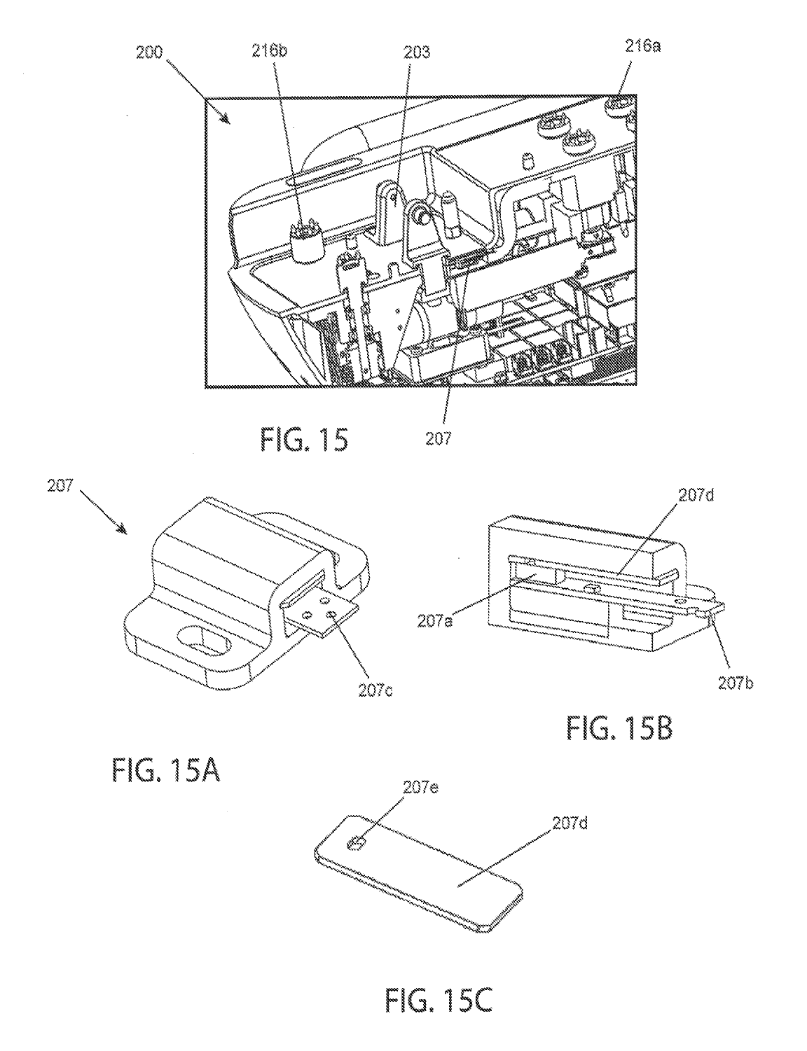

FIG. 14 is a perspective cutaway view of a handle 1302 of a top assembly 300 of a feeder assembly 102 of an articulating probe system 100, according to an embodiment of inventive concepts. FIG. 15 is a perspective cutaway view of a base assembly 200 of a feeder assembly 102 of an articulating probe system 100 according to an embodiment of inventive concepts. FIGS. 15A-15C are perspective views of proximity sensor componentry, in accordance with embodiments of inventive concepts.

Referring to FIG. 14, in some embodiments, top assembly 300 can include handle 1302 that pivots at pivot 1306 to engage cam 1303 to the cam engagement assembly 203 of base assembly 200. In some embodiments, a portion of the cam 1303 can include a magnet 1307 having a magnetic field of sufficient strength for emitting the magnetic field into base assembly 200.

Referring to FIG. 15, base assembly 200 can include a proximity sensor 207 suitable for detecting the magnetic field emitted by the magnet 1307 of top assembly 300, such as a magnet 1307 positioned in a portion of handle 1302. Accordingly, proximity sensor 207 is positioned in the vicinity of the region where magnet 1307 of handle 1302 is positioned when top assembly 300 is properly seated and locked into position on the base assembly 200.

In some embodiments, a bumper 1308 (see e.g. FIG. 14) can be located on the handle 1302 to provide for tactile feedback to an operator when engaged. The bumper 1308 can comprise a rubber or soft plastic material that is slightly deformable. In some embodiments, the bumper 1308 can have a threaded base 1308a inserted into a corresponding threaded opening in the handle 1302 as shown, so that its vertical position, relative to the handle 1302 can be adjustable (e.g. to adjust the amount of tactile feedback received). In alternative embodiments, the bumper 1308 can instead be positioned at an upper surface of the base assembly 200 to contact handle 1302 as handle 1302 is moved to a seated position.

Referring to FIGS. 15A-15C, proximity sensor 207 can comprise, in some embodiments, a magnetic sensor, for example, a Hall-effect sensor 207a (FIG. 15B), seated on an electrical board 207b having electrical contacts 207c for transferring electrical signals to and from sensor 207. In some embodiments, more accurate positioning is required than available by the Hall sensor, and accordingly, as shown in FIG. 15D, a Mu-metal plate 207d or the like can be included. Mu-metal plate 207d or the like blocks all magnetic field transfer to the Hall-effect sensor 207a. An aperture 207e within plate 207d as shown allows magnetic fields from magnet 1307 to pass for example, when top assembly 300 is properly engaged with base assembly 200, effectively increasing the positioning sensitivity of the proximity sensor 207. In some embodiments, the Mu-metal plate 207d can have two apertures 207e, one at each end, so that the plate 207d is thereby symmetric e.g. to allow placement in manufacturing in either direction, and potentially with either side oriented up. Such an embodiment may ease manufacturing constraints, eliminating the possibility of erroneous insertion of the plate 207d.

Although the illustrative embodiments depict the magnet 1307 positioned on top assembly 300 and the proximity sensor 207 positioned on base assembly 200, in other embodiments, their positioning can be reversed; namely, the magnet 1307 can be positioned on base assembly 200 and the proximity sensor 207 positioned on top assembly 300. Further, although the above embodiments depict magnet 1307 and sensor 207 positioned in a region of the cam 1303 and cam engagement assembly 203, their placement in other regions of top assembly 300 and base assembly 200 for establishing a coupling are also applicable to the inventive concepts.

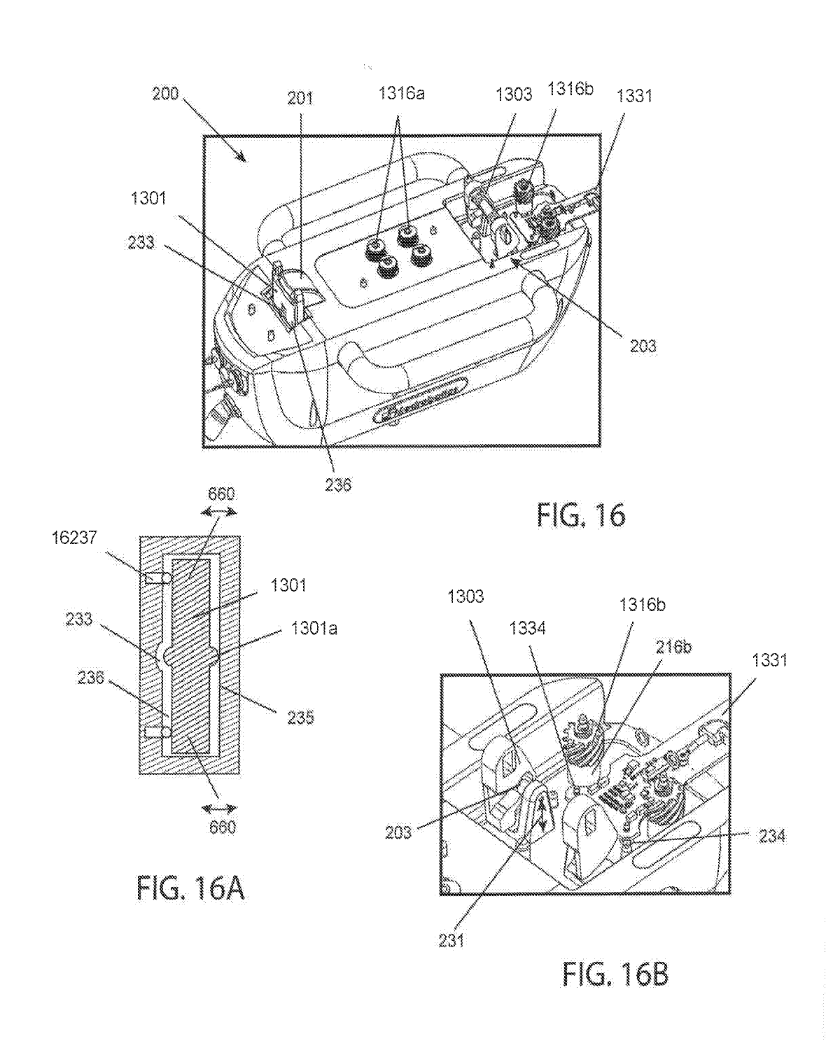

FIG. 16 is a perspective partial cutaway view of a base assembly 200 of a feeder assembly 102 of an articulating probe system 100 according to an embodiment of inventive concepts. FIG. 16A is a section view of a base assembly 200 and of the interaction of the heel 1301 and base cutout 233 according to an embodiment of inventive concepts. FIG. 16B is a close-up perspective view of the cam engagement assembly 203 of the base, in accordance with embodiments of inventive concepts.

Referring to FIGS. 16 and 16B, a partial cutaway view of the base assembly 200 is shown, along with certain components of top assembly 300 engaged with corresponding components of base assembly 200, including the heel 1301, bobbins 1316a, carriage gears 1316b, cam 1303, and electronics module 1331 of top assembly 300. Capstans 216a of base assembly 200 are engaged with bobbins 1316a but hidden from view in FIG. 16. Capstans 216b of base assembly 200 are engaged with carriage gears 1316b but also hidden from view in FIG. 16 (shown in FIG. 16b). It is assumed that top assembly 300 is properly mounted and secured to the base assembly 200. Referring to FIG. 16B it can be seen that the cam 1303 mates with the cam engagement assembly 203 when top assembly 300 is properly installed.

As described herein the cam engagement assembly 203 can be spring-biased in a vertical direction to allow for mechanical play in the seating and securing process, shown by arrow 231. Alignment pins 1334 of the top assembly 300 mate with corresponding holes 234 of base assembly 200 to ensure proper electrical connectivity between the base assembly connector 232 and top assembly connector 1332 (see FIGS. 12 and 13).

Referring to FIG. 16A, it can be seen that the heel 1301 of top assembly 300 is engaged with the hook 201 of base assembly 200. In some embodiments, the interaction of the heel 1301 and hook 201 can be the first point of contact in the seating process of top assembly 300 relative to base assembly 200. As described herein the heel 1301/hook 201 interface can provide the pivot point of top assembly 300 during seating and release, and serve as an emergency release feature, by providing pivot of top assembly 300 up and away from the patient, as described herein. In some embodiments, the hook 201 and/or heel 1301 can be spring-loaded to allow for mechanical play in the seating and securing process.

In some embodiments, the heel 1301 can include a ridge feature 1301a at its center portion. The ridge feature 1301a can operate as a contact point with a corresponding datum plate 235 surface of the receiving slot 236 of base assembly 200. This configuration longitudinally aligns top assembly 300 with base assembly 200 while allowing for a minimum, predetermined amount of angular offset in their positioning, for example, in a direction of rotation indicated by arrows 660. Such play in angular offset accommodates the alignment process during seating of top assembly 300 relative to base assembly 200. Ball plungers 16237 may be included in the receiving slot 236 opposite the datum plate 235 to maintain or bias the heel 1301 against the datum plate 235.

As described herein, during an emergency release of top assembly 300 and probe 400 relative to base assembly 200, the handle 1302 can be lifted, such that top assembly 300 is then free to rotate about the hook 201 of base assembly 200. As described herein, top assembly 300 rotates in a direction indicated by arrow 610 of FIG. 11, up and away from the patient location 608. As top assembly 300 pivots, bobbins 1316a and gears 1316b are lifted off the capstans 216a, 216b, respectively. This, in turn, releases tensions in all cables of probe 400, allowing safe removal of probe 400 from the patient, as the probe becomes “limp” and/or at least malleable. At the same time, upon pivoting, magnet 1307 is no longer detected by the proximity sensor 207, so electronic subsystems of system 100 can become aware of the release. Alignment pins 205, 1334 become disengaged from their corresponding holes 1305, 234. Electronics become disengaged at connectors 232, 1332, cutting power to the system camera and/or other systems electronics.

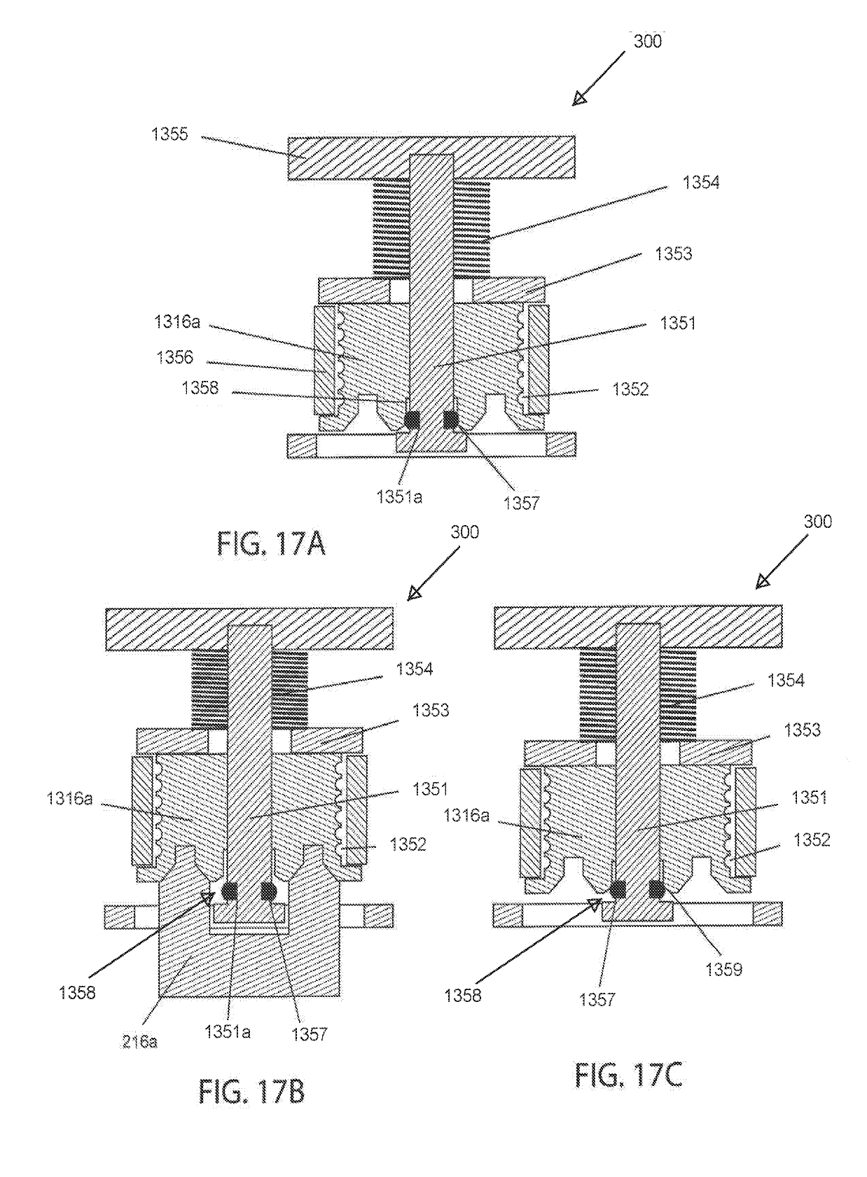

FIG. 17A is a side view of a cable bobbin of a feeder top assembly 300 in a shipping condition according to an embodiment of inventive concepts. FIG. 17B is a side view of a cable bobbin of the top assembly 300 in an operating condition according to an embodiment of inventive concepts. FIG. 17C is a side view of a cable bobbin of the top assembly 300 in a release condition according to an embodiment of inventive concepts.

Referring to FIG. 17A, a cable bobbin 1316a rotates about a bobbin axle 1351. The cable bobbin 1316a includes cable grooves 1352 for receiving a cable, for example, a cable wrapped helically about the bobbin 1316a. In some embodiments, the cable can comprise a steering and locking cable for controlling the outer link mechanism 440, or a locking cable for controlling the inner link mechanism 420 (see, e.g. FIG. 2). Alternatively or additionally, the cable can comprise a steering and locking cable for controlling, manipulation, or otherwise steering the inner link mechanism 420. The cable bobbin 1316a is seated on a bobbin washer 1353 in turn interfacing with a bobbin spring 1354. The bobbin spring 1354 is seated in a bobbin plate 1355, and allows for vertical travel of the bobbin 1316a relative to the bobbin plate 1355. In some embodiments, during manufacture, the cables are coupled to a distal link of the probe 400 at a first end and wound about the bobbins 1316a at a second end. The winds of the cable can be established by the cable groves 1352. During shipping, it is desired that the cables not lose tension or become released.

In some embodiments, to prevent release of the cable from cable grooves 1352, a cable clip can be included, such as clip 1356 shown, which rotatably engages bobbin 1316a allowing cable to be collected onto bobbin 1316a and paid out from bobbin 1316a while maintaining the cable surrounding bobbin 1316a in close proximity to bobbin 1316a.

In some embodiments, to prevent an unintentional release of the cable from the cable grooves 1352 and/or to otherwise prevent de-tensioning (e.g. unwinding) of the cables prior to attachment of a top assembly 300 to a base assembly 200 (e.g. during shipment of one or more top assemblies 300 to a clinical or other operator site), an o-ring 1357 can be fixedly attached or otherwise seated about a neck region of the bobbin axle 1351, such as in groove 1351a of axle 1351 as shown. In this embodiment, the bobbin 1316a can be provided with a counter bore 1358 of an inner diameter slightly less than an outer diameter of the o-ring 1357. The frictional relationship between the o-ring 1357 and the counter bore 1358 operates to resist rotation of the bobbin 1316a, and therefore resist de-tensioning of the cables prior to attachment of top assembly 300 to base assembly 200 (e.g. during shipment of one or more top assemblies 300). The force of the spring 1354 operating on the washer 1353 maintains the o-ring 1357 in the counter bore 1358 until top assembly 300 is ready to be attached to a base assembly 200 to perform a clinical or other procedure.

Referring to FIG. 17B, after the top assembly 300 is attached to a base assembly 200 (e.g. during a clinical procedure), the capstan 216a from the base assembly 200 pushes the bobbin 1316a in an upward direction, compressing the spring 1354 and removing frictional engagement between o-ring 1357 and bobbin 1316a. As a result, the bobbin 1316a operates in response to its corresponding capstan 216a and capstan drive assembly, without frictional resistance being applied to bobbin 1316a, (since o-ring 1357 is no longer in frictional engagement with bobbin 1316a).

Referring to FIG. 17C, after a release of top assembly 300 from base assembly 200 (e.g. after procedure completion or after an emergency release), the capstan 216a is no longer in contact with the bobbin 1316a. Accordingly, the spring 1354 operates to push the bobbin washer 1353 and bobbin 1316a in downward direction as shown. The o-ring 1357 once again engages an upper surface of the counter bore 1358, providing a slight, but not full, resistance to bobbin 1316a movement. A chamfer 1359 may be included on the exit of counter bore 1358 as shown, such that when o-ring 1357 is biased against chamfer 1359 by spring 1354 (as shown in FIG. 17C and resulting after top assembly 300 is removed from base assembly 200), some (minimal) frictional engagement between bobbin 1316a and o-ring 1357 is present (but less than occurs in the configuration of FIG. 17A).

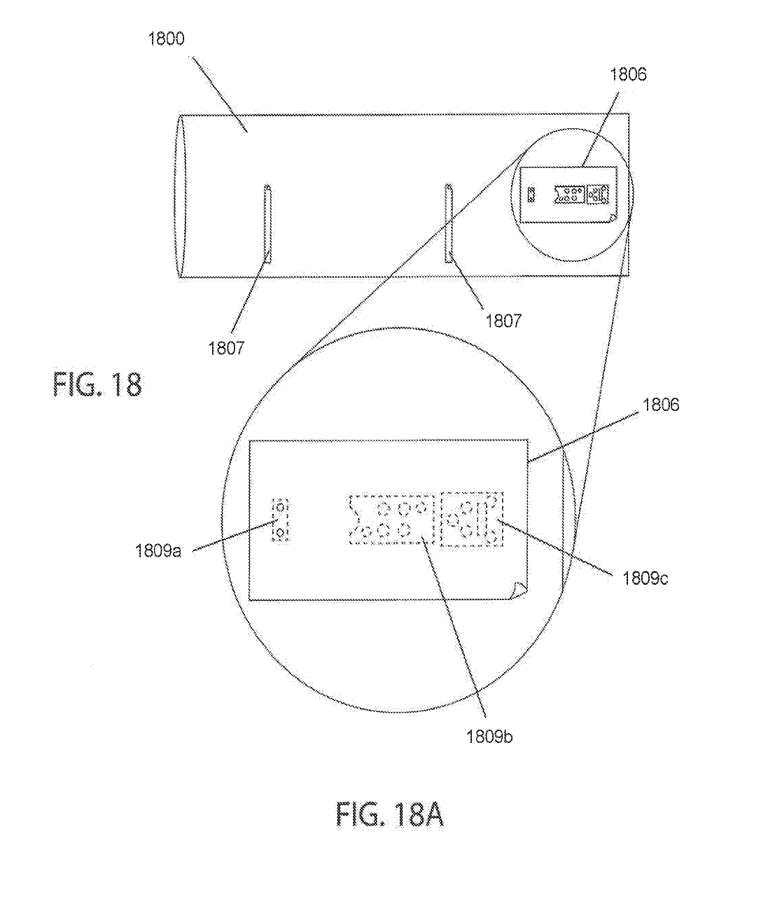

FIG. 18 is a top view of a sterile drape assembly 1800 according to an embodiment of inventive concepts. FIG. 18A is a magnified view of a portion of the drape assembly 1800 of FIG. 18. In some embodiments, the sterile drape can comprise High Density Polyetlene (HDPE) or other flexible, sterilizable material. As described herein, the sterile drape 1800 is provided during a procedure, to maintain sterility in the sterile environment, and to shield non-sterile portions of the system. One or more alignment plates 1809, such as alignment plates 1809a, 1809b and 1809c shown, are provided to align the pass-through regions of the base assembly 200 and top assembly 300 of the feeder assembly 102. Alignment plates 1809a, 1809b, 1809c include the pass-through regions (e.g. openings through which one or more components of top assembly 300 and/or base assembly 200 can pass). One or more straps 1807 may be provided for attaching the drape 1800 to features of the system console and feeder aim.

In preparation for a procedure, it is desired that the sterile drape be applied about the base assembly 200. After this, a certain amount of time may pass before top assembly 300 is mounted to the base assembly 200. During this time, maintenance of sterility is desired.

Accordingly, embodiments of the present inventive concepts provide a removable plate cover 1806 that covers the region of the alignment plates 1809. The removable plate cover 1806 can be removed just prior to attachment of the top assembly 300 to the base assembly 200. In some embodiments, the removable plate cover 1806 can cover the pre-formed openings in the alignment plates 1809. In some embodiments, the removable plate cover 1806 can be bonded to the alignment plate 1809 and/or surface of the drape 1800 using a well-known adhesive, bonding agent, or the like, and peeled therefrom by a technician or other operator just prior to use.

FIGS. 19A-19F illustrate various views of an inner link 421 of the present inventive concepts. FIG. 19A is a top view, FIG. 19B is a perspective view; FIG. 19C is a side view; FIG. 19D is a side-sectional view; and FIG. 19E is a bottom view; each of inner link 421. FIG. 19F is a side view of a distal inner link 421_{D} of the present inventive concepts.

FIGS. 20A-20F illustrate various views of an outer link 441 of the present inventive concepts. FIG. 20A is a top view; FIG. 20B is a perspective view; FIG. 20C is a side view; FIG. 20D is a bottom view; and FIG. 20E is a side-sectional view; each of outer link 441. FIG. 20F is a perspective view of a distal outer link 441_{D} of the present inventive concepts. Inner links 421 and outer links 441 can comprise similar or dissimilar materials, such as is described in detail herein. In some embodiments, inner links 421 and/or outer links 441 are constructed and arranged similar to the inner and outer links described in applicant's co-pending U.S. patent application Ser. No. 13/880,525, filed Apr. 19, 2013 and/or U.S. patent application Ser. No. 14/343,915, filed Sep. 12, 2012, the contents of each of which is incorporated herein by reference in their entirety.

In some embodiments, articulating probe 400 of the present inventive concepts comprises an inner link mechanism 420 including between 10 and 300 inner links 421, such as between 50 and 150 inner links 421, such as between 75 and 95 inner links 421, such as approximately 84 inner links 421. In some embodiments, inner links 421 comprise a length between 0.05″ and 1.0″, such as between 0.1″ and 0.5″, such as approximately 0.2″.

In some embodiments, inner links 421 comprise an effective outer diameter of between 0.1″ and 1.0″, such as an effective outer diameter of between 0.2″ and 0.8″, such as an effective outer diameter of approximately 0.35″.

In some embodiments, inner links 421 comprise a lumen, channel 422, configured to slidingly receive a cable to perform a function such as control locking and perform steering. Channel 422 can be centered in the relative geometric center of inner links 421, and can comprise a diameter between 0.01″ and 0.9″, such as a diameter between 0.02″ and 0.3″, such as a channel with a minimum diameter of approximately 0.07″ (e.g. a minimum diameter of a channel 422 with a tapered or hour-glass shaped profile as shown and described herein). In some embodiments, inner links 421 comprise multiple lumens, such as to slidingly receive a cable in each lumen, such as to allow both locking and steering of the inner link mechanism 420 of probe 400.

In some embodiments, inner links 421 comprise one or more materials configured to optimize locking of inner links 421. In some embodiments, inner links 421 comprise a high-friction material, such as an injection molded or other material comprising glass fibers. In some embodiments, inner links 421 comprise an isotropic construction, or at least one or more isotropic portions. In some embodiments, inner links 421 comprise a plastic material such as Noryl™ material.

Inner Links 421 can comprise a proximal surface 423 with a spherical geometry and/or a distal surface 424 with a spherical geometry. In some embodiments, both proximal surface 423 and distal surface 424 comprise a spherical geometry, such as to create a spherical surface to spherical surface interface between adjacent inner links 421 that maximizes locking (e.g. by increasing surface contact between adjacent inner links 421). In some embodiments, inner link 421 proximal surface 423 comprises a similar radius of curvature to distal surface 424. In some embodiments, inner link 421 proximal surface 423 comprises a radius of curvature of between 0.1″ to 1.0″, such as a radius of between 0.3″ and 0.7″, such as a radius of approximately 0.55″. In some embodiments, inner link 421 distal surface 424 comprises a radius of curvature of between 0.1″ to 1.0″, such as a radius of between 0.3″ and 0.7″, such as a radius of approximately 0.55″.

In some embodiments, inner links 421 comprise one or more working channel recesses or related curvatures, such as the three recesses 425 shown. Inner link 421 recesses 425 align with outer link 441 recesses 445 described herein. Recesses 425 can comprise a geometry constructed and arranged to receive a tool with a diameter between 1.0 mm and 10.0 mm, such as a diameter between 2.0 mm and 5.0 mm, or a diameter of approximately 2.5 mm (e.g. corresponding to a recess 425 diameter of approximately 3.3 mm).

In some embodiments, the most distal inner link comprises a different geometry than the more proximal inner links, such as distal inner link 421_{D}, whose side view is illustrated in FIG. 19F. Distal inner link 421_{D} can comprise a different geometry than inner links 421, such as the bullet-nose geometry shown in FIG. 19F. For example, distal inner link 421_{D} can comprise an opening 426 (e.g. a spherical shelf or other tapered opening) configured to receive an anchoring member (not shown but such as a ferrule) positioned on the distal end of a cable inserted through the series of inner links 421. Distal inner link 421_{D} can comprise a larger taper (e.g. less blunt) on its distal surface 424 than the distal surfaces of other inner links 421, such as to provide a sufficiently tapered distal end of inner link mechanism 420, such as to ease advancement of inner link mechanism 420 within a lumen of outer link mechanism 440. In some embodiments, distal inner link 421_{D} comprises a different (e.g. stronger) material than other inner links 421, such as a metal such as stainless steel or aluminum, such as to prevent damage to distal inner link 421_{D} at opening 426 due to forces exerted by anchoring a cable extending through the inner link mechanism 420 of the probe 400.

In some embodiments, articulating probe 400 of the present inventive concepts comprises an outer link mechanism 440 including between 5 and 150 outer links 441, such as between 10 and 100 outer links 441, such as between 20 and 80 outer links 441, such as approximately 56 outer links 441. In some embodiments, articulating probe 400 comprises more inner links 421 than outer links 441, such as at least 10% more inner links 421, such as at least 50%, 100%, 200%, 300% or 500% more inner links 421. The larger proportion of inner links 421 can correlate to a shorter relative length of inner link 421 which can reduce binding or other translation issues that otherwise might be encountered during advancement and/or retraction of inner link mechanism 420 within outer link mechanism 440. In some embodiments, outer links 441 comprise a length between 0.1″ and 2.0″, such as between 0.2″ and 1.0″, such as approximately 0.4″.

In some embodiments, outer links 441 comprise an effective outer diameter of between 0.2″ and 2.0″, such as an effective outer diameter of between 0.4″ and 1.6″, such as an effective outer diameter of approximately 0.68″.

In some embodiments, outer links 441 comprise two or more lumens, such as the three channels 442 shown, each configured to slidingly receive a cable to control both locking and steering of outer link mechanism 440. Channel 442 can be positioned with equal circumferential spacing (e.g. the approximately 120° spacing shown) within outer links 441, and can comprise a diameter between 0.06″ and 0.4″, such as a diameter between 0.01″ and 0.2″, such as a channel with a minimum diameter of approximately 0.047″ (e.g. a minimum diameter of a channel 442 with a tapered or hour-glass shaped profile as shown and described herein).

In some embodiments, outer links 441 comprise one or more materials configured to optimize both locking and steering of outer links 441. In some embodiments, a set of two or more outer links 441 positioned in a distal portion of outer link mechanism 440 comprise different materials (e.g. more lubricious materials configured to improve steering) than the materials used in two or more outer links 441 positioned in a proximal portion of outer link mechanism 440. In some embodiments, between 2 and 10 (e.g. between 2 and 7) outer links 441 positioned in a distal portion of outer link mechanism 440 comprise a more lubricious material than outer links 441 positioned in a more proximal portion of outer link mechanism 440, such as when the articulating probe 400 of the present inventive concepts is constructed and arranged to steer between 2 and 10 (e.g. between 2 and 7) outer links 441 simultaneously (e.g. an operator determined number of outer links 441 selected for steering). In some embodiments, the more lubricous material comprises one or more of: Ultem material; Ultem EFL 36 or similar material; Ultem 1000 or similar material; a Teflon additive; a material selected for enhanced rigidity of outer link 441; a material selected for minimal compression of outer link 441; and combinations of these. In some embodiments, the most distal outer link 441 comprises Ultem 1000 or similar material. In some embodiments, the less lubricious material of the more proximal outer links 441 comprises a material selected from the group consisting of: a liquid crystal polymer; IXEF or similar material; Noryl or similar material; and combinations of these. In some embodiments, the geometry and/or material of the more proximal outer links 441 is configured to lock outer link mechanism 440 and the geometry and/or material of the more distal outer links 441 is configured to both lock and steer outer link mechanism 440.

In some embodiments, one or more outer links 441 comprise a glass fiber material, such as an outer link 441 which includes approximately 30% glass fiber fill. In some embodiments, the most distal outer link 441_{D} does not comprise a glass fiber fill (or comprises less fiber fill).

In some embodiments, one or more outer links 441 (e.g. the most distal outer link 441_{D}) comprise an opaque material, such as to prevent light from passing through the outer surface of one or more portions of outer link mechanism 440. Additionally or alternatively, one or more outer links 441 can comprise a matte and/or dark finish, such as to prevent or minimize glare off of the outer surface of one or more portions of outer link mechanism 440.

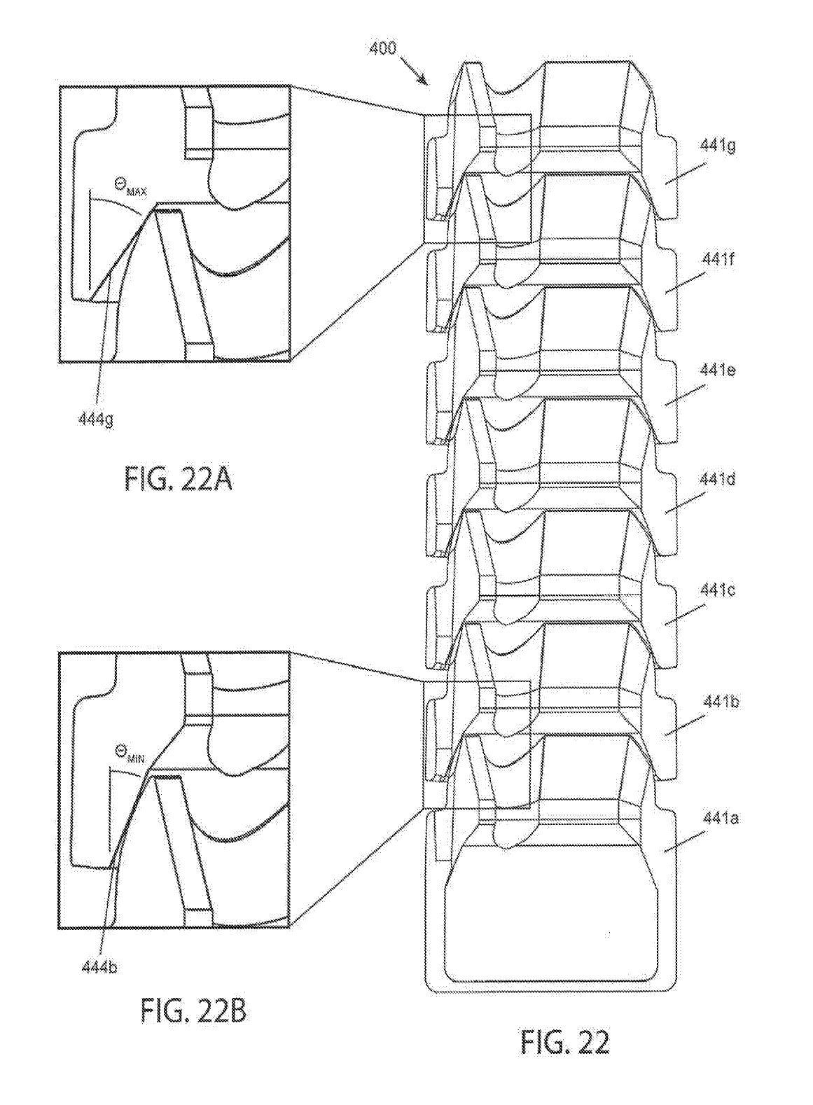

In some embodiments, a series of outer links 441 in a distal portion of outer link mechanism 440 are configured to articulate (e.g. during steering) in a cascading order (e.g. from distal to proximal), such as is described in detail in reference to FIG. 22 herebelow.

Outer Links 441 can comprise a proximal surface 443 with a spherical geometry (shown) and/or a conical geometry. In some embodiments, distal surface 444 comprises a dissimilar geometry, such as a conical geometry (shown), such as to create a conical surface to spherical surface interface between adjacent outer links 441 that enhances steering (e.g. by reducing surface contact between adjacent outer links 441 in a manner to reduce sticking). Alternatively, distal surface 444 can comprise a similar geometry, such as a spherical geometry similar to a spherical geometry of proximal surface 443. In some embodiments, outer link 441 proximal surface 443 comprises a radius of curvature of between 0.1″ to 1.0″, such as a radius of between 0.3″ and 0.8″, such as approximately 0.57″. In some embodiments, outer link 441 distal surface 444 comprises a cone with a taper between 5° to 70°, such as a taper of between 10° and 65°, such as a taper of approximately 23°.

In some embodiments, outer links 441 comprise one or more working channel recesses, such as the three recesses 445 shown. Outer link 441 recesses 445 are constructed and arranged to align with inner link 421 recesses 425 described hereabove. Recesses 445 can comprise a geometry constructed and arranged to receive a tool with a diameter between 1.0 mm and 10.0 mm, such as a diameter between 2.0 mm and 5.0 mm, or a diameter of approximately 2.5 mm (e.g. corresponding to a recess 445 diameter of approximately 3.3 mm). The working channel recesses 445 and 425 are configured to accommodate the translation of tools within them at all potential configurations of articulating probe 400 (e.g. all potential minimum and maximum radius of curvatures for multiple curved segments of inner link mechanism 420 and outer link mechanism 440).

In some embodiments, two or more outer links 441 comprise anti-rotation elements, such as pin 446 and slot 447 shown. The anti-rotation elements can be constructed and arranged to prevent one or more of the following events (e.g. during steering and/or during translation of the inner link mechanism 420 or the outer link mechanism 440); changes in working channel shape; pinching of tools or filaments passing through a working channel; moving of tools or filaments passing through a working channel; pinching of cables passing through channels 422 and/or 442; pinching or binding of inner link mechanism 420 as inner link mechanism 420 translates (e.g. advances or retracts) within outer link mechanism 440; and combinations of these. In some embodiments, pin 446 (FIG. 20B) and slot 447 (FIG. 20E) are constructed and arranged in a same or similar manner as described in applicant's co-pending U.S. patent application Ser. No. 14/343,915, filed Sep. 12, 2013, the content of which is incorporated herein by reference in its entirety.

In some embodiments, the most distal outer link comprises a different geometry than the more primal outer links, such as distal outer link 441_{D}, whose perspective view is illustrated in FIG. 20F. Distal outer link 441_{D} can comprise one or more function elements, such as a component selected from the group consisting of: a camera such as camera 448a, one or more light emitting components such as LEDs such as LEDs 448c; an electronics module; an irrigation lumen and/or nozzle such as irrigation port 448b; and combinations of these. Distal outer link 441_{D} can comprise one or more side ports, such as the two side ports 450 shown (e.g. configured to receive a tool support as described herein). Thus, an overall width of the distal outer link 442_{D} is greater than that of the other outer links 441 due to the side ports 450 which may be integrated with the main body, or otherwise coupled to the main body. In some embodiments, one or more (non-distal) outer links 441 can include one or more similar side ports, (not shown) but such as is side ports 455 described herein.

The channels (i.e. lumens) and working channel recesses of inner links 421 and/or outer links 441 can comprise an hour-glass or otherwise tapered profiles. The tapered profiles can be configured to prevent pinching of one or more filaments passing therethrough. In some embodiments, recesses 425 (as shown), recesses 445 (as shown), channels 422 (as shown) and/or channels 442 comprise an hour-glass profile. The hour-glass profile can be used to minimize the maximum diameter of the channel or recess, such as would be necessary if the channel or recess had a single, straight taper. In some embodiments, one or more of recesses 425, recesses 445, channels 422 and/or channels 442 comprise a tapered profile such as is described in applicant's co-pending U.S. patent application Ser. No. 13/880,525, filed Apr. 19, 2013, the content of which is incorporated herein by reference in its entirety.

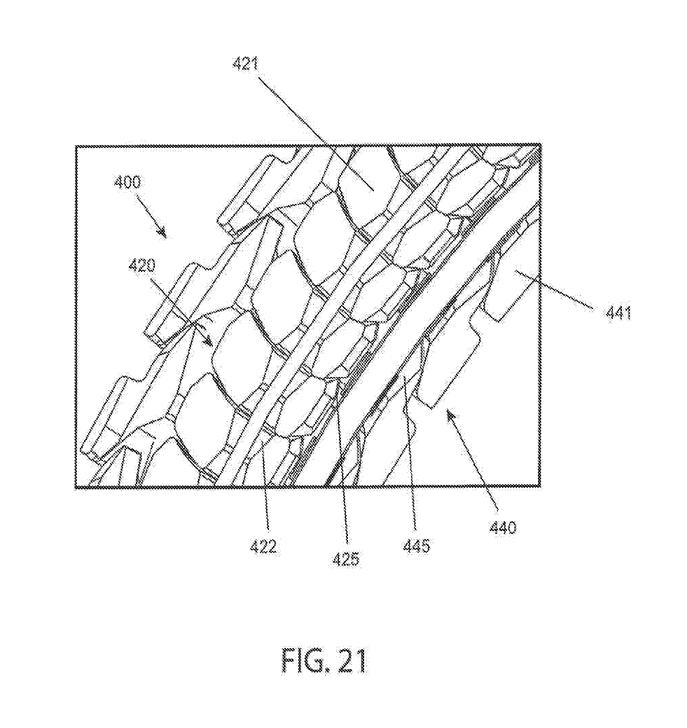

In FIG. 21, the hour-glass profiles within articulating probe 400 are illustrated in a side sectional view. Articulating probe 400 comprises inner link mechanism 420 and outer link mechanism 440. Inner links 421 and outer links 441 comprise geometries defining hour-glass profiles in channels 422 and the working channels created by recesses 425 and 445. In the embodiment of FIG. 21, channels 442 of outer link mechanism 440 comprise a linear tapered profile. In some embodiments, channels 442 of outer link mechanism 440 also comprise an hour-glass profile.

Referring now to FIG. 22, a side sectional view of the distal portion of an outer link mechanism 440 of articulation probe 400 is illustrated, consistent with the present inventive concepts. FIGS. 22A and 22B illustrate two magnified views of the conical to spherical interface of the outer link of FIG. 22, consistent with the present inventive concepts. A distal portion of articulating probe 400 comprises a series of seven outer links 441a through 441g (singly or collectively outer links 441), arranged distally to proximally (i.e. 441a the most distal). Distal link 441a can be constructed and arranged similar to distal outer link 441_{D} described hereabove. Articulating probe 400 can be configured such that at least distal outer link 441a and outer link 441b can be steered, while allowing additional adjacent links of outer links 441 to be steered, such as up to the seven outer links 441 shown. The contacting surfaces between conical distal surface 444 and the adjacent spherical proximal surface 443 defines a circle, reducing the surface area in each interface as described hereabove.

In some embodiments, the set of distal outer links 441 to be steered are constructed and arranged such that during steering, distal outer link 441a begins to articulate prior to next link 441b, which articulates prior to next link 441c and so on in a cascading fashion. This cascading series of initial articulation can be created in numerous ways. In some embodiments, the taper angle of each distal surface 444 of outer links 441b through up to 441g (e.g. to allow 7 segment steering) increases from taper angle θₘᵢₙ for link 441B to 0ₘₐₓ for link 441G (as shown in FIGS. 22A and 22B, respectively) causing an increased mating force (e.g. due to a resultant force vector change) between each set of sequential outer links 441. Since the mating force between outer links 441a and 441b is the smallest due at least in part to the smallest taper angle θₘᵢₙ for link 441B, followed by the mating force between outer links 441b and 441c, and so on, articulation during steering initiated with outer link 441a, and sequentially cascades distally. In these embodiments, the taper angle can comprise a set of taper angles selected from any group of increasing angles between 10° and 65°, such as a set of two or more taper angles (e.g. to support steering of two or more outer links 441) increasing from 10° in 1° increments or a set of two or more taper angles increasing from 10° in 5° increments. Alternatively or additionally, other characteristics of outer links 441 can be varied between 441a and 441g, such as a characteristic selected from the group consisting of: other geometric changes such as a geometric change affecting interface force; material change such as a sequential set of lubricity that decreases from 441a to 441g; changes in contacting surface area that cause the desired cascade; and combinations of these.

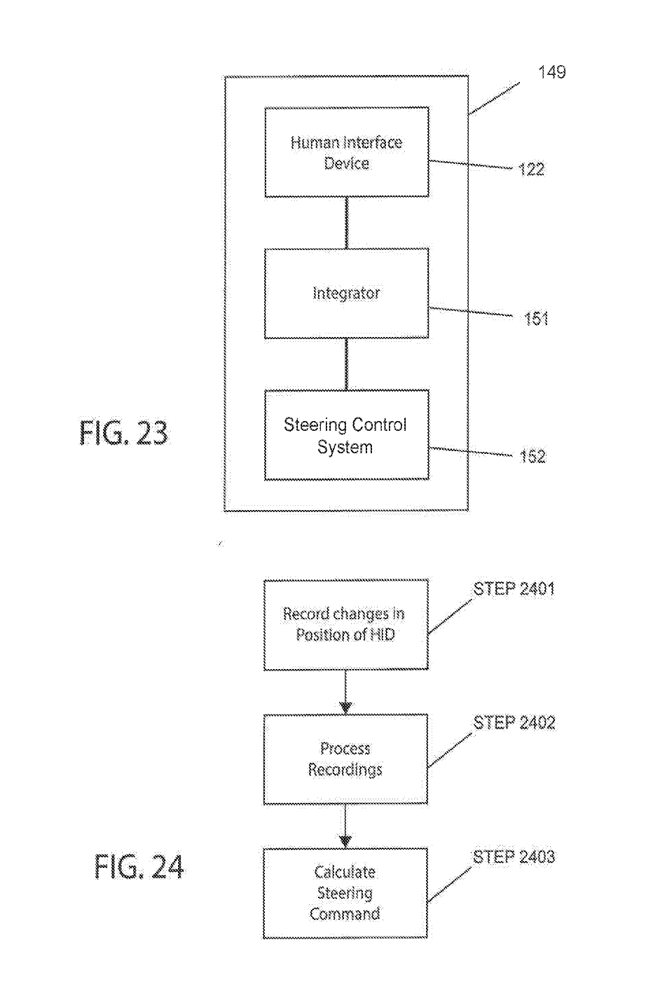

System 100 (e.g. feeder unit 100a and/or interface unit 100b ) can comprise one or more techniques, methods, or the like, for example, derived from algorithms, and used to provide safe and effective operation of articulating probe 400. In some embodiments, system 100 comprises one or more techniques, methods, or the like, for example, embodied as software code which may be stored in a memory and executed by one or more special purpose processors, or modules, and/or hardware only, or in combination with software, described herebelow in reference to one or more of FIGS. 23 through 28.

Referring now to FIGS. 23 and 24, a schematic view of a steering module 149, and a flow chart of a steering method, respectively, are illustrated, consistent with the present inventive concepts. Steering module 149 may comprise in some embodiments a Human Interface Device, (HID) 122, an integrator 151 and a steering method 152. Steering module 149 can be positioned in one or more of feeder unit 100a and interface unit 100b. In STEP 2401, a change in position (e.g. a velocity) recorded by HID 122 can be monitored. In STEP 2402, the recordings are processed, such as a mathematical process including integrating the velocity measurements that are recorded. In STEP 2403, a steering command is calculated based on the analysis of STEP 2402.

Steering module 149 and/or the method of STEPS 2401 through 2403 can be configured to improve steering of articulating probe 400, such as to filter or otherwise compensate for tremor or other unintended motion (e.g. unintended reciprocal or small motion of the HID) that may be present when an operator such as a surgeon controls HID 122. During the operation of probe system 100, movement commands from HID 122 can be monitored by steering module 149 at a pre-determined rate, such as a rate of between 1 Hz and 10,000 Hz, such as a rate of approximately 1000 Hz. High sampling rates can result in detection of input errors such as those caused by operator tremor, and can correlate to undesired motion of articulating probe 400. Integration of motion data of HID 122, such as an integration of the velocity of motion of HID 122 can be used to reduce this undesired motion of articulating probe 400 and/or otherwise produce a smooth output. By changing the interval of integration, the filtering parameters can be changed to allow either more or less of the high frequency input to pass down to the distal tip of probe 400.

In some embodiments, a scale factor is applied upon operator input commands received from HID 122. In some embodiments, the scale factor is adjustable, such as adjustable between a range of 0.1 and 1.0. Scale factors can be utilized to adjust between fine (small scale factor) and coarse (large scale factor) motion control by HID 122.

Referring now to FIG. 28, a flow chart of a calibration procedure is illustrated, consistent with the present inventive concepts. Probe system 100 can comprise one or more calibration procedures, such as a calibration procedure used to calibrate one or more load cells used to monitor tension in a locking and/or steering cable of the present inventive concepts. STEPs 2801 through 2805 describe an embodiment of a calibration procedure that improves accuracy of measurement of cable tension by a load cell, such as load cells 221 described herein. One or more load cells can be configured to measure cable tension, such as when the load cell is engaged with a motor assembly rotatably attached to a base assembly 200 and configured to drive a pulley containing the cable, such as is described hereabove. The calibration procedure of steps 2801 through 2805 can be performed multiple times, on different load cells, such that different calibration parameters can be generated for each. Multiple calibration procedures can be performed simultaneously or sequentially. The rotational force applied by the motor assembly to the load cell correlates to tension in the cable. In these and other configurations, the load cell may also measure one or more undesired loads (e.g. not desired for cable tension measurement) that is not related to cable tension, such as a load due to a force applied by the weight (e.g. due to gravity) of a motor assembly, such as a motor assembly comprising motor 212 and/or motor mount 218 described hereabove. This motor assembly weight-driven load on the load cell may be variable, based on the relationship of the motor assembly to the force of gravity. The calibration procedure of FIG. 28 can be preformed to determine the specific load due to the weight of the motor assembly that is present at the time of use (e.g. based on the geometric position of the motor assembly relative to the force of gravity).

In STEP 2801, a determination was made by a computer processor of the probe system 100 whether the calibration is to be performed. Calibration can be performed based on an event selected from the group consisting of: use of a feeder assembly is about to occur and calibration has not yet been performed; a system start or restart has occurred; top assembly 300 is attached to base assembly 200; a calibration has been performed but the feeder assembly has subsequently been reoriented (e.g. as detected by a position sensor such as sensor 225 described herein); an undesired state has been detected by the system; a calibration is requested by an operator; and combinations of these.

In STEP 2802, the motor assembly may be driven to cause rotation of a cable pulley such that cable is advanced a preset length, such as to slacken (“pay out”), causing a condition in which little or no force is applied to the load cell due to cable tension.

In STEP 2803, an optional step of calculating the feeder assembly and/or motor assembly orientation can be performed, such as by using a signal provided by sensor 225. This orientation information can be recorded (e.g. stored in electronic memory), and used for future comparisons and/or for use in one or more algorithms implemented in program code and executed by a computer processor of the probe system 100 that compensate for and/or otherwise use the orientation information. This orientation information can include yaw, pitch and/or roll of the base assembly 200.

In STEP 2804, zero-tension data from the load cell is recorded (e.g. a number of samples). The zero-tension data can comprise a set of data that is averaged or otherwise mathematically processed. This zero-tension data can correlate to a correction factor (e.g. offset) used to determine cable tension. This zero-tension data can correlate to a load applied to the load cell due to the weight of the motor assembly (i.e. since cable tension is currently zero). The zero-tension data can be used to produce a more accurate load cell measurement of the cable tension during use of the system 100.

In STEP 2805, operation of the probe assembly is initiated, including steering, advancement, retraction, locking and un-locking of the articulating probe 400, such as operation based on measured cable tension whose measurement compensates for any or all undesired loads on the one or more load cells 221, as described herein. In some embodiments, the tension in each cable is brought to a predetermined value prior to any advancement or steering maneuver, such as a tension of 1N, 3N, 5N, 7N or 10N or more. In some embodiments, the amount of tension in one or more cables (e.g. each steering and/or locking cable) is kept above a minimum force, such as a minimum force above 1N, 3N, 5N, 7N or 10N or more. Maintenance of the minimum force can be configured to prevent any undesired hysteresis effects or other undesired effect, such that might otherwise be encountered as the force on the load cell transitions around zero force.

The calibration procedure of STEPs 2801 through 2805 can be performed on multiple cable-driving motor assemblies, simultaneously or sequentially, such as the four motor assemblies described herein. Alternatively or additionally, a calibration procedure is performed on one or more carriage assembly driving motor assemblies.

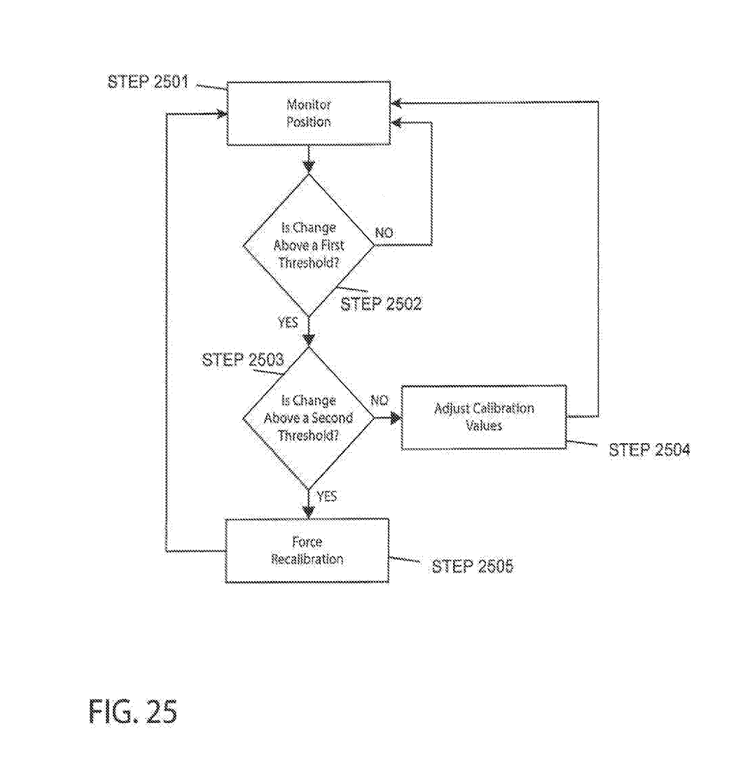

Referring now to FIG. 25, a flow chart of a safety method for performing a calibration is illustrated, consistent with the present inventive concepts. In STEP 2501, the position of feeder unit 100a is monitored (e.g. a monitoring of a position and/or a change in position), such as with one or more sensors, such as sensor 225 described herein. The sensor can comprise an accelerometer or other movement sensor used to measure displacement of feeder unit 100a or a sensor configured to measure the position of feeder unit 100a from which displacement of feeder unit 100a can be calculated. The sensor can comprise a gravitational and/or other static position sensor, such as a static position sensor comprising multiple mercury switches or similar switches oriented and arranged to determine the position of an object relative to the force of gravity. The static position sensor can be monitored over time such that a displacement of feeder unit 100a can be determined based on a change in the static position.

In STEP 2502, the magnitude of displacement of feeder unit 100a can be compared to a threshold, such as a pre-determined and/or operator settable first threshold. If the measured displacement does not exceed the first threshold, STEP 2501 can be repeated. If the measured displacement does exceed the first threshold, STEP 2503 can be performed in which the measured displacement is compared to a second threshold, such as a threshold of greater magnitude than the first threshold. If the measured displacement is less than the second threshold (but greater than the first threshold), STEP 2504 can be performed in which an adjustment of one or more calibration values is made, such as to adjust the amount of compensation for the effective weight of a motor assembly upon a load cell (e.g. adjusting for the weight of motor 212 and/or motor mount 218 upon a load cell 221, as described hereabove, for example, at FIG. 8A). If the measured displacement is more than the second threshold (as well as the first threshold), STEP 2505 is performed in which a second calibration procedure is required, such as a calibration procedure similar to the procedure described hereabove in reference to FIG. 28.

In some embodiments, an alarm or alert condition is entered (e.g. and notified to the operator such as via visual and/or audio signal), when the first threshold and/or the second threshold is reached. In some embodiments, the first and/or second threshold correlate to an undesired position of and/or impact to feeder unit 100a, such that feeder unit 100a needs to be repositioned and/or checked for damage prior to normal operation being initiated.

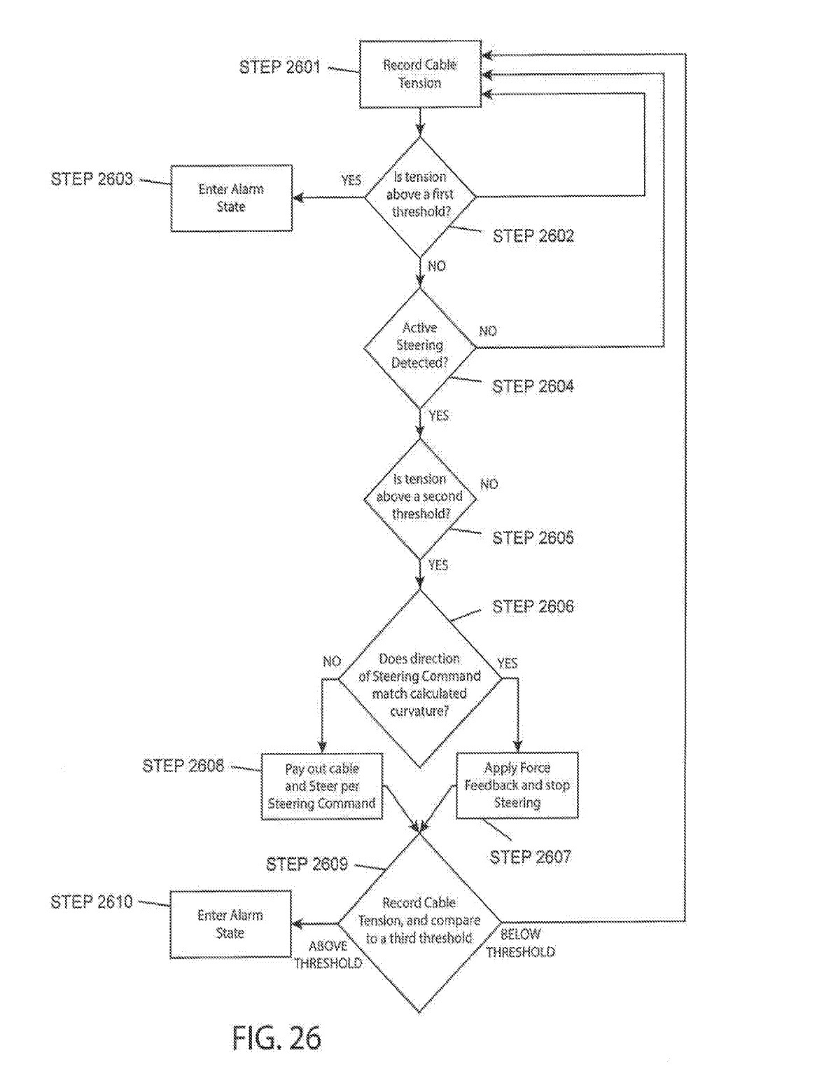

Referring now to FIG. 26, a flow chart of a method for preventing and/or detecting excessive force is illustrated, consistent with the present inventive concepts. Some of all of the method can be performed by one or more computer processors of the system 100 STEPs 2601 through 2610 illustrate a series of steps used to prevent and/or detect undesired force placed and/or otherwise being present on a cable, such as a cable used to steer and/or lock articulating probe 400. Cable tension can be monitored in numerous ways, such as via load cells 221 described herein and/or by monitoring motor current, motor rotation such as via a motor encoder, and the like. In some embodiments, system 100 is configured to prevent the tension in any cable from exceeding approximately 50% of the expected break force of the associated cable.

In STEP 2601, tension in one or more cables is recorded, such as has been described hereabove. In step 2602, the recorded cable tension is compared to a first threshold, such as a threshold of at most 50 lbs for an inner link mechanism 420 (locking) cable or at most 15 lbs for an outer link mechanism 440 (locking and steering) cable. If the tension is above the first threshold, STEP 2603 is performed, in which the system 100 enters an alarm state, e.g. an alarm state in which operation of the articulating probe is stopped, an alert is given to the operator, power to cable motors 212 is removed, and/or tension in one or more cables is reduced. If the tension is determined not to be above the first threshold, STEP 2604 is performed. In some embodiments, the cable tension is compared to the first threshold in hardware circuitry connected to a load cell, such that when the first threshold is identified by the hardware circuitry, a hardware-driven alarm state results in STEP 2603. In these embodiments, the maximum tension can comprise a threshold of no more than 12 lbs, 15 lbs, 18 lbs, 21 lbs or 24 lbs (e.g. for a cable 1350 of outer link mechanism 440) or no more than 44 lbs, 54 lbs, 64 lbs, 74 lbs or 84 lbs (e.g. for a cable 1350 of inner link mechanism 420). Alternatively, or additionally, the cable tension is compared to the first threshold using a software algorithm of system 100 that receives a signal from a load cell, such that when the first threshold is identified by a software program, an alarm state results in STEP 2603. In these embodiments, the maximum tension can comprise a threshold of no more than 91 lbs, 12 lbs, 15 lbs, 18 lbs or 21 lbs (e.g. for a cable 1350 of outer link mechanism 440) or no more than 30 lbs, 40 lbs, 50 lbs, 60 lbs or 70 lbs (e.g. for a cable 1350 of inner link mechanism 420).

In STEP 2604, a check for being in an (active) steering mode is performed. If steering is not being performed, STEP 2601 is repeated. If steering is being performed, STEP 2605 is performed.

In STEP 2605, the recorded tension (of STEP 2601) is compared to a second threshold, such as a threshold less than the first threshold. In some embodiments, the second threshold comprises a threshold of no more than 3 lbs, 5 lbs, 7 lbs, 91lbs, 111lbs, 131lbs or 151lbs. If the recorded tension is not above the second threshold, STEP 2601 is repeated. If the recorded tension is above the second threshold, STEP 2606 is performed. In some embodiments, STEP 2605 is only performed for cables of an outer link mechanism 440.

In STEP 2606, the direction of steering (e.g. a steering command entered by an operator into HID 122) is compared to the calculated curvature of articulating probe 400, such as curvature geometry using inverse kinematics (e.g. calculated at each advancement, retraction and/or steering of articulating probe 400 to determine its three dimensional geometric configuration). If the direction of steering matches the calculated curvature of the distal portion of articulating probe 400, step 2607 is performed. If the direction of steering does not match the calculated curvature of the distal portion of articulating probe 400, STEP 2608 is performed.

In STEP 2607, force feedback is presented to the operator (e.g. via a force-feedback based HID 122), and steering is stopped (e.g. all motion of articulating probe 400 is stopped). Subsequently, STEP 2609 is performed. Note that the system will remain with the steering stopped until a different steering command from the operator is received.

In STEP 2608, cable is paid out (i.e. the cable with the tension above the threshold is advanced). The cable being paid out can comprise one or more cables (e.g. of three) that are not being retracted during the current steering maneuver (e.g. one or more cables that may be transitioning from the inside of a curve to an outside of a curve due to the current steering maneuver). The amount of cable paid out can comprise a length of approximately 2.5 mm, 5 mm, 10 mm, 15 mm and/or 20 mm. In some embodiments, cable was already being paid out (e.g. automatically, as determined by a steering algorithm and due to the direction of desired steering), and the amount of cable being paid out in STEP 2608 is in addition to a “standard” amount based on the steering command (i.e. an extra amount delivered to prevent excessive tension in the cable). Subsequently, STEP 2609 is performed.

In STEP 2609, cable tension is again recorded and compared to a third threshold. In some embodiments, the third threshold is similar to or the same as the second threshold. In some embodiments, the third threshold can be different than the second threshold, such as higher than the first threshold. In some embodiments, the third threshold is similar to the first threshold. If the cable tension is not above the third threshold, a return to STEP 2601 is performed. If the cable tension is above the third threshold, STEP 2610 is performed in which the system enters an alarm state, such as a similar or dissimilar alarm state to STEP 2603, e.g. an alarm state in which operation of the articulating probe is stopped, an alert is given to the operator, power to cable motors 212 is removed, and/or tension in one or more cables is reduced.

In some embodiments, the comparison of STEP 2602 and related steps are not performed. In some embodiments, the comparison of STEP 2606 and related steps are not performed. In some embodiments, STEP 2602 is performed after STEP 2606. In some embodiments, in addition or as an alternative to cable tension excessive force monitoring, excessive force applied to one or more carriage assemblies is monitored (e.g. by monitoring the force on a carriage assembly drive motor), such as to reduce the force on the carriage assembly and/or enter an alarm state.

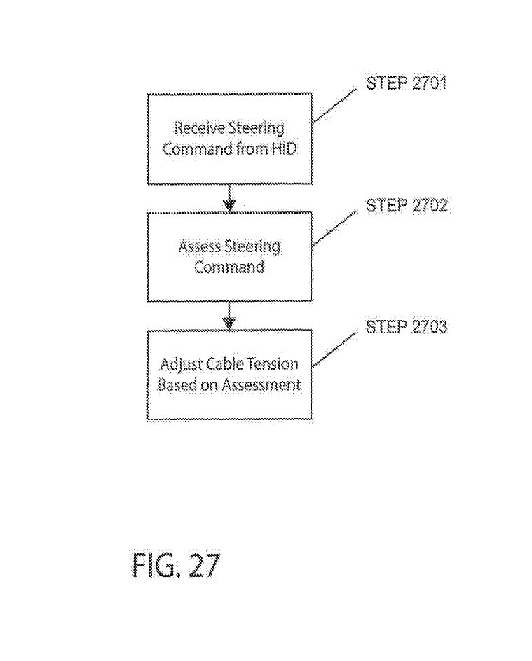

Referring now to FIG. 27, a method for detecting and/or reducing unintended motion of articulating probe 400 is illustrated, consistent with the present inventive concepts.

Some or all of the method can be performed by one or more computer processors or the system. In some embodiments, the motion of the distal end of articulating probe 400 is reduced when inner link mechanism 420 and/or outer link mechanism 440 transitions between locked and unlocked states. In these embodiments, program code of STEPS 2701 through 2703 described herein can be configured to anticipate an upcoming transition to locked mode, and confirm and/or cause each of the locking cables to be at a tension level approaching the locked tension level. A transition from a steering mode to a locked mode can be anticipated when a user input command correlates to a desired rate of motion of probe 400 of less than a threshold (e.g. 5 mm/sec). When a user input command correlates to a desired rate of motion higher than the threshold, system 100 can enter a steering mode, for example when tension in one or more steering cables is reduced, such as by paying out additional cable (e.g. by paying out 1 mm, 2 mm, 3 mm, 4 mm or 5 mm of cable), to allow for proper steering performance. When a user input command correlates to a desired rate of motion lower than a threshold (e.g. 5 mm/sec), system 100 can enter an “anticipation” mode, for example when tension in on or more steering cables is increased, such as by taking up cable (e.g. by taking up 1mm, 2 mm, 3 mm, 4 mm or 5 mm of cable), to pretension cables for locking, while still allowing fine adjustments of probe 400.

In STEP 2701, a steering command is received from an operator via HID 122. In STEP 2702, the steering command is assessed to quantify and/or qualify the steering command. In some embodiments, the assessment of STEP 2702 comprises an assessment of the “aggressiveness” of the steering command, such as an assessment correlating to the velocity and/or acceleration of movement of an operator on an input component of HID 122.

IN STEP 2703, tension within one or more steering cables can be adjusted based on the assessment performed in STEP 2702. For example, if it is determined that aggressive steering is being performed, and one or more cables need to be paid out (i.e. advanced), more cable may be paid out than if less aggressive steering was detected by the assessment.

The program code of FIG. 27 is configured to actively manage a cable payout offset that is applied to the two or more (e.g. three) outer mechanism 440 tensioning cables such that 1) when steering “quickly” (as determined by a velocity or acceleration assessment, such as when beginning or in the middle of a steering maneuver), the outer links 441 are loosely tensioned with a larger cable payout offset, and 2) when steering “slowly” (e.g. at the end of a steering maneuver), the outer links 441 are more tightly tensioned with a smaller cable payout offset. Thus, the method illustrated in FIG. 27 constantly monitors the steering input from the user and smoothly varies the cable tension to anticipate the end of a steering move by tightening the tensioning cables as the steering command slows. Once the steering command ends, articulating probe 400 is already in a partially locked state, thus reducing the additional tension that is required to fully lock articulating probe 400 e.g. reducing unwanted motion caused by applying tension to cables. The program code of FIG. 27 can be configured to smoothly ramp cable payout from low to high tension based on the assessment performed in STEP 2702 e.g. slower payout when less aggressive steering detected.

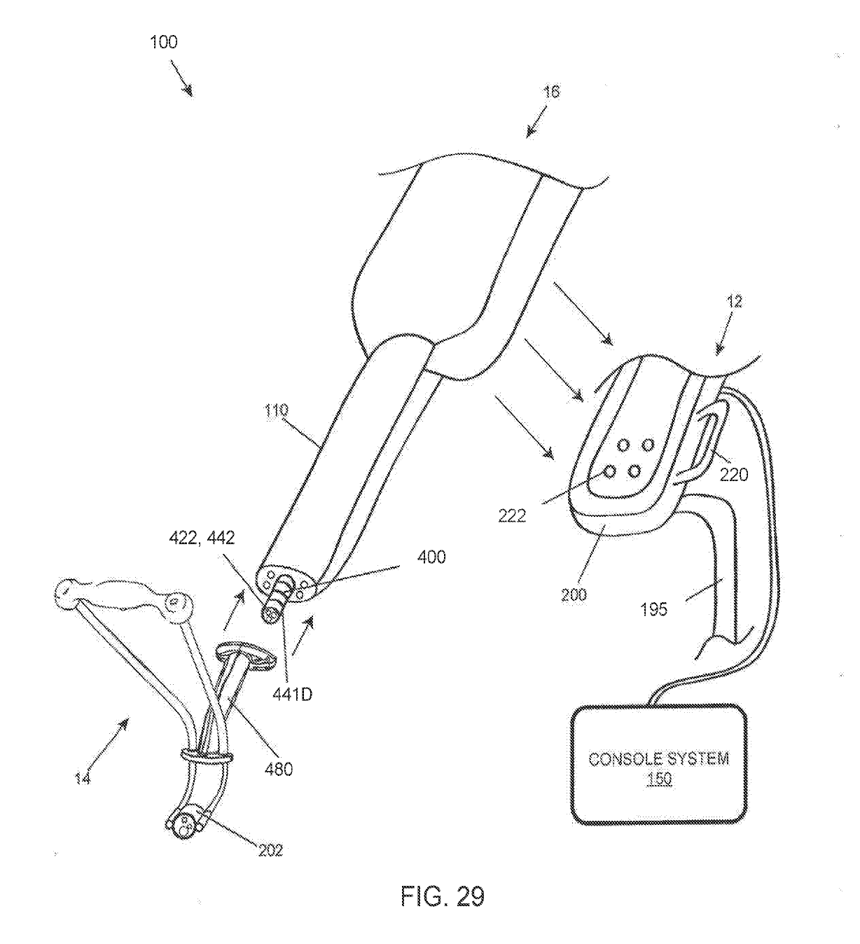

FIG. 29 is a perspective view of an articulating probe system 100, in accordance with embodiments of the present inventive concepts. The articulating probe system 100 can be constructed and arranged to perform a medical procedure, such as a transoral robotic surgery procedure. The articulating probe system 100 may include one or more features of a surgical positioning and support system, for example, described in PCT Application serial number PCT/US2011/044811, filed Jul. 21, 2011, PCT Application serial number PCT/US2012/32279, filed Apr. 5, 2012, PCT Application No. PCT/US2013/054326, filed Aug. 9, 2013, and PCT Application No. PCT/US2012/070924, filed Dec. 20, 2012, the contents of each of which are herein incorporated by reference in their entirety.

The articulating probe system 100 is constructed and arranged to position one or more tools (not shown) for performing a medical procedure on a patient, for example, a transoral robotic surgery procedure or the like, or other surgical procedure that includes inserting one or more tools into a cavity of the patient, or a region of the patient formed by an incision or related opening. A surgical procedure can include one or more transoral procedures, including but not limited to resections at or near the base of a tongue, tonsils, a base of a skull, hypopharynx, larynx, trachea, esophagus and within the stomach and small intestine. Other medical procedures can include but not be limited to single or multiple transaxilla procedures, such as a laryngectomy, single or multiple thoracoscopic procedures, such as a mediastinal nodal dissection, single or multiple pericardial procedures, for example, related to measuring and treating arrhythmias, single or multiple laparoscopic procedures, such as revision of bariatric lap-band procedures, single or multiple transgastric or transenteric procedures, such as a cholecystectomy or splenectomy, and/or single or multiple transanal or transvaginal procedures, such as a hysterectomy, oophorectomy, cystectomy and colectomy.

The articulating probe system 100 includes a first assembly 12, a second assembly 14, and a third assembly 16. In some embodiments, second assembly 14 described herein is of similar construction and arrangement to second assembly 14, described herein. The first assembly 12 is constructed and arranged to be used a plurality of times in one or more medical procedures. The second assembly 14 is constructed and arranged to be used fewer times than the first assembly 12. The third assembly 16 is constructed and arranged to be used in one or more medical procedures, but fewer times than the second assembly 14. In some embodiments, the third assembly 16 is constructed and arranged for a single use. In some embodiments, the third assembly 16 is constructed and arranged for multiple uses, but fewer uses than the second assembly 14.

The term “use” can refer to a use of the first, second, and/or third assembly in one or more procedures for a particular patient. For example, the third assembly 16 can be used to perform one or more medical procedures on one patient, removed from the system 100, and replaced with a different third assembly 16 that is used to perform one or more medical procedures on a different patient. In another example, the third assembly 16 can be used to perform a procedure on one patient, removed from the system 100, and replaced with a different third assembly 16 that is used to perform a different procedure on the same patient.

The first, second, and/or third assemblies 12, 14, 16 can include a processor and a memory for storing program code for performing one or more features and functions described herein. For example, program code for performing a camera calibration such as a gamma correction, or for counting the number of clinical uses of an assembly, can be stored in the memory.

The second and third assemblies 14, 16 are typically sanitized (e.g. cleaned, disinfected and/or sterilized) for each use. Unlike the second and third assemblies 14, 16, in some embodiments, the first assembly 12 is not positioned in an environment that requires sterilization after each use, for example, sterilization that would be required between medical procedures performed on different patients. In other embodiments, one or more portions of first assembly 12 are covered by one or more sterile barriers, such as a sterile drape positioned between first assembly 12 and third assembly 16. The second assembly 14 can be sanitized (e.g. cleaned, disinfected and/or sterilized) between uses. In some embodiments, the third assembly 16 is sanitized, typically sterilized, for a single use, and is removed from the first and third assemblies 12, 16, and disposed of, after its single use.

The first assembly 12 includes a base assembly 200 comprising a cable control assembly 222 that controls a movement of an articulating probe assembly 400 of the third assembly 16, described below. In some embodiment, the cable control assembly, 222 can include the capstans 216a, 216b, as shown, eg in FIGS. 8a, 12. The base assembly 200 can include other elements similar to those described in PCT Application No. PCT/US2012/070924, filed Dec. 20, 2012, or U.S. patent application Ser. No. 14/364,195, filed Jun. 10, 2014, the contents of which are incorporated herein by reference in their entirety.

The first assembly 12 includes a base stand 195, or related brace, which attaches the base assembly 200 to a floor, a patient operating table, or other supporting object. For example, in some embodiments, the base stand 195 may be of a form related to the feeder aim 106 of FIG. 1. A handle 220 can extend from the base assembly 200 that permits an operator to move the articulating probe system 100 relative to the supporting structure to which the base stand 195 is coupled, for example, a floor, a patient operating table, etc., before or during a medical procedure, or between different procedures. The first assembly 12 comprises a console system 150. The console system 150 includes a monitor and a human interface device (HID) for example shown in connection with FIG. 1. Elements of the console system 150 may be the same as or similar to the interface unit 100b described herein for example in connection with FIG. 1. The monitor may be configured to display images and/or sensor readings from tools or related devices, e.g., cameras, probes, sensors, which are coupled to or otherwise provided with the articulating probe assembly 400, the second assembly 14 and/or one or more other components of the system 100. The console system 150 may further include an input device, such as a keyboard, touch screen, touch pad and/or pointing device, for communicating with elements of the articulating probe system 100 100, such as the articulating probe assembly 400.

An operator, such as a surgeon or other medical professional, may control the articulating probe system 100 via a HID to manipulate or otherwise control the functions and movement of the articulating probe assembly 400, for example, steer, advance, retract or otherwise control the functions and movement of articulating probe assembly 400. The HID may include a hand-operated control device, such as a joystick.

The first assembly 12 can be coupled to one or more different third assemblies 16, for example, over the lifetime of the first assembly. Features of an exemplary third assembly are described at PCT Application No. PCT/US2012/070924, filed Dec. 20, 2012, the contents of which are incorporated by reference above.

The third assembly 16 can be coupled between the first assembly 12 and the second assembly 14, such as a coupling in the directions shown by the arrows. The third assembly 16 comprises a probe feeder 110 that is removably coupled between the first assembly 12 and the second assembly 14. The articulating probe assembly 400 of the third assembly 16 is removably coupled to the second assembly 14. The probe feeder 110 can include a carriage, guide rails, cables, gears, and/or other mechanical devices that communicate with the cable control assembly 222 of the base assembly 200 of the first assembly 12 to control a movement of the articulating probe assembly 400, and/or one or more tools in communication with the articulating probe assembly 400. For example, the base assembly 200 can include motor driven wheels, which engage and drive bobbins, gears, or the like, which in turn can advance and retract a carriage of the probe feeder 110.

The articulating probe assembly 400 can include a plurality of links that are constructed and arranged to facilitate a manipulation of the probe assembly 400, which in turn can guide one or more surgical tools during a medical procedure. The links can be constructed and arranged to form at least one multi-link inner probe (not shown) and a multi-link outer probe, similar to a probe assembly described in PCT Application No. PCT/US2012/032279, filed Apr. 5, 2012, the content of which is incorporated herein by reference above. The inner probe can include a plurality of inner links and the outer probe can include a plurality of outer links, for example similar to the embodiments shown in FIG. 2. The inner probe and the outer probe can communicate with each other by a plurality of steering cables (not shown), which are steerable by the cable control assembly 222, for example, which can advance or retract the links with respect to one another during manipulation of the articulating probe assembly 400. The steering cables can be used to releasingly tighten to lock or stiffen either or both of the plurality of inner links or the plurality of outer links. Accordingly, the inner probe and the outer probe can be configured in one of a limp mode and a rigid mode so as to facilitate the manipulation of the articulating probe assembly 400. For example, the inner and outer links may be configured in one of the limp mode and the rigid mode by steering or adherence controlling a movement of one or more steeling cables of the articulating probe assembly 400 through an introduction device 480

The articulating probe assembly 400 includes a connecting link 441_{D} at a distal end of the outer links, also referred to as a distal link, which is removably coupled to a portion of the second assembly 14, as described herein. The connecting link 441_{D} can include one or more working channels 422, 442 for transferring electrical signals and/or tools to the second assembly 14. The working channels 422, 442 may extend through some or all of the articulating probe assembly 400, for example, in a channel between the inner and outer links, from a proximal end to a distal end of the articulating probe assembly 400. The working channels 422, 442 can be aligned with working channels extending through a distal link extension assembly of the second assembly 14, as described herein.

The second assembly 14 includes the introduction de(also referred to as an introducer) constructed and arranged to slidingly receive the articulating probe assembly 400. The second assembly 14 is also constructed and arranged to position and/or provide support to one or more tools (not shown) for performing a medical procedure on a patient. The second assembly 14 can be coupled over its lifetime to at least two different third assemblies 16, for example, where each third assembly 16 is constructed and arranged to perform a single use, while the second assembly 14 is constructed and arranged for reuse. In an embodiment, the second assembly 14 includes a distal link extension assembly 202 for coupling with the connecting link 441_{D} at the distal end of the articulating probe assembly 400 of the third assembly 16.

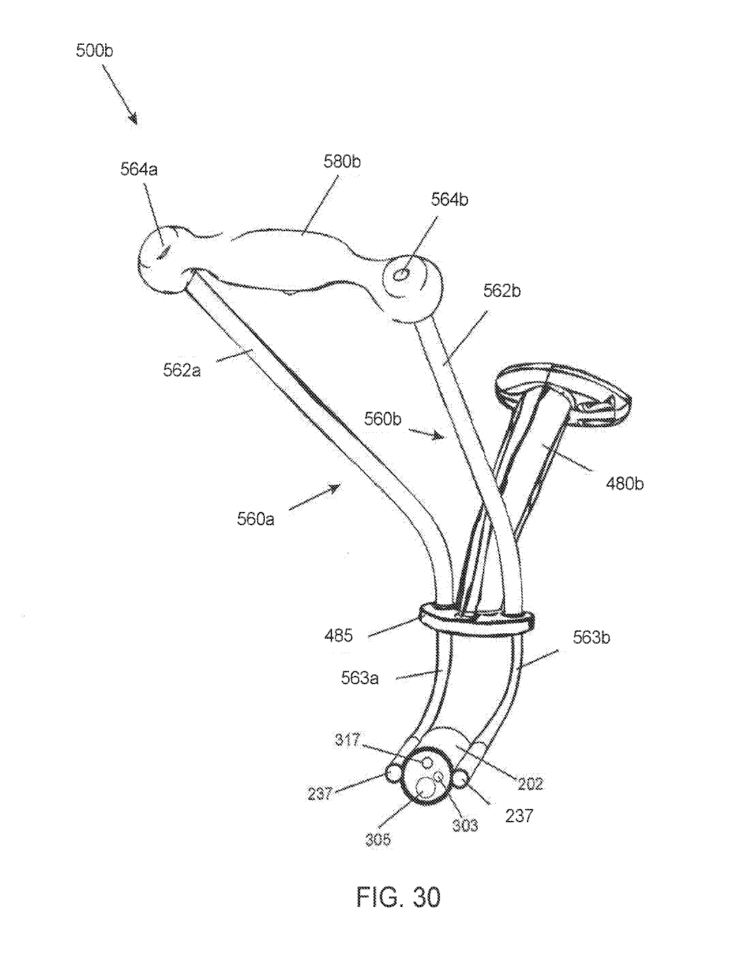

FIG. 30 is a perspective view of a tool positioning system 500b, in accordance with an embodiment. The tool positioning system 500b can be the same as or similar to the second assembly 14 of FIG. 29. As described herein, the tool positioning system 500b comprises an introduction device 480b. The tool positioning system 500b also comprises a first tool guide tube 560a, and a second tool guide tube 560b, also referred to as tool supports. Although two tool guide tubes 560a, 560b (generally, 560) are shown, the tool positioning system 500b can be constructed and arranged to include more than two tool guide tubes 560 or, alternatively, can include a single guide tube 560. Each tool guide tube 560 is constructed and arranged to slidingly receive a tool or other elongate object used in a medical procedure.

The first tool guide tube 560a can include an outer guide tube 562a and an inner guide tube 563a that is slidingly received by the outer guide tube 562a. The second tool guide tube 560b can include an outer guide tube 562b and an inner guide tube 563b that is slidingly received by the outer guide tube 562b. Accordingly, each of the tool guide tubes 560 can have an inner guide tube 563a, b (generally, 563) that movably extends from the outer guide tube 562a, b (generally, 562), for example, in a telescoping configuration.

At least a portion of each inner guide tube 563 can be flexible. To achieve this, an inner guide tube 563 can include one or more hinged sections. At least a portion of each outer guide tube 562 is rigid, with limited or no flexibility. The inner guide tubes 563 can be formed of plastic or related material. Materials can include but are not limited to fluoropolymers (e.g., polytetrafluoroethylene), fluorinated ethylene propylene, polyether block amide, high density polyethylene, low density polyethylene and/or nickel titanium alloy. The inner guide tubes 563 can comprise laser cut tubes, e.g. polymer or metal tubes with cuts placed to provide flexibility, and/or coils or braids of plastic or metal. In some embodiments, an inner guide tube 563 comprises a polytetrafluoroethylene liner. In some embodiments, an inner guide tube 563 comprises a stainless steel coil. In some embodiments, an inner guide tube 563 comprises a coil covered by a polyether block amide. In some embodiments, an inner guide tube 563 comprises a varying stifihess along its length.

The tool positioning system 500b can include a base 485. The base 485 can comprise a collar that surrounds at least a portion of the introduction device 480, and is fixedly attached to the surface of the introduction device 480. The collar can extend in a lateral direction relative to a direction of extension of the introduction device 480b. The collar has first and second openings. The outer guide tubes 562 of the tool guide tube 560 can be coupled to one side of the first and second openings, and the inner guide tubes 563 can extend from the first and second outer guide tubes 562, respectively, at a second side of the first and second openings. The first tool guide tube 560a and the second tool guide tube 560b are coupled to the base 485 to maintain a relative position between the first tool guide tube 560a and the second tool guide tube 560b and/or maintain a fixed orientation, and separation distances between the first tool guide tube 560a and the second tool guide tube 560b. The base 485 can also comprise an opening for receiving, and holding in place against, the introduction device 480b and/or an articulating probe 400, such as probe assembly 400 of system 100, advanced therethrough.

One or more tool guide tubes 560 can rotatably engage the base 485. The tool guide tube 560 can be coupled to the base 485 by a gimbal or other pivoted or ball and joint mechanism (not shown), permitting the tool guide tube 560 to rotate relative to the base 485, for example, allowing for three degrees of freedom between tool guide tube 560 and base 485, which can include two-dimensional (X-Y) movement plus rotation.

In other embodiments, the first and second tool guide tubes 560a, 560b are fixedly coupled to a surface of the introduction device of a base, for example, via welding points, adhesives, or other bonding mechanisms. The connection at the introduction device 480 maintains a fixed distance and/or a fixed orientation between the first tool guide tube 260a and the second tool guide tube 560b. In some embodiments, the first and second tool guide tubes 560a and 560b can be rotatably attached to each other and/or a base for maintaining a fixed distance but not a fixed orientation. The first tool guide tube 560a and the second tool guide tube 560b can be fixed in position relative to each other. Accordingly, positions of the first and second tool guide tubes 560a, 560b can be maintained during an operation of the articulating probe system 100.

The tool positioning system 500b can include a guide tube support 580b coupled to the first tool guide tube 560a and the second tool guide tube 560b. The guide tube support 580b is constructed and arranged to maintain a relative position between the first tool guide tube 560a and the second tool guide tube 560b. In some embodiments, guide tube support 580b is constructed and arranged to maintain a relative orientation between the first tool guide tube 560a and the second tool guide tube 560b. In an embodiment, the guide tube support 580 includes a connector, for example, a dogbone connector described with reference to PCT Application No. PCT/US2013/054326, filed Aug. 9, 2013, incorporated by reference above. The guide tube support 280 can be removably attached to the tool guide tubes 560a, 560b. Accordingly, in some embodiments, the guide tube support 580b is used with two or more different tool positioning system 500b, depending on the medical procedure. For example, in a first medical procedure, the guide tube support 580b is attached to a tool positioning system 500b. After the first medical procedure, the guide tube support 580b can be sanitized, and used in a second medical procedure, where the guide tube support 580b is attached to a different tool positioning system 500b.

The guide tube support 580b can comprise a rigid structure. Alternatively, the guide tube support 580b can comprise a malleable or flexible structure. The guide tube support 580 can comprise at least a portion that is flexible. The guide tube support 580 can comprise an operator shapeable structure. The guide tube support 580b can comprise two segments connected by a hinge, such as a butt hinge, a butterfly hinge, a barrel hinge or a hinge comprising a flexible portion positioned between two rigid portions. The guide tube support 580 can comprise a telescopically adjustable structure, such as to allow separation of tool supports 560a and 560b. The guide tube support 580 can comprise two segments connected by a rotatable connector, such as a universal joint.

The guide tube support 580 can be constructed and arranged to be shaped, molded, or the like, such as after the application of heat to a material used to form the guide tool support 580. The guide tube support 580 can be constructed and arranged to be attachable to at least one of the first tool guide tube 560a or the second tool guide tube 560b. The guide tube support 580 can be constructed and arranged to be detachable to at least one of the first tool guide tube 560a or the second tool guide tube 560b.

The guide tube support 580 comprises a first opening 564a and a second opening 564b (generally 564), each constructed and arranged to operably engage an outer guide tube 562a, 562b of the first and second tool supports 560a, 560b, respectively. The first opening 564a and the second opening 564b can be constructed and arranged to position the first tool guide tube 560a and the second tool guide tube 560b in a non-parallel configuration. At least one of the first opening 564a or the second opening 564b can comprise a funnel-shaped opening, for example, for receiving an outer guide tube 562. In this manner, an uninterrupted tool path can extend from an opening 564 at the guide tube support 580 through a tool guide tube 560b to a tool exit at a side port 238 of the distal link extension assembly.

In embodiments where a tool guide tube 560 is slidably adjustable, thus allowing for a shortening of a portion of the guide tube 560 that attaches to the guide tube support 580, the guide tube support 580b may require adjustability of the distance between connector openings. Depending on the desired relative orientation of one guide tube 560 to the other, parallel or angled, then the adjustability in the guide tube support 580 for the distance between openings can occur along a straight or curved path. The tool guide tube 560 can be locked in a fixed position relative to the base 485. The tool positioning system 500b can include a locking mechanism (not shown) to lock the at least one tool guide tube 560 in the fixed position. The locking mechanism may be constructed to secure a position of the tool guide tubes 560 with respect to the base 485, thus preventing the tool guide tubes 560 from sliding or otherwise moving axially during movement of the tools by one or more operators.

An outer guide tube 562b can have a funnel-shaped proximal end (not shown). The inner guide tube 563b can likewise have a funnel shaped proximal end (not shown). Either or both funnels can be configured to readily and a traumatically introduce tools to the tool guide tube 560. A funnel shaped proximal end of each tool guide tube 560 can be positioned about an opening 564a, b (564) in a guide tube support 580. In this manner, an uninterrupted tool path can extend from an opening 564 through a tool guide tube 560 to a tool exit at a side port 238 of tool positioning system 500b.

The introduction device 480 can be constructed and arranged to slidingly receive the articulating probe assembly 400 of the third assembly 16, and support, stabilize, and/or guide the articulating probe assembly 400 to a region of interest. The region of interest may be a lumen of a body of a patient, such as a cavity at the patient's head, e.g., a nose or mouth, or an opening formed by an incision. In clinical applications, typical regions of interest can include but not limited to the esophagus or other locations within the gastrointestinal tract, the pericardial space, the peritoneal space, and combinations thereof. The region of interest may alternatively be a mechanical device, a building, or another open or closed environment in which the articulating probe assembly 400 can be used.

In an embodiment, the tool positioning system 500b includes a distal link extension assembly 202 for coupling with the connecting link 115 at the distal end of the articulating probe assembly 10 of the third assembly 16. The connecting link 115 coupled to the distal link extension assembly 202 provides stability between the tool positioning system 500b and the third assembly 16, and also permits a transfer of electrical signals, power, light, liquid and/or energy between the distal link extension assembly 202 and the connecting link 115. The distal link extension assembly 202 and the connecting link 115 can comprise multiple elements constructed and arranged to mechanically attach the two components together, such as one or more snaps, threads or magnetic couplers.

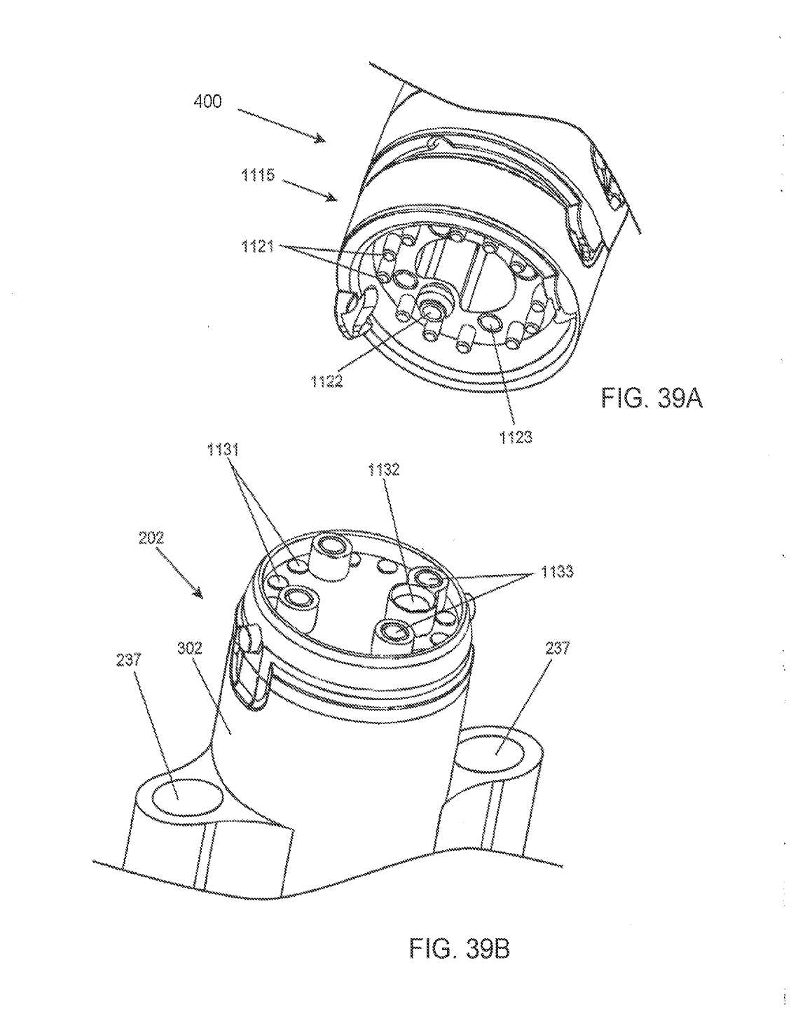

FIG. 39A is a perspective view of the distal end of an articulating probe assembly 400 including a set of attaching elements, in accordance with an embodiment. FIG. 39B is a perspective view of the proximal end of a distal link extension assembly 202 including a set of attaching elements that can mate with the attaching elements of the articulating probe assembly 10, in accordance with an embodiment.

In an embodiment, the articulating probe assembly 400 includes a distal link 1115, also referred to as a distal connecting link or distal outer link. In some embodiments. Distal link 1115 may be similar to distal outer link 441_{D} described herein. The distal link 1115 can include one or more electrical connectors 1121. The electrical connectors 1121 can comprise frictionally engaging pins, such as pogo pins configured to electrically engage opposing electrical contacts such as one or more electrical contacts 1131 extending from the distal link extension assembly 202.

The distal link 1115 further includes a male connector 1122 constructed and arranged to couple with a female connector 1132 of the distal link extension assembly 202. Mating connectors 1122 and 1132, when coupled together, can extend a working channel 317 (working channel 317), which can provide electrical signals, wiring, fiber optics, or the like to electrical elements of the distal link extension assembly 202, described herein. In some embodiments, connectors 1122 and 1132 may include fluid tight connectors, for example when a working channel 317 includes an irrigation channel or other fluid transfer channel.

The distal link 1115 and the distal link extension assembly 202 can also include one or more fasteners 1123 and 1133, respectively, for securing the distal link extension assembly 202 to the distal link 1115. One or more fasteners may include fasteners selected from the group consisting of: magnets; snap fit connectors; threaded connectors; or combinations of these. One or more fasteners can be configured to ensure a proper alignment of the distal link 1115 and the distal link extension assembly 202.

Referring again to FIG. 30, at least one side port 237 can extend from an outer surface of the distal link extension assembly 202. In an embodiment, a first side port 237 is coupled to the first tool guide tube 560a and a second side port 237 is coupled to the second tool guide tube 560b. Each side port 237 can provide a guide for an inner guide tube 563. An outer guide tube 562 and/or inner guide tube 563 can be constructed and arranged to guide or otherwise provide a support for a tool shaft so that it can be guided from the guide tube support 580 to a side port 237 extending from the distal link extension assembly 202.

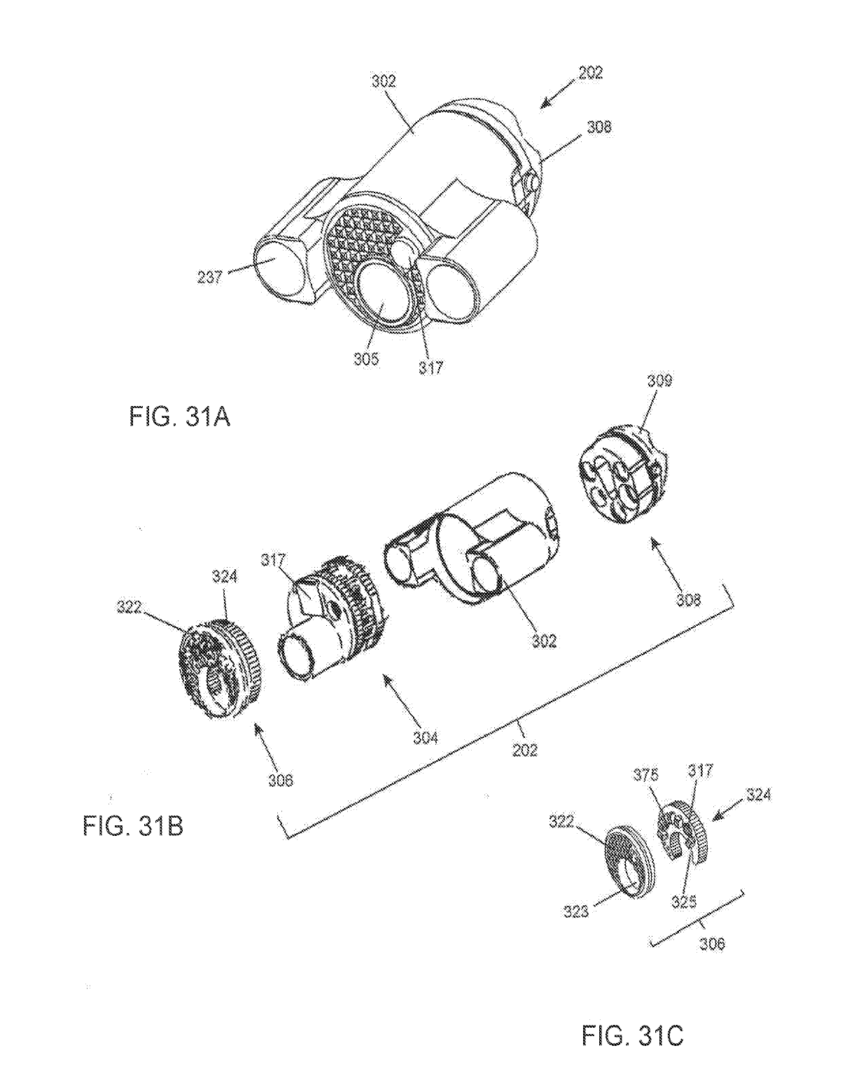

The distal link extension assembly 202 can also include one or more working channels 317 that are aligned with working channels 422, 442 of a connecting link 115. Any number of surgical tools or related accessories may be slidingly received by the working channels 422, 442 and/or the side ports 237, including but not limited to a cameras, light or other radiation sources, cutters, graspers, scissors, energy appliers, suturing assemblies, biopsy removal elements, ventilators, lasers, cautery, clip appliers, scissors, needles, needle drivers, scalpels, RF energy delivery devices, cryogenic energy delivery devices, drug delivery devices, EKG electrodes, pressure sensors, a blood sensors, magnets, heating elements, or combinations thereof. As shown in FIG. 31A, the distal link extension assembly 202 can include a camera lens 305 and a lighting source 303, such as an LED light source, which can be collocated with at least one working channel 317.

In an embodiment, at least one side port 237 includes a working channel at which a tool is positioned. In another embodiment, a lighting fiber assembly extends through the working channel of the side port 237 for transmitting light from a light source positioned proximal the lighting fiber. The lighting fiber assembly can be steerable, so that light can be directed to a working area. In an embodiment, the lighting fiber assembly can be for a single use. In another embodiment, the lighting fiber assembly can be configured for a plurality of uses.

Referring to FIG. 30 the tool positioning system 500b can include at least one fixation point (not shown) for attaching to the introduction device 480, the base 485, the first tool guide tube 561g, second tool guide tube 561h, the guide tube support 580b, and/or a combination thereof. A brace (not shown) can be attached between a fixation point and an operating room floor, a patient operating table, and/or an articulating probe feeder such as the feeder 110. The brace can include a clamping device or the like, for clamping to a floor, table or other supporting object. Multiple braces can be coupled to different fixation points. For example, a brace (not shown) can be coupled between a fixation point at the base 485 and a fixation point at the first tool guide tube 561b. Another brace can be attached to the feeder 110 and can be clamped or otherwise attached to a floor, table or other object providing stability.

FIG. 31A is a perspective view of the distal link extension assembly 202, in accordance with an embodiment. FIG. 31B is an exploded view of the distal link extension assembly 202 of FIG. 31A, in accordance with an embodiment. FIG. 31C is an exploded view of a lighting assembly 306 of FIG. 31B, in accordance with an embodiment.

The distal link extension assembly 202 includes a distal link body 302, a camera assembly 304, a lighting assembly 306, and a link connector 308. The distal link body 302 has a central opening that is configured so that the camera assembly 304 and lighting assembly 306 can be removably positioned in the distal link body 302. Some or all of the distal link extension assembly 202 can be removed from the tool positioning system 500b of FIG. 30, and replaced, for example, during a resterilization between uses of the tool positioning system 500b. A camera lens 305 and a diffusing lens 322 can be exposed at one end of the distal link body 302. In other embodiments, the camera assembly 304 and/or the lighting assembly 306 are external to the distal link body 302, for example, positioned at the surface of the distal link body 302. The link connector 308 can be coupled to the other end of the distal link body 302. The distal link body 302 can include one or more side ports 237 that extend from an outer surface of the distal link body 302.

The link connector 308 can have a body portion 309 that movably mates with the connecting link 115 at the distal end of the articulating probe assembly 10. For example, the body portion 309 can have a convex portion that is positioned in a cavity in the connecting link 115. Accordingly, the connecting link 115 and the distal link extension assembly 202 can articulate relative to each other during operation.

The lighting assembly 306 is positioned between the camera assembly 304 and a field of view. The lighting assembly 306 includes a diffusing lens 322 or related camera lens filter that diffuses or scatters light produced by the lighting assembly 306, for providing a uniform field of view. The diffusing lens 322 can be coupled to a printed circuit board (PCB) 324 having one or more light sources 375. The light sources 375 may include electron stimulated light sources such as electron stimulated luminescence light sources, incandescent light sources such as incandescent light bulbs, electroluminescent light sources such as light-emitting diodes (LEDs), and gas discharge light sources such as fluorescent lamps, or related sources that produce high power light. An electron stimulated light source can include an electron stimulated luminescence light source, an incandescent light source, an electroluminescent light source, and/or a gas discharge light source. An incandescent light source can include an incandescent light bulb. A gas discharge light source can include a fluorescent lamp.

An LED can be constructed and arranged to produce a predetermined amount of electromagnetic energy, for example, between 1-250 lumens of light. One or more LEDs can be constructed and arranged to provide a color temperature range between 2700K and 7000K A single LED or multiple discrete LEDs providing different fauns of light that collectively produce a desired effect. An LED can be constructed and arranged to produce at least one of infrared light or ultraviolet light or other range of frequencies known to those of ordinary skill in the art. An LED can be a multicolor LED. Thus, one or more LEDs with multicolor capabilities can generate a desired color temperature, or be used in conjunction with filters to produce desired emphasis or accentuate certain features/colors/tissue. Multiple LEDs, such as two or more independently controlled LEDs, can display differing colors to produce a desired color, color temperature, or effect.

In other embodiments, a light source 375 includes a laser light source, for example, a vertical cavity surface emitting laser (VCSEL). The laser light source can be excited by use of another laser through an optical fiber or the like to energize a VCSEL, thereby eliminating an electric shock risk from the light source.

The PCB 324 may further include optical fibers, which can be configured to transmit light to and from the articulating probe assembly 400 and/or another component of the articulating probe system 100. The diffusing lens 322 can include an opening 323. The PCB 324 can likewise include an opening 325. The diffusing lens 322 and the PCB 324 are coupled together so that the diffusing lens opening 323 is aligned with the PCB opening 325 for receiving a camera lens 305 of the camera assembly 304, and so that the diffusing lens 322 is positioned in front of a light source 375, for example, an LED.

In another embodiment, the light source 375 is at a different location than a lens at a distal end of the distal link extension assembly 202. The light source 375 is coupled to an optical fiber or other transmitter, which in turn is coupled to the distal lens. Here, light or other electromagnetic radiation is generated at the light source 375 and transmitted to the distal lens via the optical fiber.

The distal link extension assembly 202 can include at least one working channel 317 that extends through the camera assembly 304 and the link connector 308 to provide electrical signals, wiring, fiber optics, or the like to the lighting assembly 306.

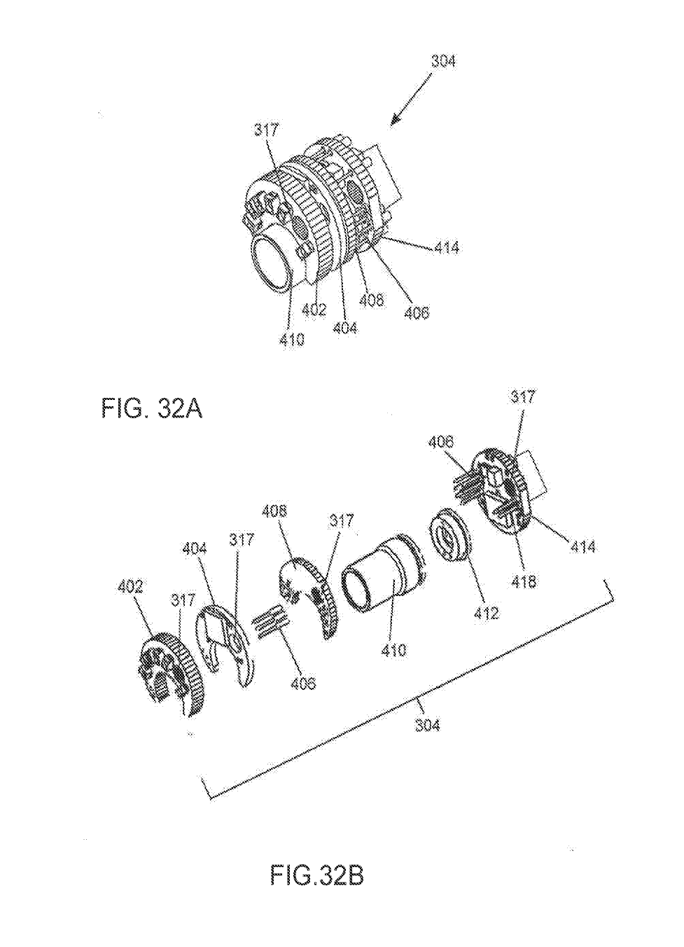

FIG. 32A is a perspective view of the camera assembly 304, in accordance with an embodiment. FIG. 32B is an exploded view of the camera assembly 304.

The camera assembly 304 includes a lens assembly 410 that focuses images of objects, which can be detected by a visual camera or other sensor device and transmitted to a console system, for example console system 150, stored on a media, or otherwise used in a manner that is well-known to those of ordinary skill in the art. The objects are related to a medical procedure, for example, taken of a patient undergoing a treatment. The lens assembly 410 can be removed from the camera assembly 304, and replaced, for example, during a sanitization, e.g. a resterilization, between uses performed by the tool positioning system 500b. A calibration adjustment nut 412, also referred to as a lens mount, can be threaded into the lens assembly 410 for adjusting a lens focus or calibrating the lens assembly 410, for example, during manufacturing. A PCB 414 having an image sensor 418 is coupled to one end of the lens assembly 410. The image sensor 418 can include a charge coupled device (CCD), CMOS sensor, or related sensing device for processing an image provided by the lens assembly 410.

The camera assembly 304 can include multiple PCBs, such as a first PCB 402, a second PCB 404, and a third PCB 408 each performing various function related to the operation of the camera assembly 304. Multiple PCBs can be used to fit necessary imaging, image processing, power and/or other electronic components within a constrained dimension, such as a maximum diameter, while expanding the assembly in a less constrained axial direction. The camera assembly 304 can include a plurality of connecting pins 406 for electrically and/or mechanically coupling the second and third PCBs 404, 408 with each other, and a plurality of connecting pins 406 for electrically and/or mechanically coupling the third PCB 408 and PCB 414 with each other. For example, as illustrated herein, the working channel 317 extends through the camera assembly 304. Although three PCBs are shown and described the camera assembly 304 may have a different number of PCBs.

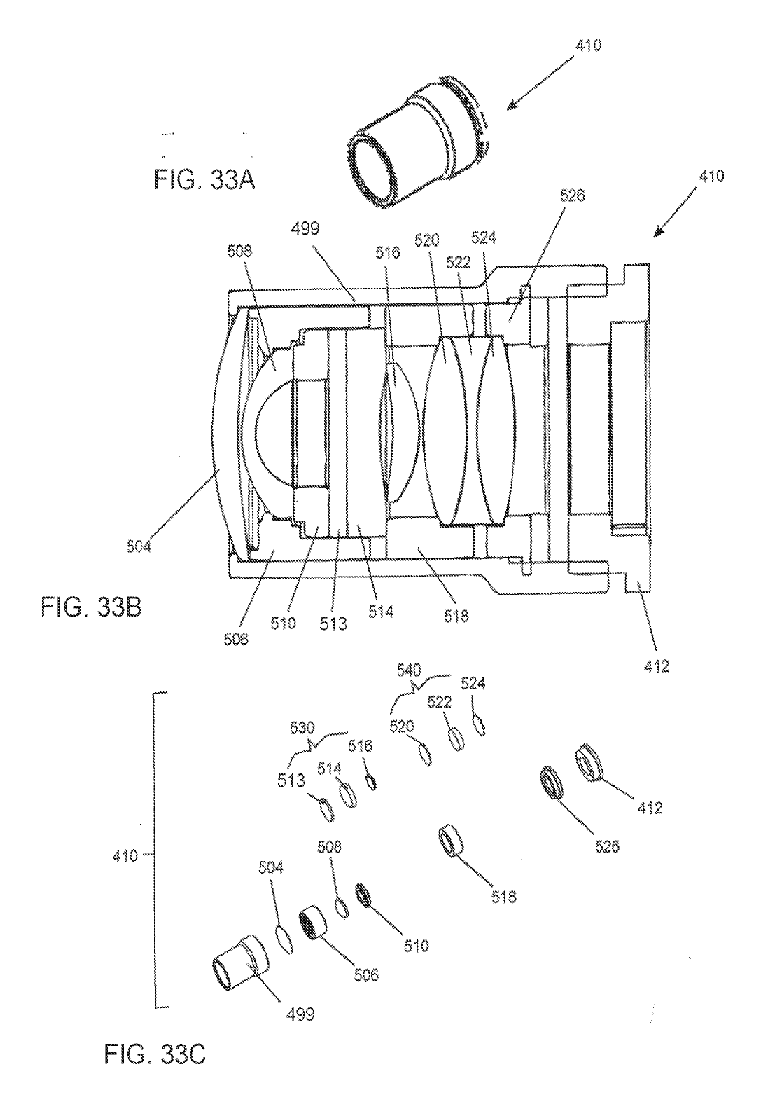

FIG. 33A is a perspective view of the lens assembly 410, in accordance with an embodiment. FIG. 33B is a cross-sectional view of the lens assembly 410, in accordance with an embodiment. FIG. 33C is an exploded view of the lens assembly, in accordance with an embodiment.

The lens assembly 410 includes a lens barrel 499 having an interior region that houses and provides for a precise alignment of one or more optics, spacers, and related elements, each described herein. One of the optics includes a front lens 504, which is fixed in place in the lens barrel 499 by a mounting structure that includes one or more spacers, for example, spacer 506, and/other elements described herein. The lens barrel 499 is constructed and arranged for positioning optics such as the one or more lenses to their required accuracy, while protecting the optics from environmental conditions such as temperature, stress, vibrations, or biological contaminates. The lens barrel 499 can include a seat, for example, a tangential seat, at which the front lens 504 can be radially and/or axially aligned by a tangent contact with respect to an optical surface of the front lens 504.The front lens 504 can collect electromagnetic radiation such as visible light or outer wavelength spectrum from a predetermined field of view, for example a field of view between 50° and 135°, such as a field of view of approximately 82°.

The lens assembly 410 can include one or more additional optics such as a polarizing or filtering lens, which can be constructed and arranged to control glare, reduce reflected lights from instruments (e.g. laser flare), or reduce other undesirable effects. One or more lenses described herein can filter one or more wavelengths (e.g. IR or visible light wavelengths) such as to accentuate features, colors, etc., to reduce or eliminate external light, and/or to provide a trigger signal. In an embodiment, a filtering lens can be constructed and arranged to allow particular wavelengths to pass ranging from 400 nm to 700 nm. In an embodiment, the filtering lens can be constructed and arranged to block infrared wavelengths, e.g. wavelengths ranging from 700 nm to 1105 nm. In an embodiment, the filtering lens can be constructed and arranged to block ultraviolet wavelengths ranging from 1 nm to 400 nm. In an embodiment, the filtering lens can be constructed and arranged to block LISA laser wavelengths for example, 2000 nm wavelength.

The spacer 506 provides an axial and/or radial alignment for the meniscus lens 508, the spacer 510, and an aperture/filter assembly 530. The meniscus lens 508 can direct light or other electromagnetic radiation at the camera aperture. Radial and/or axial alignment of the meniscus lens 508 can be established by a tangent contact of the spacer 506 with its optical surface. The spacer 510 provides an axial location for aperture/filter assembly 530, which comprises a filter glass 513, a lens 514, and a lens 516. In some embodiments, the lens 514 is a plano-concave lens (as shown) configured to accept light from the filter glass 513 and direct light into the lens 516. The lens 516 can comprise a meniscus lens (as shown) that is mounted to the lens 514 (e.g. cemented) such that light exiting the lens 514 is directed toward the concave surface of the lens 516. In an embodiment, the filter glass 513 prevents predetermined wavelengths from being transmitted, for example, a 2 μm wavelength. The filter glass 513 can include an opaque coating that creates an aperture to limit an amount of light reaching an image sensor, such as the image sensor 418. The spacer 506 can provide a radial alignment of the filter glass 513. The spacer 510, in particular, a flat surface of the spacer 510, can provide an axial alignment of the filter glass 513. In some embodiments, a radial and axial alignment of the aperture /filter assembly 530 with respect to the filter glass 513 is set during manufacturing.

The spacer 518 can provide an axial and/or a radial alignment for a triplet assembly 540, which comprises a lens 520, a lens 522, and a lens 524. The light exiting lens 516 is directed into a triplet assembly 540. In some embodiments, lens 520 is a convex-convex lens (as shown) which receives the light exiting lens 516 and directs light toward the lens 522. The lens 522 can comprise a concave-concave lens (as shown) which receives the light from the lens 522 and directs light onto the lens 524. The lens 524 can comprise a convex-convex lens (as shown) which receives the light from the lens 522 and directs light towards an image sensor, such as the image sensor 418. The triplet assembly 540 provides color correction and focuses light on the sensor 418. The triplet assembly 540 can set a radial and axial alignment by tangent contact of spacer 518 with an optical surface.

The lens retainer 526 compresses the lens stack together to maintain their respective alignments. Lens retainer 526 can be constructed and arranged to sufficiently compress the multiple lenses of the lens assembly 410. The lens retainer 526 can also provide centering from a rear of the triplet assembly 540 by contacting lens 524 at a tangent with respect to the optical surface.

The lens mount 412, also referred to as a calibration adjustment nut, attaches the lens assembly 410 to the camera assembly 304. The lens mount 412 is aligned with the sensor 418 by a close tolerance fit rectangular cavity that surrounds the sensor 418, thus providing an accurate alignment of the lens assembly 410 to the sensor 418. The lens mount 412 can include a thread for attaching to the lens assembly 410, allowing focus adjustments to be made, for example, by rotating the lens assembly 410 to achieve an optimal optical distance to the sensor 418.

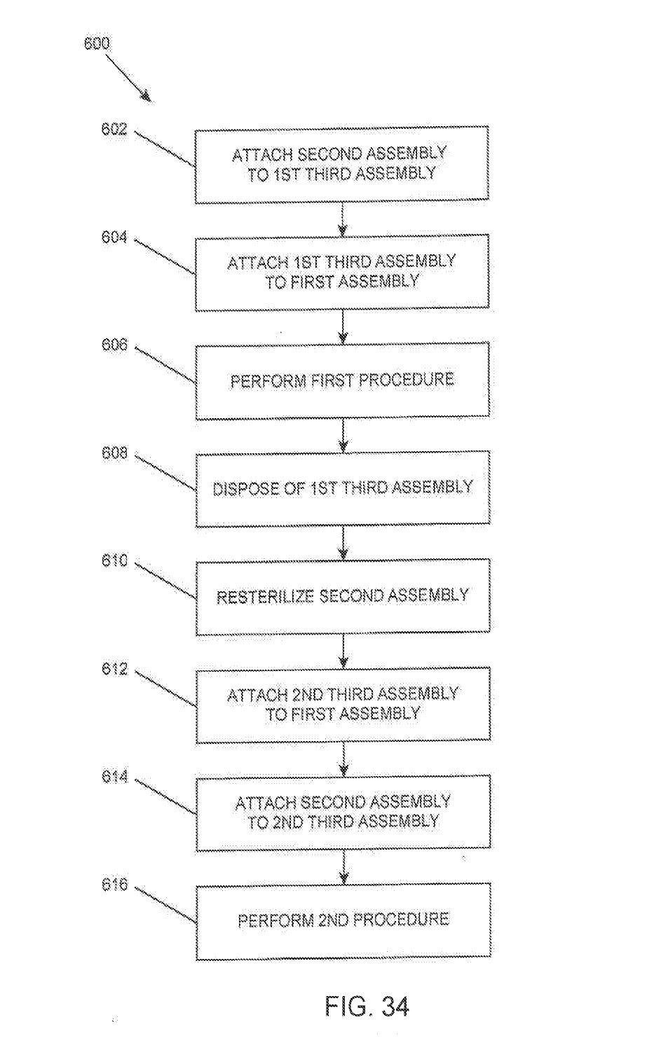

FIG. 34 is a flowchart illustrating a method 600 for assembling an articulating probe system 100 to perform an operation, in accordance with an embodiment the articulating probe system 100 may include robotic introducer 480 described in FIG. 3. Although the method 600 refers to a sequence of blocks, or steps, the method 600 is not limited to this sequence. In other embodiments, various blocks can be performed in a different order. For example, block 604 can be performed prior to block 602. Some or all of the method 600 can be performed by an articulating probe system in accordance with some embodiments.

At block 602, the second assembly 14 is attached to a third assembly 16 of one or more different third assemblies 16 used with second assembly 14, for example, over its lifetime. This attachment can include extending the connecting link 115 of the articulating probe assembly 400 of the third assembly 16 through the introduction device 250 to the second assembly 14 to the distal link extension assembly 202.

At block 604, the third assembly 16 is attached to the first assembly 12. This manipulation can include attaching a carriage, guide rails, cables, gears, and/or other mechanical devices (not shown) of the probe feeder 110 of the third assembly 16 to the cable control assembly 222 of the first assembly 12. Accordingly, the articulating probe system 100 is operational by attaching the first assembly 12, the second assembly 14, and the third assembly 16 to each other. In some embodiments, a sterile barrier such as a sterile drape is placed between first assembly 12 and third assembly 16.

At block 606, a first procedure can be performed by the articulating probe system 100, for example, a medical procedure, such as a transoral robotic surgery procedure.

At block 608, the third assembly 16 is removed from the articulating probe system 100. In some embodiments, the third assembly 16 is constructed for a single use, and is sanitized (e.g. sterilized) one time prior to that single use. In these embodiments, after the single use, i.e., the first procedure, is completed, the third assembly 16 is disposed of.

At block 610, the second assembly 14 can be sanitized (e.g. sterilized) after the first procedure, and prior to a subsequent procedure performed by the articulating probe system 100.

At block 612, another third assembly that is different than the third assembly 16 referred to at blocks 602, 604, 606, and 608 is attached to the first assembly 12.

At block 614, the sanitized second assembly 14 is attached to the new third assembly 16. Accordingly, the articulating probe system 100 is operational by attaching the first assembly 12, the second assembly 14, and the third assembly 16 to each other.

At block 616, a second procedure can be performed by the articulating probe system 100, for example, a medical procedure, such as a transoral robotic surgery procedure.

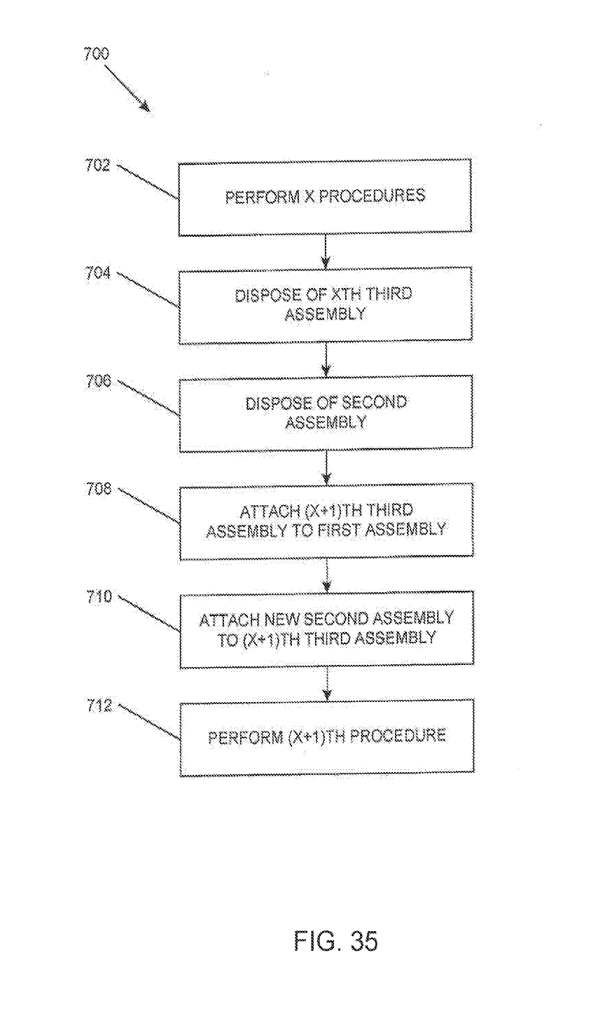

FIG. 35 is a flowchart illustrating a method 700 for assembling a robotic introducer system to perform an operation, in accordance with an embodiment. Some or all of the method 700 can be preformed by an articulating probe system in accordance with some embodiments.

At block 702, X procedures are performed, where X is an integer greater than 0. Each procedure of the X procedures can be performed in accordance with one or more steps of the method 600 described above. Accordingly, each procedure of the X procedures can include replacing a third assembly 16 with a different third assembly 16, e.g., a new third assembly 16. The second assembly 14 is constructed and arranged for reuse after each procedure of the X procedures. After each use, the second assembly 14 is sanitized as described herein.

At block 704, an Xth third assembly is removed from the articulating probe system 100 and disposed of.

At block 706, the second assembly 14 is disposed of after X procedures are performed.

At block 708, a new third assembly, i.e., an (X+1)th third assembly, is attached to the first assembly 12.

At block 710, a new second assembly is attached to the (X+1)th third assembly. Accordingly, the articulating probe system 100 is operational.

At block 712, an (X+1) procedure can be performed by the articulating probe system 100, for example, a medical procedure.

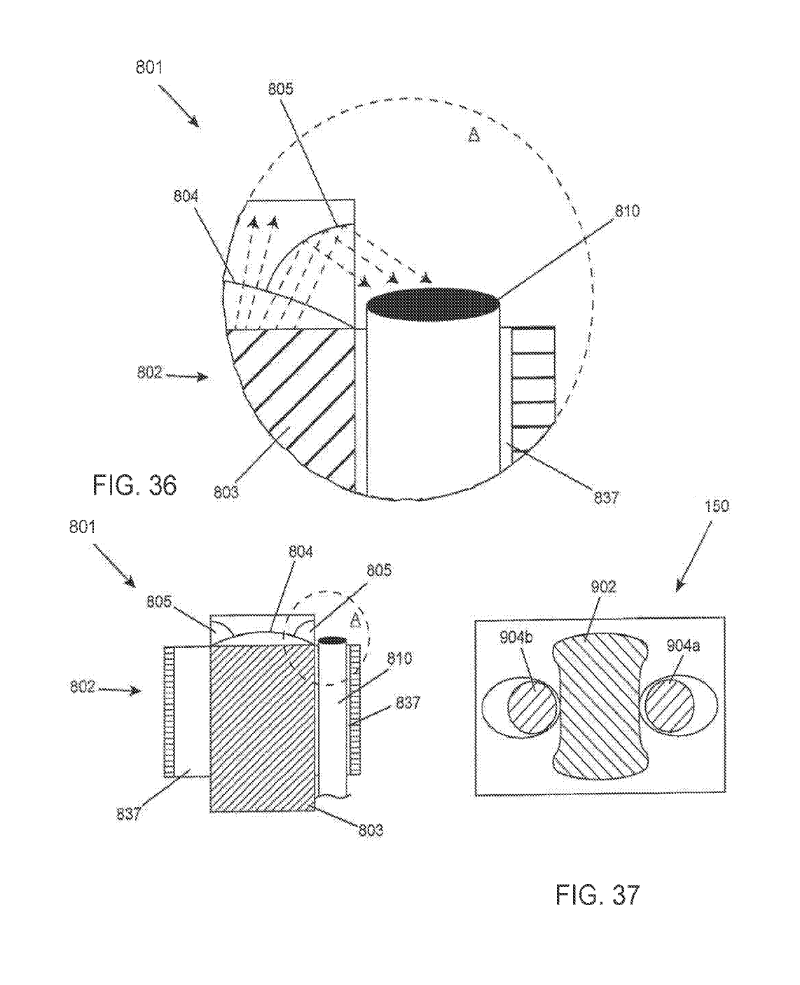

FIG. 36 is a cross-sectional view of an optical assembly 801, in accordance with an embodiment. The optical assembly 801 can be constructed and arranged to be part of a distal link extension assembly 802 coupled to a distal end of a probe assembly, for example, the articulating probe assembly 400 described. The distal link extension assembly 802 can be similar to the distal link extension assembly 202. Repetitive details of the distal link extension assembly 802 will not be repeated for brevity. The optical assembly 801 can include elements similar to the camera assembly 304 and/or the lighting assembly 306. Accordingly, details will not be repeated for brevity

The distal link extension assembly 802 can include a distal link body 803. At least one side port 837 extends from the distal link body 803. The side port 837 is constructed and arranged to receive a tool 810, for example, a cutter, a grasper, an energy delivery probe, a lighting fiber, etc. The optical assembly 801 can include a lens 804 that provides a first field of view, for example, collects images taken during a procedure. The optical assembly 801 can include an optical redirector 805 such as a mirror or prism that is adjacent the lens 804, and positioned so that an output of the lens 804, for example, optical pathways, are reflected from the optical redirector. For example, some of the optical pathway is directed towards the optical redirector 805, where it is then redirected toward the side port 837.

In this manner, the optical element 805 provides a second field of view that complements the first field of view of the lens 804. The combination of the lens 804 and the optical element 805 can provide a combined field of view that is up to 180°, and in some cases, greater than 180°. This feature permits an operator to view multiple images proximate distal link extension assembly 802. For example, as shown in FIG. 37, console system 150 can produce multiple images 902, 904a and 904b. Image 902 represents an image of a region in front of lens 804. Image 904a and 904b represent images of the side ports 837 on either side of lens 804. In some embodiments, the optical element 805 is configured to allow viewing of a tool initially exiting a side port 837, where an image can be outside of the first field of view of lens 804, such as tool 810 shown.

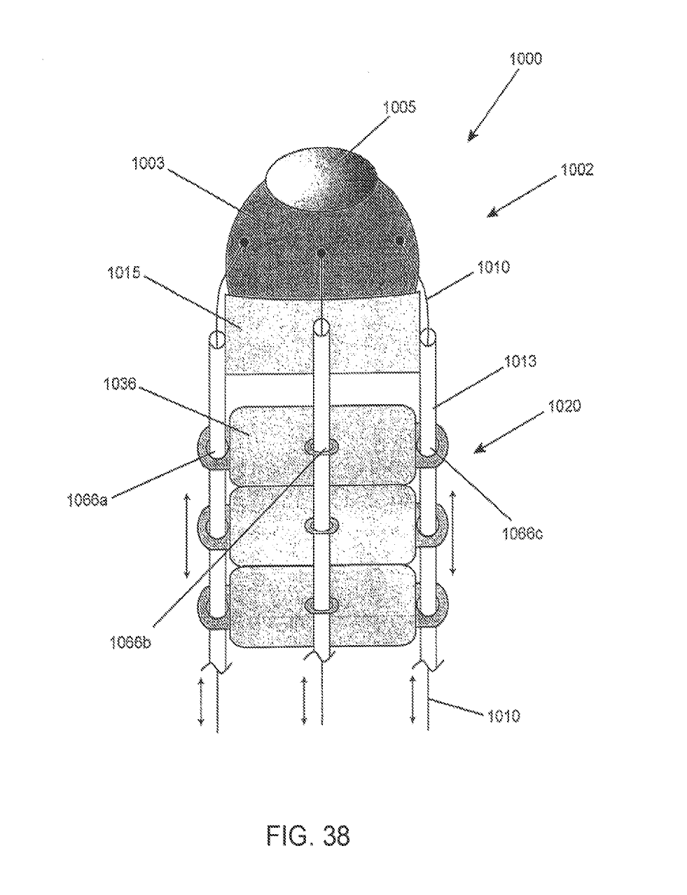

FIG. 38 is a cross-sectional view of a robotic introducer system 1000 comprising a distal link extension assembly 1002, in accordance with an embodiment. The distal link extension assembly 1002 is coupled to a distal end of an articulating probe assembly 1020. The probe assembly 1020 can include elements that are the same as or similar to the articulating probe assembly 400 described herein, and will not be repeated for brevity.

The distal link extension assembly 1002 includes a base 1015, a body 1003 movably positioned in the base 1015, and an optical lens 1005 coupled to the body 1003. A plurality of body articulating cables 1010 extend along the probe assembly 1020 and the base 1015. A distal end of each articulating cable 1010 is attached to the body 1003. The articulating cables 1010 can be advanced or retracted in response to a force applied to the cables 1010 to move the body 1003 for changing a field of view of the lens 1005. The articulating probe assembly 1020 and the body 1003 are independently controllable. For example, the articulating cables 1010 can be advanced and retracted to move the body 1003 relative to an axis along which the robotic introducer system 1000 extends while the articulating probe assembly 1020 remains stationary along the axis.

The articulating probe assembly 1020 includes a plurality of probe links, for example, inner probe links and outer probe links similar to the probe assembly 400 described above and/or described in PCT Application No. PCT/US2012/032279, filed Apr. 5, 2012, the content of which is incorporated herein by reference above. The distal link extension assembly 1002 is adjacent a distal link 1036 of the probe links. The articulating probe assembly 1020 can include at least one steering cable that extends through the links and terminates at the distal link 1036. The steering cable and the body articulating cables 1010 are independently controllable.

The base 1015 can include a concave region, which can mate with a convex lower region of the body 1003. In an embodiment, the body 1003 is ball-shaped and is positioned in a cavity of the base 1015. Alternatively, the base 1015 can include a convex region, which can mate with a concave lower region of the body 1003. The coupling of the base 1015 and the body 1003 in this manner permits a rotation of the body 1003 relative to the base 1015 in response to a force applied to the body articulating cables 1010. The body 1003 and/or the base 1015 can have a cavity or a protruding region having other shapes, for example, semi-spherical, semi-ellipsoidal, or parabolic shape.

A plurality of guide holes 1066 (1066a-c shown) can extend from the probe assembly 1020. The articulating body cables 1010 can extend through the guide holes 1066. In an embodiment, each link 1036 in the articulating probe assembly 1020 has a guide hole 1066a, 1066b, 1066c (generally, 1066). Two or more guide holes 1066, for example, guide holes 1066a, 1066c can be aligned with each other to receive an articulating body cable 1010. A plurality of flexible tubes 1013 can extend through the guide holes 1066 along the probe assembly 1020. The tubes 1013 can be spaced equidistantly with respect to each other about the probe assembly 1020. The tubes 1013 can advance and retract with respect to the probe assembly for articulating the probe assembly 1020. The tubes 1013 can move in concert with, or independently of, a movement of the steering cables (not shown) extending through an interior of the links 1036. The body articulating cables 1010 and the tubes 1013 can operate to pan or tilt, the lens 1005 coupled to the body 1003. Alternatively or additionally, body articulating cables 1010 and the tubes 1013 can operate to zoom lens 1005 (e.g. by advancing the base 1015). A distal end of each of the tubes 1013 is coupled to the base 1015. A body articulating body cable 1010 extends through each tube 1013.

The lens 1005 can be part of a camera assembly, for example, a camera assembly described herein, such as a camera assembly contained in whole or in part in body 1003. Details of the camera assembly are not repeated for brevity. The body 1003 can include a hollow interior or include a cavity in which the camera assembly can be positioned. The lens 1005 is positioned at a top region of the body 1003 for providing a field of view.

FIG. 40 is a top view of a tool positioning system 500 for performing a medical procedure, in accordance with embodiments of the present inventive concepts. The tool positioning system 500 is constructed and arranged to position one or more tools (not shown) for performing a medical procedure on a patient P, for example, a transoral robotic surgery procedure or the like. The medical procedure can include a surgical procedure that includes inserting one or more tools into a cavity of the patient (P), or a region of the patient (P) formed by an incision or related opening. A surgical procedure can include one or more transoral procedures. Typical transoral procedures include resections or other procedures performed at or near a location selected from the group consisting of: base of a tongue; tonsils; base of skull; hypopharynx; larynx; trachea; esophagus; stomach; small intestine; and combinations of these. Other procedures can include but not be limited to single or multiport transaxilla procedures, such as a laryngectomy, single or multiport thoracoscopic procedures, such as a mediastinal nodal dissection, single or multiport pericardial procedures, for example, related to measuring and treating arrhythmias, single or multiport laparoscopic procedures, such as revision of bariatric lap-band procedures, single or multiport transgastric or transenteric procedures, such as a cholecystectomy or splenectomy, and/or single or multiport transanal or transvaginal procedures, such as a hysterectomy, oophorectomy, cystectomy and colectomy.

The tool positioning system 500 comprises an introduction device 480, a first tool support 560a, and a second tool support 560b. Although two tool supports 560a, 560b (generally, 560) are shown, the tool positioning system 500 can be constructed and arranged to include more than two tool supports 560. In one embodiment, the tool positioning system 500 includes two, three, or four tool supports 560, each constructed and arranged to slidingly receive a tool, for example, a shaft of a tool. In other embodiments, the tool positioning system 500 includes five or more tool supports 560, each constructed and arranged to slidingly receive a tool.

In some embodiments, an introduction device 480 is constructed and arranged to slidingly receive an articulating probe such as the articulating probe 400, and support, stabilize, and/or guide the articulating probe to a region of interest. The region of interest may be a lumen of a body of a patient (P), such as a cavity at the patient's head (H), e.g., a nose or mouth, or an opening formed by an incision. In clinical applications, typical regions of interest can include but not be limited to the esophagus or other locations within the gastrointestinal tract, the pericardial space, the peritoneal space, and combinations thereof. A region of interest may alternatively be a mechanical device, a building, or another open or closed environment in which probe 400 can be used.

The articulating probe 400 may be configured to guide one or more surgical tools, for example, during a medical procedure. The articulating probe 400 may include inner and outer sleeves, which can advance or retract with respect to one another during manipulation of the articulating probe 400. For example, the inner and outer sleeves of the articulating probe 400, which may include a plurality of inner links and a plurality of outer links can be configured in one of a limp mode and a rigid mode so as to facilitate the manipulation of the articulating probe 400. For example, the inner and outer sleeves may be configured in one of the limp mode and the rigid mode via one or more steering cables (not shown) of the articulation probe 400.

The articulating probe 400 can be a highly articulated probe, for example, a highly articulated probe as described in U.S. Patent Application Publication No. 2009-0171151 entitled STEERABLE, FOLLOW THE LEADER DEVICE, U.S. Patent Publication No. 2008-0039690 entitled STEERABLE MULTI LINKED DEVICE HAVING MULTIPLE WORKING PORTS, or PCT Application No. PCT/US2011/044811 entitled “SURGICAL POSITIONING AND SUPPORT SYSTEM, each incorporated by reference in their entirety herein. The articulating probe 400 may include one or more light sources, image capturing devices, e.g., a camera, provided at the distal end of the articulating probe 400 and/or proximal the distal end of the tool supports 560.

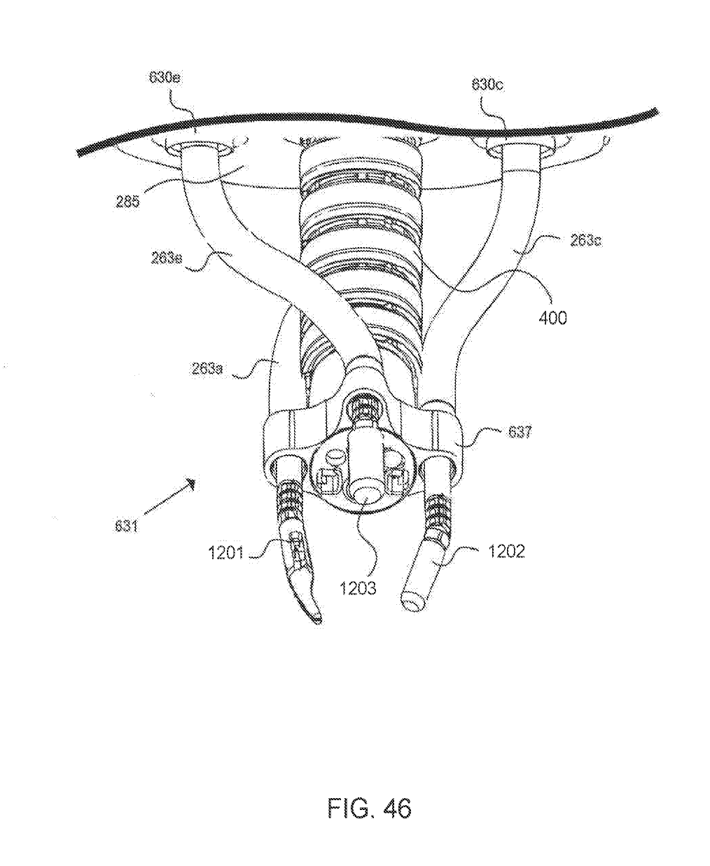

The articulating probe 400 comprises a feeder 110, for example, described herein, which controllably advances one or more cables within an outer sleeve of the probe 10, such as a cable (not shown) extending to a distal link, for example, a distal link 631 shown in FIG. 46. The feeder 110 can comprise one or more cable control assemblies such as bobbin-driven motors or the like and one or more link translating assemblies such as linearly advanceable carts.

The first tool support 560a can be constructed and arranged to slidingly receive a shaft of a tool (not shown). The first tool support 560a is oriented toward a first operator location (L1). The second tool support 560b can also be constructed and arranged to slidingly receive a shaft of a tool (not shown). The second tool support 560b is oriented toward a second operator location (L2). The first and second tool supports 560a, 560b can have similar configurations, or different configurations such as different lengths. First and second tool supports 560a, 560b can be attached to one or more locations on the distal end of probe 400. In some embodiments, tool supports 560a, 560b are on opposite sides of the distal end of probe 400. In some embodiments, tool support 560a is attached to the same side of the distal end of probe 400 as operator location L1 is positioned (e.g. the left side of the page as shown), and tool support 560b is attached to the same side of the distal end of probe 400 as operator location L2 is positioned, e.g. the right side of the page as shown. Alternatively, tool support 560a is attached to the opposite side of the distal end of probe 400 as operator location L1 is positioned (e.g. the right side of the page), and tool support 560b is attached to the opposite side of the distal end of probe 400 as operator location L2 is positioned, e.g. the left side of the page. One operator can control a first tool at one side of the introduction deat which extends from a distal end of the articulating probe 400. Another operator can control a second tool positioned at another side of the distal end of the articulating probe 400. In another embodiment, both operators can have tools positioned at both sides of the introduction device 480 and the distal end of the articulating probe 400.

The tool positioning system 500 can include a base 485. The base 485 can comprise openings for receiving the tool supports 560 and the introducer 480, which can be attached to the base 485 at their midportions, or at distal ends thereof. The first tool support 560a and the second tool support 560b are coupled to the base 485 to maintain a relative position between the first tool support 560a and the second tool support 560b and/or maintain a fixed orientation between the first tool support 560a and the second tool support 560b.

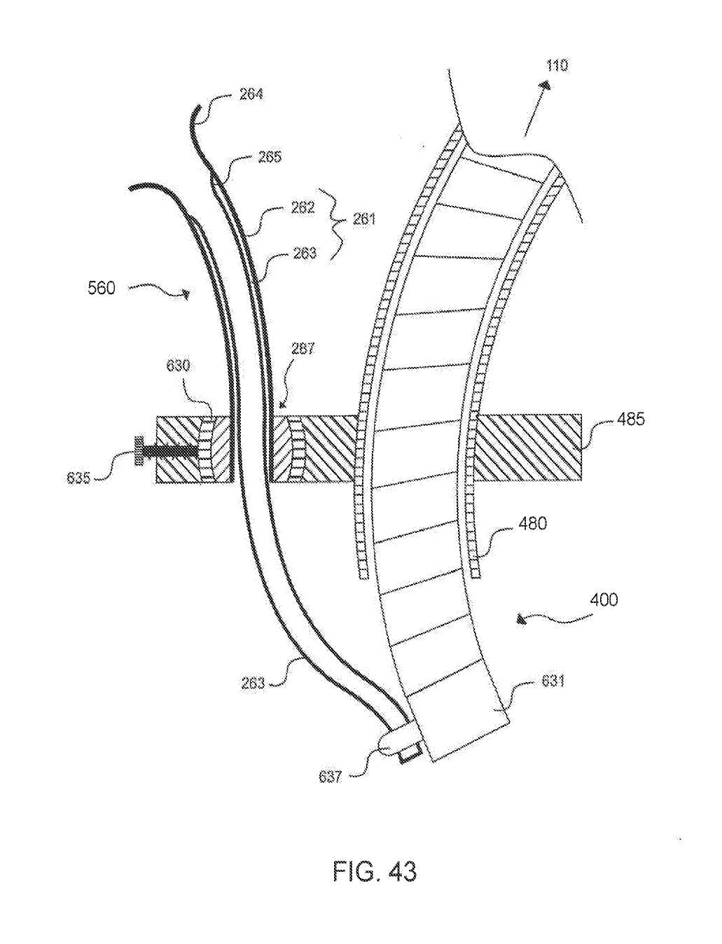

The base 485 can comprise a collar or the like that surrounds at least a portion of the introduction device 480. The collar can extend in a lateral direction relative to a direction of extension of the introduction device 480. As shown in FIG. 43, the base 485 can have an opening 287 aligned with a guide element 561 of each tool support 560. The guide element 561 can be affixed to the opening 287 of the base 485.

The tool positioning system 500 can include a connector 580, also referred to as a dogbone connector, coupled to the first tool support 560a and the second tool support 560b. The connector 580 is constructed and arranged to maintain a relative position between the first tool support 560a and the second tool support 560b. In some embodiments, connector 580 is constructed and arranged to maintain a relative orientation between the first tool support 560a and the second tool support 560b.

The connector 580 can comprise a rigid structure. The connector 580 can comprise at least a portion that is flexible. The connector 580 can comprise an operator shapeable structure. The connector 580 can comprise a malleable structure. The connector 580 can comprise two segments connected by a hinge, such as a butt hinge, a butterfly hinge, a barrel hinge or a hinge comprising a flexible portion positioned between two rigid portions. The connector 580 can comprise a telescopically adjustable structure, such as to allow separation of tool supports 560a and 560b. The connector 580 can comprise two segments connected by a rotatable connector, such as a universal joint.

The connector 580 can be constructed and arranged to be shaped, molded, or the like, such as after the application of heat. The connector 580 can be constructed and arranged to be attachable to at least one of the first tool support 560a or the second tool support 560b. The connector 580 can be constructed and arranged to be detachable to at least one of the first tool support 560a or the second tool support 560b.

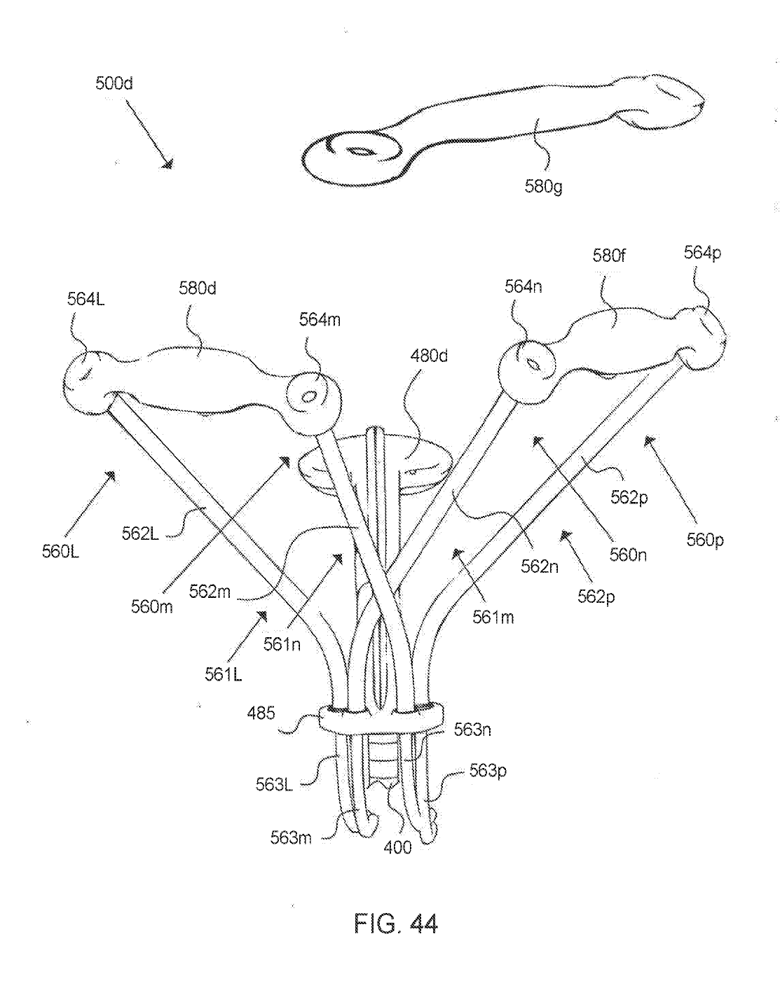

An alternative connector can be provided, for example, connector 580d shown in FIG. 44, that is attachable to the first tool support 560a and the second tool support 560b. The alternative connector 580d can be constructed and arranged to maintain a relative position between the first tool support 560a and the second tool support 560b. The original connector 580 can be constructed and arranged to position the first tool support 560a and the second tool support 560b in a first geometry. The alternative connector can be constructed and arranged to position the first tool support 560a and the second tool support 560b in a second geometry different than the first geometry. The original connector 580 can differ from the alternative connector 580d by at least one of length, shape, curvature, or other geometry or configuration.

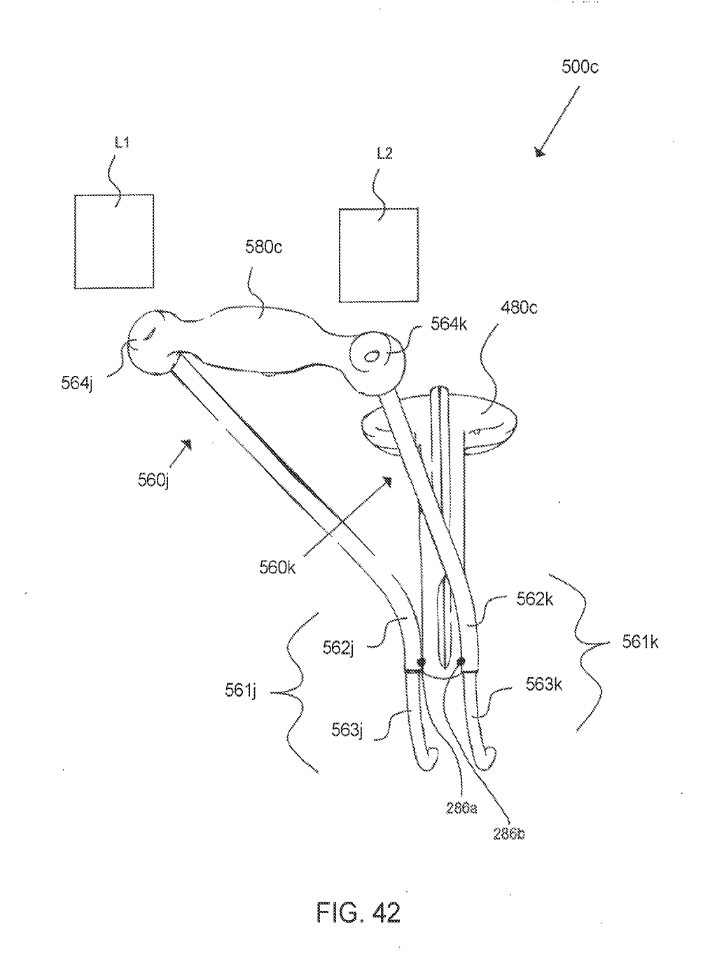

The connector 580 comprises a first opening and a second opening, each constructed and arranged to operably engage a guide element of the first and second tool supports 560a, 560b. The first opening and the second opening can be constructed and arranged to position the first tool support 560a and the second tool support 560b in a non-parallel configuration. At least one of the first opening or the second opening can comprise a funnel-shaped opening, for example, for receiving a guide element 561, more specifically, a funnel-shaped proximal end opening 564 of an outer guide element 562 as shown in FIG. 42.

As shown in FIG. 40, the tool positioning system 500 can include at least one fixation point 133a-e shown (generally, 133), each constructed and arranged to attach to a stabilizing brace. Other fixation points not shown may nevertheless apply, for example, at different locations of the tool positioning system 500. A fixation point 133a can be positioned at the introduction device 480. A fixation point 133b can be positioned at the base 485. A fixation point 133c can be positioned at the first tool support 560a. A fixation point 133d can be positioned at the second tool support 560b. A fixation point 133e can be positioned at the connector 580. A brace 432, also referred to as a support, can be attached to the fixation point 133a. Another end of the brace 432 can be attached to other locations related to the tool positioning system 500, such as an operating room floor, the patient operating table (T) and/or an articulating probe feeder 110. The brace 432 can include a clamping device and the like for clamping to a floor table or other supporting object. Multiple braces can be coupled to different fixation points 133. For example, a brace (not shown) can be coupled between the fixation point 133b at the base 485 and a fixation point 133c at the first tool support 560a. Another brace 431 can be attached to the feeder 110 and can be clamped or otherwise attached to a floor, table or other object providing stability.

The system 500 can include a first human interface device (HID) 80a and a second HID 80b that communicate with a controller 85. As shown in FIG. 40, the first HID 80a can be proximate to or oriented toward the first operator location (L1) and the second HID 80b can be proximate to or oriented toward the second operator location (L2). In other embodiments, the first and second HIDs 80a, 80b can be part of a same hardware platform, and can be at a single or multiple operator location, for example, location (L1), and can permit an operator at either location L1 or L2 to access the HIDs 80a, 80b at the same location. Some or all of the first HID 80a and/or the second HID 80b can be integrated into one or more tools inserted at a tool support 560. In an embodiment, the system 100 includes a third HID 80c attached to integral with dogbone connector 580, HID 80c in wired or wireless communication with the controller 85.

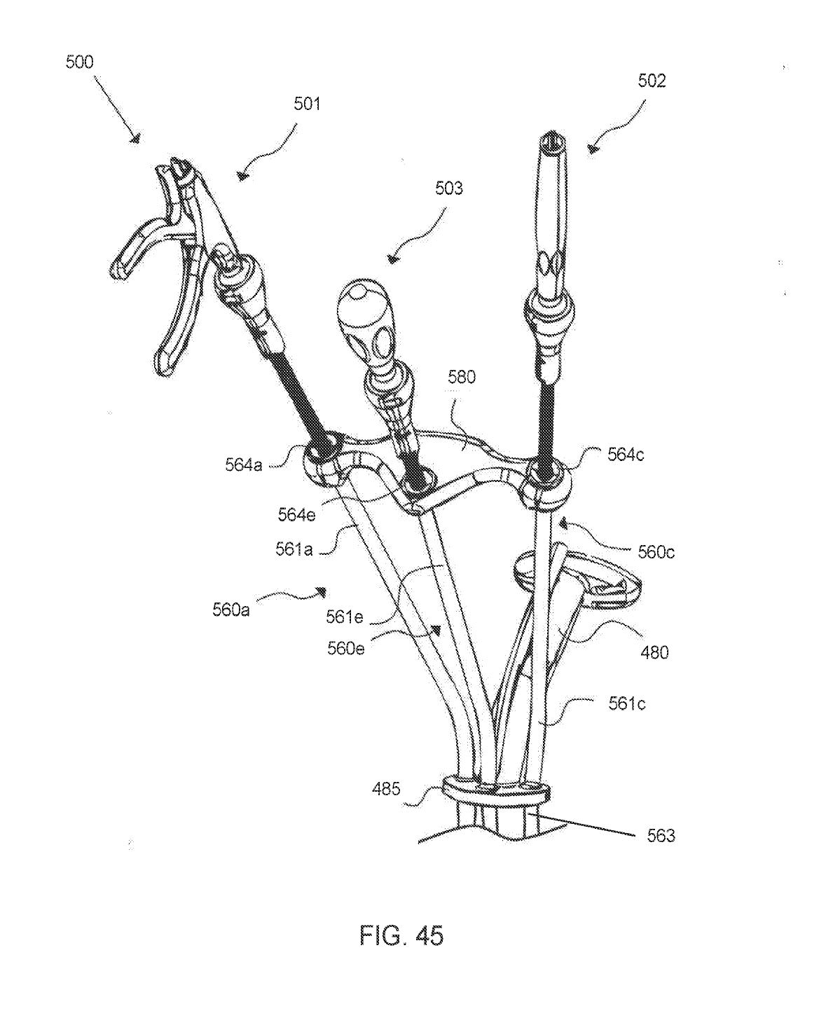

One or more HIDs 80a, b, c (generally, 80) can be constructed and arranged to manipulate the articulating probe 400, the tool supports 560, one or more tools inserted into tool supports 560, or a combination thereof. In system 100 of FIG. 40, the first HID 80a is oriented toward the first operator location (L1). The second HID 80b is oriented toward the second operator location (L2). A first operator, such as a medical professional, may control the articulating probe 400 via the HID 80a to steer, advance, retract or otherwise control the functions and movement of articulating probe 400 via commands sent to the controller 85. A light source, camera, or other device attached to the articulating probe may be activated in response to a control signal generated by the HID 80a. Alternatively or additionally, a second operator may control the articulating probe 400 via the second HID 80b, to steer, advance, retract or otherwise control the functions and movement of the articulating probe 400 via commands sent to the controller 85. A light source, camera, or other device attached to the articulating probe may be activated in response to a control signal generated by the HID 80b. The first HID 80a and/or the second HID 80b may include a device selected from the group consisting of: a haptic controller, a joystick, a track ball, a mouse and an electromechanical device. The articulating probe 400 may be controlled via an HID 80, and the surgical tools may be controlled via a tool handle, for example, a tool handle as shown in FIG. 45. One or more HIDs 80 can communicate with the controller by a physical connector, such as a conductive wire, or by a wireless connection, for example, a Bluetooth™ connection. An HID 80 can include switches, joystick, buttons, and the like for applying forces related to the movement of an articulating probe 400 shown in FIG. 43. In other embodiments, an HID 80 can include sensors such as strain gauges or other force sensors, which can detect forces applied to a dogbone connector 580, for example, push, pull, and/or twist forces. Other sensors may be applied to other elements of the system 500, for various reasons such as those described herein. For example, such forces can be applied for controlling the articulating probe 400 shown in FIG. 44, for example, to advance, retract, or steer the probe 400.

During a medical procedure, the patient (P) can lie on an operation table (T), for example, face up as shown in FIG. 40. In an embodiment, the first operator location (L1) and second operator location (L2) can be side-by-side, or neighboring each other in a manner that permits two or more operators to each maneuver one or more tools. The first tool support 560j and/or the second tool support 560k can be constructed and arranged to provide tool access to a patient's head (H). For example, the first tool support 560j can provide tool access to a patient's esophagus via the patient's mouth. The first tool support 560j and/or the second tool support 560k can be constructed and arranged to provide tool access to at least one of a patient chest or a patient abdomen

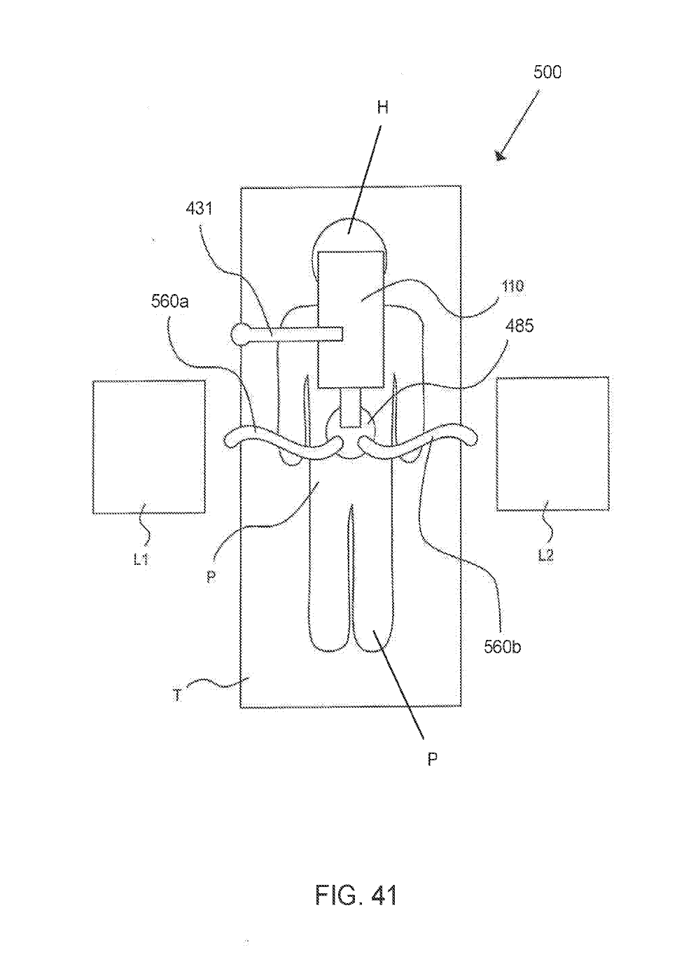

FIG. 41 is a top view of a tool positioning system 500 for performing a medical procedure, in accordance with other embodiments of the present inventive concepts.

In the embodiment, the first operator location (L1) and the second operator location (L2) are at face-to-face locations, for example, at opposite sides of an operating table (T) so that an operator at the first operator location (L1) and an operator at the second operator location (L2) can face each other. The first tool support 560a can extend in a direction towards the first operator location (L1) at a first side of the table (T) and the second tool support 560b can extend in a direction towards the second operator location (L2) at a second side of the table (T) opposite the first side. The first tool support 560a and/or the second tool support 560b can be constructed and arranged to provide tool access to a region of the patient's (P) body, for example, at least one of a patient chest or a patient abdomen, or head (H).

As shown in FIG. 42, the first tool support 560j and the second tool support 560k can be fixedly coupled to a surface of the introduction device 480c instead of a base. In an embodiment, the first tool support 560j and/or the second tool support 560k are directly coupled to the introduction device 480c by attachment mechanisms, for example, welding points 286a, 286b, respectively. Alternatively, other bonding techniques, for example, adhesives and the like, can be applied. The connection at the introduction device 480c maintains a fixed distance and/or a fixed orientation between the first tool support 560j and the second tool support 560k. In some embodiments, the tool supports 560j and 560k can be rotatably attached to each other and/or a base for maintaining a fixed distance but not a fixed orientation. The first tool support 560j and the second tool support 560k can be fixed in position relative to each other. Accordingly, positions of the first and second tool supports 560j, 560k are maintained during an operation of the tool positioning system 500c.

At least one of the first tool support 560j and the second tool support 560l can include first and second guide elements 561ja, 561kb, respectively. The first guide element 561j a can include an outer guide element 562j, also referred to as a proximal guide element, and an inner guide element 563j, also referred to as a distal guide element. The second guide element 561k can include an outer guide element 562j and an inner guide element 563k. At least a portion of the inner guide element 563j, k (generally, 563) is flexible. The inner guide element 563 can be formed of plastic or related material. Materials can include but are not limited to fluoropolymers (e.g., polytetrafluoroethylene), fluorinated ethylene propylene, polyether block amide, high density polyethylene, low density polyethylene and/or nickel titanium alloy. Inner guide element 563 can comprise laser cut tubes (e.g. polymer or metal tubes) and/or coils or braids of plastic or metal. In some embodiments, inner guide element 563 comprises a polytetrafluoroethylene liner. In some embodiments, inner guide element 563 comprises a stainless steel coil. In some embodiments, inner guide element 563 comprises a coil covered by a polyether block amide. In some embodiments, inner guide element 563 comprises different varying stiffness along its length, such as when comprising a tube of varying durometers along its length. At least a portion of the outer guide element 562j, 562k (generally, 562) is rigid, with limited or no flexibility. The outer guide elements 562j, 562k can be directly anchored to the introduction device 480c by a weld 286a, 286b, respectively.

The outer guide elements 562 can include a first tube. The inner guide elements 563 can include a second tube, a portion of which can be positioned in, and move relative to, the first tube of the outer guide element 562. In this manner, the inner guide element 563 can movably extend from the outer guide element 562, for example, in a telescoping configuration.

As shown in FIG. 43, a tool support 560 can rotatably engage the base 485. A single tool support 560 is shown in FIG. 43, however any tool support described herein (e.g. first tool support 560a, second tool support 560b, third tool support 560c, and/or fourth tool support 560d) can be configured as shown in FIG. 44. The tool support 560 can be coupled to the base 485 by a gimbal 630, permitting the tool support 560 to rotate relative to the base 485, for example, allowing for three degrees of freedom between tool support 560 and base 485, which can include two-dimensional (X-Y) movement plus rotation. The gimbal 630 or other pivoted or ball and joint mechanism permits the guide element 261 of the tool support 560 to rotatably or fixedly engage the base 485, for example, at a mid-portion of the guide element 261. In embodiments where a tool support 560 is slidably adjustable, thus allowing for a shortening of a portion of the support 560 that attaches to the dogbone connector 580, the dogbone connector 580 may require adjustability of the distance between connector openings. Depending on the desired relative orientation of one support 560 to the other, parallel or angled, then the adjustability in the connector 580 for the distance between openings can occur along a straight or curved path. Alternatively, the guide element 261 of the tool support 560 can be fixedly attached to a base, for example, at a mid-portion of the guide element 261. The tool support 560 can be locked in a fixed position relative to the base 485. The system 100 can include a locking mechanism 635 to lock the at least one tool support 560 in the fixed position. The locking mechanism may be constructed to secure a position of the tool supports 560 with respect to the base 485, thus preventing the tool supports 560 from sliding or otherwise moving axially during movement of the tools by one or more operators.

The outer guide element 262 of the guide element 261 of a tool support 560 can be constructed and arranged to have a hollow elongate member. The hollow elongate member can be constructed and arranged as a structure known to those of ordinary skill in the art, for example, a hollow tube; a coil such as a helical coil, or combinations thereof. In an embodiment, the entire hollow elongate member is rigid. In another embodiment, at least a portion of the hollow elongate member can be rigid. The inner guide element 263 can be likewise constructed and arranged to have a hollow elongate member. In an embodiment, the entire hollow elongate member can include a flexible tube. Alternatively, the hollow elongate member can include at least a flexible portion. The inner guide element 263 can slide along an inner surface in the opening of the outer guide element 262 in which the inner guide element 263 is positioned.

The outer guide element 262 can have a funnel-shaped proximal end 264. The inner guide element 263 can likewise have a funnel shaped proximal end 265. Either or both funnels 264, 265 can be configured to readily and a traumatically introduce tools to the tool support 260. As shown in FIG. 42, a funnel shaped proximal end opening 564j, k of each tool support 560j, k, respectively, can be positioned about an opening in a connector 580c.

The outer guide element 262 and/or inner guide element 263 can be constructed and arranged to guide or otherwise provide a support for a tool shaft so that it can be guided to a side port 637 coupled to an outer surface of the articulating probe 400.

In some embodiments, the side port 637 is coupled to a distal link 631 of the articulating probe 400, but not limited thereto. For example, in other embodiments, the side port 637 can be formed at a flange, or lobe, of a link at the articulating probe 400. Multiple side ports may be positioned along the outer sleeve of the articulating probe 400 so as to provide a guide for one or more guide elements 261 that articulate in common with the articulating probe 400. Alternatively, the inner guide element 263 can be fixedly attached to the outer surface of the articulating probe 400, for example, the distal link 631, such as with an adhesive or mechanical fastener.

FIG. 44 is a perspective view of a tool positioning system 500d having multiple connectors 580d, 580f, in accordance with an embodiment.

The tool positioning system 500d can also comprise a first tool support 560L, a second tool support 560m, a third tool support 560n and a fourth tool support 560p. Each of tool supports 560l, m, n, and p can include a funnel-shaped opening, 5641, m, n, p respectively, on its proximal end. The tool supports 560l, m, n, p and the introduction device 480d are fixedly attached to base 485d. The third tool support 560n can comprise at least one guide element 561n, which can be similar to the guide elements 561L and 561m described herein. For example, the guide element 561n can include an outer guide element 562n and an inner guide element 563n. The fourth tool support 560p can comprise at least one guide element 561p, which can be similar to the guide elements 561L and 561m described herein. For example, the guide element 561p can include an outer guide element 562p and an inner guide element 563p.

The first tool support 560L and the third tool support 560n can be oriented in a same or similar direction, for example, toward a first operator location. The second tool support 560m and the fourth tool support 560p can be oriented in a same or similar direction, for example, toward a second operator location. Tools (not shown) extending from the first and third tool supports 560L, n, respectively, are shown positioned at a first side and a second side of a distal end of the articulating probe 400, and tools (not shown) extending from the second and fourth tool supports 560m, p, respectively, are shown positioned at the first side and the second side of the distal end of the articulating probe 400, where the first side is opposite the second side. In another embodiment, not shown, tools extending from the first and third tool supports 560L, n, respectively, can be positioned at a first side of a distal end of the articulating probe 400, and tools extending from the second and fourth tool supports 560m, p, respectively, can be positioned at a second side of the distal end of the articulating probe 400.

The outer guide element 562L of the first tool support 560L and the outer guide element 562n of the third tool support 560n can be oriented in a same or similar direction, for example, toward a first operator location. The outer guide element 562m of the second tool support 560m and the outer guide element 562p of the fourth tool support 560p can be oriented in a same or similar direction, for example, toward a first operator location. However, the first and second inner guide elements 563L, m can be collocated, and the third and fourth inner guide elements 563n, p can be collocated.

The tool positioning system 500 can comprise a connector 580d, f attached to proximal ends of the first tool support 560 and the third tool support 560n. The connector 580d, f is constructed and arranged to maintain a relative position between the first tool support 560L and the third tool support 560n. The tool positioning system 500 can also comprise a second connector 580f attached to proximal ends of the second tool support 560m and the fourth tool support 560p. The connector 580f is constructed and arranged to maintain a relative position between the second tool support 560m and fourth tool support 560p. In another embodiment, the first connector 580d can be attached to proximal ends of the first tool support 560L and the second tool support 560m, and the second connector 580f can be attached proximal ends of the third tool support 560n and the fourth tool support 560p.

The connector 580d, also referred to as a first connector or first dogbone connector, and/or the connector 580f, also referred to as a second connector or second dogbone connector, can be removed from the tool supports 560 and replaced with a different connector 580g, which can have different configuration parameters than the connectors 580d, 580f, for example, a different length or openings for receiving a funnel shaped guide element 561.

FIG. 45 is a perspective view of a tool positioning system 500 having three tools 501, 502, 503 in communication with a connector 580, in accordance with an embodiment. A single operator can operate tool positioning system 500, including any or all three tools 501, 502, 503. Alternatively, two or more operators can operate tool positioning system 500, including any or all three tools 501, 502, 503.

Three tool supports 560a, 560c, 560e extend between a base 485 and a connector 580. Each of tool supports 560a, 560c and 560e can include a funnel-shaped opening, 564a, 564c and 564e (generally, 564) respectively, on their proximal end. The base 485 includes a collar having first, second, and third openings aligned with the first, second, and third tool supports 560a, 560c, 560e, respectively. The guide elements 561a, 561c, 561e (generally, 561) of the first, second, third and tool supports 560a, 560c, 560e, respectively, can extend through the first, second, and third openings so that mid-portions of the guide elements 561 are positioned in the openings during operation. The base 485 can include a fourth opening for receiving an introduction device 480. At least one tool 501, 502, 503 can have a shaft, shown inserted into tool supports 560a, 560c and 560e, respectively, constructed and arranged to be slidingly received by a corresponding tool support 560. One or more tool 501, 502, 503 can be selected from the group consisting of: suction device; ventilator; light; camera; grasper; laser; cautery; clip applier; scissors; needle; needle driver; scalpel; RF energy delivery device; cryogenic energy delivery device; and combinations thereof. A tool 501, 502, 503 can include a rigid and/or a flexible tool shaft.

The connector 580 is attached to first, second, and third tool supports 560a, 560c, 560e and can be constructed and arranged to maintain a relative distance between the tool supports 560a, 560c, 560e. The connector 580 can be fixedly attached to one or more of the tool supports 560. Alternatively, the connector 580 can be rotatably attached to one or more of the tool supports 560. The connector 580 maintains a relative position of the third tool support 560e relative to the first tool support 560a and the second tool support 560c.

The base 485 can be fixedly attached to one or more of the tool supports 560. Alternatively, the base 485 can be attached, for example, movably or rotatably attached, to one or more of the tool supports 560. A gimbal can be at the base 485 which engages, for example, movably or rotatably engages, one or more guide elements 561 at the base 485. A single operator can operate one or more of: the tool 501 extending from the first tool support 560a, the tool 502 extending from the second tool support 560c, and/or the tool 503 extending from the third tool support 560e, for example, from a single operator location. Alternatively, one operator can operate two tools of the tool 501, 502, 503 and another operator can operate the remaining tool of the tool 501, 502, 503. As shown in FIG. 46, a first tool 1201 is positioned at a first side of a distal end of the articulating probe 400, i.e., the left side of the page as shown, and a second tool 1202 is positioned at a second side of the distal end of the articulating probe 400, i.e., the right side of the page as shown, opposite the first side. A third tool 1203 can optionally be positioned between the first and second tool 1201, 1202 at the distal end of the probe 400. First tool 1201 may be inserted through a first tool support including inner guide element 263a. Inner guide element 263a passes through base 285, such as via a gimbal not shown but positioned behind probe 400. Second tool 1202 may be inserted through a second tool support including inner guide element 263c. Inner guide element 263c passes through base 285 via gimbal 630c. Third tool 1203 may be inserted through a third tool support including inner guide element 263e. Inner guide element 263e passes through base 285 via gimbal 630e. The first tool 1201 can be controlled by an operator at the corresponding side of the articulating probe 400, i.e., the first side or left side of the page as shown. Alternatively, the first tool 1201 can be controlled by an operator on the opposite side of the articulating probe 400, i.e., the second side or right side of the page as shown. The second tool 1202 can be controlled by an operator at the corresponding side of the articulating probe 400, i.e., the second side or right side of the page as shown. Alternatively, the second tool 1202 can be controlled by an operator on the opposite side of the articulating probe 400, i.e., the first side or left side of the page as shown. The operator at the first and second sides can be the same operator, or different operators at different locations, for example, side-by-side as shown in FIG. 40 or face-to-face as shown in FIG. 41.

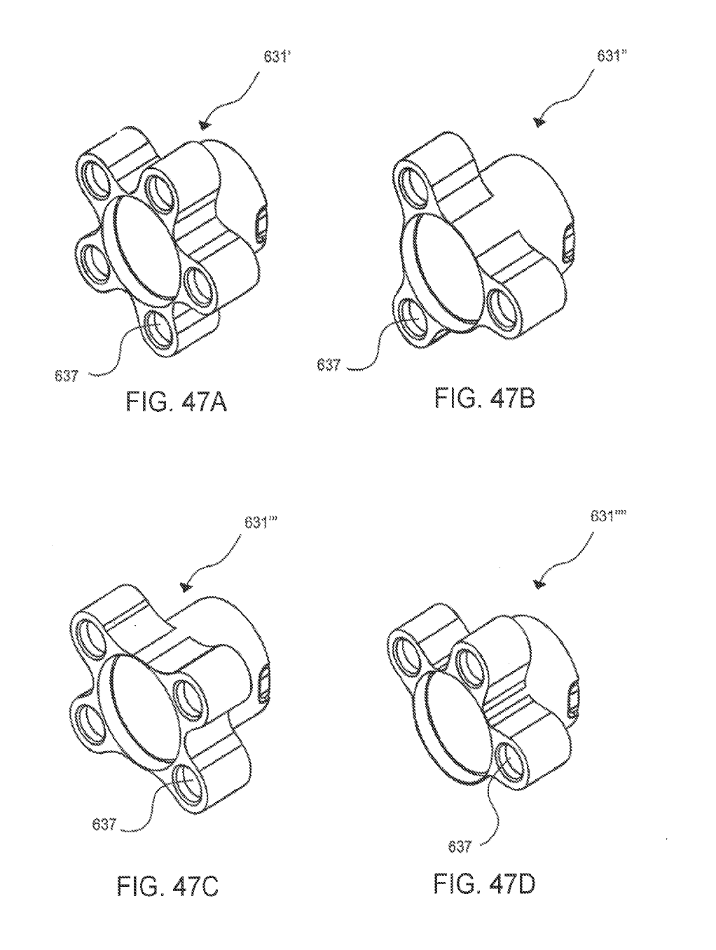

As described above, the articulating probe 400 comprises a distal link 631, which can receive and be positioned about an articulating probe 400. In some embodiments, the distal link 631 comprises at least three side ports 637. In FIG. 47B, the distal link 631″ can include three side ports 637 that can each be coupled to a tool support, for example, tool supports 260a, c, e, respectively.

In another embodiment, as shown in FIG. 47C, a distal link 631′″ comprises four side ports 637, which can each be coupled to a tool support, for example, tool supports 560L, m, n, p shown in FIG. 44.

In another embodiment, as shown in FIG. 47A, a distal link 631′ comprises five side ports 637, which can each be coupled to a tool support for a total of five tool supports, for example, two tool supports oriented toward one operator location, and three tool supports oriented toward another operator location.

In an embodiment, as shown in FIG. 47B, the side ports 637 are symmetrically spaced about a periphery of the distal link 631″. In an embodiment, as shown in FIG. 47D, the side ports 637 are asymmetrically spaced about a periphery of the distal link 631″″.

The side ports 637 can be positioned 30° to 180° apart from each other about a periphery of the connector 280. For example, as shown in FIG. 47D, first and second side ports 637 can be less than 180° apart from each other, such as 150° apart, and a third side port 637 can be positioned between the first and second side ports, such that the third side port 637 is less than 90° apart from each of the first and second side ports 637. The side ports 637 can be attached to one or more tool supports 260 oriented toward an operator location on a similar or dissimilar side as the side port 637.

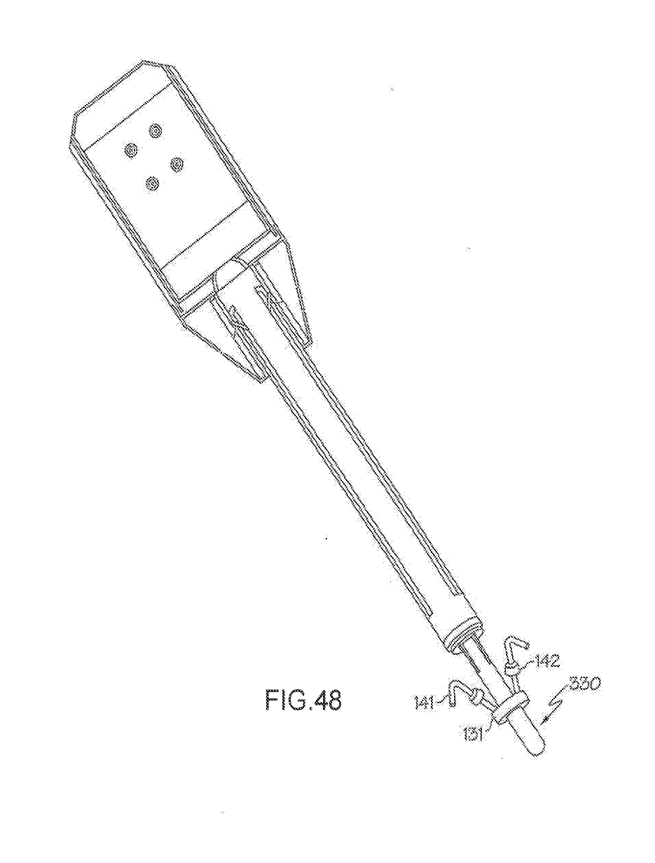

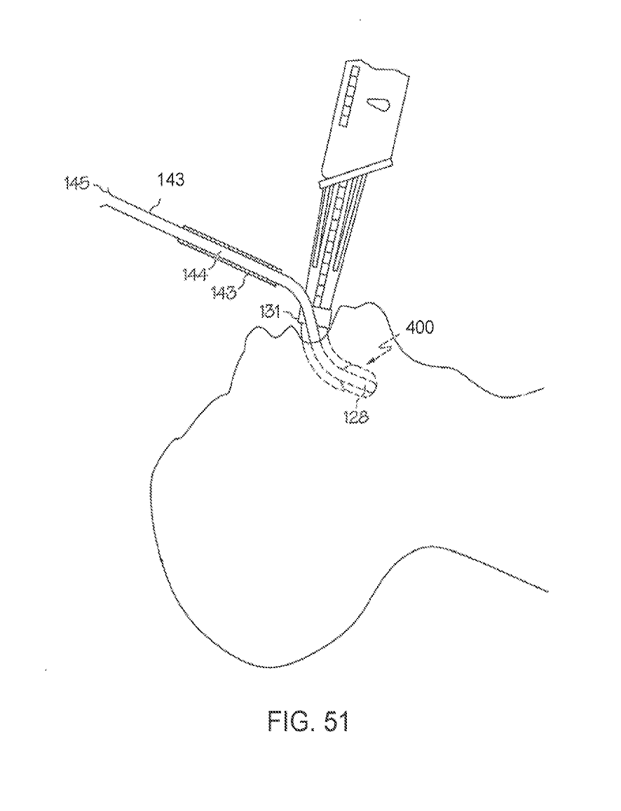

An introduction device 480, such as that shown in FIGS. 48-56, can be configured to support, stabilize and guide an articulated probe, such as the articulated probe 400 described above, to a region of interest. The region of interest may be a lumen, a patient's body, a mechanical device, a building, or any other open or closed environment in which the probe 400 can be used. In clinical applications, typical regions of interest include but are not limited to: the esophagus and other locations within the gastrointestinal tract; the pericardial space; the peritoneal space; and combinations thereof.

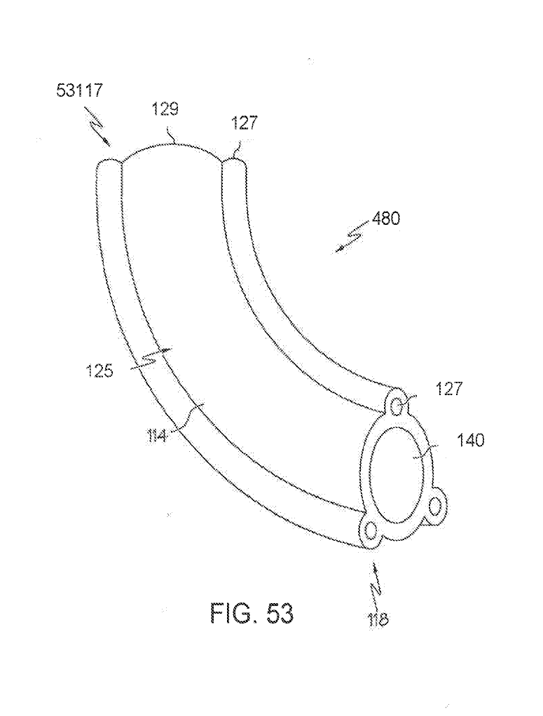

As shown in FIG. 53, the introduction device 480 includes hollow tube 114, which includes a lumen or other hollow passageway that is surrounded by luminal walls forming a support member 125. The lumen and support member 125 extend between an entrance 129 positioned at a proximal end 117 and an exit 140 positioned at a distal end 118. The introduction device 480 can be configured to improve access to regions of interest and provide for fast, safe and/or accurate advancement of the articulated probe 400.

The entrance 129 of the introduction device 480 is configured to receive an articulated probe 400, regardless of a state of the links of the probe 400, for example, a limp mode or rigid mode described herein. The entrance 129 guides the articulated probe 400 so that the articulated probe 400 comes into close proximity or contact with the support member 125. For example, the entrance 129 may guide an articulated probe 400 from a feeder unit 100a into proximity with the support member 125. Accordingly, the entrance 129 guides the articulated probe 400 into the introduction device 480 and into proximity with the support member 125.

The exit 140 (e.g., FIG. 53) of the introduction device 480 is configured to receive the articulated probe 400 from the lumen of introduction device 480. In addition, the exit 140 introduces the articulated probe 400 into a region of interest. For example, the exit 140 may guide the articulated probe 400 from the introduction device 480 into a region of interest such as a body lumen, an esophagus as shown, a subxiphoid space, a colon, or an intracranial space. Thus, the exit 140 facilitates introduction of the articulated probe 400 into a region of interest.

The support member 125 can have any configuration that is capable of supporting or otherwise resisting movement of an articulated probe 400. For example, the support member 125 can be either rigid or flexible. In an example embodiment where the support member is rigid, the support member 125 may be formed from a rigid material, such as machined metal or molded plastic. In an example embodiment where the support member is flexible, the support member 125 may be formed from one or more flexible materials and can include one or more internal malleable members configured to plastically deform so as to maintain an operator formed shape of introduction device 480 or a portion thereof. In other embodiments, the support member can be configured so as to be elastically deformable.

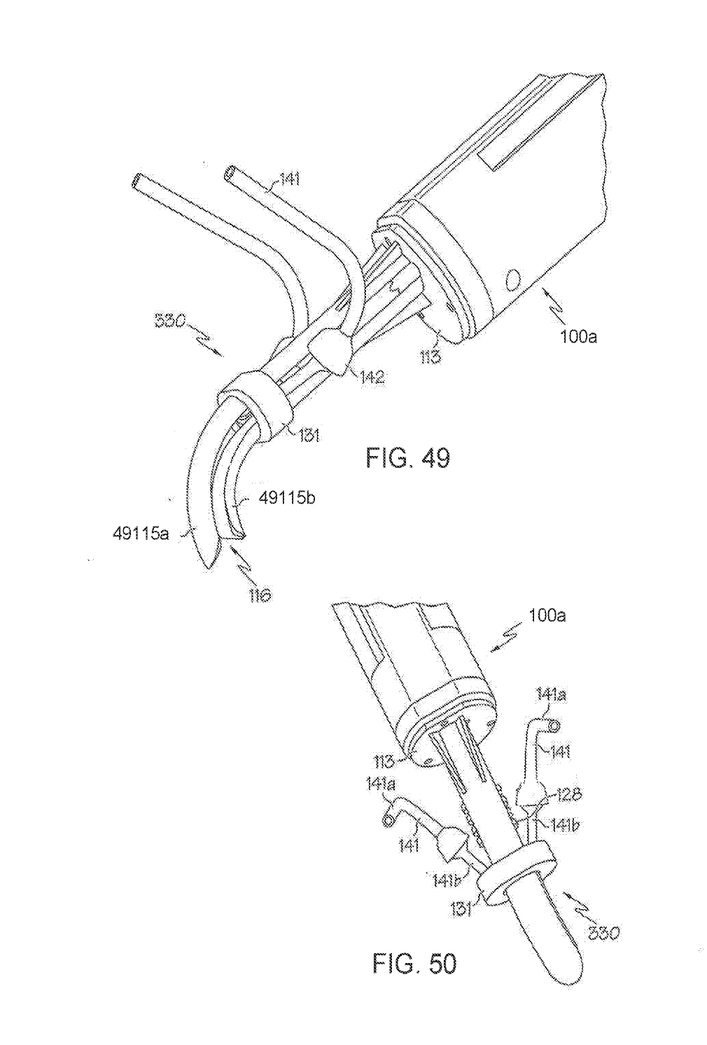

Several possible configurations of the support member 125 are shown in FIGS. 49-56. The support member 125 can be an axially curved member as shown. Alternatively, the support member 125 may be a straight or substantially straight member (not shown). According to one embodiment, the support member 125 has a cylindrical shape, such as a hollow tube 114. The cylindrical shaped support member 125 has an internal diameter. The internal diameter of the support member 125 is larger than the outer diameter of the articulated probe 400. Preferably, the support member 125 diameter is determined by the following formula:

$$ID \geq {l}_{2} + {R}_{1} - {R}_{1} cos [ {sin}^{- 1} [ \frac{{l}_{1}}{2 {R}_{1}} ] ]$$

where l₁ is the segment length, l₂ is the segment diameter, and R₁ is the inner radius of the curvature of the introducer along the axis of the scope. Other configurations determined by the foregoing formula may equally apply, but not limited thereto.

In some embodiments, the introduction device 480 can have an outer diameter that is smaller than the diameter of an opening of the region of interest in which the probe 400 will be used.

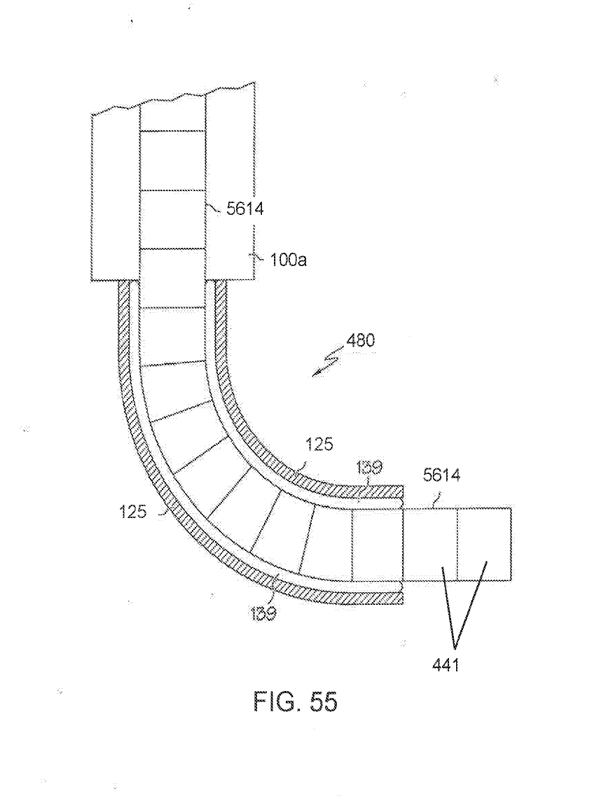

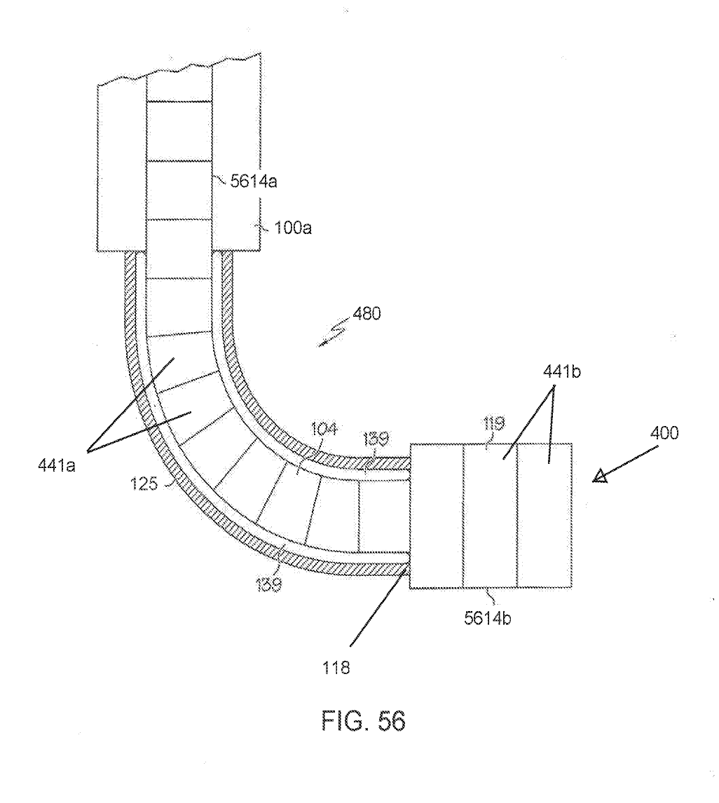

According to one embodiment, the support member 125 can be formed from two opposed and elongated curved surfaces 49115a, 49115b separated by gap 116. In some embodiments, the concave side of one curved surface 49115a opposes the concave side of the other curved surface 49115b so that, in combination, the curved surfaces 49115a, 49115b encompass, or otherwise partially surround and guide the articulated probe 400. Alternatively, a single, elongated curved surface may be used. The support member 125 may have a collar (also known as an attachment mechanism, or attachment collar) 485 disposed circumferentially about the two elongated curved surfaces 49115a, 49115b so as to secure the two elongated curved surfaces 49115a, 49115b, and maintain them at a desired distance apart from each other, and thereby control the width of the gap 116 and the internal diameter of support member 125. The base, or collar 485 can use an interference fit to remain attached to the two elongated curved surfaces 49115a, 49115b or it may be attached using a fastener or an adhesive. Introduction device 480 may include one or more side channel tool ports 560, constructed and arranged to receive a tool shaft or a guide tube for a tool shaft. Side channel tool ports may be similar or dissimilar (e.g. different diameters, stiffnesses, etc.), such as to accommodate similar or dissimilar tools and/or tool shafts. The base or collar 485 may be rotatably attached to support member 125 such as to allow repositioning of tools passing through the side channel tool ports 560. The base or collar 485 may be rotatably attached to support member 125 such as to allow one, two, or more, degrees of freedom of rotation of tool ports 560. In one embodiment, The base or collar 485 provides a single degree of freedom, rotating about the outer diameter of introduction device 480. According to one embodiment as shown in FIG. 55, a clamp 139 is located on support member 125. The clamp 139 further minimizes potential motion of articulated probe 400 and thus further stabilizes the articulated probe 400 as it is positioned within a region of interest. The clamp 139 may be any clamp that can be located proximate articulated probe 400 and/or support member 125 such as to limit motion of probe 400, such as when a force is applied to a distal portion of probe 400. Clamp 139 may be of various forms including a lever, a cam, an expandable member such as a balloon; a piston such as a hydraulic or pneumatic piston; an electromagnetically activated actuator such as a solenoid; and combinations of these. Clamp 139 can be configured to apply a force on a portion of outer sleeve 5614 comprising outer links 441 such as a force applied to an area of at least 1 mm2, at least 10 mm2 or at least 100 mm2. In some embodiments, the clamp 139 comprises a balloon that can be controllably expanded and contracted, such as via one or more controls, not shown but preferably on a proximal portion of probe 400, feeder mechanism 16, and/or a control unit for probe 400. Delivery or removal of one or more fluids (e.g. air), such as through an inflation lumen, not shown but in fluid communication with clamp 139, can causes expansion and contraction, respectively, of clamp 139. When the balloon is in its expanded state, the outer surface of the balloon exerts pressure on the outer surface of the articulated probe 400. This minimizes the ability of the articulated probe 400 to move both radially and axially relative to the support member 125, stabilizing probe 400 within the introduction device 480. Alternatively or additionally, clamp 139 may be constructed and arranged to minimize the ability of the articulated probe 400 to rotate relative to the support member and/or introduction device 480. Stabilization of probe 400 may be of particular importance when manipulating the distal portion of probe 400 within a body cavity such as the esophagus. Stabilization of probe 400 may also be of particular importance when manipulating one or more tools passed through or alongside probe 400, such as when a tool applies a force to a tissue surface such as the esophageal wall of a patient. Accordingly, the support member 125 is configured to support and guide an articulated probe 400 both during advancement to one or more regions of interest as well as and thereafter such as during tool manipulation.

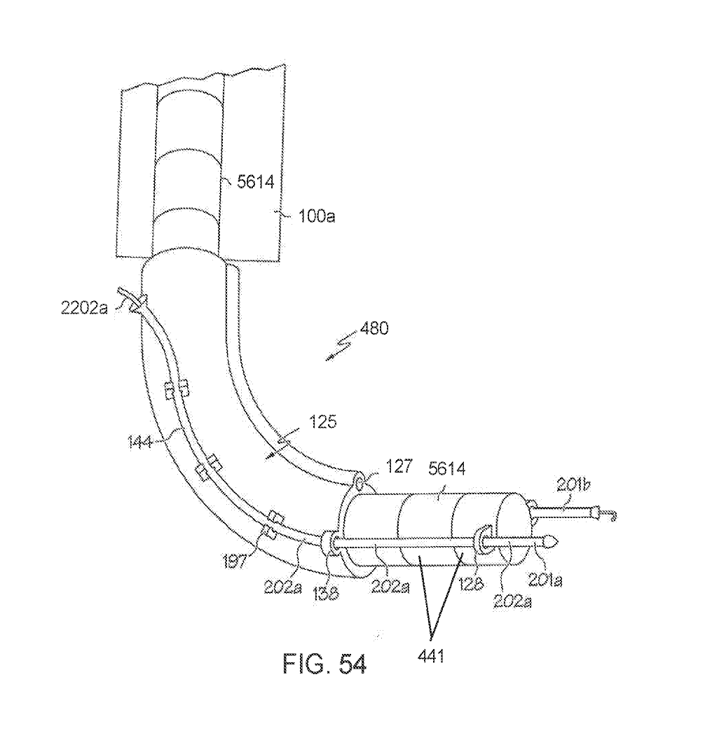

As shown in FIG. 53, the introduction device 480 may have at least one channel 127 that extends along the longitudinal axis of the support member 125. The channel 127 can be integral with an outside wall of the hollow tube 114 of introduction device 480. The channel 127 is configured to allow a filament 2202a, not shown but described in reference to FIG. 54, such as a tool shaft guide tube or a tool shaft, to pass through the channel 127. Accordingly, filament 2202a, and tools attached thereto, can also be introduced into a region of interest via the introduction device 480. Introduction device 480 may be rigid, flexible, or include both rigid and flexible portions. Introduction device 480 may include a malleable, or plastically deformable, member (not shown), which can be configured to allow introduction device 480 to be bent, twisted or otherwise reshaped such that the new configuration is maintained by a supporting force of the malleable member. In one embodiment, introduction device 480 has a corrugated construction permitting flexing while maintaining one or more internal lumen diameters.

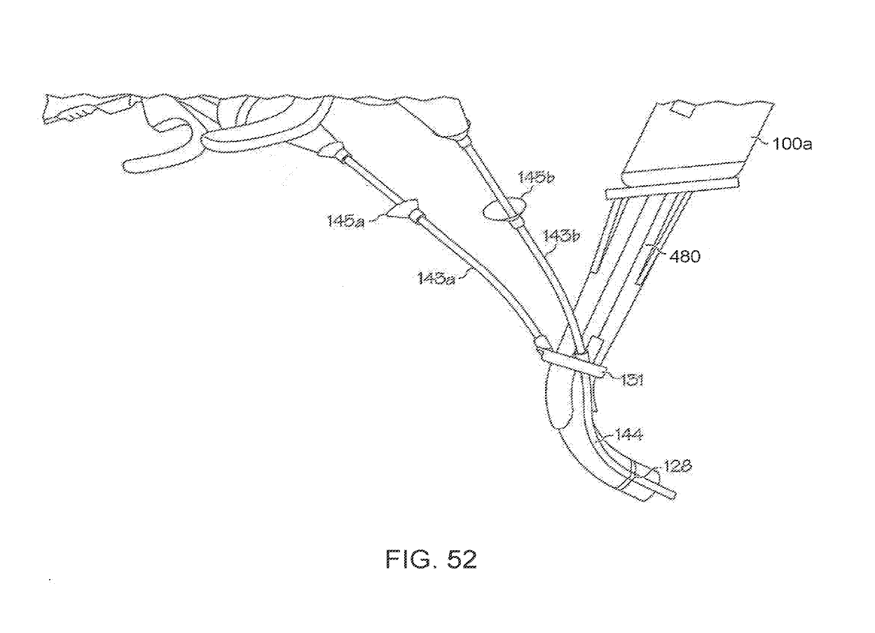

The introduction device 480 may include several configurations for guiding a filament 2202a (see FIG. 54) such as a tool guide tube or a tool shaft, to a tool side port 128 located on a distal portion of articulated probe 400. The introduction device 480 may include a side channel tool port 560. As shown in FIG. 52, the introduction device 480 may include multiple coaxial tubes including tool tube 143a, 143b which slidingly receives, or otherwise communicates with, flexible tube 144. In some embodiments, tool tube 143a, 143b is more rigid than flexible tube 144 such that flexible tube 144 flexes and tool tube 143a, 143b remains relatively rigid when a tool shaft or other filamentous device that has been inserted into flexible tube 144, has a load applied to it.

FIGS. 48-50 depict an embodiment in which the side channel tool port 560 comprises a first section 561 and a second section 563. A joint 142 is positioned between the first section 561 and the second section 563. Preferably, the joint 142 is a spherical joint, a hinged joint, or combinations thereof. Alternatively or in addition to joint 142, first section 561 and/or second section 141b may be flexible or deformable, or may include flexible or deformable sections. The joint 142 allows rotation, or articulation, of the first section 141a relative to the second section 563. Rotation of the first section 561 may also allow the corresponding tool 1201a, 1201b to rotate, such as to allow an operator to position or reposition the proximal end of a tool without positioning or repositioning introduction device 480 or outer sleeve.

According to another embodiment, FIGS. 51 and 52 illustrate a rigid tube 143 for guiding a filament 2202a to a tool side port 128 located on a distal portion of articulated probe 400. A flexible tube 144 may be disposed inside the rigid tube 143. According to the embodiment shown in FIG. 52, the rigid tube 143a, 143b (generally, 143) has a tool funnel 145a, 145b (generally, 145), configured to readily and a traumatically introduce tools into the rigid tube 143. FIG. 54 illustrates a flexible tube 144 attached to an outer surface of introduction device 480 and extending along a longitudinal axis of the introduction device 480. The flexible tube 144 is configured to guide or otherwise provide a support for filament 2202a so that it can be guided into a tool side port 128 located on an outer surface of the articulated probe 400. The flexible tube 144 can be secured, for example snap-fit, to the outer surface of introduction device 480 using “c”-shaped supports 197 located on the outer surface of introduction device 480. Alternatively or additionally, supports 197 may be a closed-loop configuration such that flexible tube 144 can be slidingly received therethrough. In the snap-fit configuration, supports 197 are constructed and arranged to allow flexible tube 144 to be inserted through the application of a light pressing force relatively orthogonal to the outer surface of introduction device 480. Supports 197 may be further configured to allow flexible tube 144 to be detached through the application of a slight tension force in a direction away from the outer surface of introduction device 480. The flexible tube 144 is configured to guide a filament 2202a along the body of the introduction device 480 and through a side channel 138 positioned on the outer surface of introduction device 480. The side channel 138 is configured to allow a filament 2202a, such as a tool guide tube or a tool shaft, to pass through the side channel 138. The side channel 138 guides a filament 2202a into a tool side port 128 located on an outer link of the articulated probe 400. The filament 2202a passes through both the side channel 138 located on the outer surface of introduction device 480 and the side port 128 located on the probe 400. Thus, the introduction device 480 facilitates the introduction of tools passed through the side channel 138 of the introducer and side port 128 of the articulated probe 400. Flexible tube 144 may be fixedly attached to side channel 138 (e.g. with adhesive or a mechanical fastener). Alternatively, flexible tube 144 may be allowed to slide through side channel 138.

FIG. 56 shows an introduction device 480 in which the probe 400 has several distal outer links 441b on its distal end, for example, outer sleeve 5614b, that are larger than the outer links 441a on a more proximal portion of the outer sleeve 5614a of articulated probe 400. The distal outer links 441b may be larger in diameter than an opening of the introduction device 480 such that a proximal side of one of the distal outer links 441b can contact the distal end 118 of the introduction device 480. In this configuration, because the diameter of the distal outer links 441b is greater than the opening of the introduction device 480, the articulated probe 400 cannot fully retract into the introduction device 480. The smaller outer links 441a may be constructed and arranged to have a smaller radius of curvature than that of the larger outer links 441b, for example in a case where the introduction device 480 has a smaller radius of curvature than that of the larger outer links 441b. The larger outer links 441b can be advanced forward of introduction device 480, or simply steered. Larger outer links 441 can provide numerous advantages including improved stability when one or more forces are applied to the distal end of the outer sleeve 5614.

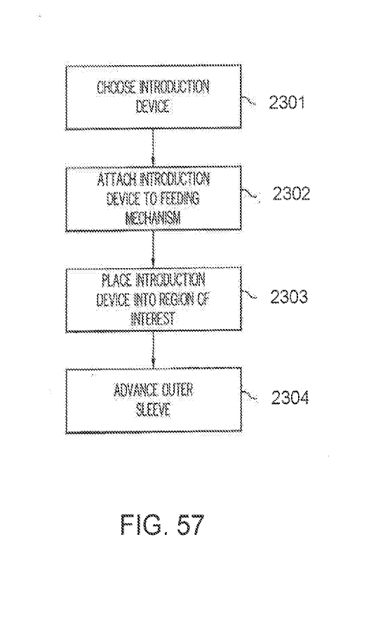

Referring to FIG. 57, a method of introducing an articulated probe to a region of interest is illustrated. Some or all of the method may be performed by elements of an articulating probe system in accordance with some embodiments. As with other methods described in other embodiments, the method of FIG. 57 shall not be construed as being order-specific. Accordingly, the sequence of steps in FIG. 57 may be performed in a different order.

In STEP 2301, an introduction device is chosen, such as an introduction device described herein. The introduction device can be chosen based on one or more parameters such as a parameter associated with the region to be accessed by the articulated probe. In a particular embodiment, the articulated probe is used on a patient and the introduction device is chosen based on patient anatomy, such as the esophageal geometry of the patient. Numerous forms and geometries of introduction devices may be made available to an operator such as a clinician, such as in a kit form for patient and/or application specific selection. In STEP 2302, the introduction device 480 is attached to a feeding mechanism 16. Specifically, the proximal end 117 of the introduction device 480 is attached to the feeding mechanism 16. According to one embodiment, the introduction device 480 has an attachment surface 113. The attachment surface 113 can be permanently attached or integral to the feeder unit 100a or can be removably attached to the feeding mechanism 16. The feeder unit 100a can be any feeding mechanism known in the art for feeding an articulated probe 400. Preferably, the feeder unit 100a is the feeding mechanism shown herein and described above, and used to independently cause both an inner link mechanism 420 and outer link mechanism 440 of probe 400 to transition from rigid to flexible states; as well as independently advance and retract the inner link mechanism 420 and outer link mechanism 440. The articulated probe 400 can be fed and pre-loaded from the feeder unit 100a into the introduction device 480, such as when both an inner link mechanism 420 and outer link mechanism 440 of probe 400 are in a flexible state.

In STEP 2303, a distal portion of introduction device 480 is placed into a region of interest such as a location internal to a patient. In one method, outer link mechanism 440 may be advanced into introduction device 480 (e.g. until the distal end of outer link mechanism 440 is proximate the distal end of introduction device 480), prior to placing introduction device 480 into the patient. Subsequently, both the introduction device 480 and outer link mechanism 440 are advanced to the region of interest simultaneously. In a different method, outer link mechanism 440 is advanced into and/or through introduction device 480 after the distal end 118 of introduction device 480 has been placed into the patient. Outer link mechanism 440 may be advanced through introduction device 480 at an accelerated rate, such as a rate faster than is used during surgical or other high-precision manipulations.

The accelerated rate may be achieved by increasing the speed of cable tensioning (inner core and outer sleeve transitioning from flexible to rigid states) and/or cart movement (advancement and retraction of the inner core and outer sleeve) of probe 400, as has been described in detail herein. Alternatively or additionally, outer, link mechanism 440 may be advanced through introduction device 480 and/or with probe 400 in a flexible state (e.g. outer sleeve in a flexible state or inner core and outer sleeve in flexible states). These accelerated advancements of probe 400 through introduction device 480 simplify use of probe 400, and greatly reduce procedure time.

In STEP 2304, outer link mechanism 440 is advanced into the patient, in a direction that is away from the distal end of introduction device 480, such as been described in detail hereabove. When the region of interest is a lumen, the introduction device 480 may work in conjunction with a retractor, such as a mouth retractor. The size and shape of the introduction device 480 may vary based on the region of interest. In the case of a body lumen region of interest, the size and shape of the introduction device 480 may vary based on the anatomy, size, and shape of the patient or the body lumen of the patient.

In an alternative embodiment, introduction device 480 may be inserted into a patient or other region of interest prior to attachment to the feeding mechanism 16. Subsequent to insertion, introduction device 480 may be attached to the feeder unit 100a and distal end of outer link mechanism 440 advanced through introduction device 480 and into the region of interest.

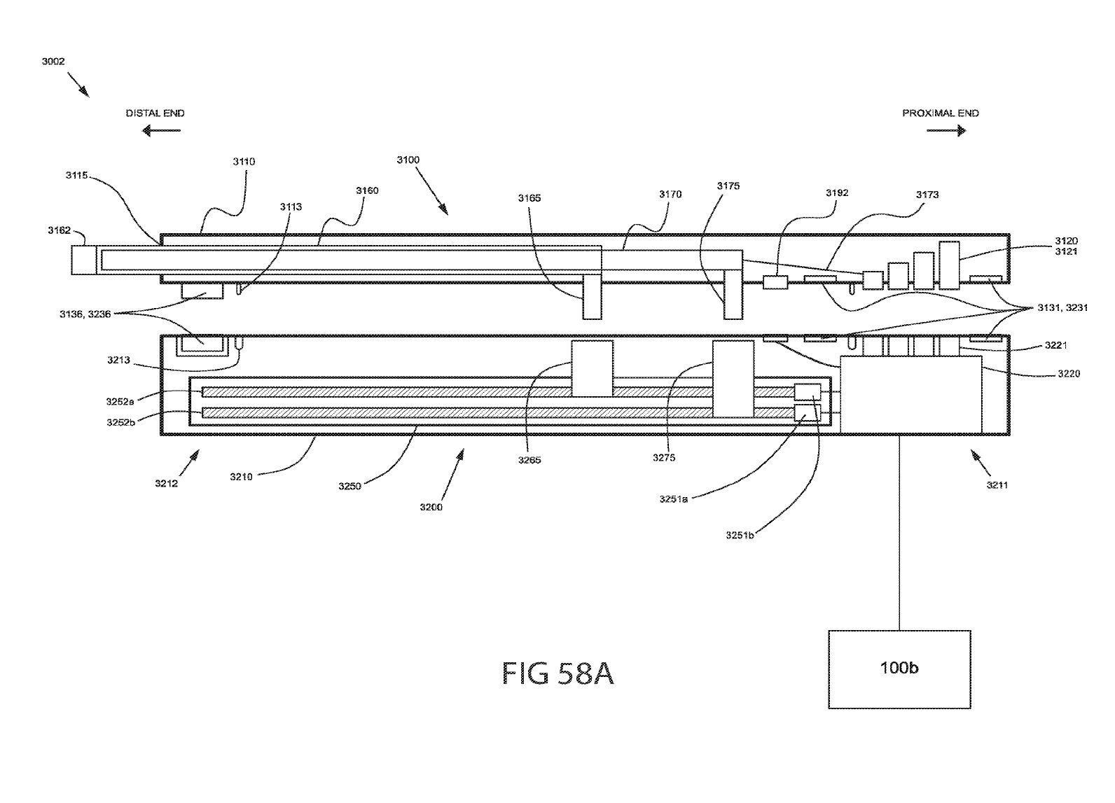

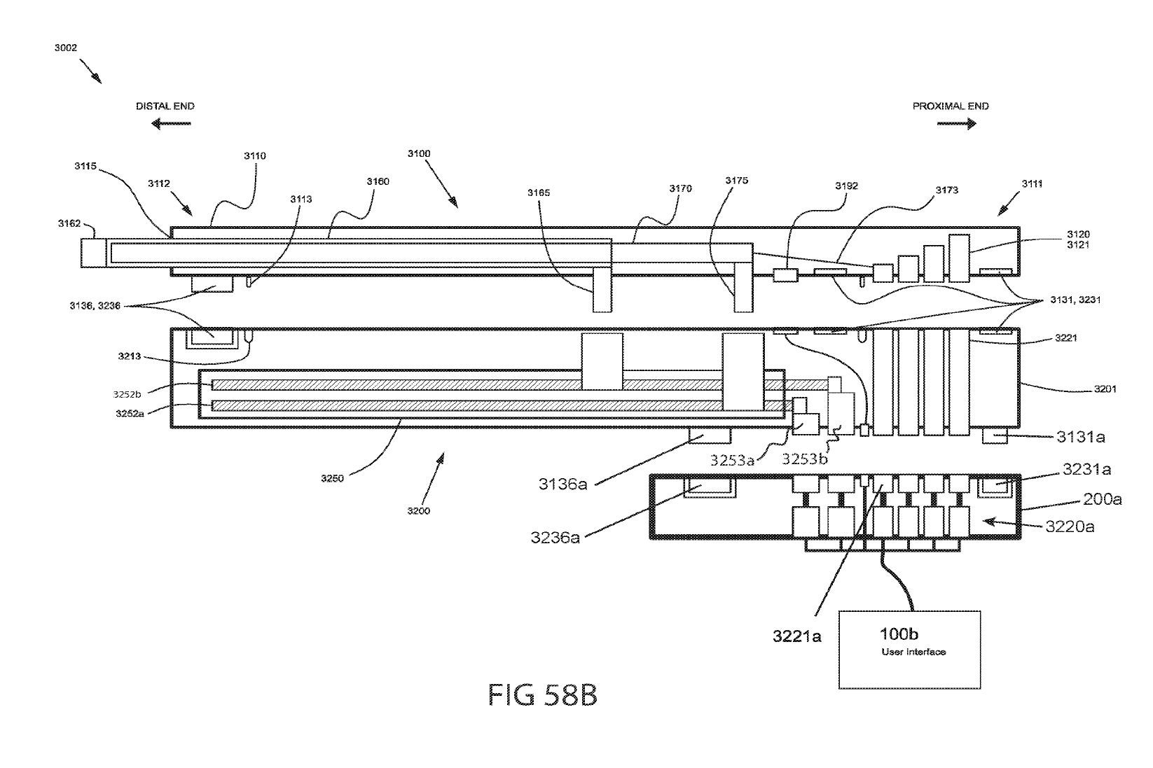

FIGS. 58A and 58B are schematic diagrams of embodiments of a robotic introducer system including a first assembly and a second assembly in accordance with the present inventive concepts.

Referring to FIG. 58A, in some embodiments, a robotic introducer system 3002 includes a first assembly 3100 and a second assembly 3200 that can be removably coupled to each other. In some embodiments, the second assembly 3200 can include a drive assembly 3220 including a plurality of drive capstans 3221 that mate with corresponding cable bobbins of the pulley assembly 3120 of the first assembly 3100. The drive assembly receives command signals from a user interface 100b for controlling operation of the system. In some embodiments, the first assembly 3100 includes a first housing 3110 and the second assembly includes a second housing 3210.

The second assembly 3200 can further include a linear drive assembly 3250. Linear drive assembly 3250 can comprise one or more linear drive mechanisms configured to advance and/or retract a component or assembly, such as a mechanism selected from the group consisting of: lead screw; ball screw; hydraulic piston; pneumatic piston; magnetic drive; inch-worm drive; belt drive; and combinations of one or more of these. In some embodiments, the linear drive assembly extends to a distal portion 3212 of the second assembly and can include first and second lead screws 3252a, 3252b first and second lead screw motors 3251a, 3251b, and inner and outer probe carriages 3265, 3275. The inner and outer probe carriages 3265, 3275 mesh with the threads of the lead screws 3252a, 3252b to induce linear motion in the carriages, as driven by the first and second lead screw motors 3251a, 3251b. In some embodiments, the lead screw motors are positioned in a proximal region 3211 of the second assembly 3200.

In some embodiments, the second assembly 3200 includes the lead screws 3252a, 3252b, inner and outer probe carriages 3275, 3265 and associated lead screw motors 3251a, 3251b so that these units, as well as any supporting hardware can be partitioned from the first assembly 3100, and potentially re-used for multiple procedures, while the componentry remaining in the first assembly 3100 can be disposed of after use in a single procedure, or, alternatively, exposed to a re-sterilization procedure. In this manner, by positioning the linear drive assembly 3250 in the second assembly 3200, the single-use componentry of the disposable first assembly 3100 is further reduced, improving the overall cost and performance of the disposable first assembly 3100 and improving environmental impact.

In some embodiments, the first assembly 3100 includes a pulley assembly 3120 including cables 3173 driven by bobbins 3121, the bobbins 3121 in turn driven by the drive capstans 3221. The first assembly further includes an articulating probe assembly 3150 comprising an inner probe 3170 of multiple inner links that are slidable within an outer probe 3160 of multiple outer links. In some embodiments, a first cable 3173 tensions the inner probe 3170 and multiple cables (not shown in FIG. 58A) tension and steer the outer probe 3160, in accordance with embodiments described herein. In alternative embodiments, one or more cables (e.g. a single cable of inner probe 3170) can be tensioned by a linear actuator such as a solenoid.

In some embodiments a proximal latch assembly 3131 and a distal latch assembly 3136 of the first assembly 3100 engage corresponding latches 3231, 3236 on the second assembly to removably couple the first assembly 3100 to the second assembly 3200. Alignment pins 3113 and corresponding receiving holes 3213 can be employed at one or more locations along the interface to ensure alignment and stability of the first and second assemblies 3100, 3200 (e.g. with respect to each other) when latched. In some embodiments, at least one of the proximal latch assembly 3131 and a distal latch assembly 3136 may comprise a magnetic coupling mechanism, for example including engagement between a magnet (e.g. an electromagnet) and a plate of magnetically attractive material, for example a steel plate. In some embodiments, a magnetic-based latching mechanism can be configured to avoid penetration of a sterile drape positioned between first assembly 3100 and second assembly 3200.

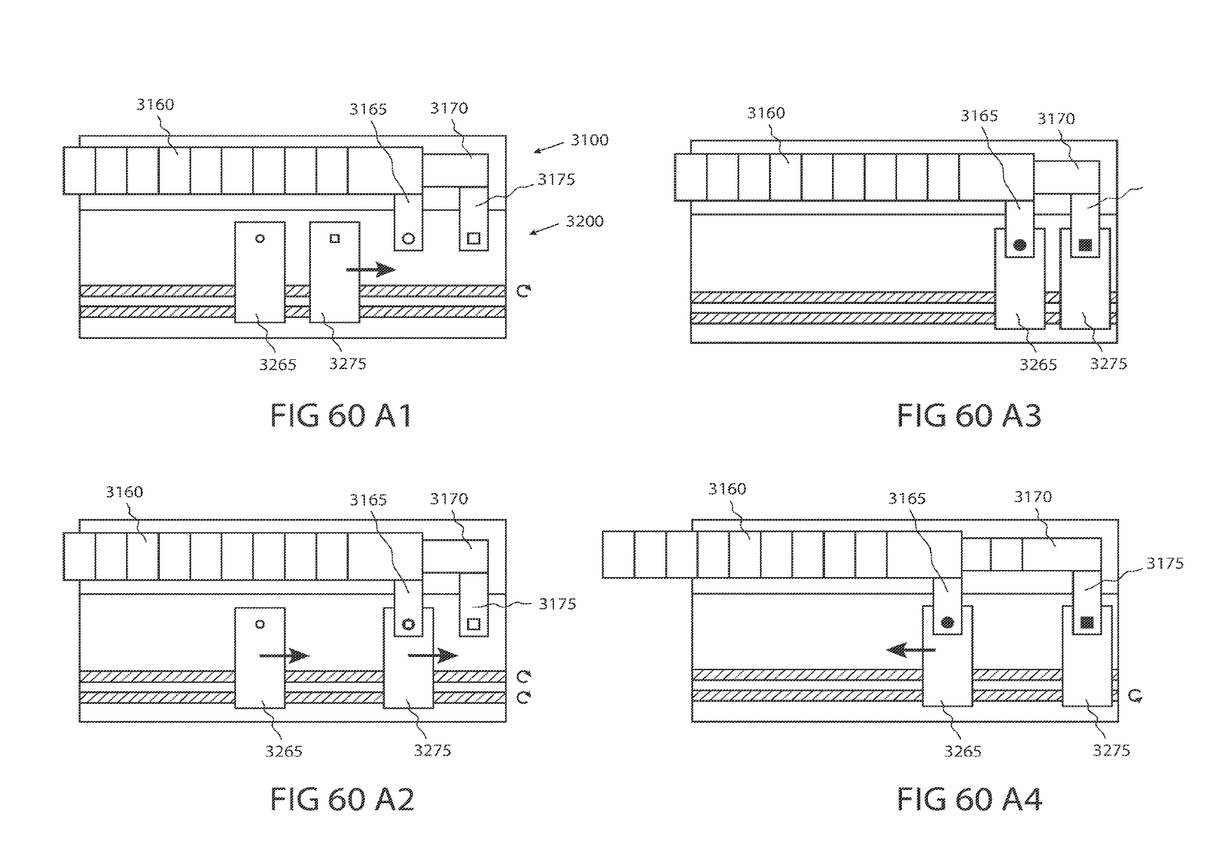

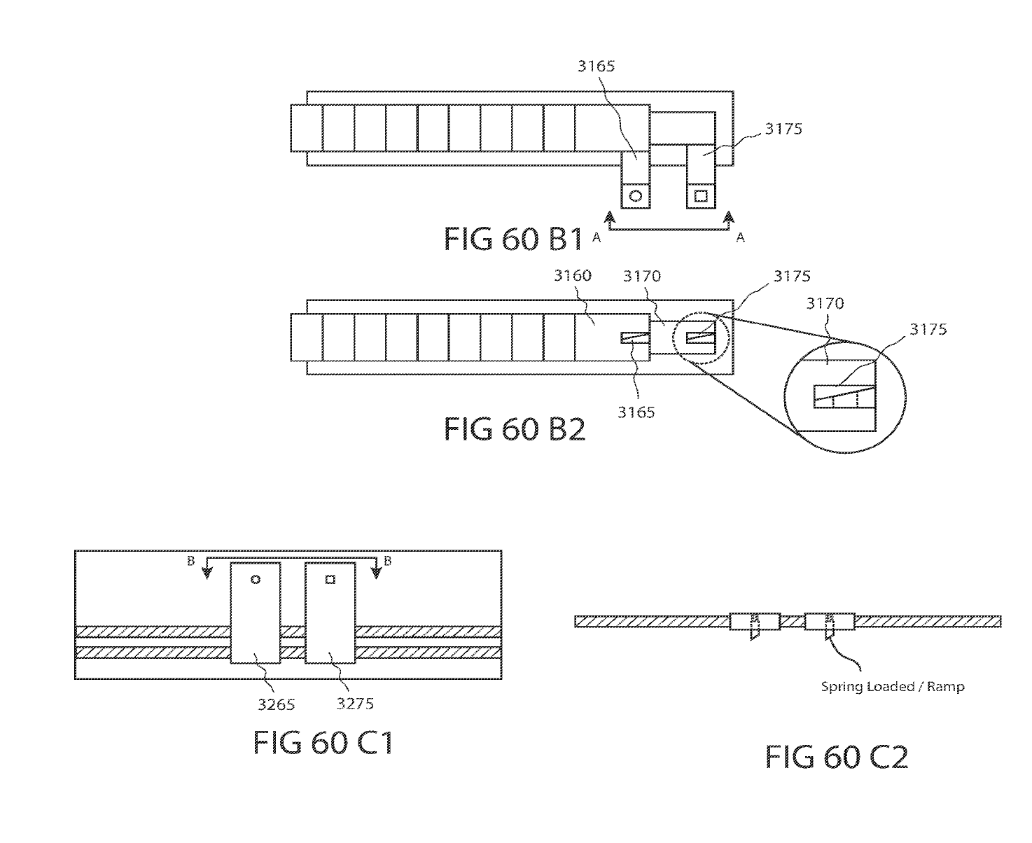

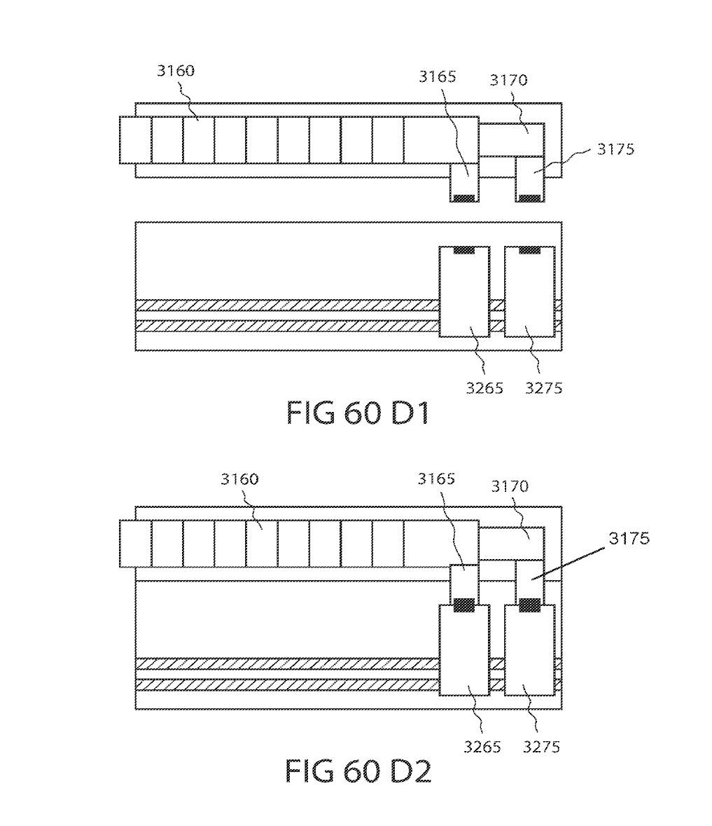

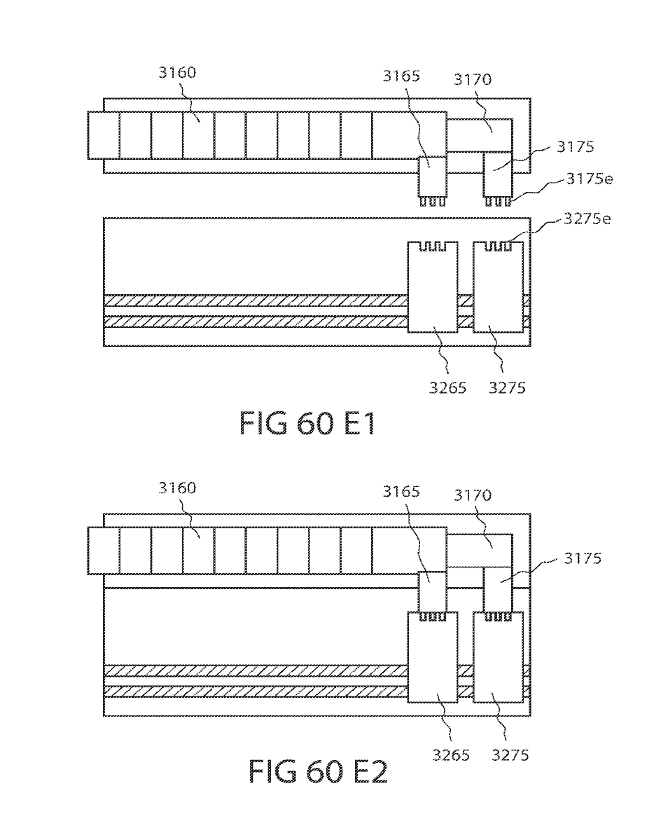

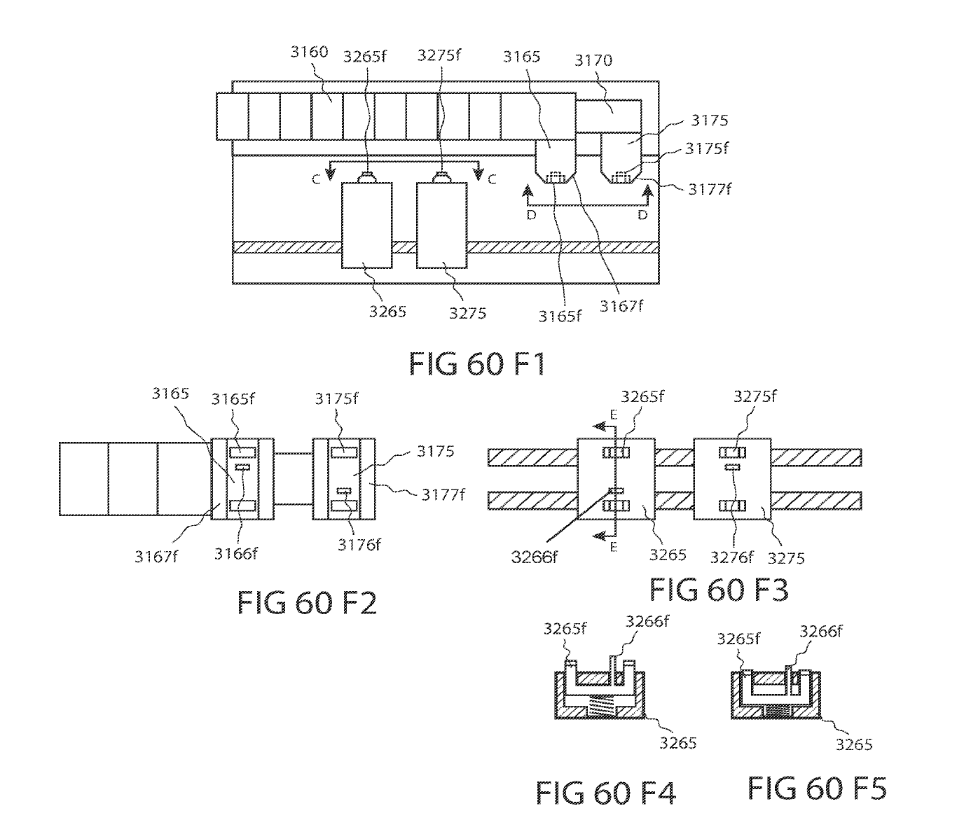

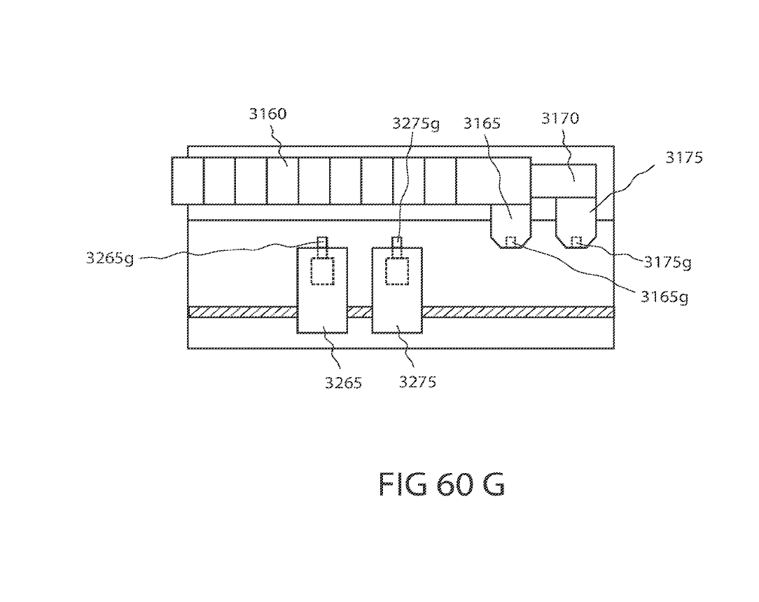

In some embodiments, the inner probe 3170 includes an inner probe connector 3175 and the outer probe 3160 includes an outer probe connector 3165. In some embodiments, the inner probe connector 3175 selectively engages the inner probe carriage 3275 of the second assembly 3200. In some embodiments, the outer probe connector 3165 selectively engages the outer probe carriage 3265 of the second assembly 3200. Selective engagement between connectors 3175 and/or 3165 and associated carriages 3275 and/or 3265, respectively, can be achieved in various ways, as are described herebelow in reference to FIGS. 60A-G. For example, mechanical keying can be used to prevent undesired engagement and achieve desired engagement. Alternatively or additionally, one or more connectors 3175 and/or 3165 can be offset (e.g. horizontally and/or vertically offset) from an undesired carriage 3275 and/or 3265, respectively, and aligned (e.g. horizontally and vertically) with a mating carriage 3275 and/or 3265, respectively.

In some embodiments, system 3002 is configured to measure one or more forces between an inner probe connector 3175 and inner probe carriage 3275 and/or to measure one or more forces between an outer probe connector 3165 and an outer probe carriage 3265, such as to determine adequate or inadequate engagement between the mating components. In these embodiments, system 3002 can be configured to realign or otherwise adjust to cause an adequate engagement to result, as described herein.

In some embodiments, the selective engagement of the inner/outer probe connector 3175, 3165 with the inner/outer probe carriage 3275, 3265 allows for advancement of the inner/outer probe 3170, 3160 in the distal direction and allows for retraction of the inner/outer probe 3170, 3160 in the proximal direction.

In some embodiments, the selective engagement of the inner/outer probe connector 3175, 3165 with the inner/outer probe carriage 3275, 3265 allows for advancement of the inner/outer probe 3170, 3160 in the distal direction and while not applying a force for providing retraction of the inner/outer probe 3170, 3160 in the proximal direction. In such an embodiment, retraction of the inner/outer probe 3170, 3160 can be accomplished by tensioning of the probe assembly cables 3173. In addition to controlling to the tension of cables 3173, drive assembly 3220 of second assembly 3200 can provide position information, such as position information related to outer probe connector 3165 and/or inner probe connector 3175 (e.g. to automatically or semi-automatically perform an alignment or other positioning procedure during use).

In some embodiments, first assembly 3100 comprises an electronics module, such as electronics module 3192, which can comprise an EEPROM circuit on a printed circuit board (PCB) or the like (e.g. an electronic circuit comprising one or more memory components). Electronics module 3192 may include one or more special-purpose hardware processors that can provide second assembly 3200 with identification information such as the model number, manufacture date, and/or configuration information (e.g. probe 3150 length information) of first assembly 3100, such as to confirm acceptability of a mating second assembly 3200. Electronics module 3192 can further provide set-up and/or status information, such as activation and/or other use information, probe position information, functionality information (e.g. can alert second assembly 3200 of an error in first assembly 3100), and the like. In some embodiments, second assembly 3200 can write information to electronics assembly 3192, such that after first assembly 3100 is removed from second assembly 3200, and reattached, to the same or different second assemblies 3200, the electronics module 3192 can deliver information written during the first attachment. For example, second assembly 3200 can write probe position information to electronics module 3192, such that if inner or outer probes 3170 or 3160, respectively, are not in a home position when removed from second assembly 3200, electronics module stores that error state information. Electronics module 3192 can be configured to prevent re-use in a second patient and/or it can comprise an encryption or other tamper-reducing component. Electronics module 3192 can be configured to record (e.g. and store) first assembly 3100 position information, which can be used in a re-attachment of first assembly 3100 to second assembly 3200 (e.g. after an initial use). Electronics module 3192 can be configured to control power to one or more electronic portions of first assembly 3100, such as an indicator light configured to indicate proper attachment of first assembly 3100 to second assembly 3200. Alternatively or additionally, second assembly 3200 (e.g. adaptor 3201 described herebelow in reference to FIG. 58B) can comprise an electronics module, such as an electronics module comprising an EEPROM circuit configured to store information (e.g. use information, patient information, configuration information, and the like).

Electronics module 3192 can comprise electronics, for example, EEPROM or the like, forming one or more sensors, such as a proximity sensor (e.g. magnetic or mechanical), temperature sensor, force sensor (e.g. strain gauge), at the like. A sensor of electronics module 3192 can comprise one or more sensors configured to indicate the position of connector 3165 (e.g. the position of outer probe 3160) and/or the position of connector 3175 (e.g. the position of inner probe 3170). Electronics module 3192 can comprise one or more indicator lights or other status-indicating elements, as described herein.

User interface 100b can comprise one or more user input and/or user output components, such as a component selected from the group consisting of: joystick; keyboard; mouse; switch; touchscreen; touch pad; trackball; display such as a touchscreen or standard display; audio element such as speaker or buzzer; light such as an LED; and combinations of one or more of these.

First assembly 3100, second assembly 3200, and/or another component of system 3002 can comprise one or more stabilizing elements, as described hereabove, such as to prevent or at least reduce undesired twisting or other undesired movement of elements of the system 3002 (e.g. twisting or other movement caused during tensioning of one or more inner or outer cables 3173). Stabilizing elements can comprise a plate (e.g. a metal plate), a rib (e.g. a rib projecting from housing 3110 or 3210), drive assembly 3250 (e.g. when drive assembly 3250 comprises one or more rails configured to prevent twisting as described hereabove), and the like.

System 3002 can comprise one or more introducers, also referred to as introduction devices, configured to provide a pathway to support at least a portion of probe assembly 3150, such as introducer 3306 described herebelow. In some embodiments, system 3002 comprises multiple introducers with different features, such as different lengths and/or different trajectories. Introducer 3306 comprises a pathway which aligns with opening 3115 of housing 3110. Introducer 3306 can be constructed and arranged to be operator attachable to housing 3110 and/or housing 3210. Introducer 3306 can be constructed and arranged to be used in multiple clinical procedures, such as to be used in more procedures than first assembly 3100. Introducer 3306 can comprise one or more clips for attaching to a cable, such as a cable of a camera system as described herebelow. Introducer 3306 can include an opening, also as described herebelow, such as an opening configured to receive a projection of outer probe 3160 and/or a cable such as a camera cable. Introducer 3306 can be attached to one or more tool supports, as described herebelow.

Referring to FIG. 58B, in some embodiments, the second assembly 3200 of the system 3002 comprises an adaptor 3201 that can be removably coupled to a base assembly 200. The first assembly can be removably coupled to the adaptor 3201, for example in manner similar to the manner in which the first assembly 3100 is described as being removably coupled to the second assembly 3200 as described in connection with the embodiment of FIG. 58A. In some embodiments, elements of the base assembly 200 may be similar to or the same as those described in connection with the embodiment of FIG. 11 herein.

In the embodiment of FIG. 58B, the drive assembly 3220 is positioned in the base assembly 200a and receives communication signals from the user interface 100b, as described herein. Motor capstans 3221a are provided for inducing rotational motion in the cable bobbins 3121 of the first assembly and the lead screw linkages 3253a, b of the first and second lead screws of the adaptor 3201. The adaptor 3201 includes a plurality of capstans 3221 that operate as “pass-through” capstans that rotate freely and transfer rotational motion, induced by the base capstans 3221a to the bobbins 3121 of the pulley assembly 3120 of the first assembly 3100. In some embodiments, the adaptor 3201 includes hardware for latching or securing the adaptor 3201 to the base assembly 200a, for example, according to the mechanisms described herein. In some embodiments, such hardware can include proximal and/or distal latching mechanisms. In some embodiments a proximal latch assembly 3131a and a distal latch assembly 3136a of the adaptor assembly 3201 engage corresponding latches 3231a, 3236a on the base assembly 200 to removably couple the adaptor assembly 3201 to the base assembly 200a.

In a configuration where the second assembly comprises an adaptor, the first assembly 3100 of the type indicated in FIG. 58A can be retrofit to the base assembly 200 of the type described in connection with FIG. 11 to permit use of the first assembly 3100 with base assembly 200. This configuration allows for realization of an improvement in system cost-per-use, while allowing for compatibility of the first assembly with pre-existing base-assemblies.

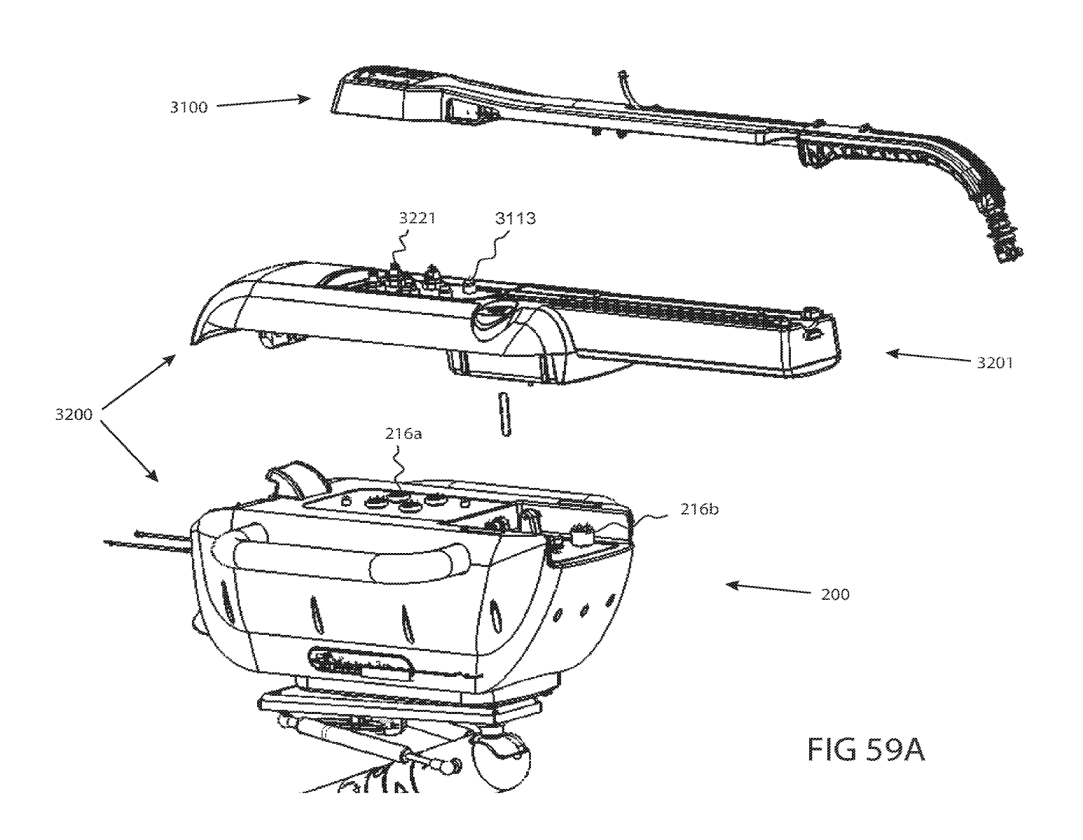

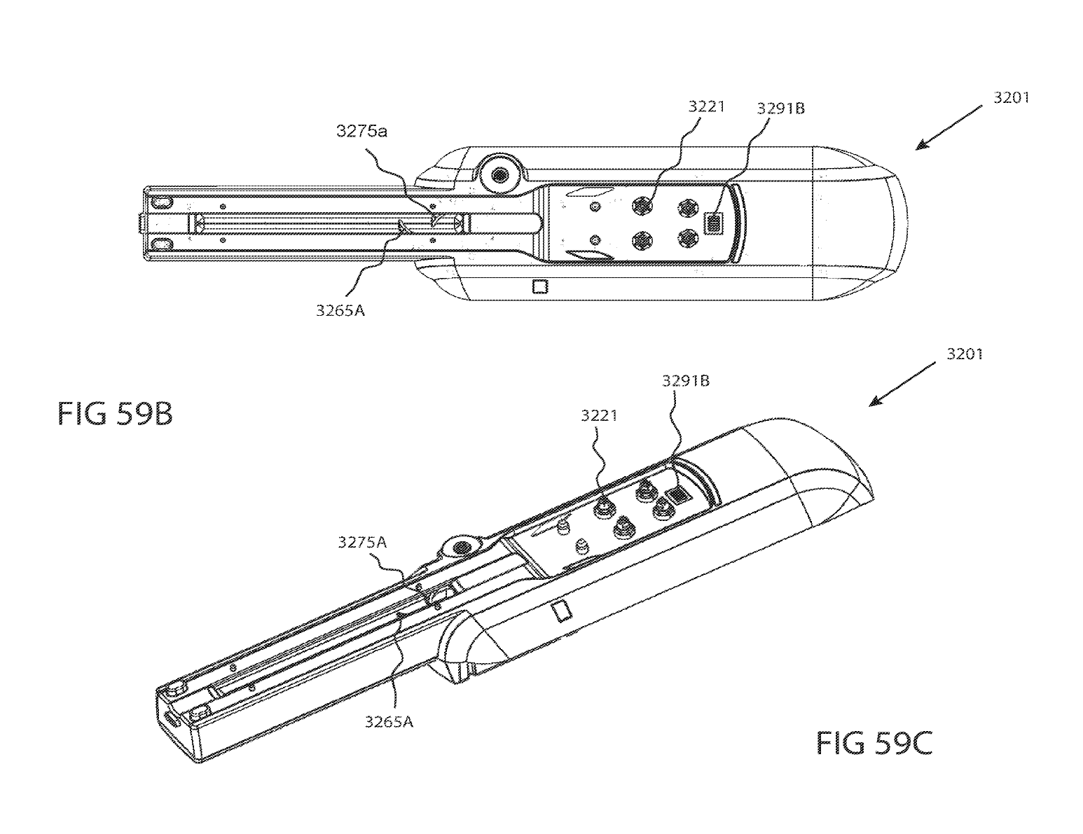

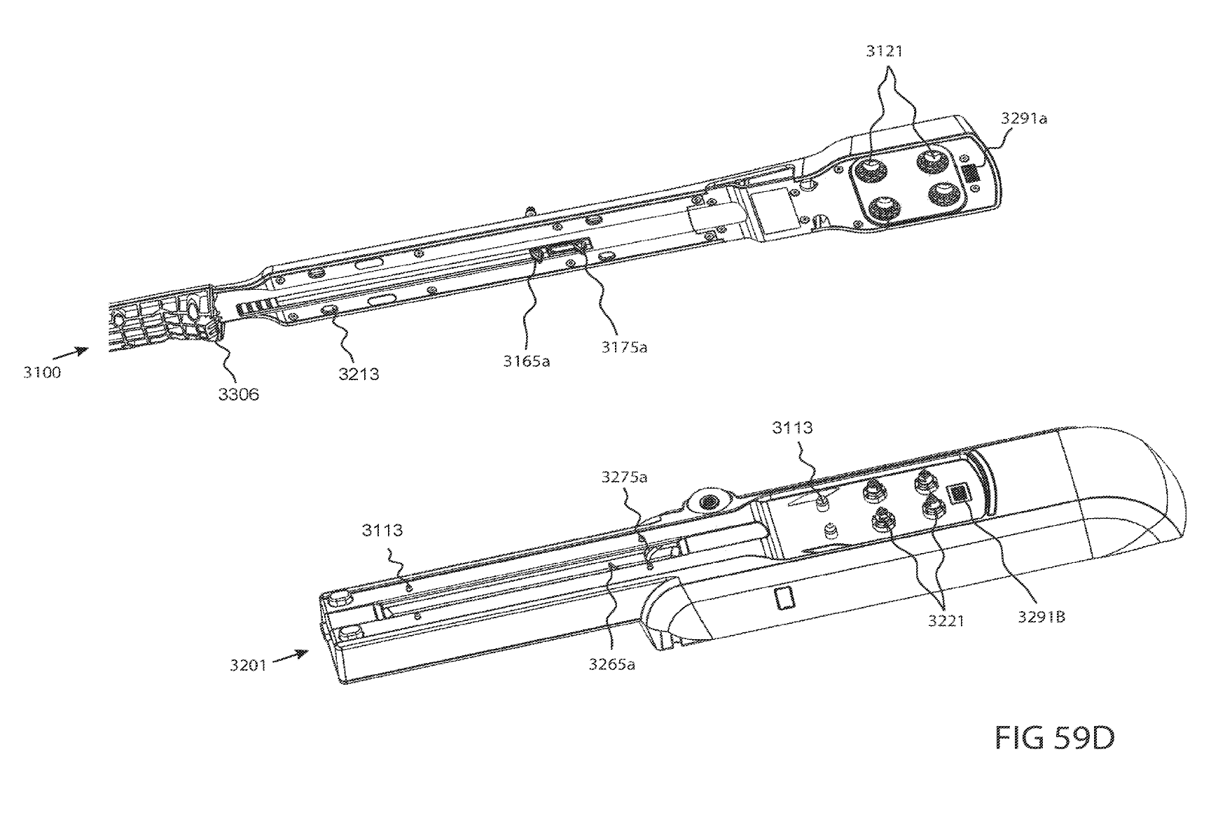

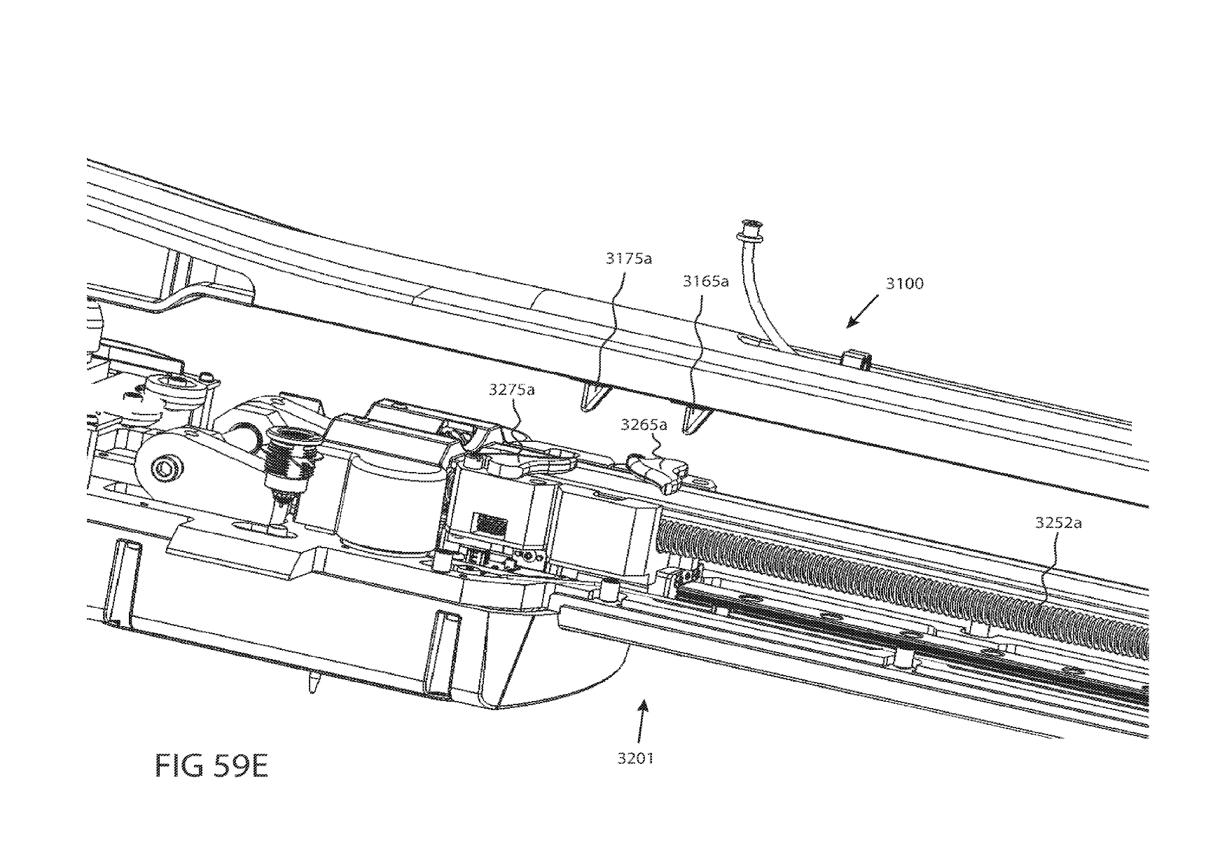

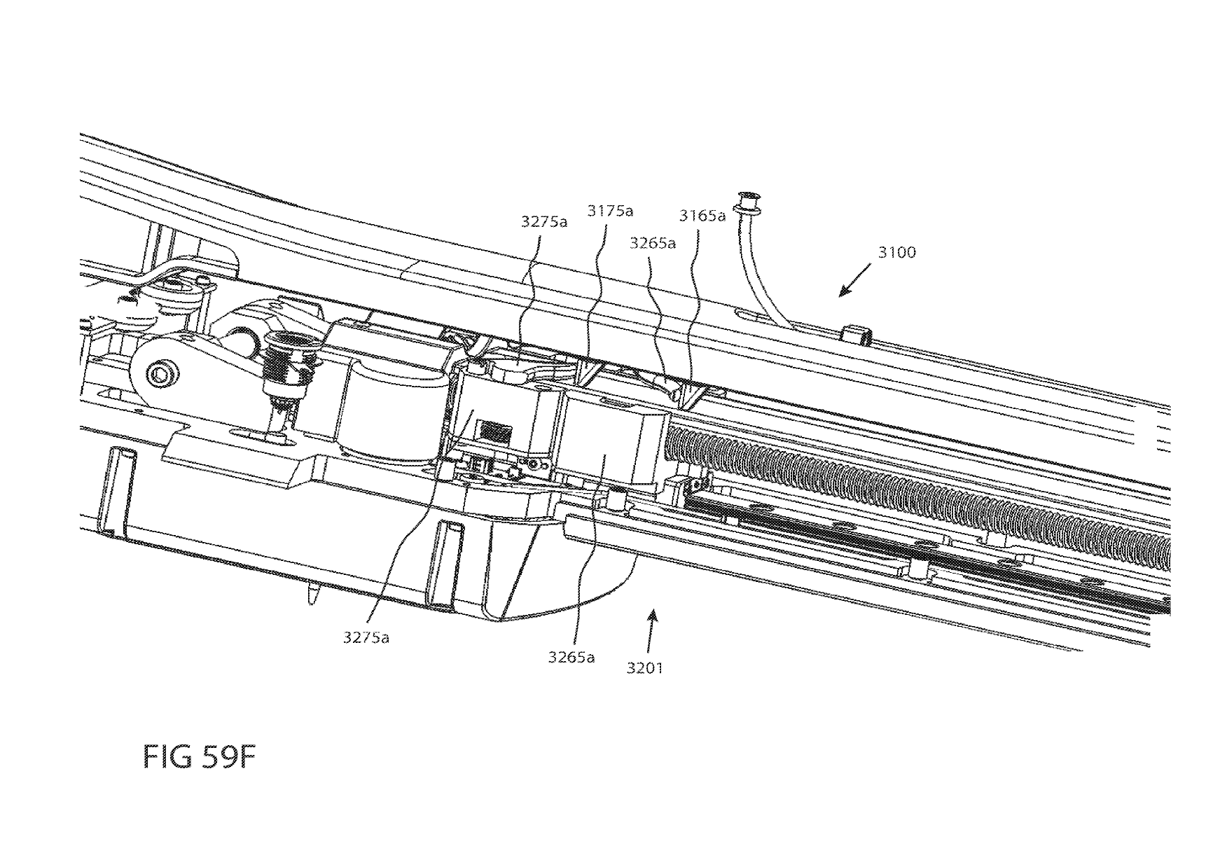

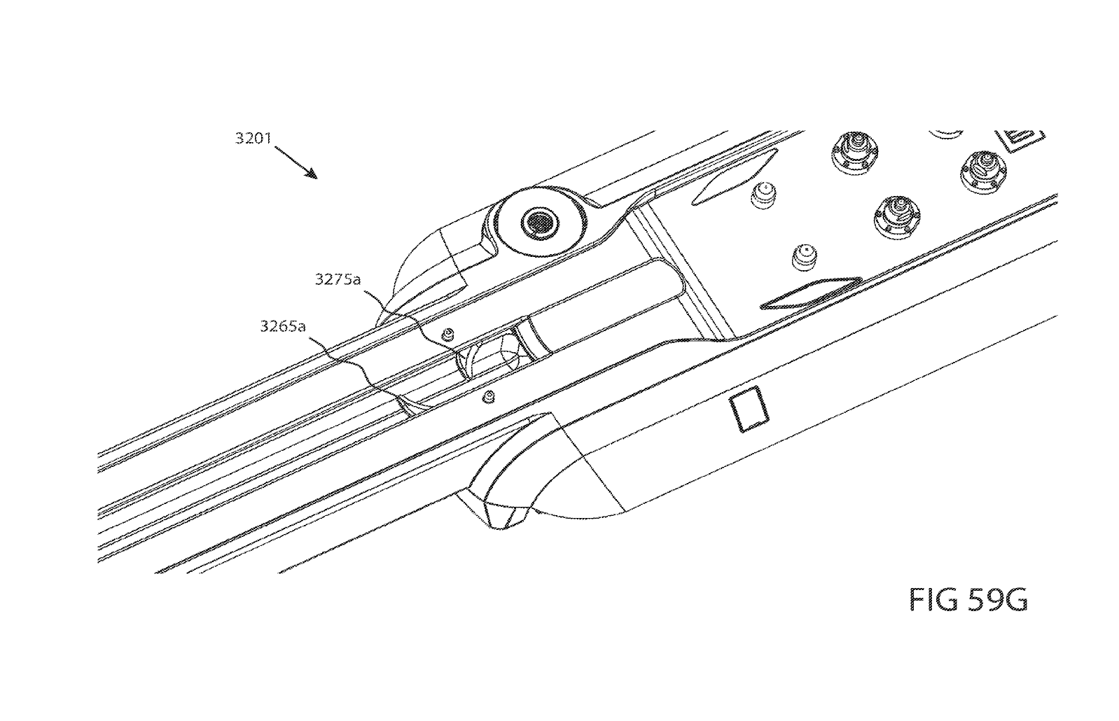

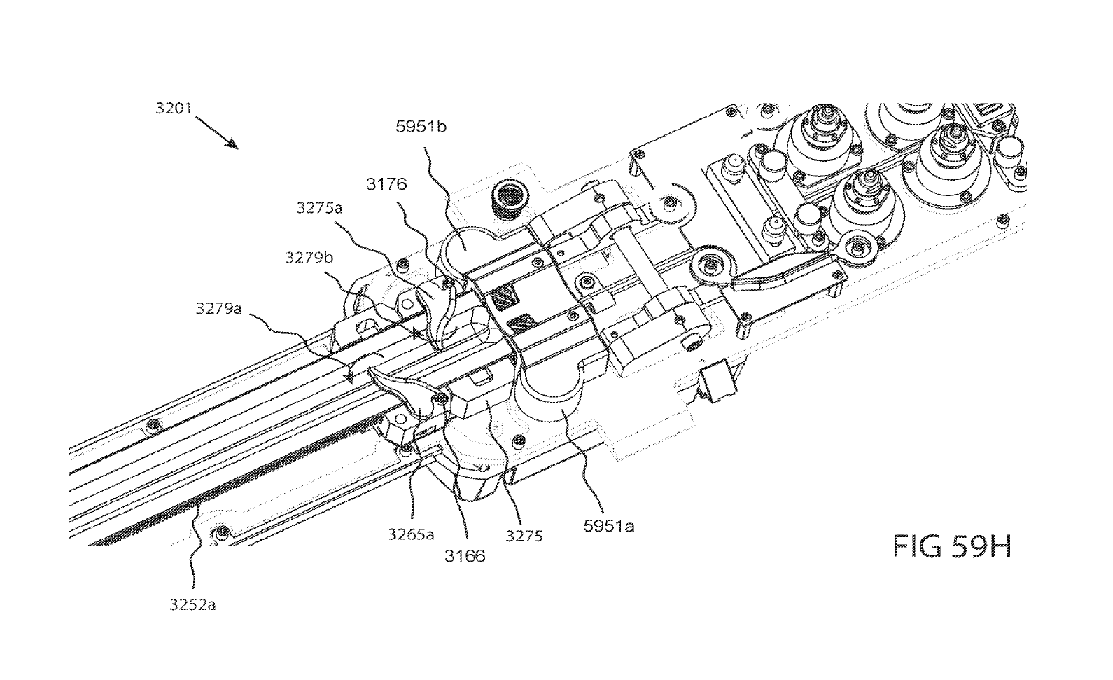

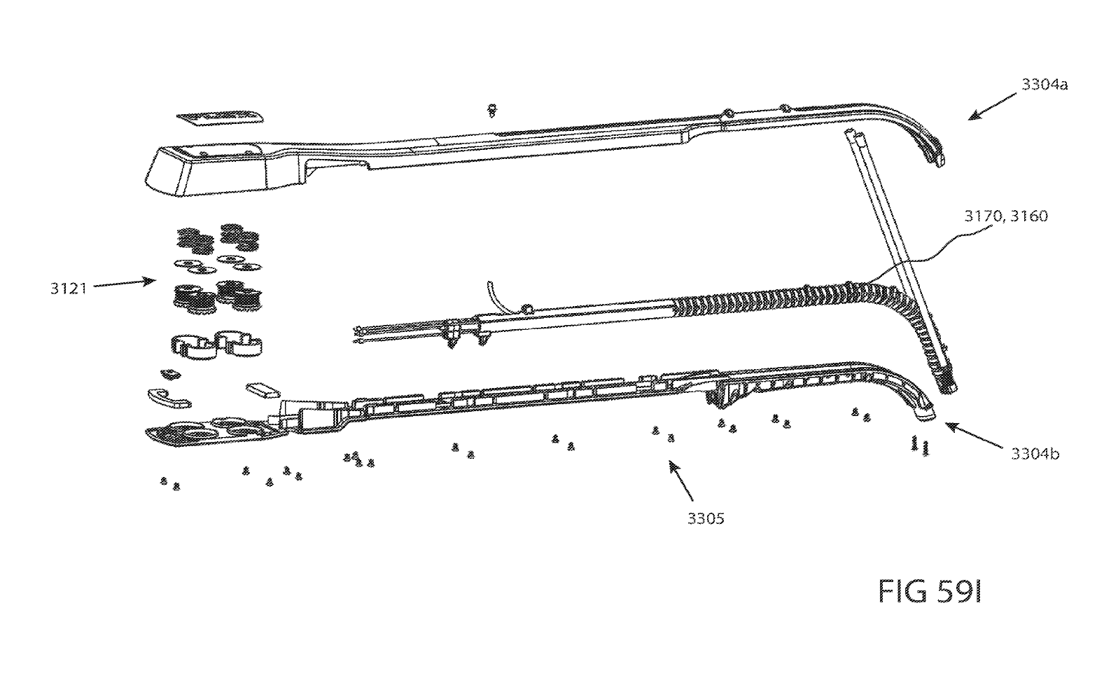

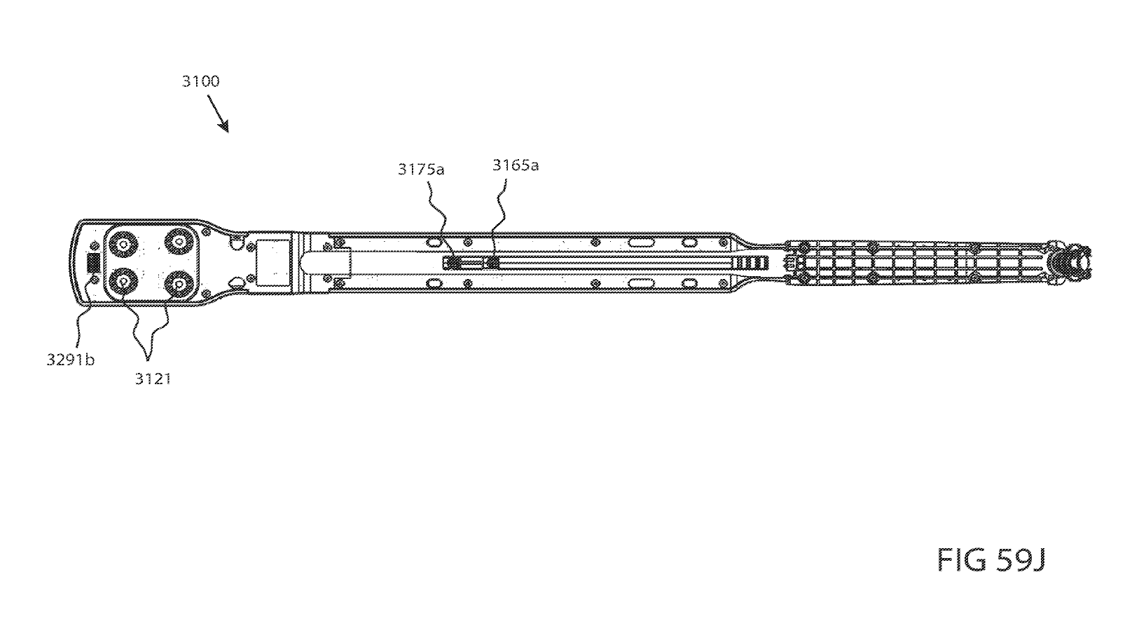

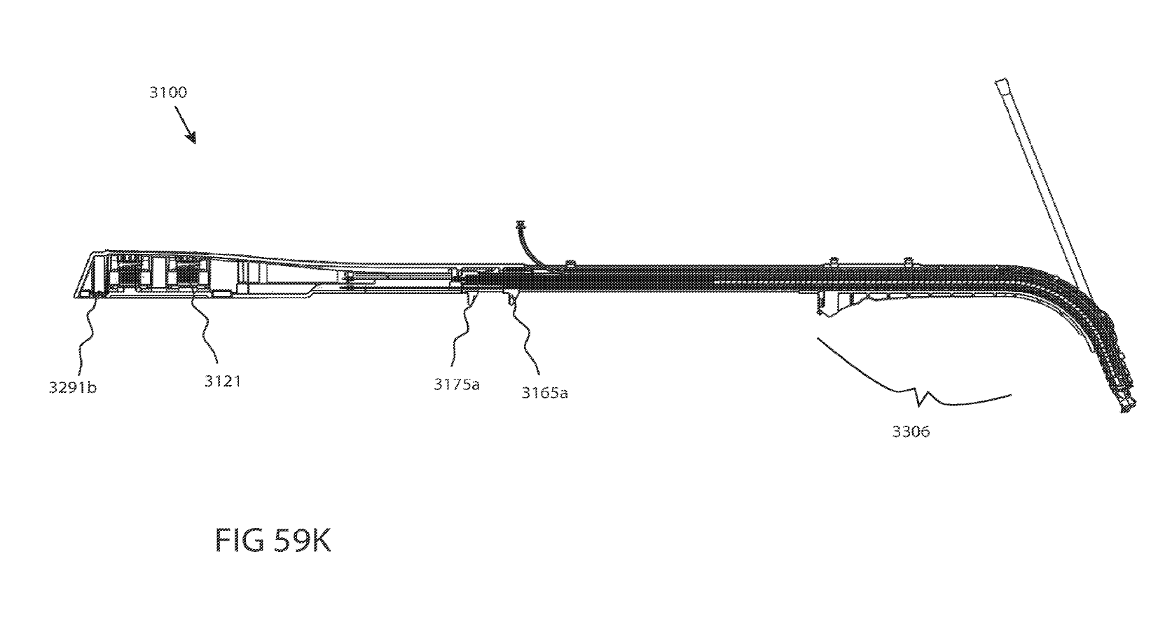

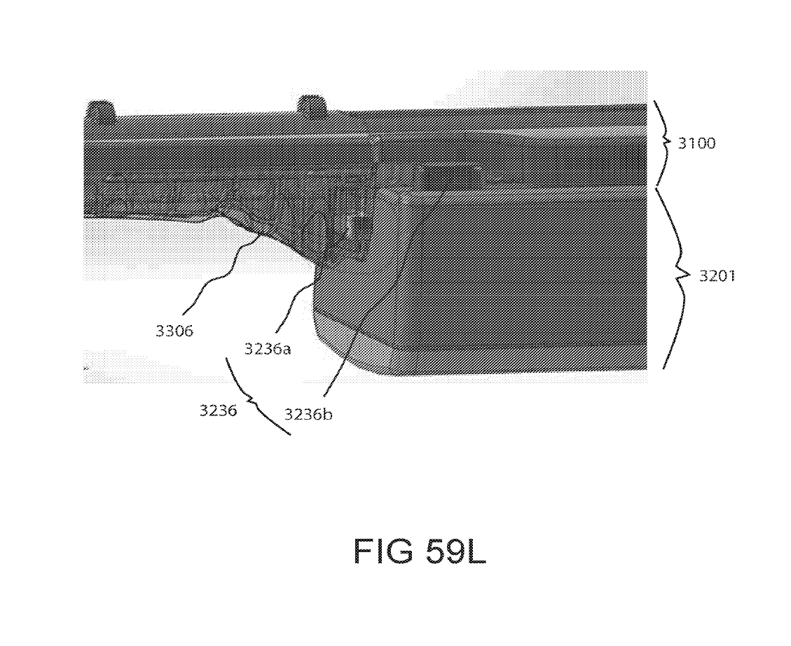

In some embodiments, the first assembly 3100 can be considered a single-use disposable assembly, whereby the componentry and housing of which are used for a single procedure on a single patient and discarded following a single use. In a case where the second assembly 3200 comprises an adaptor 3201 as shown in FIGS. 58B and 59A, the adaptor can, in some embodiments, be considered a single-use disposable assembly, whereby the componentry and housing of which are used for a single procedure on a single patient and discarded following a single use. In this embodiment, the base assembly 200a to which the adaptor 3201 is coupled can be considered a re-usable assembly, whereby the componentry and housing of which can be re-used for more than one procedure with more than one adaptor 3201 and/or more than one first assembly.

In some embodiments, the first assembly 3100 can be considered a single-use disposable assembly, whereby the componentry and housing of which are used for a single procedure on a single patient and discarded following a single use. In a case where the second assembly 3200 comprises an adaptor 3201 as shown in FIGS. 58B and 59A, the adaptor can, in some embodiments, be considered a re-usable assembly, whereby the componentry and housing of which can be re-used for a multiple procedures for example on multiple patients. In this embodiment, the base assembly 200a to which the adaptor 3201 is coupled can likewise be considered a re-usable assembly, whereby the componentry and housing of which can be re-used for more than one procedure with more than one adaptor 3201 and/or more than one first assembly. In some embodiments the first assembly can be used for one procedure, the adaptor 3201 can be used for a first number of procedures that is greater than the first assembly, and the base assembly 200a can be used for a second number of procedures that is greater than or equal to the first number of procedures.

In some embodiments, the first assembly 3100 can be considered a multiple-use assembly, whereby the componentry and housing of which are used for a multiple procedures on multiple patients and then discarded. In a case where the second assembly 3200 comprises an adaptor 3201 as shown in FIG. 58B, the adaptor can, in some embodiments, be considered a re-usable assembly, whereby the componentry and housing of which can be re-used for a multiple procedures for example on multiple patients. In this embodiment, the base assembly 200 to which the adaptor is coupled can likewise be considered a re-usable assembly, whereby the componentry and housing of which can be re-used for more than one procedure with more than one adaptor 3201 and/or more than one first assembly. In some embodiments the first assembly can be used for a first number of multiple procedures, the adaptor 3201 can be used for a second number of procedures that is greater than the first number of the first assembly, and the base assembly 200a can be used for a third number of procedures that is greater than or equal to the second number of procedures.

In some embodiments, the first assembly 3100 can be considered a single-use disposable assembly, whereby the componentry and housing of which are used for a single procedure on a single patient and discarded following a single use. The second assembly 3200 can, in some embodiments, be considered a re-usable assembly, whereby the componentry and housing of which can be re-used for a multiple procedures for example on multiple patients and in connection with more than one first assembly. In some embodiments the first assembly can be used for one procedure, and the second assembly 3200 can be used for a first number of procedures that is greater than or equal to the first number of procedures of the first assembly.

FIG. 59A is an exploded perspective view of an embodiment of the robotic introducer system of FIGS. 58A, B, in accordance with the present inventive concepts. In a description of embodiments of an introducer system herein, the system is described as including the adaptor 3201, in concert with the schematic description of the system as described in connection with FIG. 58B. It will be understood, however that where the system is described as having an adaptor, the principles of the inventive concepts will apply equally well to the system generally described in connection with the schematic diagram of FIG. 58A, wherein the componentry of the linear drive assembly 3250 is instead integrated into the base assembly 200a.

The robotic introducer system 3002 in this embodiment includes a first assembly 3100 and a second assembly 3200. The second assembly in turn includes a base assembly 200 and an adaptor 3201. The base assembly 200 can comprise a base assembly as described herein, for example as described herein in connection with FIGS. 11 and 12. Capstans 216a of the base assembly 200 are engaged with pass-through capstans 3221 of the adaptor 3201 which are, in turn engaged with bobbins 3121 of the first assembly. Capstans 216b of the base assembly 200 are engaged with lead screw gears 5951a and 591b (see FIG. 59H) of the lead screws of the adaptor 3201. In the present embodiment, the adaptor 3201 can mate with the base assembly 200 in a manner similar to the manner in which the top assembly 300 of FIG. 11 mates with the base assembly 200 of FIG. 11.

FIGS. 59B and 59C are a top view and side perspective view, respectively, of the second assembly of the embodiment of the robotic introducer system of FIG. 59A, in accordance with the present inventive concepts. In this view, it can be seen that the adaptor 3201 includes the pass-through capstans 3221 for transferring motion to the bobbins 3121 of the first assembly 3100 and further includes electrical connector port 3291b for transferring electrical signals between the base assembly 200, the adaptor 3201 and the first assembly 3100.

FIGS. 59D is a bottom perspective view of the first assembly of the embodiment of the robotic introducer system of FIG. 59A, in accordance with the present inventive concepts. Referring to FIG. 59D, it can be seen that in the present embodiment, the first assembly 3100 includes bobbins 3121 that mate with the pass-through capstans 3221 of the adaptor assembly. In addition, the first assembly 3100 can include a mating electrical connector port 3291a that mates with port 3291b of the adaptor 3201.

In addition, the perspective view of FIG. 59D, it can be seen that the first assembly 3100 further includes an inner probe connector 3175a and an outer probe connector 3165A. In the present example embodiment, the inner and outer probe connectors 3175a, 3165A are in the form of projections having an interface surface configured to interface with corresponding interface surfaces 3275A, 3265A of the inner and outer probe carriages 3275, 3265. The inner and outer probe connectors 3175a, 3165A are in turn coupled to the inner and outer probes 3170, 3160 respectively, for example proximal links of the inner and outer probes 3170, 3160 respectively.

The interface surfaces 3275A, 3265A are coupled to the inner and outer probe carriages 3275, 3265, which, when driven by the inner and outer lead screws 3252b, 3252a in the distal direction, apply a distal-oriented force on the inner and outer probe connectors 3175a, 3165A. Selective application of such a force by the drive mechanism in turn selectively moves the inner and outer probes 3170, 3160 in the distal direction, accommodating travel and steering operations of the inner and outer probes 3170, 3160.

FIGS. 59E, 59F, 59G, and 59H are side perspective views of the interaction of the first assembly and second assembly of the embodiment of the robotic introducer system of FIG. 59A, in accordance with the present inventive concepts. In FIG. 59H it can be seen that one or both interface surfaces 3275A, 3265A of the carts, in the present example embodiment take the form of fingers that pivot on an axle. In some embodiments, the inner cart finger 3175a can pivot freely in the clockwise direction shown by arrow 3279b; however the inner cart finger 3175a is restrained from pivoting in the counter-clockwise direction by stop 3176. Similarly, outer cart finger 3165A can pivot freely in the counter-clockwise direction shown by arrow 3279a; however the outer cart finger 3165A is restrained from pivoting in the clockwise direction by stop 3166. In some embodiments, stops 3166 and/or 3176 can be temporarily retracted or otherwise repositioned to allow free pivoting of outer cart finger 3165A and/or inner cart finger 3176, respectively in the direction previously prevented. In some embodiments, stops 3166 and/or 3176 are configured to be retracted or otherwise repositioned to allow a release of tension in one or more cables 3173. In some embodiments, stop 3166, 3176 and/or one or more separate components are configured to measure a force (e.g. a torque) exerted by and/or upon outer cart finger 3165A and/or inner cart finger 3175a. This arrangement of the inner and outer cart fingers 3165A, 3175a allows for a “homing” procedure to be performed on a newly attached first assembly prior to performing the medical procedure. In this manner, when a first assembly 3100 is attached to a second assembly 3200, the second assembly 3200 may possibly not have its carts 3275, 3265 properly positioned in a “home” position (e.g. a fully retracted or other known position of the inner and outer probes 3170, 3160 and/or carts 3275, 3265). Accordingly, the inner and outer carts 3275, 3265 and inner and outer cart fingers 3175a, 3165A may be positioned distal the inner and outer probe cart connectors 3175a, 3165A at the time of attachment of the first assembly 3100. In a homing procedure, the inner and outer carts 3275, 3265 can be returned to their home positions, without interference from the inner and outer probe cart connectors 3175a, 3165A as a result of the inner and outer cart fingers 3175a, 3165A freely pivoting relative to the inner and outer probe cart connectors 3175a, 3165A when they come in contact with each other during the homing procedure.

FIGS. 59I, 59J, and 59K are exploded perspective, bottom, side and cutaway side views, respectively, of the first assembly 3100 of the embodiment of the robotic introducer system of FIG. 59A, in accordance with the present inventive concepts. The first assembly includes an upper housing 3304a and lower housing 3304b coupled to each other using attachment means, for example, screws 3305 or other well-known connectors. Bobbins 3121 coordinate related drive and steering cables 3173 are coupled to the inner and outer probes 3170, 3160 as discussed herein. Electrical connector port 3291b communicates with corresponding connector port 3291a on the adaptor 3201 for effecting the transfer of electrical signals between the base assembly 200, the adaptor 3201 and the first assembly 3100. In some embodiments, distal ends of the upper housing and lower housing join to provide an esophageal introducer, for example, described in embodiments herein.

While the embodiments illustrated in connection with FIGS. 59A-59L depict that the projection surfaces 3175a, 3165A are located on the first assembly 3100 and the interface surfaces 3275A, 3265A are on the carts of the second assembly 3200, in other embodiments of the present inventive concepts, the positions one or both of the projection surfaces 3175a, 3165A relative to the interface surfaces 3275A, 3265A can be reversed.

In some embodiments, the first assembly 3100 has a mass that is less than a mass of the second assembly 3200.

In some embodiments, a distal link 3162 of the outer probe 3160 can comprise a camera assembly to provide visual feedback to an operator of the system.

In some embodiments, upon the system determining that a proper registration of the first assembly 3100 relative to the second assembly has occurred, an auditory or visual feedback unit can be activated. In some embodiments, the auditory feedback can comprise a “beep” or other sound. In some embodiments, the visual feedback can comprise an activation of an illumination element, such as an LED indicator.

In some embodiments, while the dual linear drive assembly is depicted herein as being a lead-screw-based assembly, other types of linear drive assemblies apply equally well to the principles of the present inventive concepts. These include systems based on magnetic drive, hydraulic/pneumatic piston drive, belt drive, or any other suitable drive system configured to independently advance or retract the interface surfaces 3275A, 3265A.

In some embodiments, the drive assembly 3220 comprises a plurality of electrically driven motors, for example, closed-loop servomotors.

FIGS. 59L is a perspective view of a latching mechanism for securing the first assembly of the embodiment of the robotic introducer system of FIG. 59A to the second assembly, in accordance with the present inventive concepts. A proximal end of the introducer 3306 includes a capture feature that mates with a corresponding latch mechanism 3236 located at a distal end of the adaptor assembly to provide for mechanical registration of the first assembly 3100 with the second assembly 3200. In some embodiments, the latch 3236A of the latch mechanism 3236 can be engaged and released by manual activation of a pushbutton 3236B. Pushbutton 3236B can comprise one or more pushbuttons, such as a first pushbutton positioned on one side of an attached first assembly 3100 and a second pushbutton positioned on the opposite side of an attached first assembly 3100, such that either or both (in combination) can be configured to be depressed to detach first assembly 3100 from second assembly 3200.

FIGS. 60A1-60A4 are schematic views depicting an alternative embodiment for the interface of the inner and outer carts 3275, 3265 with the inner and outer probe connectors 3175, 3165. In this embodiment, referring to FIG. 60A1, the carts 3275, 3265 are initially located distal the inner and outer probe cart connectors 3175, 3165 of the probes 3170, 3160. The inner cart 3275 in this embodiment is keyed to register exclusively with the inner probe connector 3175 of the inner probe. This is represented by the “square” key shown in the drawing. Similarly, the outer cart 3265 in this embodiment is keyed to register exclusively with the outer probe connector 3165 of the outer probe. This is represented by the “circle” key shown in the drawing.

During a homing procedure, as shown in FIG. 60A2, the inner cart 3275 is moved in the proximal direction and comes in contact with the outer probe cart connector 3165. Because they are keyed differently, the inner cart 3275 (square) fails to register with the outer probe cart connector 3165. Accordingly, as shown in FIG. 60A3, the inner cart 3275 continues to travel in the proximal direction and eventually registers properly with the inner probe cart connector 3175. Similarly the outer cart 3265 continues to travel in the proximal direction and eventually registers properly with the outer probe cart connector 3165. As shown in FIG. 60A4, when the inner and outer carts 3275, 3265 have made proper registration with the inner and outer probe cart connectors, 3175, 3165 a procedure inducing travel of the probes 3170, 3160 in the distal direction can commence.

FIGS. 60B1-60B2 and 60C1-60C1 are schematic views detailing the features of the embodiment of FIGS. 60A. It can be seen that the keying mechanism can travel along a ramp element of a mating junction. When the key is proper, the pin and junction mate. When the key is improper (e.g. a projection is larger than a receiving hole), the pin continues to travel upward until the end of the ramp element is reached, at which point the pin continues past the ramp element. FIG. 60C2 shows that the keyed pins can be spring-loaded, such as to facilitate traveling past and/or engaging a proper junction.

FIGS. 60D1-60D2 are schematic views depicting an alternative embodiment for the interface of the inner and outer carts 3275, 3265 with the inner and outer probe connectors 3175, 3165. In this embodiment, registration occurs through magnetic interaction on surfaces of the carts and probes. In some embodiments, if the cart magnets comprise electromagnetics, selective coupling can occur as a result of selective activation of the electromagnets. For example, during a homing procedure, assuming the outer cart probe connector 3165 is positioned in the vicinity of the inner cart 3275, and, assuming its position is known, for example, using a positional encoder, the electromagnet can be deactivated to permit the inner cart 3275 to pass by the outer cart probe connector 3165 without engagement. When proper registration is affirmed, the electromagnets can be activated to couple the inner cart 3275 to the inner cart probe connector 3175 and to couple the outer cart 3265 to the outer cart probe connector 3165. When desired, the electromagnets can be deactivated to permit de-coupling thereof. In some embodiments, magnetic pairs of components are oriented (e.g. the magnetic poles are oriented) such that a repelling force is generated when improper components are in proximity to each other, and an engaging, attracting force is generated when the proper components are aligned.

FIGS. 60E1-60E2 are schematic views depicting an alternative embodiment for the interface of the inner and outer carts 3275, 3265 with the inner and outer probe connectors 3175, 3165. In the present embodiment, mating engagement portions 3175e, 3275e when engaged, can provide for registration of the inner and outer carts 3275, 3265 and inner and outer cart probe connectors 3175, 3165, for example in the manner described here. In some embodiments, the mating engagement portions 3175e, 3275e can provide for mechanical registration and coupling as well as a location for electrical coupling or optical coupling between the inner and outer carts 3275, 3265 and inner and outer cart probe connectors 3175, 3165. In some embodiments, the mating engagement portions 3175e, 3275e can provide for a liquid or gas coupling to allow for the exchange of fluids between the inner and outer carts 3275, 3265 and inner and outer cart probe connectors 3175, 3165.

FIGS. 60F1-60F5 are schematic views depicting an alternative embodiment for the interface of the inner and outer carts 3275, 3265 with the inner and outer probe connectors 3175, 3165. FIG. 60F2 is a schematic top view taken along section line C-C of FIG. 60F1. FIG. 60F3 is a schematic top view taken along section line D-D of FIG. 60F1. FIG. 60F4 and FIG. 60F5 are schematic cross-sectional diagrams taken along section line E-E of FIG. 60F3. In the present embodiment, spring-loaded pins 3275f, 3265f positioned on the inner and outer carts 3275, 3265 selectively mate with corresponding recesses 3175f, 3165f on the inner and outer cart probe connectors 3175, 3165. In the present embodiment a leading ramp 3177f, 3167f presses the spring-loaded pins 3275f, 3265f as the components begin to communicate. When a key pin 3276f, 3266f mates with a corresponding key hole 3176f, 3166f, the spring-loaded pins 3275f, 3265f mate with the key holes 3176f, 3166f, and the carts 3275, 3265 become coupled to the probe connectors 3175, 3165. When a key pin 3276f, 3266f fails to mate with a corresponding key hole 3176f, 3166f, the spring-loaded pins 3275f, 3265f likewise fail to mate with the key holes 3176f, 3166f, and the probe connectors 3175, 3165 do not become coupled to the carts 3275, 3265.

FIG. 60G is a schematic view depicting an alternative embodiment for the interface of the inner and outer carts 3275, 3265 with the inner and outer probe connectors 3175, 3165. The present embodiment is similar to that of FIGS. 60F1-60F5 in that pins 3275g, 3265g positioned on the inner and outer carts 3275, 3265 selectively mate with corresponding recesses 3175g, 3165g on the inner and outer cart probe connectors 3175, 3165. However, in the present embodiment, the pins 3275g, 3265g are selectively actuated by a solenoid, a linear actuator or other electro-mechanical apparatus. In some embodiments, positional locators can be used to determine whether proper positional registration of the inner and outer cart probe connectors 3175, 3165 with the inner and outer carts 3275, 3265 has occurred. If so, the solenoid can be engaged to couple the units.

In some embodiments, embodiments of the interface of the inner and outer carts 3275, 3265 with the inner and outer probe connectors 3175, 3165 or embodiments with similar interface arrangements that permit the mutual capture of the carts and probes may be used to drive one or both of the inner and outer probe in the distal direction and to retract the one or both of the inner and outer probe in the proximal direction. In some embodiments, the interface of the inner and outer carts 3275, 3265 with the inner and outer probe connectors 3175, 3165 or similar interface arrangements that permit the mutual capture of the carts and probes may be used to drive one or both of the inner and outer probe in the distal direction while retraction of the one or both of the inner and outer probe in the proximal direction can be performed by retracting the locking and steering cables passing through the inner and outer probes.

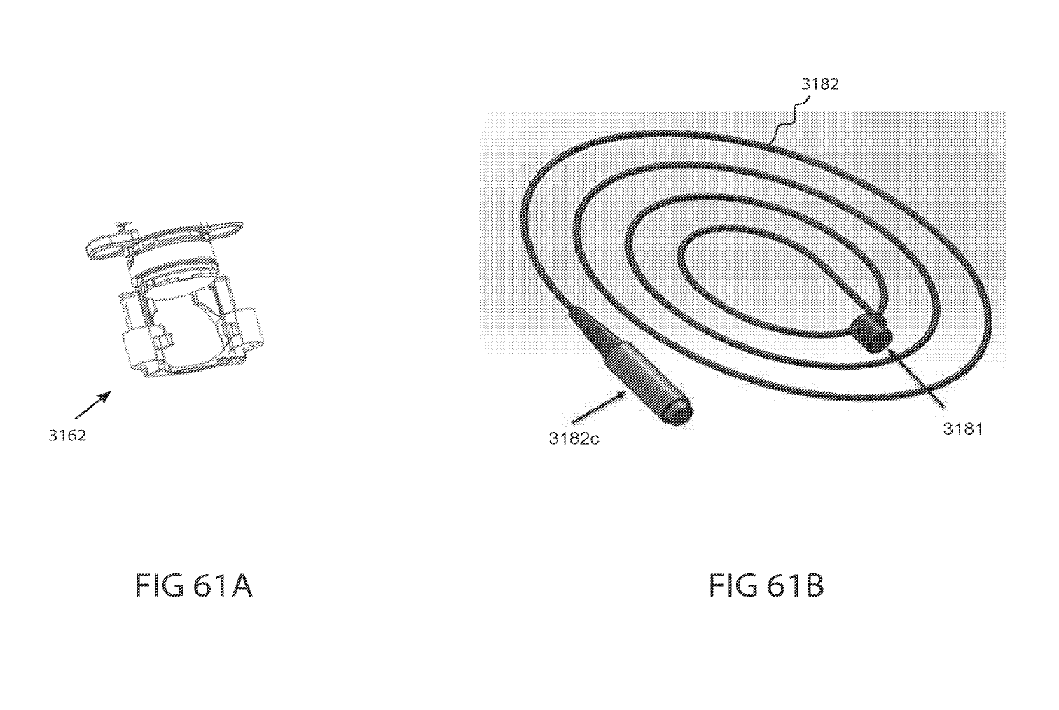

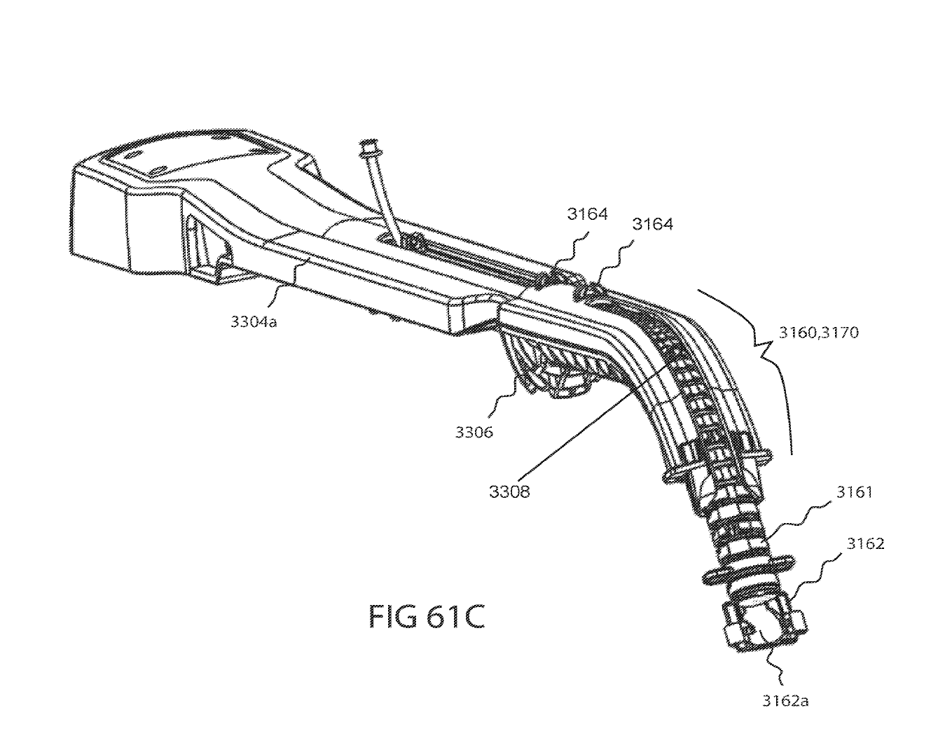

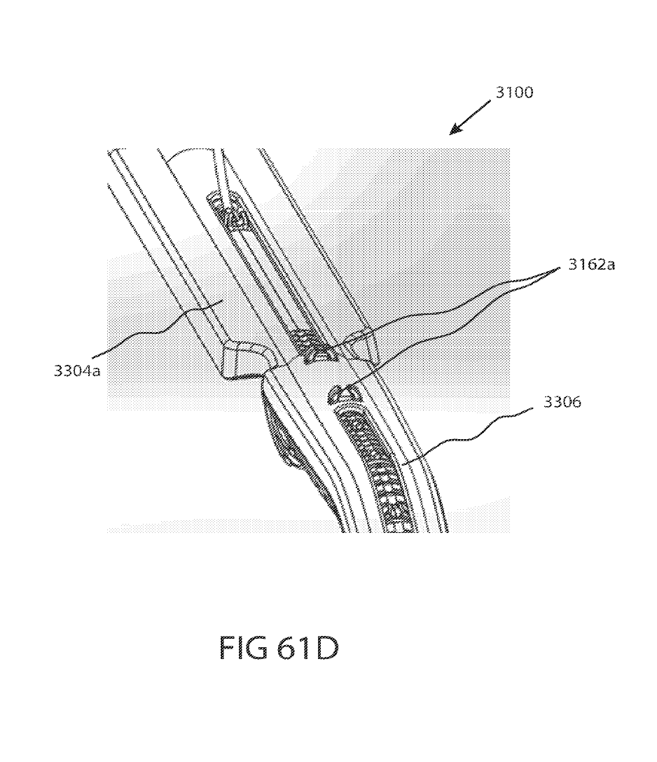

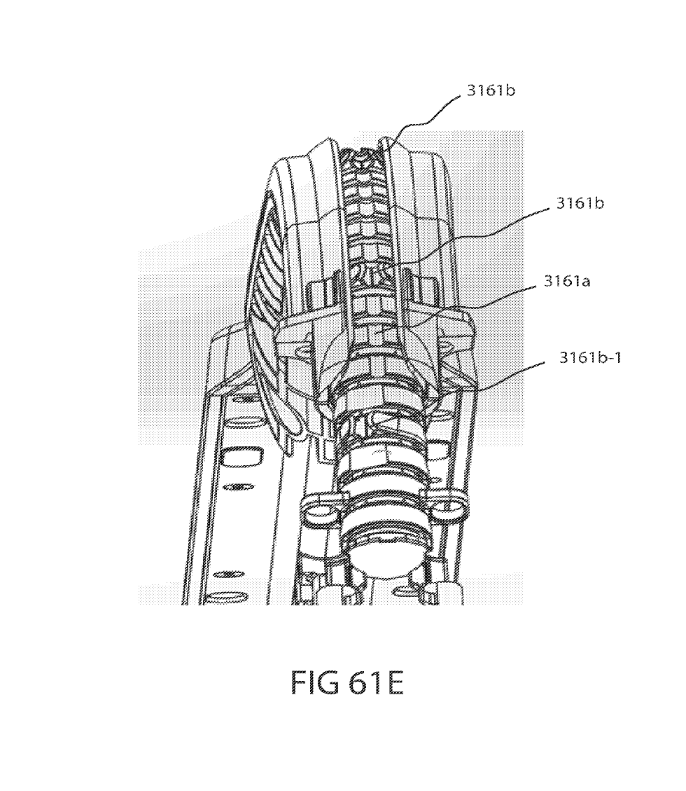

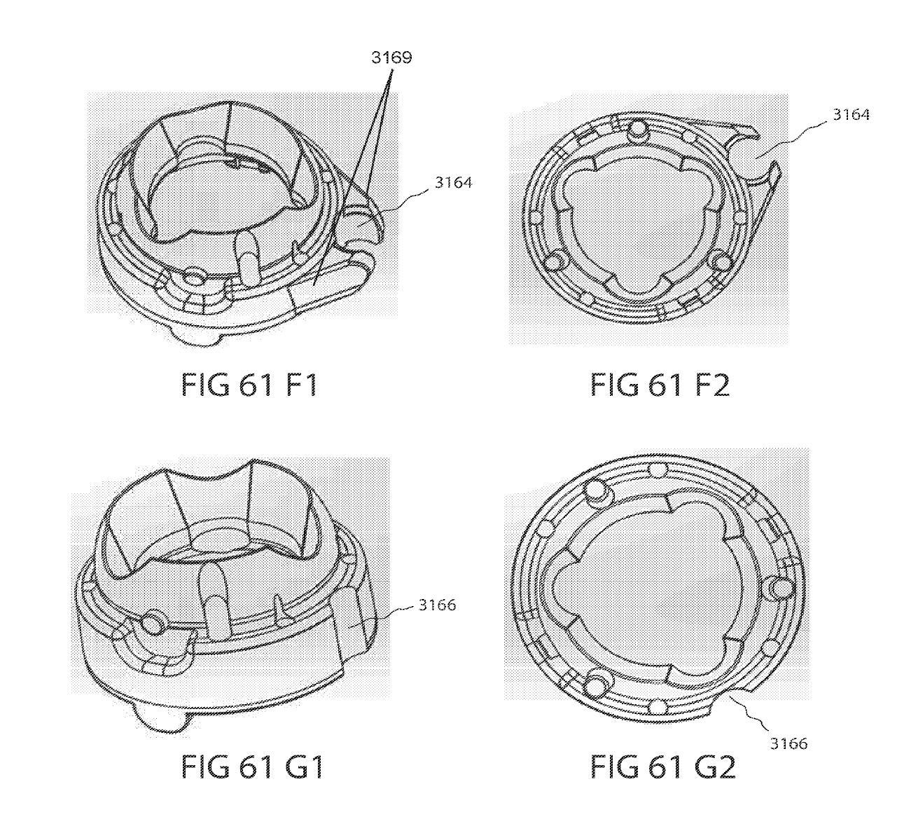

FIG. 61A is a perspective view of an embodiment of a distal outer link 3162 of the outer probe 3160 in accordance with the present inventive concepts. FIG. 61B is a perspective view of a camera system in accordance with the present inventive concepts. FIG. 61C is a perspective view of a first assembly 3100 including the distal outer link of FIG. 61A and suitable for receiving a camera system in accordance with the present inventive concepts. FIGS. 61D and 61E are close-up perspective views of the first assembly in accordance with the present inventive concepts. FIG. 61F-1 and FIG. 61F-2 are perspective and top views of an embodiment of an outer link including a camera cable clip in accordance with the present inventive concepts. FIG. 61G-1 and FIG. 61G-2 are perspective and top views of an embodiment of an outer link including a camera cable recess in accordance with the present inventive concepts

Referring to FIGS. 61A, 61B and 61C the first assembly 3100 includes a downwardly curved introducer assembly 3306, as described herein, at a distal end, and the probe system, including the inner and outer probes 3170, 3160 extend through the introducer. A distal link 3162 of the outer probe 3160 includes a camera seat 3162a at which a camera 3181 (see FIG. 61B) can be positioned and secured. In some embodiments, the camera 3181 can be snap-fit into the camera seat 3162a. In other embodiments, other suitable approaches for securing the camera in the camera seat can be employed.

Referring to FIGS. 61D and 61E, one or more of the outer links of outer probe 3160 include a camera cable clip feature 3164 (see FIGS. 61F-1, 61F-2). The clip feature 3164 is adapted for receiving and retaining a body of the camera cable 3182 (see FIG. 61B). Several of the outer links along the length of the outer probe 3160 can be included with this feature to periodically allow for retention of the camera cable along the top of the first assembly 3100. Outer links 3161A neighboring the cable clip links 3161B-1 can be provided with a recess 3166 (see FIGS. 61G-1, 61G-2) for receiving a portion of the body of the camera cable in a low-profile arrangement. A top surface of the introducer 3306 or housing 3304a of the first assembly can be provided with similar clip features for further location/organization of the camera cable. Referring to FIG. 61E a cable clip link 3161B-1 closest to the distal link 3162 may have a clip feature that is angled slightly toward a side of the probe since, in some embodiments, the camera cable enters the camera at a side of the camera. This configuration allows for the cable to gradually transition from a top of the probe to a side of the probe. In some embodiments, the clip features can have a sloped outer surface 3169 to mitigate snagging.

In the present embodiment, it can be seen that the introducer 3306 includes an open channel 3308 on its upper portion along the body of the probe 3160, 3170. In this manner, the camera system 3181 can be coupled to a first assembly 3100 having a pre-installed probe. Similarly, the camera system 3181 can be removed from the first assembly 3100 without removal or disassembly of the probe. Such a configuration accommodates re-use of the camera system, even in a case where the first assembly may be destined for single use. Alternatively or additionally, system 3002 can comprise a kit of multiple camera systems 3181 each comprising a different camera parameter such as depth of field, field of view, resolution, dimensional capability (e.g. 2D or 3D), and the like.

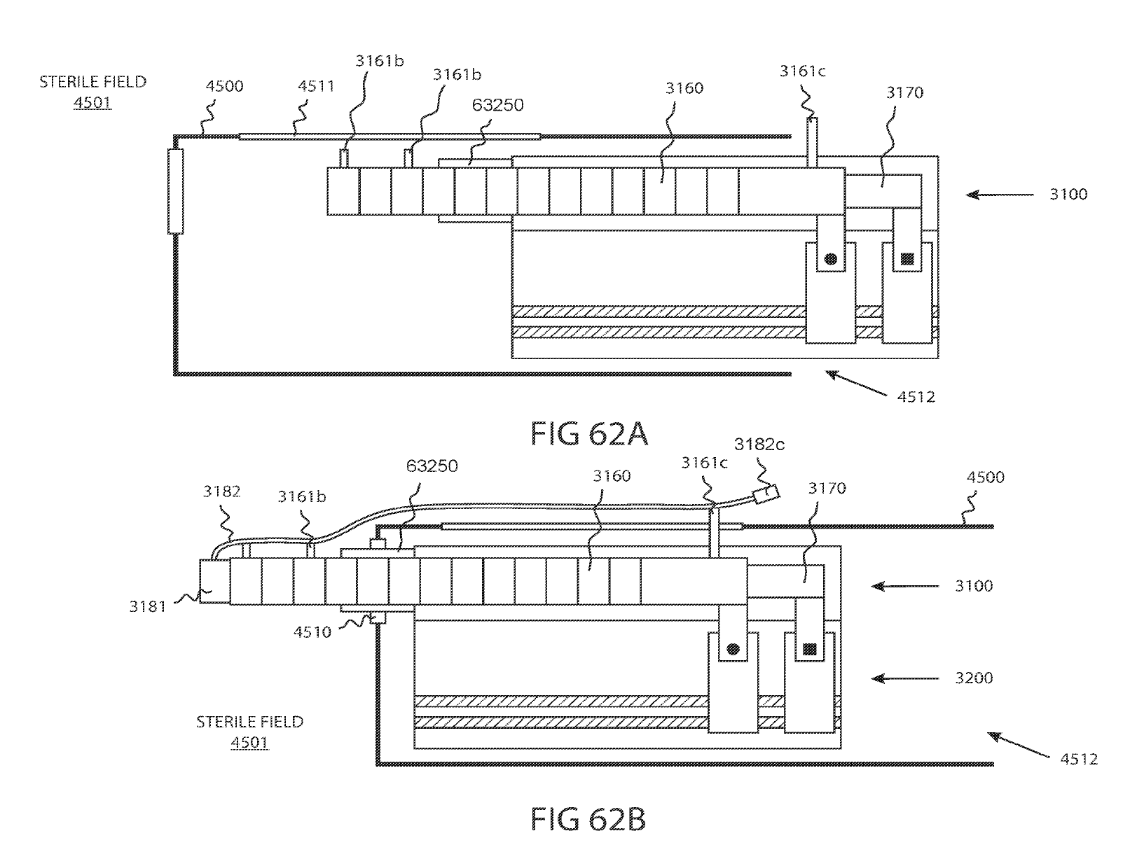

Referring to FIGS. 62A and 62B, side views of a sterile drape assembly according to an embodiment of the inventive concepts are illustrated partially and fully draped, respectively, over a robotic system. Sterile drape 4500 can comprise one or more openings, such as distal opening 4510, dorsal opening 4511, and proximal opening 4512 shown. In some embodiments, drape 4500 can comprise HDPE or other flexible, serializable materials. As described herein, drape 4500 is provided during a sterile, clinical procedure, to maintain sterility in the sterile environment 4501, and to shield non-sterile portions of the system. In a first step, drape 4500 is applied about first and second assemblies 3100 and 3200 respectively, such as when first and second assemblies 3100 and 3200 have been operably attached to each other as described herein. At least a portion of outer probe 3160 and inner probe 3170 pass thru sterile drape 4500 (e.g. at distal opening 4510), and drape 4500 can be secured to a portion of assembly 3100, such as a portion of introducer 63250, such as with one or more straps or elastics about distal opening 4510.

Dorsal opening 4511 can be aligned with a top portion of top assembly 3100 such as to provide access to one or more portions of assembly 3100. In a second step, an operator, such as a nurse or clinician, can operably attach a reusable camera, camera 3181, to outer probe 3160 as shown and described hereabove. Camera cable 3182 can be attached to one or more cable connectors 3161b, and attach to one or more proximal camera connectors 3182c, such as connectors 3182c protruding through dorsal opening 4511 of drape 4500. In these embodiments, camera assembly 3181 resides entirely within sterile field 4501.

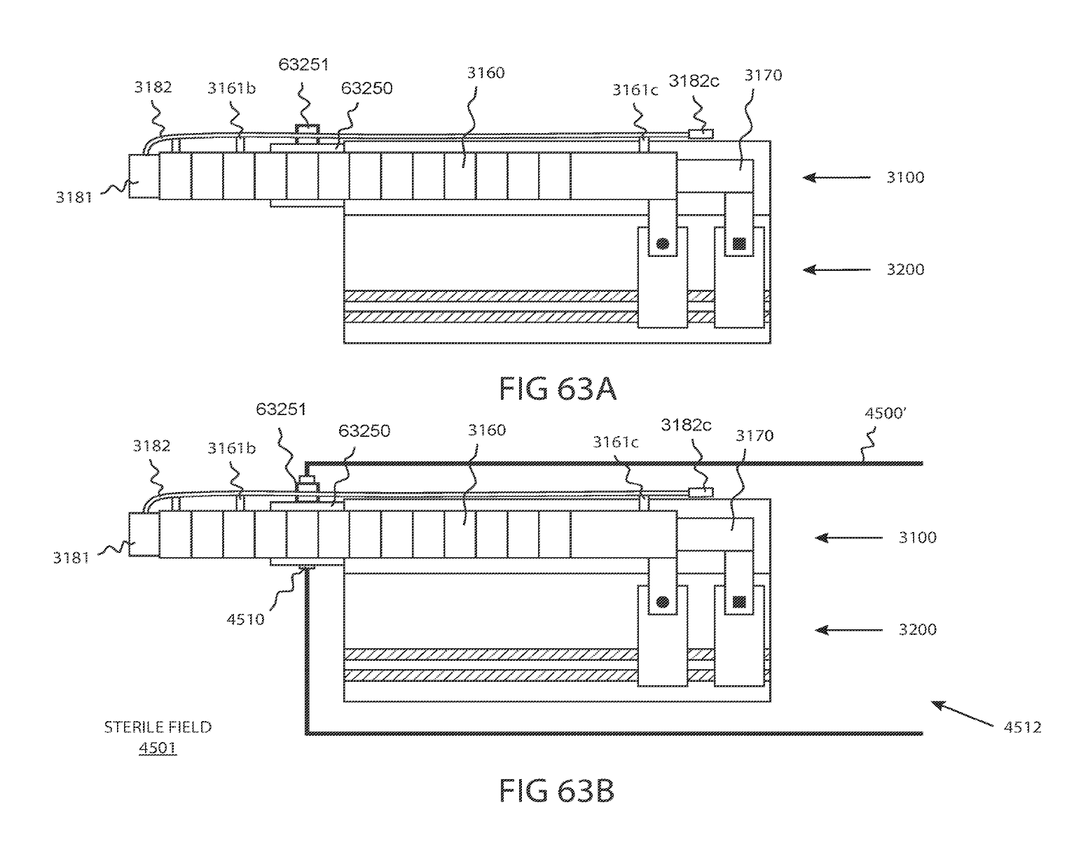

Referring to FIGS. 63A and 63B, sterile drape 4500′ can be similar or dissimilar to drape 4500 of FIGS. 62A and 62B, comprising a distal opening 4510 and a proximal opening 4512. In a first step, an operator, such as a nurse or clinician, can operably attach a reusable camera, camera 3181, to outer probe 3160 as shown and described hereabove. Camera cable 3182 can be attached to one or more cable connectors 3161b, and attach to one or more proximal camera connectors 3182c. Camera cable can be fed thru one or more portions of introducer 63250, such as one or more projections 63251, configured to allow opening 4510 of drape 4500′ to be secured about introducer 63250 without impinging camera cable 3182.

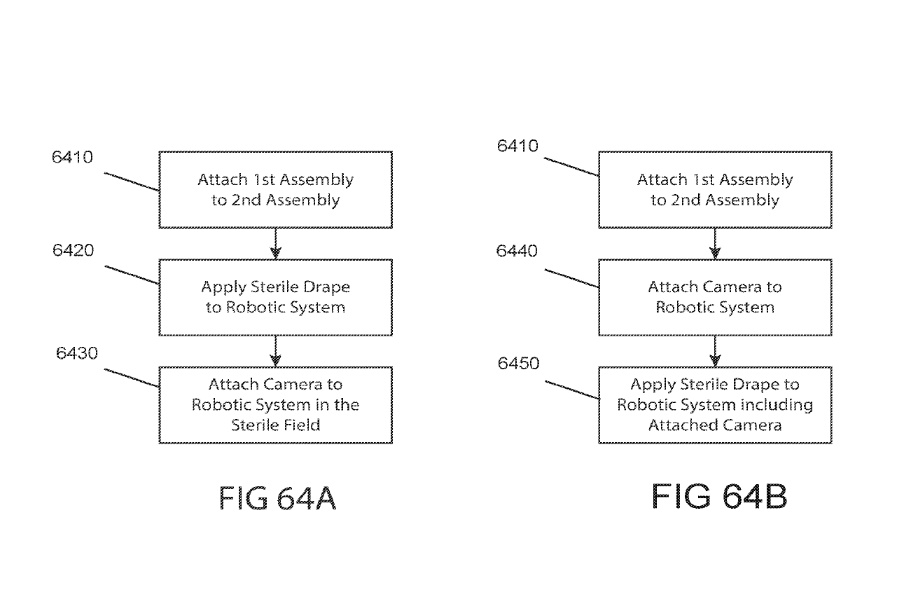

Referring now to FIG. 64A, a method of applying a sterile drape is described. In STEP 6410, an operator, such as a nurse or clinician attaches a first assembly 3100 to a second assembly 3200 as described herein. In STEP 6420, the operator applies the sterile drape as described hereabove, covering the robotic system with the drape, aligning dorsal opening 4511 with the top of first assembly 3100. In STEP 6430, the user attaches the camera assembly to the robotic system, such that the camera assembly resides entirely in the sterile field 4501.

Referring now to FIG. 64B, an alternative method of applying a sterile drape is described. In STEP 6410, again an operator, such as a nurse or clinician attaches a first assembly 3100 to a second assembly 3200 as described herein. In STEP 6440, the operator attaches the camera assembly to the robotic system, prior to applying sterile drape 4500′. In STEP 6450, the operator applies the sterile drape, as described hereabove, covering at least a portion of camera cable 3182.

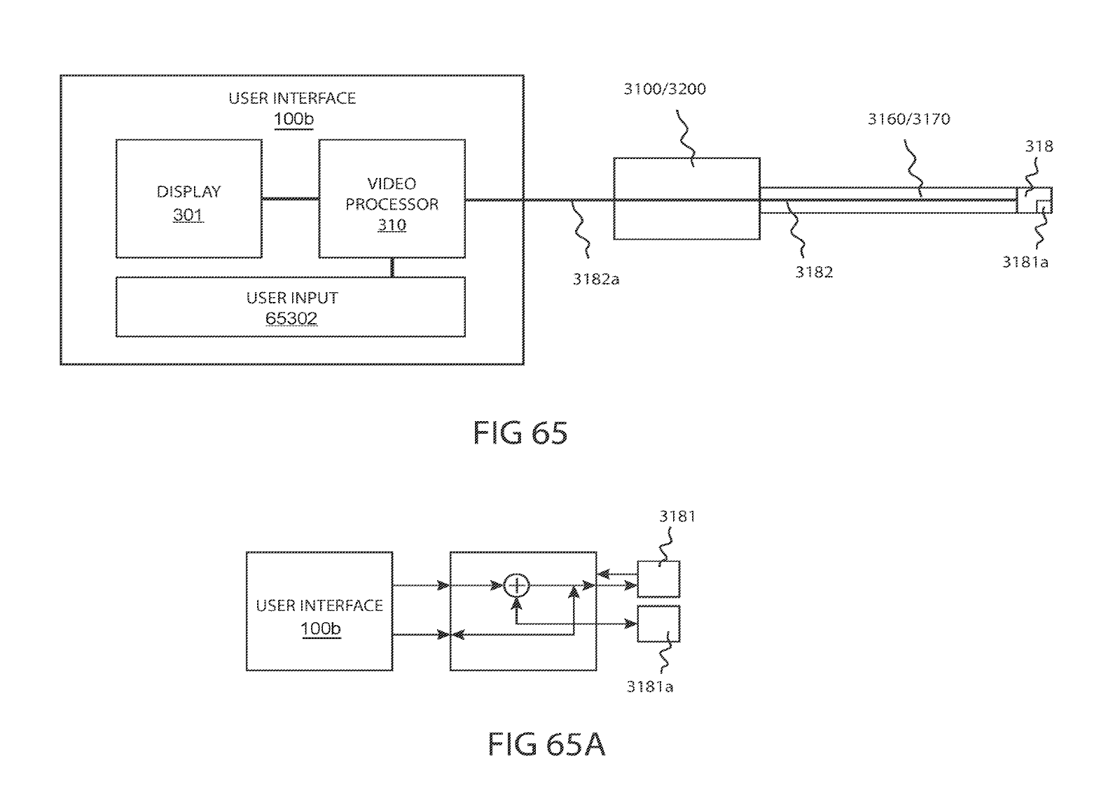

Referring now to FIGS. 65 and 65A, a schematic view of the system of the present inventive concepts is illustrated, including user interface 100b comprising display 301, user input 65302, and a controller comprising a video processor 310. FIG. 65 shows a systematic control loop for the control of brightness of the image displayed on display 301, described herebelow. The robotic system, including first assembly 3100 and second assembly 3200, controlling inner probe 3170 and outer probe 3160, comprises camera 3181 and light 3181a attached to the distal end of outer probe 3160. Cables 3182 and 3182a connect camera 3181 and light 3181a, through the first and second assemblies, to video processor 310 of user interface 100b. Video processor 310 uses one or more algorithms and/or processes for controlling camera 3181, light 3181a and/or processing information collected by camera 3181 such as to display an image based on the information onto display 301, as described herebelow.

Video processor 310 uses a feedback loop to adjust tone mapping and gamma correction to enhance dark regions of the image as viewed by camera 3181. User input 65302 can comprise a control configured as a slider, allowing the user to adjust the tone mapping and gamma correction with a single control. Alternatively or additionally, user input 65302 can comprise multiple controls to allow the user to manipulate the image parameters, such as brightness, gamma levels, contrast, and the like. In some embodiments, user interface 100b can comprise one or more modes, such as an “expert” mode, which allows the user to manipulate more parameters than in a normal operating mode.

Video processor 310 can provide an unsharp masking filter, configured to provide local contrast enhancement in the displayed image. Video processor can further provide one or more sharpening filters, such as one or more filters which enhance edge features, and or one or more filters to enhance the visualization of blood, blood vessels and/or other anatomic features. Automatically or via user input commands, video processor 310 can manipulate the color balance of the displayed image, for example by manipulating the contrast, the RGB gamma correction, and/or the individual RGB gain.

In some embodiments, video processor 310 can be configured to allow the user to zoom the image displayed on display 301. Video processor 310 can be further configured to allow the user to and/or automatically rotate the image, based on the orientation of the probe and/or camera 3181. Video processor 310 can digitize and packetize the video information collected by camera 3181, such that the signal generated by video processor 310 can be displayed on a screen with a lesser native resolution than the information gathered by camera 3181.

In some embodiments, camera 3181 provides a sync signal to video processor 310, such that video processor 310 can detect delays or other issues with signal provided by camera 3181. In the event of an error, video processor 310 can display a warning message to the user on display 301, and/or trigger an alert state, such as an alarm state in which the user cannot manipulate the probe until the alert state has been cleared, such as when the sync signal has returned to normal.

Referring specifically to FIG. 65A, a PID loop can be employed to provide an auto illumination feature for video or other images displayed on display 301 of user interface 100b. Video processor 310 can monitor brightness levels of the camera data by averaging total light collected by the sensor, and determining a brightness value. Based on this determination, video processor 310 can adjust the image, by adjusting brightness, contrast, and/or gamma levels, or the processor can increase the brightness of light 3181a to increase the overall brightness of the field, such that camera 3181 collects more light, or the processor can perform a combination of increasing the intensity of light 3181a and manipulating the levels of the collected image information.

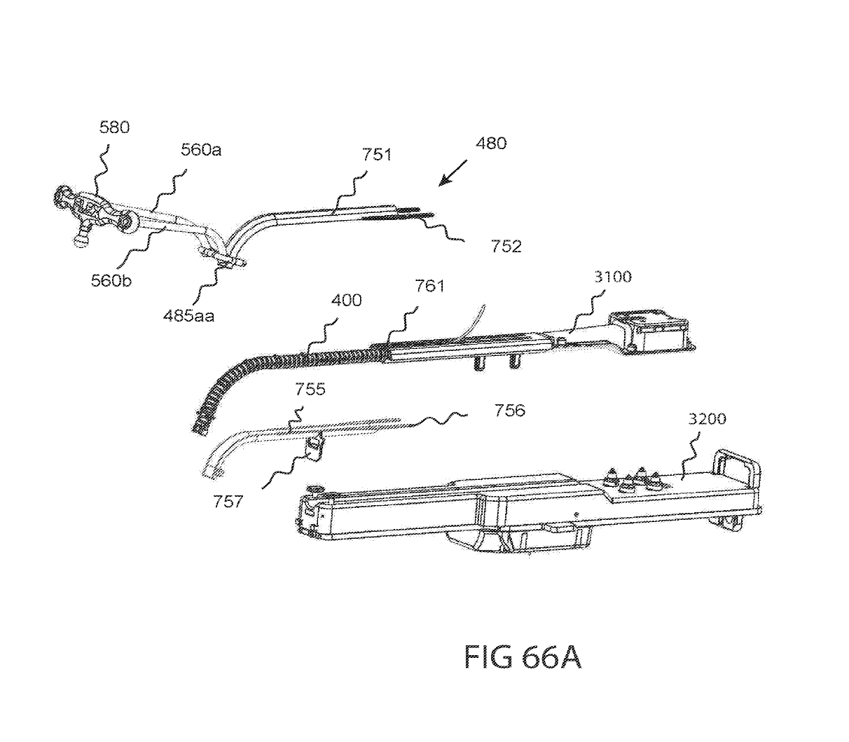

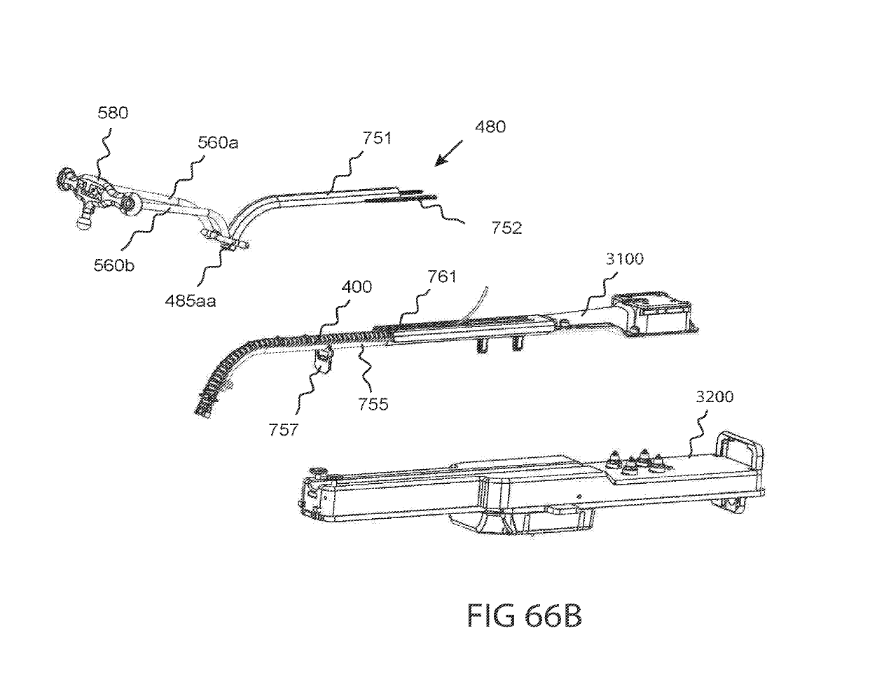

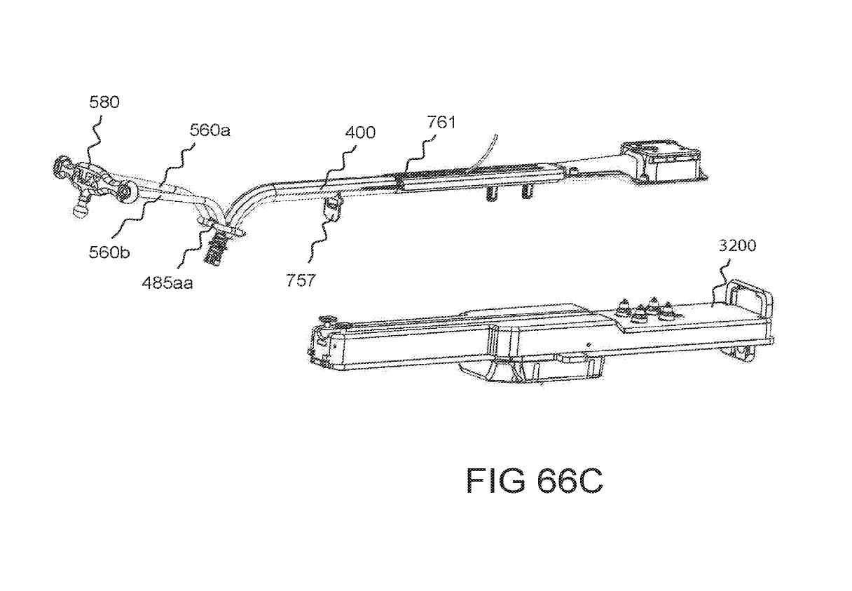

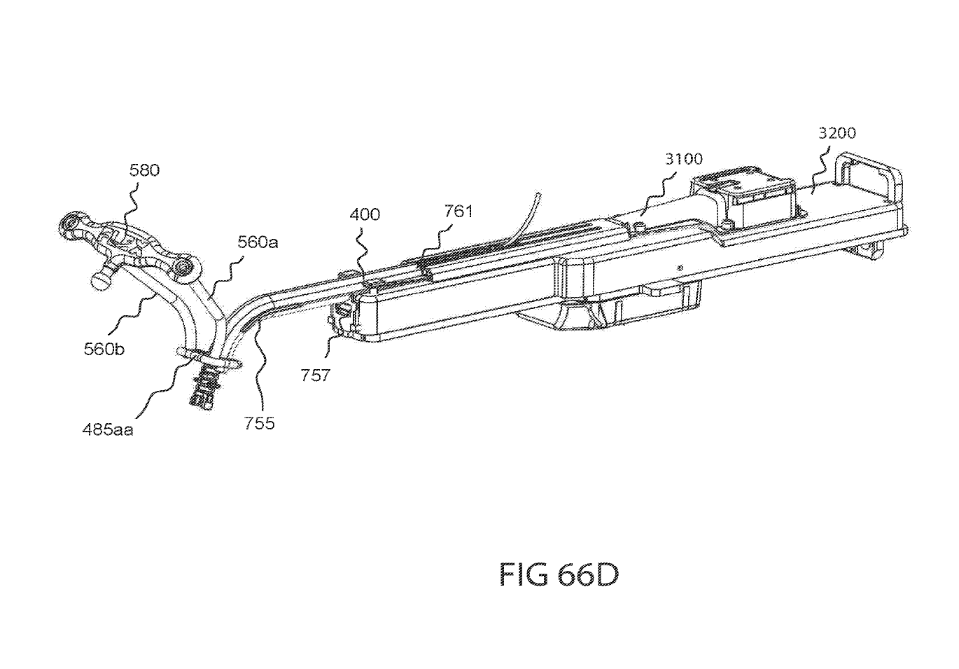

FIG. 66A is a perspective view of a removable introducer 480 having a clam-shell configuration, in accordance with other embodiments. In some embodiments, the introducer 480 is constructed and arranged to be disposable, namely, for a single use, for example, during a single medical procedure. In other embodiments, the introducer 480 is constructed and arranged for reuse, for example, multiple medical procedures. The introducer 480 can be constructed and arranged to slidingly receive an articulating probe 400, and support, stabilize, and/or guide the articulating probe 400 to a region of interest such as a body lumen. The clam-shell configuration allows attachment to the articulating probe 400, which may be disposable, and which can be similar to or the same as other articulated probe assemblies described in other embodiments herein. In embodiments where both the introducer 480 and probe 400 are disposable or single use, the combination of the introducer 480 and probe 400 can be collectively part of a disposable portion 3100 of a feeder assembly. The introducer 480 can be removably coupled to the disposable portion 3100 and/or a reusable portion 3200 of the feeder assembly, for example, an adaptor or base assembly described herein. In other embodiments where the introducer 480 is reusable, the introducer 480 can be coupled to other disposable portions of a feeder assembly. This system permits introducers of different sizes, shapes, entry trajectories (e.g. angles), and/or other configurations to be provided, and attached to the same reusable base or adaptor. The introducer 480 can be attached in an operating room or other environment without requiring complex interchanging of components, or wholesale discarding of the feeder assembly.

FIGS. 66B-D are perspective view of the removable introducer 480 of FIG. 66A in various stages of assembly, in accordance with other embodiments.

As shown, the introducer 480 can include a top snout 751 and a bottom snout 755. An attachment mechanism 485aa is coupled to the top snout 751. First and second tool supports 560a, b (generally, 560) extend between the attachment mechanism 485aa and a dogbone connector 580 coupled to first and second tool supports 560. The attachment mechanism 485aa can include a collar having an opening for receiving an end of each tool support 560. The attachment mechanism 485aa can be glued, bonded, or otherwise affixed to the end of the top snout 751. The attachment mechanism 485aa can be fixedly attached to one or more of the tool supports 560. Alternatively, one or more of the tool supports 560 can, using ball joints or the like, rotate or otherwise move relative to the attachment mechanism 485aa A gimbal (not shown) can be positioned at the attachment mechanism 485aa and rotatably engage one or more guide elements, for example, described herein, of the tool supports 560 at the attachment mechanism 485aa.

In some embodiments, a Teflon or polytetrafluoroethylene tube or the like (not shown) is inserted into and extend through the dogbone connector 580 and tool supports 560, such as to provide a lower resistance to one or more tools inserted through the dogbone connector and tool supports into the surgical field.

The connector 580, attachment mechanism 485aa, tool supports 560, and top snout 751 can form a single unit as shown in FIG. 66B, and therefore can be removed from, and connected to, the bottom snout 755 as a single unit. In other embodiments, one or more of the connector 580, attachment mechanism 485, tool supports 560, top snout 751, and introducer 480 can be removed and replaced with a different connector, attachment mechanism, tool supports 560, and/or introducer 480, for example, having different configuration parameters such as a different shape, length, size, and so on.

The bottom snout 755 of the introducer 480 is positioned along a bottom of at least a portion of the probe 400. The bottom snout 755 may include a support mechanism 757 that directly abuts an end of the reusable portion 3200, for example, base or adaptor shown in FIG. 66A, or mates with a coupling at the base or adaptor. As shown in FIG. 66A, the bottom snout 755 may include a clip 756 or related latch or coupling that mates with a corresponding coupling (not shown) at the disposable portion 3100.

As shown in FIG. 66C, the top snout 751 is removably coupled to the bottom snout 755. As part of this assembly step, the top snout 751 may be angled in a downward direction. The distal end of the snout halves 751, 755 engage, for example, hinge, first. One end of the top snout 751 includes a clip 752 or related latch or coupling that mates with a corresponding coupling 761 at the disposable portion 3100. The other end of the top snout 751 can be moved downward to lock the snout halves 751, 755 together at the proximal end. The top and bottom snout halves 751, 755 can be latched together by a clamp or other latch mechanism (not shown), which may be coupled to one of the top and bottom snout halves 751, 755, which can apply a force to the other of the top and bottom snout halves 751, 755 that causes the snout halves 751, 755 to press together.

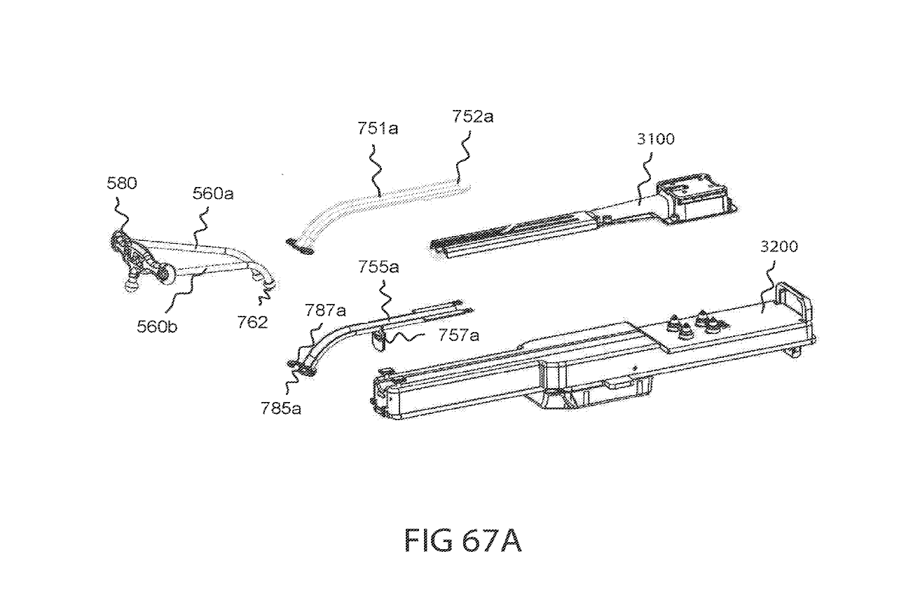

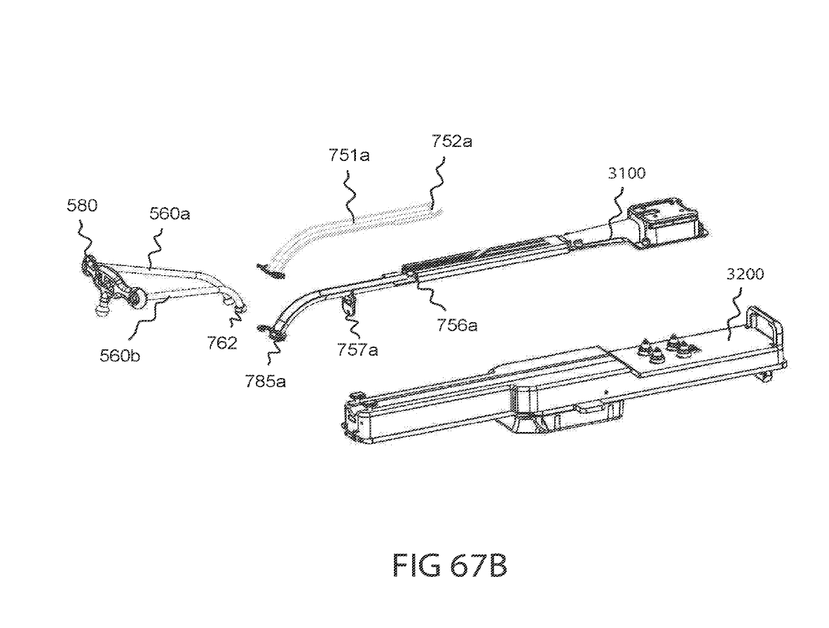

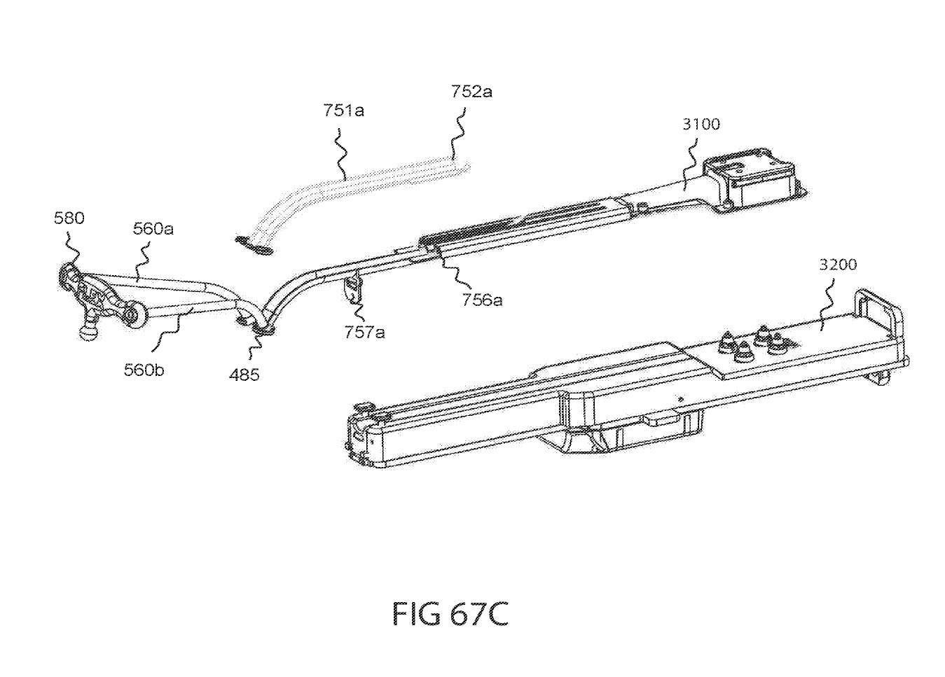

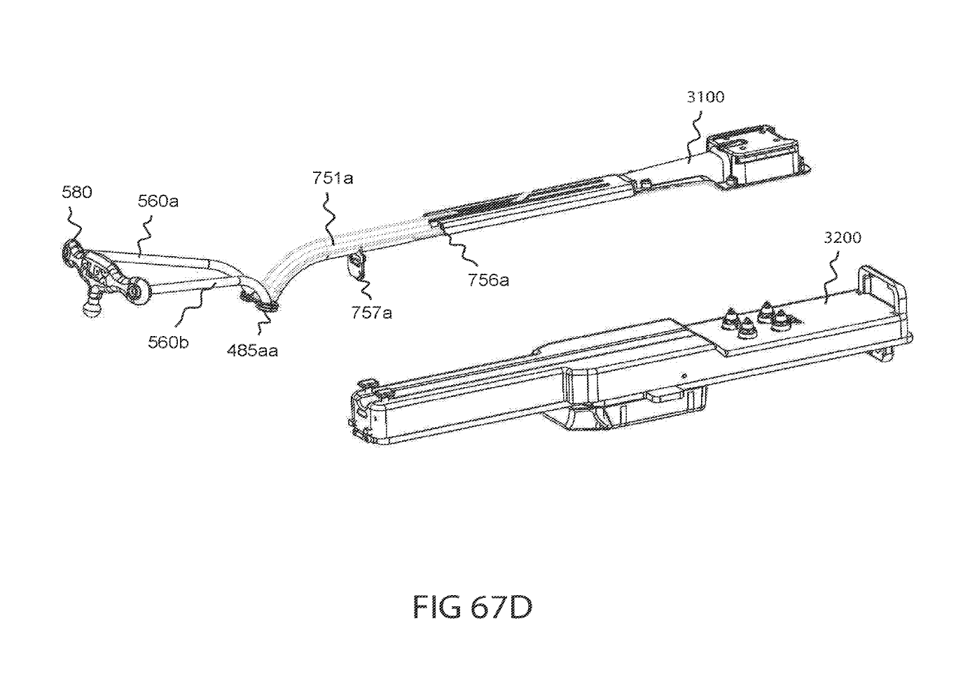

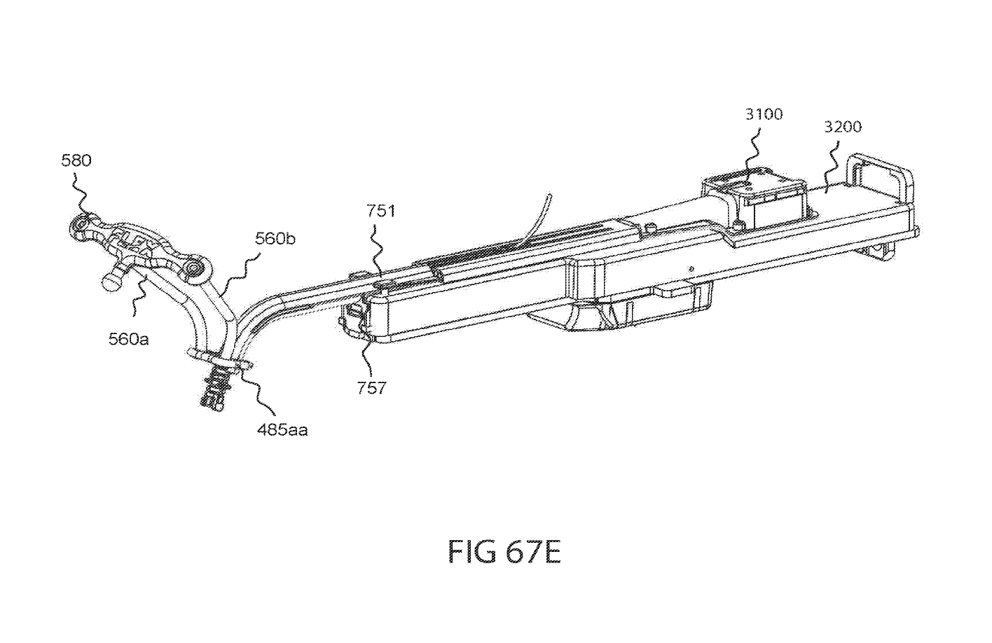

FIGS. 67A-67E are perspective views of a removable introducer 480 having a clam-shell configuration, in accordance with other embodiments.

A bottom snout 755a of the introducer 480 can be attached to the disposable portion 3100 of a feeder assembly, for example, for example, using a clip 756a, latch, or other connector that mates with a corresponding coupling at the disposable portion 3100 (similar to the bottom snout 755 of FIG. 66). The bottom snout 755a can be positioned about a bottom region of the probe 400.

An attachment mechanism 785a is coupled to the bottom snout 755a. The attachment mechanism 785a can be glued, bonded, or otherwise affixed to the end of the bottom snout 755a. The attachment mechanism 785a and bottom snout 755a can be connected to, or removed from, the disposable portion 3100 as a single unit. The attachment mechanism 785a can include a collar having an opening 787a for at least one tool support.

A connector, for example, dogbone connector 580 and tool supports 560a, b (generally, 560) can be provided, wherein the tool supports 560 each include a distal end 762 constructed and arranged, for example, as a ball joint, for conformable insertion into an opening 787 at an end of the attachment mechanism 785. The dogbone connector 580 and tool supports 560 can be connected to, or removed from, the attachment mechanism 485 as a single unit. In some embodiments, one or more of the tool supports 560 can rotate or otherwise move relative to the attachment mechanism 785a. A gimbal (not shown) can be positioned at the attachment mechanism 785a and rotatably engage one or more guide elements, for example, described herein, of the tool supports 560 at the attachment mechanism 785a.

The top snout 751a is removably coupled to the bottom snout 755a. One end of the top snout 751a can include a clip 752a or related latch or coupling that mates with a corresponding coupling at the disposable portion 3100. Then, the other end of the top snout 751a can be moved downward to lock the snout halves 751a, 755a together at the proximal end. In some embodiments, the bottom snout 755a can be latched to the reusable portion 3200, for example, by adaptor, by a latch mechanism (not shown), similar to the latch mechanism 757 of FIG. 66D.

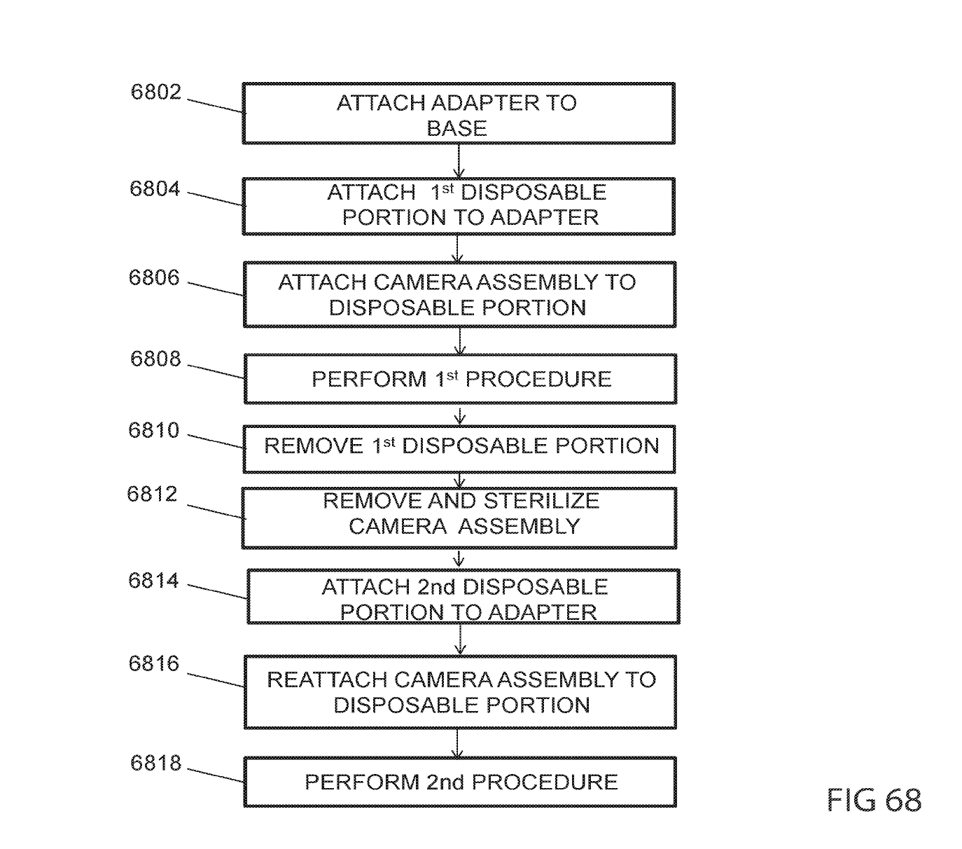

FIG. 68 is a flowchart illustrating a method 6800 for assembling a robotic system to perform one or more operations, in accordance with an embodiment. When describing the method 6800, reference is made to FIG. 68. Although the method 6800 refers to a sequence of blocks, or steps, the method 6800 is not limited to this sequence. In other embodiments, various blocks can be performed in a different order. Some or all of the method 6800 can be performed by an articulating probe system in accordance with some embodiments. The robotic system may be a robotic system described in one or more embodiments herein.

At block 6802, the adaptor is attached to the base of a feeder assembly of the robotic system. The adaptor and the base can be reusable, that is, constructed and arranged for multiple medical procedures. As described herein, the adaptor includes a set of capstans, electrical connectors, alignment mechanisms, carriages, a drive mechanism for driving a probe assembly, and at least one latch mechanism for coupling with the base and/or disposable portion, each described in detail in embodiments herein.

At block 6804, a first disposable portion is attached to the adaptor, so that the adaptor is positioned between the disposable portion and the base. As described herein, the disposable portion includes a probe, bobbins, cables, gears, arid/or other mechanical devices that mate with corresponding elements of the adaptor for controlling the probe, steering cables, and/or tools attached thereto. The disposable portion can include a removable introducer, for example, having a clam-shell configuration in accordance with other embodiments herein, to support, stabilize, and/or guide the articulating probe.

At block 6806, a reusable camera assembly is attached to the disposable portion, and can plug into the adaptor, for example, described in accordance with embodiments herein.

At block 6808, a first procedure can be performed by the robotic system, for example, a medical procedure, such as a transoral robotic surgery procedure.

At block 6810, after the first procedure is completed, the disposable portion can be removed from the adaptor. In some embodiments, the disposable portion is constructed for a single use, and is sanitized (e.g. sterilized) one time prior to that single use. In other embodiments, some or all of the disposable portion is constructed for multiple uses, but fewer uses then the adaptor and/or base.

At block 6812, the camera assembly is removed and sanitized (e.g. sterilized) after the first procedure.

At block 6814, a second disposable portion is attached to the adaptor that is different than the first disposable portion. The second disposable portion can have a same, similar, or different configuration or function than the first disposable portion.

At block 6816, the reusable camera assembly, after sanitization, is reattached to the second disposable portion.

At block 6818, a second procedure can be performed by elements of the robotic system.

In some embodiments, method 6800 includes the application of a sterile drape to one or more portions of system 3002, such as is described hereabove in reference to sterile drape 4500 of FIGS. 62A-B and/or 63A-B.

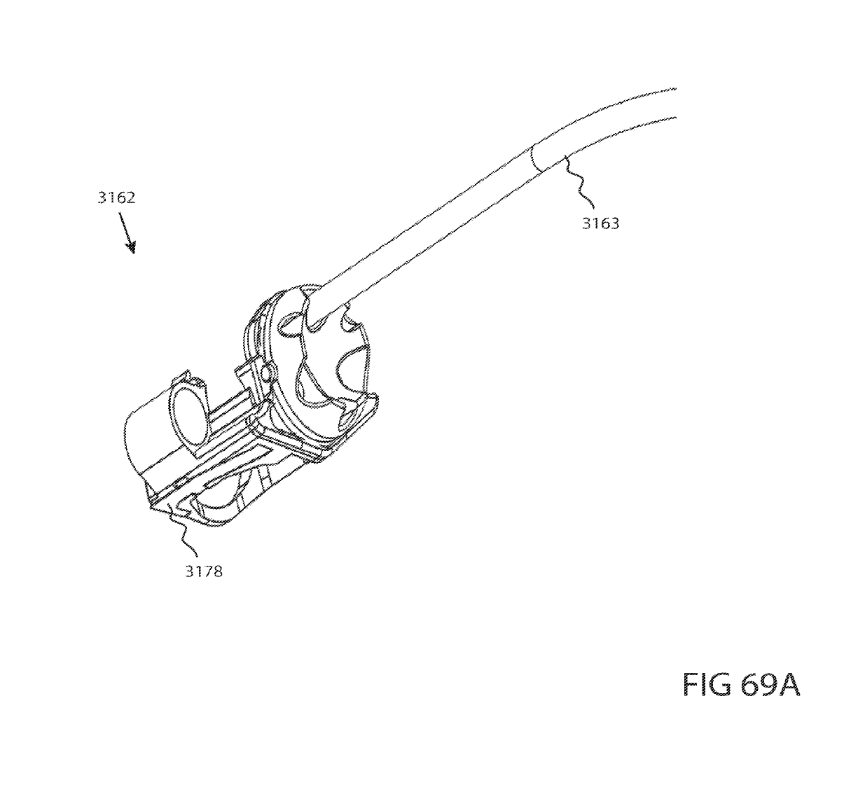

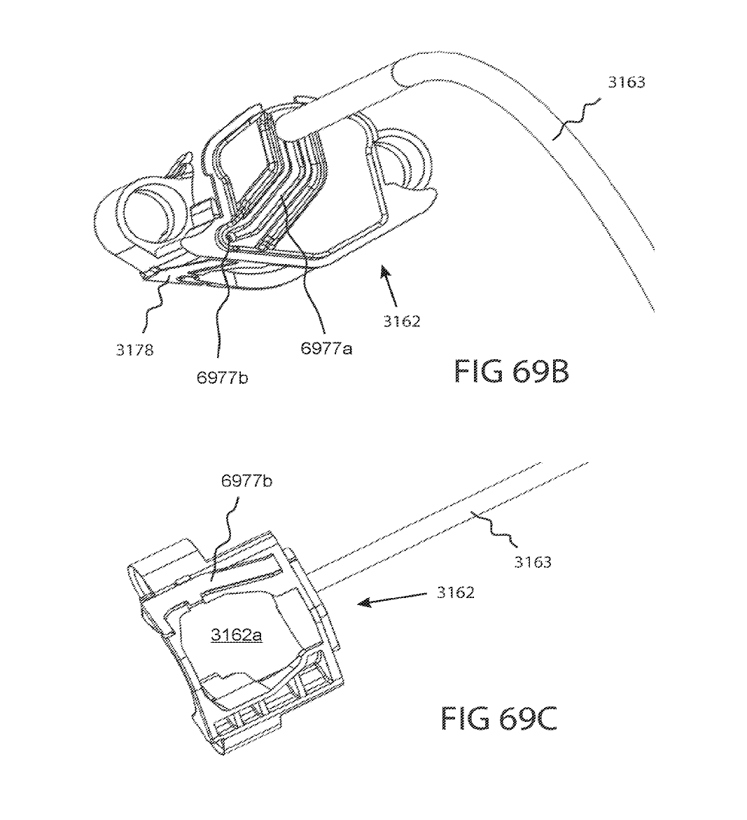

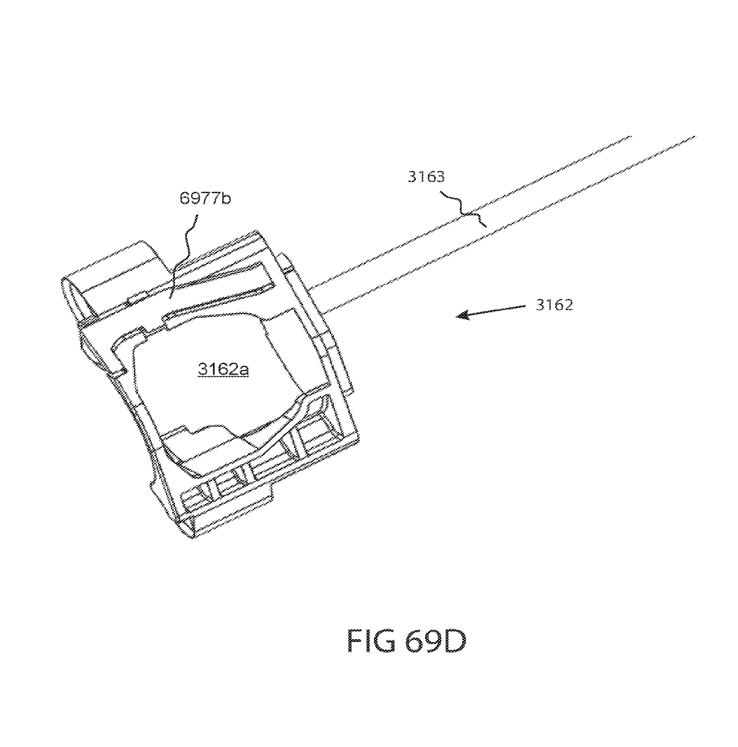

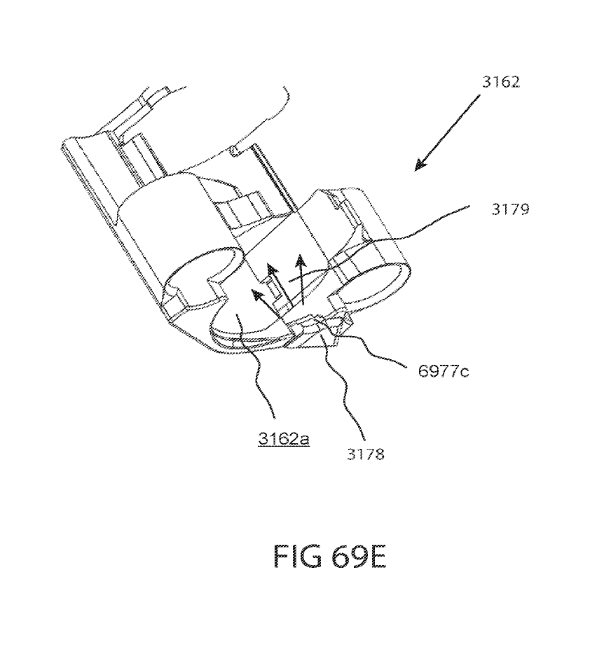

FIGS. 69A, 69B, 69C, 69D and 69E, are rear perspective, rear cutaway perspective, bottom perspective, bottom cutaway perspective, and front perspective, views of an embodiment of the distal link 3162 of the outer probe 3160. In the present embodiment of the distal link 3162, a camera seat 3162a at which a camera 3181 (see FIG. 61B) can be positioned and secured is provided. The camera optics is secured in the seat 3162a and a lens of the optics is oriented in a distal direction. During operation, the camera lens can become obstructed with foreign material from the region of the surgery. Accordingly, an irrigation channel 3163 is provided as a source of irrigation fluid for flushing the camera optics.

In an embodiment, an irrigation channel 3163 is provided in a working channel or side channel of the probe. The irrigation channel can take the form of a flexible tube suitable for transporting fluid. In an embodiment, the output end of the irrigation channel 3163 couples to a rear portion 3178 of the distal link 3162. A first irrigation segment 6977a transports the fluid from the junction of the irrigation channel tube to a lower portion of the distal link 3162. From there, a second irrigation segment 6977b provides a further pathway for transport of the fluid along the lower portion of the distal link 3162. An rear portion 3178 is provided to seal the region of the second segment 6977b. From there, the fluid is further transported to outlet 6977c at a front face of the distal link. At the outlet, the fluid is redirected so that the resulting wash 3179 is oriented in a direction toward the instered optic to provide an effective flush of the optic effectively.

In some embodiments, the camera housing and cable are independent of the distal link and irrigation system. In other words, in such embodiments, the irrigation system does not enter the camera. In this manner, the camera can be used for a procedure, removed from the system, and sterilized for re-use, independently of the irrigation system and probe, which, in some embodiments, may be intended for single-use.

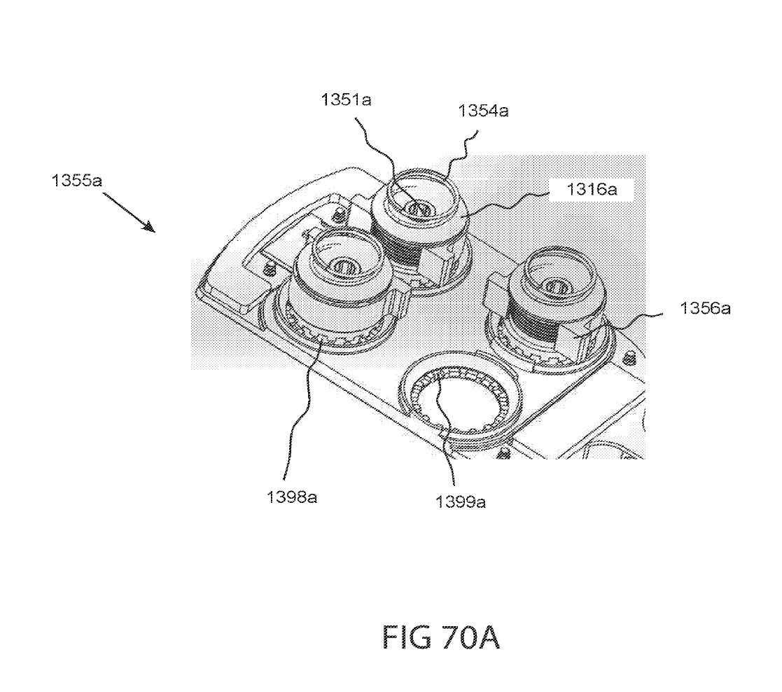

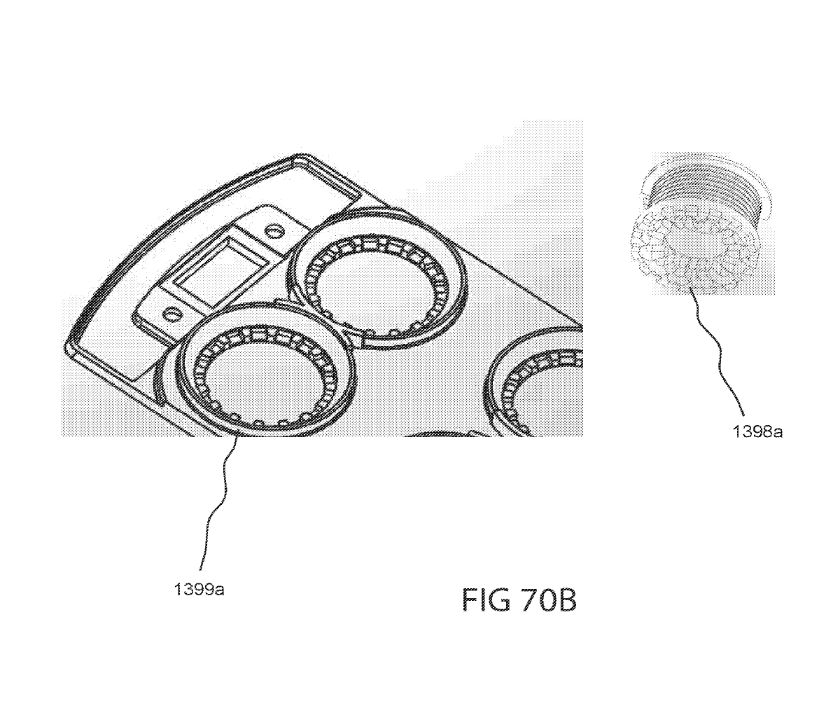

FIG. 70A is a cutaway perspective view of a disposable portion of a feeder assembly, in accordance with some embodiment. FIG. 70B is a view illustrating a bobbin having a plurality of castellation features for mating with a plurality of castellation features of a bobbin plate, in accordance with some embodiments.

A plurality of cable bobbins 1316a are each constructed and arranged to be centered about, and rotate about, a bobbin axle 1351 and to receive a cable, for example, a steering cable described herein. In some embodiments, the cable can comprise a steering and locking cable which steers and/or reversibly tightens to lock or stiffen an outer link mechanism and/or inner link mechanism of a probe, for example, similar to the configuration of FIG. 5B above. In some embodiments, a first end of each cable is coupled to a distal link of the probe, for example, a distal outer link 3162 of FIG. 58A or distal inner link 421_{D} of FIG. 19F, and a second end of each cable is wound about a bobbin 1316a. During shipment of the unit, it is desired that the cables not lose tension or become released. If the cables lose tension they may come off of the bobbin and tangle within the device. Slack in the cables could also allow the outer links to separate which may allow the inner links to rotate with respect to the outer links which would cause internal friction between the two link assemblies. The cable bobbin 1316a may be optionally seated on a bobbin washer in turn interfacing with a bobbin spring 1354a. The bobbin spring 1354a may be positioned at a first end of the bobbin 1316a. A plurality of castellation features 1398a may be at a second end of the bobbin 1316a and are constructed to mate with a plurality of castellation features 1399a in the bobbin plate 1355a.

In some embodiments, to prevent release of the cable from cable grooves 1352, a cable clip 1356a can be removable positioned about at least a portion of the bobbin 1316a, and which rotatably engages bobbin 1316a allowing cable to be collected onto bobbin 1316a and paid out or extended from bobbin 1316a while maintaining the portion of the cable surrounding bobbin 1316a or otherwise positioned in the cable grooves 1352 helically wound about the bobbin 1316a in close proximity to bobbin 1316a.

As described above, during shipment or pre-operating use, the mechanical locking mechanism, which comprises a combination of pins and recesses or the like that controls cable tension during shipment and pre-operating use of the disposable portion 3100 of the feeder assembly, is installed. In some embodiments, the mechanical locking mechanism comprises a combination of the spring 1354a, bobbin plate castellation features 1399a, and bobbin castellation features 1398a that provide an anti-rotation feature with respect to cable pulleys, capstans, and the like. In particular, to prevent rotation, and to control cable tension during shipment or pre-operating use, the bobbin plate castellation features 1399a are constructed and arranged to mate with the bobbin castellation features 1398a. The spring 1354a applies a force on the bobbin 1316a, which presses the bobbin 1316a into the castellation features 1399a of the bobbin plate 1355a, which locks the bobbin 1316a in place and prevents rotation or other movement of the bobbin 1316a, which may otherwise undesirably release cable tensioning during shipment.

During operation, a capstan in the adaptor or base (not shown) forces the bobbin 1316a into a position whereby the bobbins 1316a are separated from the castellation features 1399a of the bobbin plate 1355a, and allowing the bobbins 1316a to rotate. For example, referring to other embodiments, after the disposable portion 3100 is attached to the reusable portion 3200, a capstan 216a of the reusable portion 3200 mates with a corresponding bobbin 1316a in the disposable portion 3100, and in doing so, pushes the bobbin 1316a in an upward direction, compressing the spring 1354a and removing a frictional engagement between castellation features 1398a and 1399a by separating the castellation features from each other.

Capstan 216a includes a boss (not shown) which fits inside a bore in the bobbin. The cable tension is transferred from the cable to the bobbin to the capstan which ultimately takes the load off the cable tension. This allows load bearing mechanisms to be located in the capital equipment as opposed to the disposable.

After a disposable portion 3100 is removed from the reusable portion 3200 (e.g. after procedure completion or after an emergency release), the capstan is no longer in contact with the bobbin 1316a. Accordingly, the spring 1354a operates to apply a force that pushes the bobbin 1316a in a downward direction as shown. The castellation features 1398a and 1399a once again engage each other, providing resistance to movement.

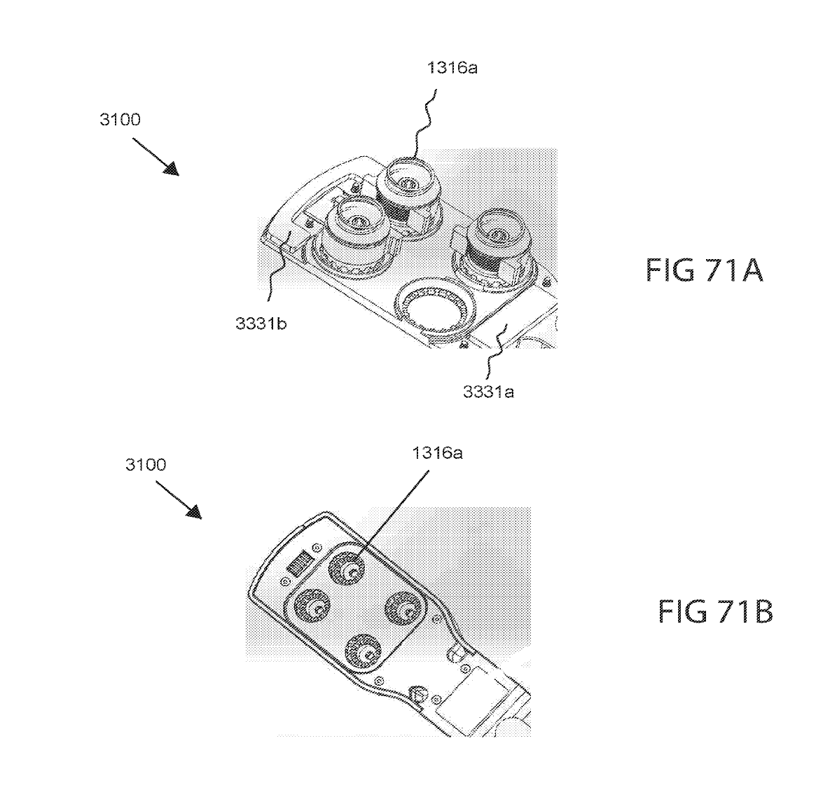

FIG. 71A is a cutaway perspective view of a magnetic latch assembly at a proximal end of a feeder disposable portion 3100, in accordance with some embodiments. FIG. 71B is a view of an underside of the feeder disposable portion 3100 of FIG. 71A. The magnetic latch assembly uses magnetic attraction to permit the first assembly 3100 to removably attach to the second assembly 3200 at the proximal end. This feature simplifies user attachment and detachment, for example, by allowing a user to vertically align the disposable portion 3100 with the reusable portion 3200 as distinguished from complicated assembly as with other coupling approaches. It also allows fixation of the cartridge to the adaptor through a sterile drape without any penetration of the drape. Another benefit of a simplified latch such as the magnetic latch described herein is the rapid detachment that may be desired in emergency situations. Location of latch mechanisms and inclusion of a magnetic latch assembly on one end allows an operator to rapidly grasp and detach first assembly 3100 from second assembly 3200 with a single hand.

One or more plates 3331a, b (generally, 3331), or bars, coin-shaped objects, or other configuration, may be positioned in housings at the disposable portion 3100. One plate 3331a may be located at one side of the drive connection region of the disposable portion 3100, for example, including bobbins 1316a, pulleys, sections of steering cables, and so on, and the other plate 3331b may be located at the other side of the drive connection region. The two plates are attracted to two magnets located at opposite ends of the plates. Another plate at the opposite end of the magnets completes a loop increasing the strength of the magnetic pull.

Although two steel plates are shown, the disposable portion 3100 may include a single plate or more than two plates. The plates 3331 can be formed of any material that is attracted to one or more magnets located at the reusable portion 3200. The magnets can be permanent magnets and/or electromagnets. The magnets provide a sufficient magnetic field to hold the disposable portion 3100 in place against the reusable portion 3200 during operation of the articulating probe system. A housing may be positioned over the disposable portion 3100 to cover the plates 3331 (but includes openings that expose the bobbins 1316a), as shown in FIG. 71B.

In other embodiments, the disposable portion 3100 includes one or more magnets which attract one or more plates, bars, and so on coupled to the reusable portion 3200. In other embodiments, each of the disposable portion 3100 and the reusable portion include plates, bars, and so on formed of ferromagnetic material, that are arranged in a mutual attraction configuration, for example, opposite polarities, to hold the disposable portion 3100 in place against the reusable portion 3200.

This mechanism allows single handed assembly and disassembly, vertical and rotational attachment, and it can be the primary or secondary engagement to the adaptor—the (previous) latch at the front is the other engagement.

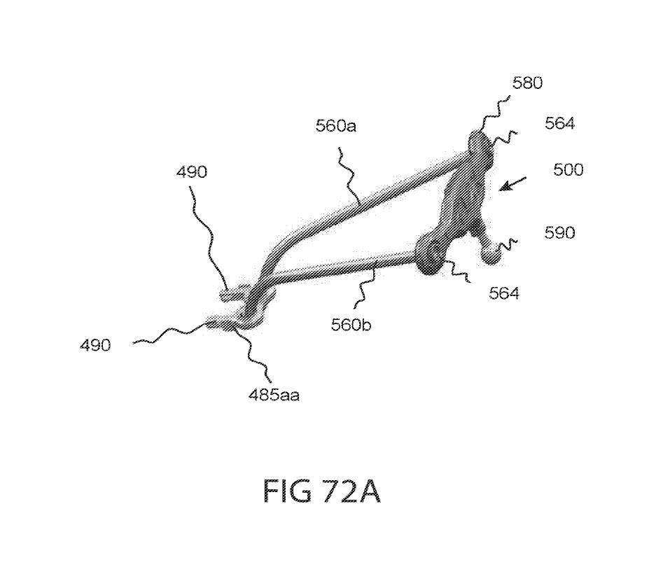

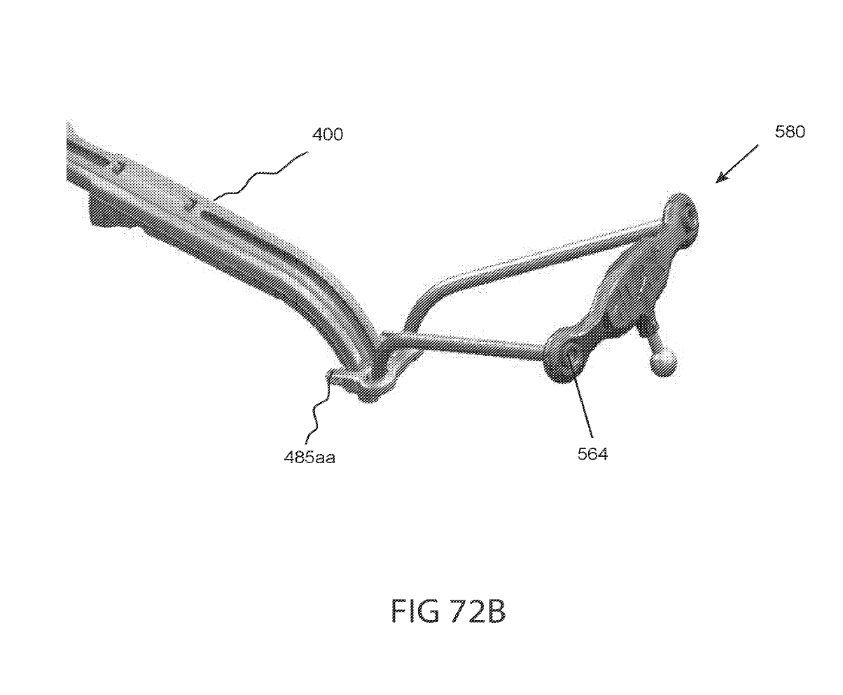

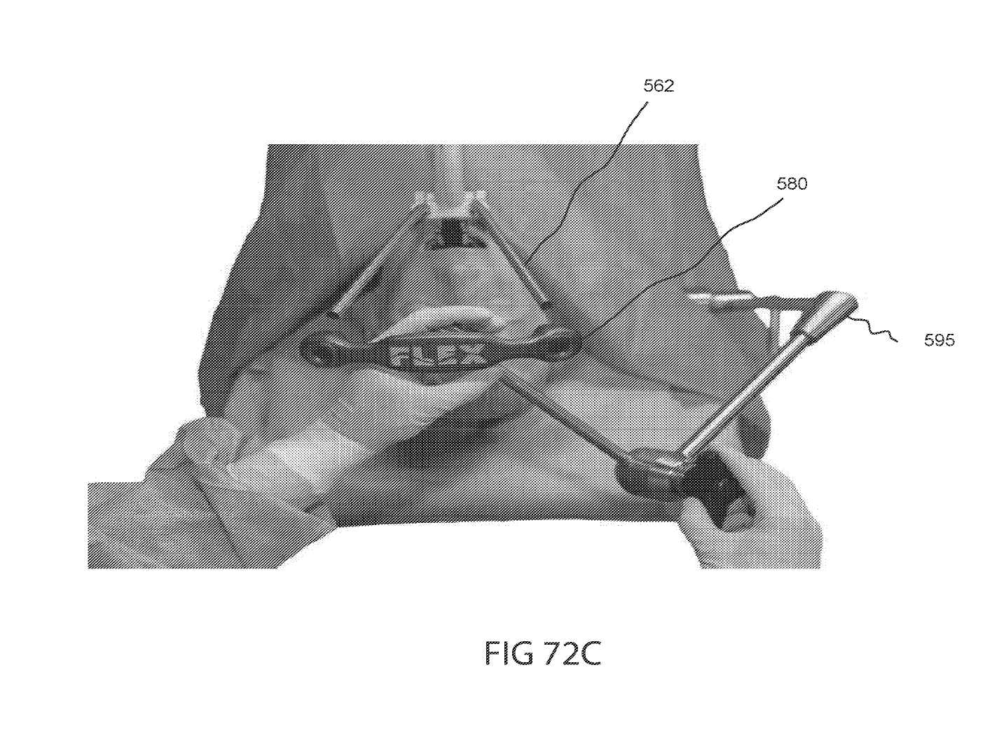

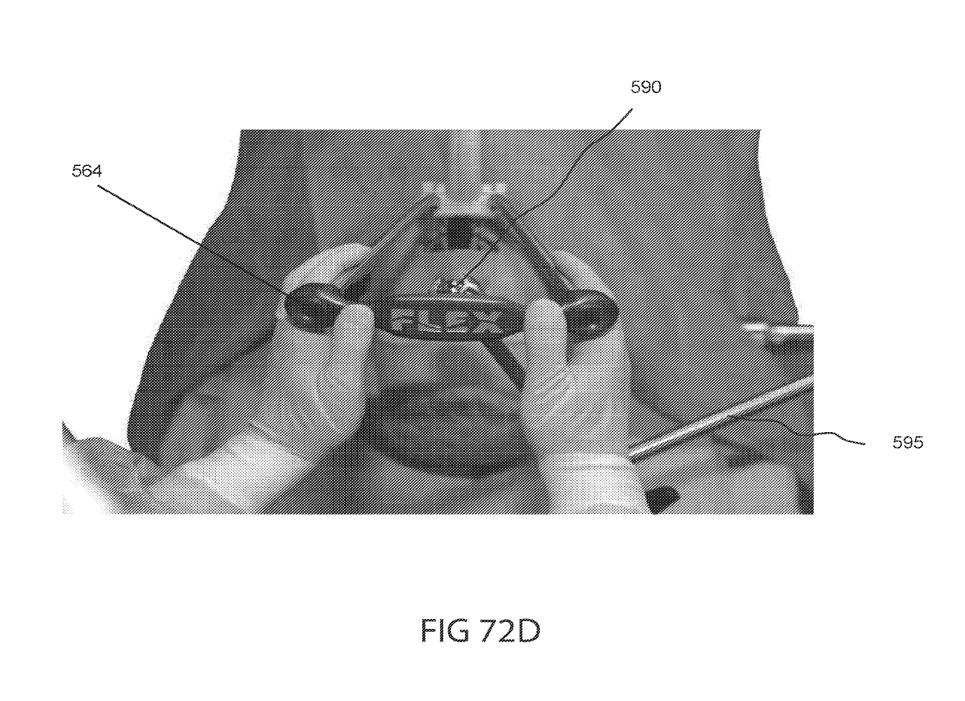

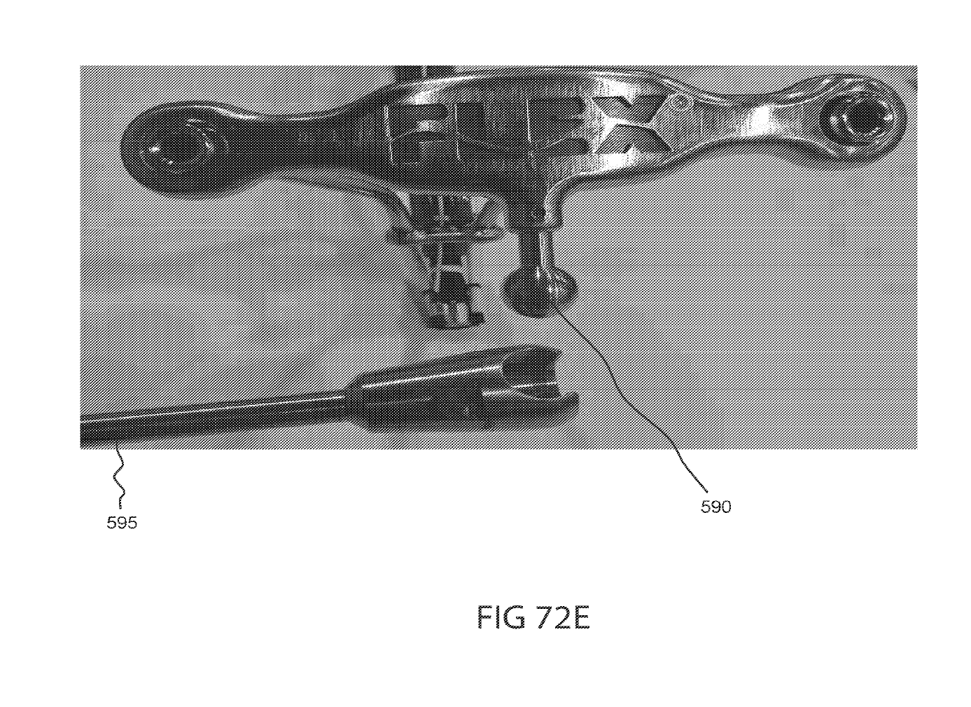

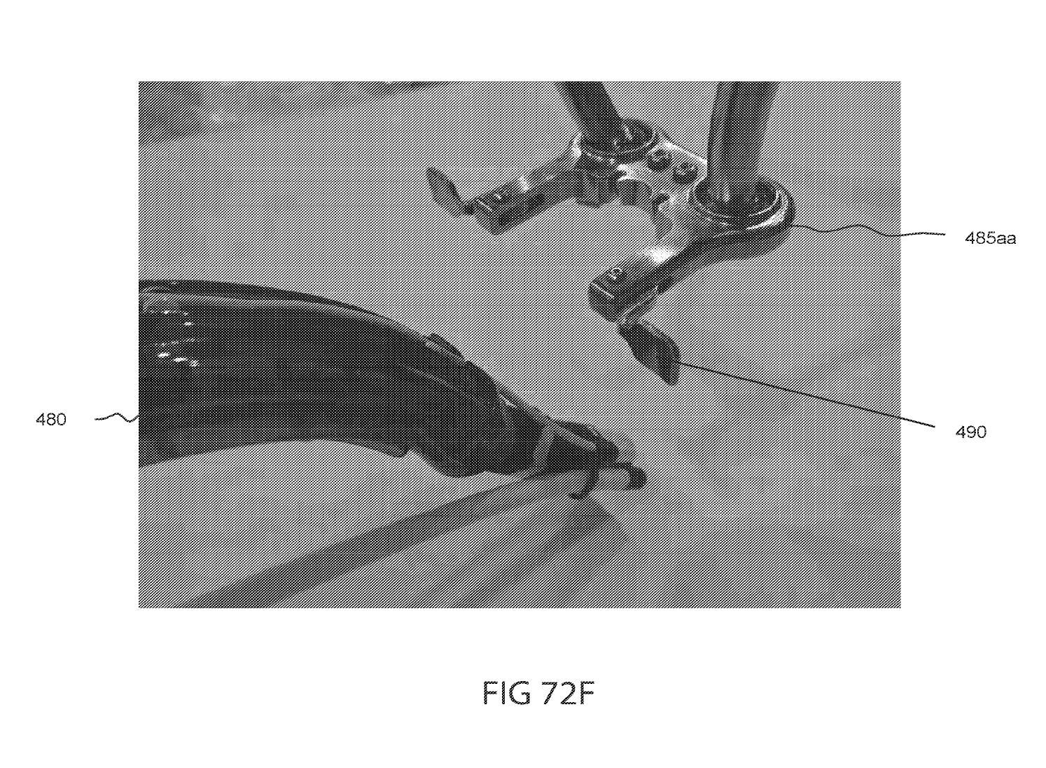

As described above, in some embodiments, a dogbone connector assembly can be attached to a disposable introducer. FIG. 72A is a perspective view of a connector assembly 500, in accordance with an embodiment of the present inventive concepts. FIG. 72B is a perspective view of the connector assembly 500 of FIG. 72A coupled to a disposable portion of a feeder assembly, in accordance with some embodiments.

The connector assembly 500 can include a connector 580, for example, a dogbone connector, which is constructed and arranged to be attached to and maintain a relative position and/or orientation between a first tool support 560a and a second tool support 560b. In some embodiments, the connector 580 comprises a rigid structure. In other embodiments, the connector 580 comprises at least a portion that is flexible or malleable. The connector 580 can comprise an operator shapeable structure. In some embodiments, the connector 580 has a one-piece design, for example, machined of a single stock, or molded as a single piece. In other embodiments, the connector 580 comprises two segments connected by a hinge or rotatable connector, such as a hinge comprising a flexible portion positioned between two rigid portions. The connector 580 can comprise a telescopically adjustable structure, such as to allow separation of tool supports 560a and 560b.

The connector 580 comprises a first opening 564a and a second opening 564b constructed and arranged to operably engage a first end of the first and second tool supports 560a, 560b, respectively. An attachment mechanism is coupled to the second ends of the tool supports 560a and 560b, respectively, and that can be removably attached to a disposable portion 3100 of the feeder assembly, for example, the introducer 480 shown in FIGS. 66A-H. In doing so, the attachment mechanism can include cams 490 or the like that can attach to the introducer 480.

The assembly 500 comprising a combination of the connector 580, tool supports 560, and attachment mechanism 485aa can be removed from the introducer 480 by separating the cams 490 from the introducer 480, and replacing the assembly 500 with replaced with a different dogbone connector assembly, which can have different configuration parameters, for example, a different length, different size openings, a different number of tool supports, and so on. The easy-to-attach attachment mechanism 485aa and the one-piece configuration of the assembly 500 obviates the need of the doctor or user to independently and painstakingly assemble the various components of the assembly 500, for example, to align each tool support 560 with a hole in the base attachment unit.

The connection made by the attachment mechanism 485aa at the introduction device 480 maintains a fixed distance and/or a fixed orientation between the first tool support 560a and the second tool support 560b. In some embodiments, the tool supports 560a and 560b can be rotatably attached to each other and/or the attachment mechanism 485aa for maintaining a fixed distance but not a fixed orientation. The first tool support 560a and the second tool support 560b can be fixed in position relative to each other. Accordingly, the positions of the first and second tool supports 560a, 560b are maintained during an operation, for example, where tools are inserted in the tool supports 560a, b, and used during a medical procedure.

At least one of the first tool support 560a and the second tool support 560b can include first and second guide elements, respectively, which in turn can include an outer guide element, also referred to as a proximal guide element, and an inner guide element, also referred to as a distal guide element. The inner guide element can be formed of plastic or related material. Materials can include but are not limited to fluoropolymers (e.g., polytetrafluoroethylene), fluorinated ethylene propylene, polyether block amide, high density polyethylene, low density polyethylene and/or nickel titanium alloy. Inner guide element can comprise laser cut tubes (e.g. polymer or metal tubes) and/or coils or braids of plastic or metal. In some embodiments, inner guide element comprises a polytetrafluoroethylene liner. In some embodiments, inner guide element comprises a stainless steel coil. In some embodiments, inner guide element comprises a coil covered by a polyether block amide. In some embodiments, inner guide element comprises different varying stiffness along its length, such as when comprising a tube of varying diameters along its length. At least a portion of the outer guide element is rigid, with limited or no flexibility. In some embodiments, the inner guide element can movably extend from the outer guide element, for example, in a telescoping configuration.

In some embodiments, the tool supports 560a, b can be coupled to the attachment mechanism 485aa by a gimbal or the like, permitting the tool supports 560a, b to rotate relative to the attachment mechanism 485aa, for example, allowing for three degrees of freedom between a tool support 560 and the attachment mechanism 485aa, which can include two-dimensional (X-Y) movement plus rotation. The gimbal or other pivot or ball and joint mechanism permits the guide element of the tool support 560 to rotatably or fixedly engage the attachment mechanism 485aa, for example, at a mid-portion of the guide element. In embodiments where a tool support 560 is slidably adjustable, thus allowing for a shortening of a portion of the support 560 that attaches to the dogbone connector 580, the dogbone connector 580 may require adjustability of the distance between connector openings.

The tool supports 560 can be locked in a fixed position relative to the attachment mechanism 485aa. The assembly can include a locking mechanism (not shown) to lock the at least one tool support 560 in the fixed position. The locking mechanism may be constructed to secure a position of the tool supports 560a, b, with respect to the attachment mechanism 485aa, thus preventing the tool supports 560a, b from sliding or otherwise moving axially during movement of the tools by one or more operators.

The tool support 560a, b, can be constructed and arranged to guide or otherwise provide a support for a tool shaft so that it can be guided to a side port coupled to an outer surface of an articulating probe received by the introduction device 480, which can support, stabilize, and/or guide the articulating probe to a region of interest. The side port can be coupled to a distal link of an articulating probe 400. The side port can be formed at a flange at the articulating probe 400.

The assembly can include one or more human interface devices (HIDs), not shown, but described herein) which may be integral with the dogbone connector 580.

As shown in FIGS. 72C-72F, the dogbone connector 580 can include an attachment rod 590 having a bulbous end that can attach to a steering rod 595. A user can therefore manipulate an articulating probe 400 coupled to the introduction device 480, while the openings 564 can receive surgical tools which can extend through the tool supports 560 that guide the tools to a side port or other location at an outer surface of the articulating probe.

While the preferred embodiments of the devices and methods have been described in reference to the environment in which they were developed, they are merely illustrative of the principles of the present inventive concepts. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

1. A system for performing a medical procedure, comprising:
a first assembly comprising:
an articulating probe assembly, comprising:
an outer probe comprising multiple articulating outer links and a first connector; and
an inner probe comprising multiple articulating inner links and a second connector;
a first housing comprising:
a proximal portion;
a distal portion; and
an opening in the first housing distal portion;
wherein at least a portion of the articulating probe is constructed and arranged to pass through the first housing opening;
and;
a second assembly comprising:
a first carriage constructed and arranged to operably engage the first connector of the outer probe;
a second carriage constructed and arranged to operably engage the second connector of the inner probe;
a dual linear drive assembly configured to independently translate the first carriage and the second carriage; and
a second housing comprising a proximal portion and a distal portion;
wherein the first assembly is constructed and arranged to operably attach to the second assembly.

2-94. (canceled)

95. The system according to claim 1, wherein the first assembly is constructed and arranged to be used in fewer clinical procedures than the second assembly.

96. The system according to claim 95, wherein the first assembly is constructed and arranged to be used in a single clinical procedure.

97. The system according to claim 1, wherein at least one of the first connector or the second connector extends beyond the first housing in a direction of the second housing.

98. The system according to claim 1, wherein the at least one of the first connector or the second connector extends into the second housing when the first assembly is attached to the second assembly.

99. The system according to claim 1, wherein the first connector and the second connector are offset from each other.

100. The system according to claim 99, wherein the first connector and the second connector are horizontally offset from each other.

101. The system according to claim 99, wherein the first connector and the second connector are vertically offset from each other.

102. The system according to claim 1, wherein the first carriage comprises a first connecting portion constructed and arranged to removably engage the first connector.

103. The system according to claim 102, wherein the first connecting portion is configured to pivot relative to the first carriage.

104. The system according to claim 103, wherein the first carriage further comprises a first retractable projection constructed and arranged to limit the pivot of the first connecting portion when the projection is in an advanced position.

105. The system according to claim 1, wherein the second carriage comprises a second connecting portion constructed and arranged to removably engage the second connector.

106. The system according to claim 105, wherein the second connecting portion is configured to pivot relative to the second carriage.

107. The system according to claim 106, wherein the second carriage further comprises a second retractable projection constructed and arranged to limit the pivot of the second connecting portion when the projection is in an advanced position.

108. The system according to claim 102, wherein the first or second connecting portion comprises a keyed geometry constructed and arranged to engage the corresponding first or second connector.

109. The system according to claim 108, wherein the first or second connecting portion keyed geometry is constructed and arranged to not engage the other of the first or second connector.

110. The system according to claim 102, wherein at least one of the first or second connecting portion or the corresponding first or second connector comprises a ramp element.

111. The system according to claim 102, wherein at least one of the first or second connecting portion or the corresponding first or second connector comprises a spring-loaded element.

112. The system according to claim 102, wherein at least one of the first or second connecting portion or the corresponding first or second connector comprises an advanceable pin.

113. The system according to claim 102, wherein the connecting portion comprises a magnetic element configured to cause a magnetic attraction force between the first or second connecting portion and the corresponding first or second connector.

114. The system according to claim 102, wherein at least one of the first or second connecting portion or the corresponding first or second connector comprises an electromagnet.

115. The system according to claim 1, wherein the first or second carriage is constructed and arranged to vertically receive and engage the corresponding first or second connector.

116. The system according to claim 115, wherein the first assembly is constructed and arranged to attach to the second assembly when the first carriage and the first connector are in a home position.

117. The system according to claim 1, wherein the first or second carriage is constructed and arranged to horizontally receive and engage the corresponding first or second connector.

118. The system according to claim 117, wherein the first assembly is constructed and arranged to attach to the second assembly when the first carriage and the first connector are in corresponding positions relative to each other.

119. The system according to claim 1, wherein the first carriage is configured to at least advance the outer probe relative to the opening in the first housing distal portion.

120. The system according to claim 119, wherein the first carriage is further configured to retract the outer probe.

121. The system according to claim 1, wherein the first carriage is configured to advance and retract the outer probe via the first connector.

122. The system according to claim 1, wherein the second carriage is constructed and arranged to advance the inner probe.

123. The system according to claim 122, wherein the second carriage is further configured to retract the inner probe.

124. The system according to claim 1, wherein the second carriage is configured to advance and retract the inner probe via the second connector.

125. The system according to claim 1, wherein the dual linear drive assembly comprises a component selected from the group consisting of: lead screw; ball screw; hydraulic piston; pneumatic piston; magnetic drive; inch-worm drive; belt drive; and combinations thereof.

126. The system according to claim 1, wherein the dual linear drive assembly comprises a first linear drive and a second linear drive, wherein the first linear drive is positioned spaced apart in a horizontal direction relative to the second linear drive.
